SEQUENCE LISTING (82 sequences)

SEQ ID NO: 1 - protein, 12 aa, Homo sapiens
  SJ25C1 CDR H2
ESKYGPPCPPCP

SEQ ID NO: 2 - DNA, 36 bp, Homo sapiens
  spacer (IgG4hinge) (nt) Homo sapiens
gaatctaagtacggaccgccctgccccccttgccct

SEQ ID NO: 3 - protein, 119 aa, Homo sapiens
  Hinge-CH3 spacer Homo sapiens
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 229 aa, Homo sapiens
  Hinge-CH2-CH3 spacer Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 282 aa, Homo sapiens
  IgD-hinge-Fc Homo sapiens
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 6 - protein, 24 aa, Artificial Sequence
  T2A artificial
LEGGGEGRGSLLTCGDVEENPGPR

SEQ ID NO: 7 - protein, 357 aa, Artificial Sequence
  tEGFR artificial
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 27 aa, Homo sapiens
  CD28 (amino acids 153-179 of Accession No. P10747) Homo sapiens
FWVLVVVGGVLACYSLLVTVAFIIFWV

SEQ ID NO: 9 - protein, 66 aa, Homo sapiens
  CD28 (amino acids 114-179 of Accession No. P10747) Homo sapiens
IEVMYPPPYLDNEKSNGTIIHVKGKHLCPSPLFPGPSKPFWVLVVVGGVLACYSLLVTVAFIIFWV

SEQ ID NO: 10 - protein, 41 aa, Homo sapiens
  CD28 (amino acids 180-220 of P10747)  Homo sapiens
RSKRSRLLHSDYMNMTPRRPGPTRKHYQPYAPPRDFAAYRS

SEQ ID NO: 11 - protein, 41 aa, Homo sapiens
  CD28 (LL to GG)  Homo sapiens
RSKRSRGGHSDYMNMTPRRPGPTRKHYQPYAPPRDFAAYRS

SEQ ID NO: 12 - protein, 42 aa, Homo sapiens
  4-1BB (amino acids 214-255 of Q07011.1)  Homo sapiens
KRGRKKLLYIFKQPFMRPVQTTQEEDGCSCRFPEEEEGGCEL

SEQ ID NO: 13 - protein, 112 aa, Homo sapiens
  CD3 zeta  Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, Homo sapiens
  CD3 zeta  Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 112 aa, Homo sapiens
  CD3 zeta  Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 335 aa, Artificial Sequence
  tEGFR artificial
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 17 - protein, 18 aa, Artificial Sequence
  T2A artificial
EGRGSLLTCGDVEENPGP

SEQ ID NO: 18 - protein, 22 aa, Artificial Sequence
  P2A
GSGATNFSLLKQAGDVEENPGP

SEQ ID NO: 19 - protein, 19 aa, Artificial Sequence
  P2A
ATNFSLLKQAGDVEENPGP

SEQ ID NO: 20 - protein, 20 aa, Artificial Sequence
  E2A
QCTNYALLKLAGDVESNPGP

SEQ ID NO: 21 - protein, 20 aa, Artificial Sequence
  F2A
QCTNYALLKLAGDVESNPGP

SEQ ID NO: 22 - protein, 10 aa, Artificial Sequence
  linker
  REPEAT (5)...(9): SGGGG is repeated 5 times
PGGGSGGGGP

SEQ ID NO: 23 - protein, 17 aa, Artificial Sequence
  linker
GSADDAKKDAAKKDGKS

SEQ ID NO: 24 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 25 - protein, 22 aa, Artificial Sequence
  GMCSFR alpha chain signal sequence
MLLLVTSLLLCELPHPAFLLIP

SEQ ID NO: 26 - protein, 18 aa, Artificial Sequence
  CD8 alpha signal peptide
MALPVTALLLPLALLLHA

SEQ ID NO: 27 - DNA, 19 bp, Artificial Sequence
  IgG constant RT primer
  modified_base (10)...(12): 2′O methylation
aggacagccgggaaggtgt

SEQ ID NO: 28 - DNA, 18 bp, Artificial Sequence
  IgL constant RT primer
  modified_base (9)...(9): 2′O methylation
  modified_base (11)...(12): 2′O methylation
gctcccgggtagaagtca

SEQ ID NO: 29 - DNA, 20 bp, Artificial Sequence
  IgK constant RT primer
  modified_base (11)...(13): 2′O methylation
ggcctctctgggatagaagt

SEQ ID NO: 30 - DNA, 21 bp, Artificial Sequence
  IgM constant RT primer
  modified_base (13)...(15): 2′O methylation
tgtgaggtggctgcgtacttg

SEQ ID NO: 31 - DNA, 21 bp, Artificial Sequence
  IgA constant RT primer
  modified_base (12)...(14): 2′O methylation
ctggctrggtgggaagtttct

SEQ ID NO: 32 - DNA, 21 bp, Artificial Sequence
  IgD constant RT primer
  modified_base (12)...(13): 2′O methylation
  modified_base (15)...(15): 2′O methylation
cacgcatttgtactcgccttg

SEQ ID NO: 33 - DNA, 20 bp, Artificial Sequence
  IgE constant RT primer
  modified_base (10)...(10): 2′O methylation
  modified_base (12)...(13): 2′O methylation
gatggtggcatagtgaccag

SEQ ID NO: 34 - DNA, 24 bp, Artificial Sequence
  TRA constant RT primer
  modified_base (15)...(16): 2′O methylation
  modified_base (18)...(18): 2′O methylation
tgtttgagaatcaaaatcggtgaa

SEQ ID NO: 35 - DNA, 18 bp, Artificial Sequence
  TRB constant RT primer
  modified_base (9)...(11): 2′O methylation
acgtggtcggggaagaag

SEQ ID NO: 36 - DNA, 22 bp, Artificial Sequence
  TRG constant RT primer
  modified_base (13)...(14): 2′O methylation
  modified_base (16)...(16): 2′O methylation
caagaagacaaaggtatgttcc

SEQ ID NO: 37 - DNA, 20 bp, Artificial Sequence
  TRD constant RT primer
  modified_base (12)...(14): 2′O methylation
tcttcttggatgacacgaga

SEQ ID NO: 38 - DNA, 19 bp, Artificial Sequence
  IgG constant RT primer
  modified_base (10)...(12): 2′O methylation
aggacagccgggaaggtgt

SEQ ID NO: 39 - DNA, 18 bp, Artificial Sequence
  IgL constant RT primer
  modified_base (9)...(9): 2′O methylation
  modified_base (11)...(12): 2′O methylation
gctcccgggtagaagtca

SEQ ID NO: 40 - DNA, 20 bp, Artificial Sequence
  IgK constant RT primer
  modified_base (11)...(13): 2′O methylation
ggcctctctgggatagaagt

SEQ ID NO: 41 - DNA, 21 bp, Artificial Sequence
  IgM constant RT primer
  modified_base (13)...(15): 2′O methylation
tgtgaggtggctgcgtacttg

SEQ ID NO: 42 - DNA, 21 bp, Artificial Sequence
  IgA constant RT primer
  modified_base (12)...(14): 2′O methylation
ctggctrggtgggaagtttct

SEQ ID NO: 43 - DNA, 21 bp, Artificial Sequence
  IgD constant RT primer
  modified_base (12)...(13): 2′O methylation
  modified_base (15)...(15): 2′O methylation
cacgcatttgtactcgccttg

SEQ ID NO: 44 - DNA, 20 bp, Artificial Sequence
  IgE constant RT primer
  modified_base (10)...(10): 2′O methylation
  modified_base (12)...(13): 2′O methylation
gatggtggcatagtgaccag

SEQ ID NO: 45 - DNA, 24 bp, Artificial Sequence
  TRA constant RT primer
  modified_base (15)...(16): 2′O methylation
  modified_base (18)...(18): 2′O methylation
tgtttgagaatcaaaatcggtgaa

SEQ ID NO: 46 - DNA, 18 bp, Artificial Sequence
  TRB constant RT primer
  modified_base (9)...(11): 2′O methylation
acgtggtcggggaagaag

SEQ ID NO: 47 - DNA, 22 bp, Artificial Sequence
  TRG constant RT primer
  modified_base (13)...(14): 2′O methylation
  modified_base (16)...(16): 2′O methylation
caagaagacaaaggtatgttcc

SEQ ID NO: 48 - DNA, 20 bp, Artificial Sequence
  TRD constant RT primer
  modified_base (12)...(14): 2′O methylation
tcttcttggatgacacgaga

SEQ ID NO: 49 - protein, 18 aa, Artificial Sequence
  Exemplary vessel barcode
NNNNWISCNNNWISCNNN

SEQ ID NO: 50 - protein, 9 aa, Artificial Sequence
  FMC63 LC-CDR3
QQGNTLPYT

SEQ ID NO: 51 - protein, 11 aa, Artificial Sequence
  FMC63 CDR L1
RASQDISKYLN

SEQ ID NO: 52 - protein, 7 aa, Artificial Sequence
  FMC63 CDR L2
SRLHSGV

SEQ ID NO: 53 - protein, 9 aa, Artificial Sequence
  FMC63 CDR L3
GNTLPYTFG

SEQ ID NO: 54 - protein, 5 aa, Artificial Sequence
  FMC63 CDR H1
DYGVS

SEQ ID NO: 55 - protein, 16 aa, Artificial Sequence
  FMC63 CDR H2
VIWGSETTYYNSALKS

SEQ ID NO: 56 - protein, 7 aa, Artificial Sequence
  FMC63 CDR H3
YAMDYWG

SEQ ID NO: 57 - protein, 120 aa, Artificial Sequence
  FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 58 - protein, 107 aa, Artificial Sequence
  FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 59 - protein, 245 aa, Artificial Sequence
  FMC63 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 60 - protein, 11 aa, Artificial Sequence
  SJ25C1 CDR L1
KASQNVGTNVA

SEQ ID NO: 61 - protein, 7 aa, Artificial Sequence
  SJ25C1 CDR L2
SATYRNS

SEQ ID NO: 62 - protein, 9 aa, Artificial Sequence
  SJ25C1 CDR L3
QQYNRYPYT

SEQ ID NO: 63 - protein, 5 aa, Artificial Sequence
  SJ25C1 CDR H1
SYWMN

SEQ ID NO: 64 - protein, 17 aa, Artificial Sequence
  SJ25C1 CDR H2
QIYPGDGDTNYNGKFKG

SEQ ID NO: 65 - protein, 13 aa, Artificial Sequence
  SJ25C1 CDR H3
KTISSVVDFYFDY

SEQ ID NO: 66 - protein, 122 aa, Artificial Sequence
  SJ25C1 VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 67 - protein, 108 aa, Artificial Sequence
  SJ25C1 VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 68 - protein, 15 aa, Artificial Sequence
  Linker
GGGGSGGGGSGGGGS

SEQ ID NO: 69 - protein, 245 aa, Artificial Sequence
  SJ25C1 scFv
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 70 - protein, 12 aa, Artificial Sequence
  FMC63 HC-CDR3
HYYYGGSYAMDY

SEQ ID NO: 71 - protein, 7 aa, Artificial Sequence
  FMC63 LC-CDR2
HTSRLHS

SEQ ID NO: 72 - protein, 18 aa, Artificial Sequence
  Linker
GSTSGSGKPGSGEGSTKG

SEQ ID NO: 73 - DNA, 735 bp, Artificial Sequence
  Sequence encoding scFv
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 74 - protein, 5 aa, Artificial Sequence
  Hinge
  VARIANT (1)...(1): X = glycine, cysteine or arginine
  VARIANT (4)...(4): X = cysteine or threonine
XPPXP

SEQ ID NO: 75 - protein, 15 aa, Artificial Sequence
  Hinge
EPKSCDKTHTCPPCP

SEQ ID NO: 76 - protein, 12 aa, Artificial Sequence
  Hinge
ERKCCVECPPCP

SEQ ID NO: 77 - protein, 61 aa, Artificial Sequence
  Hinge
ELKTPLGDTHTCPRCPEPKSCDTPPPCPRCPEPKSCDTPPPCPRCPEPKSCDTPPPCPRCP

SEQ ID NO: 78 - protein, 12 aa, Artificial Sequence
  Hinge
ESKYGPPCPSCP

SEQ ID NO: 79 - protein, 12 aa, Artificial Sequence
  Hinge
ESKYGPPCPPCP

SEQ ID NO: 80 - protein, 9 aa, Artificial Sequence
  Hinge
YGPPCPPCP

SEQ ID NO: 81 - protein, 10 aa, Artificial Sequence
  Hinge
KYGPPCPPCP

SEQ ID NO: 82 - protein, 14 aa, Artificial Sequence
  Hinge
EVVVKYGPPCPPCP

(19) United States
(12) Patent Application Publication
(10) Pub. No.: US 2020/0292526 A1
(43) Pub. Date: Sep. 17, 2020

(54) METHODS OF IDENTIFYING CELLULAR ATTRIBUTES RELATED TO OUTCOMES ASSOCIATED WITH CELL THERAPY

(71) Applicant: Juno Therapeutics, Inc., Seattle, WA (US)

(72) Inventors: Ronald James HAUSE, Jr., Seattle, WA (US); Hyam I. LEVITSKY, Seattle, WA (US); Christopher R. CLOUSER, Seattle, WA (US); Timothy G. JOHNSTONE, Seattle, WA (US)

(73) Assignee: Juno Therapeutics, Inc., Seattle, WA (US)

(21) Appl. No.: 16/644,486

(22) PCT Filed: Sep. 7, 2018
(86) PCT No.: PCT/US2018/050114

§ 371 Date: Mar. 4, 2020

Related U.S. Application Data
(60) Provisional application No. 62/555,643, filed on Sep. 7, 2017.

(51) Int. Cl. G01N 33/50

(52) U.S. Cl. CPC G01N 33/505

(57) ABSTRACT

Provided herein are methods for tracking certain cells associated with a cell therapy, such as from a starting cell composition or a sample prior to administration to a subject and from a sample following administration to a subject. In some aspects, the methods include assessing one or more parameters or attributes of such cells and methods of identifying cellular attributes associated with particular desired cells. The provided methods can be used in connection with cell therapy including adoptive transfer of engineered T cells or T cell precursors.

### CROSS-REFERENCE TO RELATED APPLICATIONS

This application claims priority from U.S. provisional applications No. 62/555,643, filed Sep. 7, 2017, entitled “METHODS OF IDENTIFYING CELLULAR ATTRIBUTES RELATED TO OUTCOMES ASSOCIATED WITH CELL THERAPY,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 735042013540SeqList.TXT, created Sep. 4, 2018, which is 42,585 bytes in size. The information in the electronic format of the Sequence Listing is incorporated by reference in its entirety.

### FIELD

The present disclosure relates to methods for tracking certain cells associated with a cell therapy, such as from a starting cell composition or a sample prior to administration to a subject and from a sample following administration to a subject. In some aspects, the methods include assessing one or more parameters or attributes of such cells and methods of identifying cellular attributes associated with particular desired cells. The provided methods can be used in connection with cell therapy including adoptive transfer of engineered T cells or T cell precursors.

### BACKGROUND

Various methods are available for preparing and administering cells or cell compositions for therapeutic use. For example, methods are available for preparing cells, including T cells, for engineering and cell therapy, and assessment of the activity of the cells upon administration. Improved strategies are needed to assess the activity and/or survival of particular sub-populations, to improve the activity and/or survival of the cells or cell compositions, to improve the manufacturing process and/or to allow improved administration. Provided are embodiments that meet such needs.

### SUMMARY

Provided herein are methods for identifying a property or attribute of a cell including (a) identifying the clonotype and/or a TCR sequence of all of a portion of a native TCR alpha and/or beta variable region or pair thereof, of at least one T cell from at least one test biological sample from a subject, said test biological sample obtained from the subject following administration of a cell therapy containing T cells expressing a recombinant receptor, wherein the T cell in the test biological sample is genetically engineered with and/or expresses the recombinant receptor; (b) identifying, from a T cell composition, a cell that has the same clonotype or the same TCR sequence as the at least one T cell identified in (a), thereby identifying an originator T cell, wherein the T cell composition contains T cells that are, or have been derived from, T cells previously obtained from the subject prior to administering the cell therapy to the subject; and (c) determining at least one property or attribute of the originator T cell.

Provided herein are methods for identifying a property or attribute of a cell including (a) identifying the clonotype and/or a TCR sequence of all of a portion of a native TCR alpha and/or beta variable region or pair thereof, of at least one T cell from at least one test biological sample from a subject, said test biological sample obtained from the subject following administration of a cell therapy containing T cells expressing a recombinant receptor, wherein the T cell in the test biological sample is genetically engineered with and/or expresses the recombinant receptor; (b) determining at least one property or attribute of a cell, from a T cell composition, that has the same clonotype or the same TCR sequence as the at least one T cell identified in (a), wherein the T cell composition contains T cells that are, or have been derived from, T cells previously obtained from the subject prior to administering the cell therapy to the subject.

In some embodiments, the genetically engineered T cell in the test biological sample exhibits a predetermined phenotype, function or parameter. In some embodiments, the predetermined phenotype, function or attribute is an effector function associated with T cell activation state, is a cell surface phenotype or is a pharmacokinetic activity. In some cases, the predetermined phenotype, function or attribute is a pharmacokinetic activity and the pharmacokinetic activity includes determining the number or relative number of recombinant receptor-expressing T cells in the sample. In some aspects, the predetermined phenotype, function or attribute is a cell surface phenotype and the cell surface phenotype is a naive phenotype or a long-lived memory phenotype.

Provided herein is a method for identifying a property or attribute of a cell including identifying the clonotype and/or a TCR sequence of all or a portion of a native TCR alpha and/or beta variable region or pair thereof of one or more T cell genetically engineered with a recombinant receptor in at least one test biological sample from a subject, wherein said clonotype is known to be, determined to be, or suspected of being present in a cell in a T cell composition, thereby identifying one or more originator T cell, wherein: the at least one test biological sample is obtained from the subject following administration of a cell therapy containing T cells expressing the recombinant receptor; and the T cell composition contains T cells that are or are derived from cells of a sample obtained from the subject prior to administering the cell therapy to the subject; and determining at least one or property or attribute of the one or more originator T cell. In some embodiments, the one or more clonotype and/or TCR sequence that is identified is present in the test biological sample at the same or increased frequency or relative frequency as in the T cell composition.

Provided herein is a method for identifying a property or attribute of a cell including identifying one or more clonotypes and/or one or more TCR sequences of all or a portion of a native TCR alpha and/or beta variable region or pair thereof that are the same in a plurality of samples, said plurality of samples selected from one or more compositions at different stages of a cell engineering process for generating a T cell therapy and/or a biological sample from a subject following administration of the T cell therapy to the subject, said T cell therapy containing T cells expressing the recombinant receptor, thereby identifying an originator T cell; and determining at least one property or attribute of the originator T cell.

Provided herein are methods for assessing clonal diversity of a sample containing T cells including identifying one or more clonotypes and/or one or more TCR sequences of all or a portion of a native TCR alpha and/or beta variable region or pair thereof in one of a plurality of samples containing T cells, at different stages of a cell engineering process for generating a T cell therapy and/or following administration of the T cell therapy to a subject, said T cell therapy containing T cells expressing the recombinant receptor and determining the clonal diversity in each of the plurality of samples. In some embodiments, the method further includes determining at least one property or attribute of the one or more cells in the plurality of samples. In some embodiments, the method further includes comparing the clonal diversity of each of the plurality of samples.

In some embodiments, the comparing includes determining the increase or decrease in clonal diversity in the plurality of samples from the same subject. In some cases, the clonal diversity is determined based on the relative frequency of the one or more clonotypes and/or one or more TCR sequences. In some embodiments, the determining the clonal diversity is represented as clonality, Shannon-adjusted clonality or top 25 clonality of each of the plurality of samples. In some embodiments, the determining the clonal diversity is represented as Shannon-adjusted clonality in each of the plurality of samples.

In some embodiments, at least one of the plurality of samples is a T cell composition at a stage of a cell engineering process, said T cell composition containing T cells that are, or have been derived from, T cells previously obtained from the subject prior to administering the cell therapy to the subject. In some of any such embodiments, at least one of the plurality of samples is a test biological sample, said test biological sample obtained from the subject following administration of a cell therapy containing T cells expressing a recombinant receptor. In some embodiments, the method further includes determining a phenotype, function or parameter of the one or more cells in the plurality of sample, prior to the identifying. In some embodiments, the genetically engineered T cell in the test biological sample exhibits a predetermined phenotype, function or parameter. In some examples, the predetermined phenotype, function or attribute is an effector function associated with T cell activation state, is a cell surface phenotype or is a pharmacokinetic property. In some cases, the predetermined phenotype, function or attribute is a pharmacokinetic property and the pharmacokinetic property includes the number or relative number of recombinant receptor-expressing T cells in the sample. In some embodiments, the predetermined phenotype, function or attribute is a cell surface phenotype and the cell surface phenotype is a naive phenotype or a long-lived memory phenotype. In some embodiments, the cell surface phenotype is determined based on surface expression of one or both of CD27 and CCR7.

In some embodiments, the test biological sample is obtained from the subject at or about or within 1 days, 3 days, 6 days, 9 days, 12 days, 15 days, 18 days, 21 days, 24 days, 27 days or 30 days, optionally at or about 12 days, 12 days, 14 days, 15 days, 16 days, 17 days, 18 days, 19 days, 20 days, 21 days, 22 days, 23 days, 24 days, 25 days, 26 days, 27 days, 28 days or 29 days following the administration of the cell therapy containing the T cells expressing the recombinant receptor. In some embodiments, the test biological sample is obtained from a subject at a time after the subject exhibits a response to the cell therapy following the administration, said response selected from a complete response (CR), progression free survival (PFS) or a partial response (PR). In some cases, the response is durable in the subject for at least at least 3 months, at least 6 months, at least 9 months or at least 12 months, and the test biological sample is obtained from the subject at a time when the response is still durable in the subject. In some embodiments, the genetically engineered T cell in the test biological sample exhibits a predetermined phenotype, function or parameter.

In some embodiments, the predetermined phenotype, function or attribute is an effector function associated with T cell activation state, is a cell surface phenotype or is a pharmacokinetic activity. In some embodiments, the predetermined phenotype, function or attribute is a pharmacokinetic activity and the pharmacokinetic activity includes the number or relative number of recombinant receptor-expressing T cells in the sample. In some embodiments, the test biological sample is obtained from the subject at a time at or immediately after a peak T cells expressing the recombinant receptor are detectable in the blood of the subject. In some embodiments, the predetermined phenotype, function or attribute is a cell surface phenotype and the cell surface phenotype is a naive phenotype or a long-lived memory phenotype. In some examples, the cell surface phenotype includes a phenotype surface negative for CD56 or CD45RO and/or a surface positive for CD27, CD45RA, or CCR7. In some cases, the cell surface phenotype is of one or both of CD27 and CCR7.

In some embodiments, the at least one T cell from the at least one test biological sample or the plurality of samples is selected or isolated from a biological sample from the subjects based on the predetermined phenotype, function, or attribute. In some embodiments, the at least one T cell from the at least one test biological sample or the plurality of samples is positive for or expresses the recombinant receptor, optionally is surface positive for the recombinant receptor. In some cases, the at least one T cell that is positive for or expresses the recombinant receptor is selected or isolated from a biological sample from a subject.

In some of any such embodiments, the method is repeated for a plurality of subjects.

In some embodiments, the method includes identifying the at least one property or parameter of originator T cells or T cells in the sample that is present in a T cell composition from a majority of subjects.

In some embodiments, the at least one property or parameter is identified as an attribute of a T cell composition that is predicted to increase likelihood or a desired property, phenotype, attribute or outcome of a cell therapy following administration to a subject.

In some of any such embodiments, the T cell composition is an input composition that does not contain T cells genetically engineered with the recombinant receptor. In some aspects, the input composition is obtained by isolating a population of cells containing the T cells from a biological sample.

In some of any such embodiments, the T cell composition is an output composition containing T cells genetically engineered with the recombinant receptor. In some cases, the output composition is the cell therapy administered to the subject. In some embodiments, the output composition is produced by a process including: (i) incubating an input composition containing T cells with an agent containing a nucleic acid molecule encoding the recombinant receptor under conditions to introduce the nucleic acid encoding the recombinant receptor into cells in the population; and (ii) stimulating the cells, prior to, during and/or subsequent to said incubation, wherein stimulating includes incubating the cells in the presence of a stimulating condition that induces a primary signal, signaling, stimulation, activation and/or expansion of the cells. In some embodiments, the process further includes, prior to (i), isolating the population of cells from a biological sample.

In some of any such embodiments, the isolating includes selecting cells (e.g. T cells) from the biological sample based on surface expression of CD3 or based on surface expression of one or both of CD4 and CD8, optionally by positive or negative selection. In some cases, the isolating includes carrying out immunoaffinity-based selection.

In some embodiments, the biological sample is or contains a whole blood sample, a buffy coat sample, a peripheral blood mononuclear cells (PBMC) sample, an unfractionated T cell sample, a lymphocyte sample, a white blood cell sample, an apheresis product, or a leukapheresis product.

In some embodiments, the stimulating condition includes incubation with a stimulatory reagent capable of activating one or more intracellular signaling domains of one or more components of a TCR complex and/or one or more intracellular signaling domains of one or more costimulatory molecules. In some aspects, the stimulatory reagent contains a primary agent that specifically binds to a member of a TCR complex and a secondary agent that specifically binds to a T cell costimulatory molecule. In some cases, the primary agent specifically binds to CD3 and/or the costimulatory molecule is selected from the group consisting of CD28, CD137 (4-1-BB), OX40, or ICOS. In some embodiments, the primary and secondary agents contain antibodies, optionally an anti-CD3 antibody and an anti-CD28 antibody. In some embodiments, the primary and secondary agent are present on the surface of a solid support, optionally a bead.

In some of any such embodiments, the stimulating the cells is carried out or is initiated prior to the incubating, optionally for 18-24 hours at or about 37 degrees Celsius, wherein the T cells have not been introduced with the nucleic acid encoding the recombinant receptor. In some cases, the stimulating condition includes a cytokine selected from among IL-2, IL-15 and IL-7. In some embodiments, the stimulating cells is carried out subsequent to the incubating, optionally for a period of time to achieve a threshold concentration. In some embodiments, the T cells including cells introduced with the nucleic acid encoding the recombinant receptor. In some embodiments, the stimulating the cells is carried out under conditions to cultivate or expand T cells introduced.

In some embodiments, the agent containing a nucleic acid molecule encoding the recombinant receptor is a viral vector, optionally a lentiviral vector or a gamma retroviral vector.

In some of any such embodiments, the incubating and/or stimulating is carried out in the presence of one or more test agents or conditions; or the process further includes culturing the input composition and/or stimulated cells in the presence of one or more test agents or conditions. In some embodiments, the one or more test agents or conditions includes presence or concentration of serum; time in culture; presence or amount of a stimulating agent; the type or extent of a stimulating agent; presence or amount of amino acids; temperature; the source or cell types of the input composition; the ratio or percentage of cell types in the input composition, optionally the CD4+/CD8+ cell ratio; the presence or amount of beads; cell density; static culture; rocking culture; perfusion; the type of viral vector; the vector copy number; the presence of a transduction adjuvant; cell density of the input composition in cryopreservation; the extent of expression of the recombinant receptor; or the presence of a compound to modulate cell phenotype. In some embodiments, the one or more test agents or conditions includes one or more compounds from a library of test compounds.

In some of any such embodiments, the test biological sample is a serum, blood or plasma sample. In some embodiments, the test biological sample is or contains a tumor sample. In some embodiments, the test biological sample is obtained from the subject greater than or greater than about 7 days, 10 days, 14 days, 21 days, 28 days, 2 months, 3 months, 4 months, 5 months, 6 months, 1 year or 2 years or more after initiation of administration of the cell therapy. In some embodiments, the test biological sample is obtained from the subject greater than or greater than about 28 days after initiation of administration of the cell therapy, optionally at or about at day 29 or greater after initiation of administration of the cell therapy.

In some of any such embodiments, the at least one test biological sample contains a plurality of test biological samples, optionally at least 2, 3, 4, 5, 6, 7, 8, 9, 10 or more test biological samples. In some aspects, each of the plurality of test biological samples is obtained from the subject on different days after initiation of administration of the cell therapy, optionally on consecutive days, every other day, every third day, or once a week for a predetermined time after initiation of administration of the cell therapy.

In some embodiments, the identified clonotype is one whose frequency or relative frequency is retained or increased among the plurality of assessed tested biological samples over the predetermined period of time. In some embodiments, the clonotype and/or TCR sequence is determined by high-throughput or next-generation nucleic acid sequencing. In some embodiments, the clonotype and/or TCR sequence is determined by high-throughput or next-generation RNA sequencing (RNAseq). In some embodiments, the clonotype and/or TCR sequence is determined by high-throughput or next-generation nucleic acid sequencing of one or more regions of the TCRα, TCRβ, TCRγ and TCRδ expressed in the T cell.

In some cases, the clonotype and/or TCR sequence is determined by high-throughput single cell immune sequencing of nucleic acid encoding natively paired TCR chains. In some aspects, the natively paired TCR chains contain TCR α-β or TCR γ-δ pairs.

In some of any such embodiments, the test biological sample contains a plurality of T cells and the one or more clonotype is identified simultaneously or from a single reaction.

In some of any such embodiments, the T cell composition contains a plurality of T cells and the one or more clonotype is identified from a single reaction.

In some embodiments, the at least one property or parameter is determined by single cell gene expression profiling and/or single cell surface phenotyping. In some cases, the at least one property or parameter is determined by single cell gene expression profiling, wherein the single cell gene expression profiling is of at least one gene product or is of the whole-transcriptome or a portion thereof. In some embodiments, the at least one gene product is selected from CD4, ICOS, FOXP3, FOXP3V1, PMCH, CD80, FOXP3Y, CD86, CD70, CD40, IL-6, CD2, CD3D, GPR171, CXCL13, PD-1 (CD279), IL-2, IL-4, IL-10, CD8B, KLRK1, CCL4, RUNX3V1, RUNX3, NKG7, CD45RA, CD45RO, CD62L, CD69, CD25, CCR7, CD27, CD28, CD56, CD122, CD127, CD95, CXCR3, LFA-1, KLRG1, T-bet, CD8, IL-7Rα, IL-2Rβ, CD3, CD14, ROR1, granzyme B, granzyme H, CD20, CD11b, CD16, HLA-DR, PD-L1, IFNγ, KIRK1, caspase 2, caspase 3, caspase 6, caspase 7, caspase 8, caspase 9, caspase 10, Bcl-2, Bax, Bad, Bid, CD196 (CCR6), CTLA-4 (CD152), TIGIT (VSIG9, VSTM3), LAG-3 (CD223), 2B4 (CD244), BTLA (CD272), TIM3 (HAVCR2), VISTA (PD1-H) and CD96.

In some embodiments, the at least one property or parameter is determined by single cell surface phenotyping of at least one T cell surface marker. In some cases, the at least one T cell surface marker is selected from CD4, CD8, CD45RA, CD45RO, CD62L, CD69, CD25, CCR7, CD27, CD28, CD56, CD122, CD127, T-bet, IL-7Rα, CD95, IL-2Rβ, CXCR3, LFA-1 or KLRG1.

In some embodiments, the single cell gene expression profiling or single cell surface phenotyping is coupled to or carried out in the same reaction as the single cell immune sequencing. In some embodiments, identification of the clonotype and/or TCR sequence includes barcoded nucleic acid sequencing.

In some of any such embodiments the recombinant receptor is or contains a chimeric receptor. In some cases, the chimeric receptor is capable of binding to a target antigen that is associated with, specific to, and/or expressed on a cell or tissue of a disease, disorder or condition. In some aspects, the disease, disorder or condition is an infectious disease or disorder, an autoimmune disease, an inflammatory disease, or a tumor or a cancer.

In some of any such embodiments, the target antigen is a tumor antigen. In some embodiments, the target antigen is selected from among ROR1, B cell maturation antigen (BCMA), carbonic anhydrase 9 (CAIX), Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag.

In some embodiments, the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the chimeric receptor is a chimeric antigen receptor (CAR). In some cases, the chimeric receptor contains an extracellular domain containing an antigen-binding domain. In some embodiments, the antigen-binding domain is or contains an antibody or an antibody fragment thereof, which optionally is a single chain fragment. In some aspects, the fragment contains antibody variable regions joined by a flexible linker. In some embodiments, the fragment contains an scFv.

In some of any such embodiments, the chimeric receptor further contains a spacer and/or a hinge region. In some cases, the chimeric receptor contains an intracellular signaling region. In some embodiments, the intracellular signaling region contains an intracellular signaling domain. In some embodiments, the intracellular signaling domain is or contains a primary signaling domain, a signaling domain that is capable of inducing a primary activation signal in a T cell, a signaling domain of a T cell receptor (TCR) component, and/or a signaling domain containing an immunoreceptor tyrosine-based activation motif (ITAM). In some cases, the intracellular signaling domain is or contains an intracellular signaling domain of a CD3 chain, optionally a CD3-zeta (CD3ζ) chain, or a signaling portion thereof.

In some of any such embodiments, chimeric receptor further contains a transmembrane domain disposed between the extracellular domain and the intracellular signaling region. In some aspects, the intracellular signaling region further contains a costimulatory signaling region. In some embodiments, the costimulatory signaling region contains an intracellular signaling domain of a T cell costimulatory molecule or a signaling portion thereof. In some cases, the costimulatory signaling region contains an intracellular signaling domain of a CD28, a 4-1BB or an ICOS or a signaling portion thereof. In some embodiments, the costimulatory signaling region is between the transmembrane domain and the intracellular signaling region.

In some of any such embodiments, the T cell composition and/or cell therapy contains CD4 and/or CD8 T cells.

In some of any such embodiments, the clonotype contains the TCR sequences of all or a portion of a native TCR alpha and/or beta variable region or pair thereof. In some embodiments, the clonotype and/or TCR sequence is of a T cell genetically engineered with or expressing the recombinant receptor. In some embodiments, the clonotype and/or TCR sequence is of a CD8+ T cell.

### BRIEF DESCRIPTION OF THE DRAWINGS

FIG. 1 depicts a schematic of an exemplary embodiment of the method.

FIG. 2A depicts a general schematic representation of the experimental design described in Example 1.

FIG. 2B shows the relative percentages of different T cell subtypes, based on flow cytometry sorting based on cell surface staining of CD45RA and CCR7, for CD8+ TCR clones determined by TCR single cell sequencing to be present among cells in the “T cell composition from subject” (before engineering) and among the “engineered cells” (after engineering).

FIG. 2C shows a trace diagram indicating changes in CD27/CCR7 based phenotype of individual CD8+ clones determined to be present in both populations, as assessed by TCR sequencing.

FIG. 3 depicts the changes in T cell clonotype repertoire and the relative abundance of identified clones, as determined by TCR sequencing, in an exemplary subject (for clonotypes that were detected in 10 or more sequenced TCR molecules in each sample, and that were detected in each of the indicated compositions), in the engineered cells, test biological samples derived from PBMC obtained from subjects on day 22 or day 29 after adoptive cell transfer (after administration of the engineered cells).

FIG. 4 shows clonal abundance of TCR clones, as determined by TCR sequencing detected above a threshold level across samples in CD4+ and CD8+ cell compositions from the subject, in engineered cells, and in post-administration samples obtained at different stages, in 2 exemplary subjects.

### DETAILED DESCRIPTION

The provided methods in some aspects involve identifying cellular attributes of cells used in adoptive cell therapy, e.g., engineered T cells. In some embodiments, the provided methods involve determining and identifying the phenotype, function, attribute, or property of cells at various stages of adoptive cell therapy, such as by clonotypic tracking of T cells, e.g. based on tracking of native TCR sequences. In some embodiments, the methods can be used to identify features or attributes of T cells, such as T cells obtained from a subject and/or cells used in connection with manufacturing or formulating a drug product, that are predicted to or likely to result in one or more advantageous or desired features associated with cell therapy upon administration of the therapeutic T cell drug product. In some embodiments, the desired feature may be associated with or related to the expansion, persistence and/or memory-like phenotype, such as long-lived memory phenotype, of such cells.

T cell-based therapies, such as adoptive T cell therapies (including those involving the administration of cells expressing chimeric receptors specific for a disease or disorder of interest, such as chimeric antigen receptors (CARs) and/or other recombinant antigen receptors, as well as other adoptive immune cell and adoptive T cell therapies) can be effective in the treatment of cancer and other diseases and disorders. In certain contexts, available approaches to adoptive cell therapy may not always be entirely satisfactory. In some contexts, optimal efficacy can depend on the ability of the administered cells to recognize and bind to a target, e.g., target antigen, to traffic, localize to and successfully enter appropriate sites within the subject, tumors, and environments thereof. In some contexts, optimal efficacy can depend on the ability of the administered cells to become activated, expand, to exert various effector functions, including cytotoxic killing and secretion of various factors such as cytokines, to persist, including long-term, to differentiate, transition or engage in reprogramming into certain phenotypic states (such as long-lived memory, less-differentiated, and effector states), to avoid or reduce immunosuppressive conditions in the local microenvironment of a disease, to provide effective and robust recall responses following clearance and re-exposure to target ligand or antigen, and avoid or reduce exhaustion, anergy, peripheral tolerance, terminal differentiation, and/or differentiation into a suppressive state.

In some aspects, currently available methods are not entirely satisfactory in one or more of these aspects. For example, in some cases, changes in cells during or following manufacturing and/or administration of a T cell therapy can result in a change in differentiation or activation state of T cells that may result and/or lead to reduced persistence in vivo when genetically engineered cells are administered to a subject. Among changes in differentiation state that may occur include, in some cases, loss of a naïve phenotype, loss of memory T cell phenotypes, and/or the promotion of exhaustion or anergy, thereby generating effector cells with an exhausted T cell phenotype. Exhaustion of T cells may lead to a progressive loss of T cell functions and/or in depletion of the cells (Yi et al. (2010) Immunology, 129:474-481). T cell exhaustion and/or the lack of T cell persistence is a barrier to the efficacy and therapeutic outcomes of adoptive cell therapy; clinical trials have revealed a correlation between greater and/or longer degree of exposure to the antigen receptor (e.g. CAR)-expressing cells and treatment outcomes.

Thus, in some aspects, cellular persistence of the administered cells in the body over time is an important attribute for achieving long term remissions. In some cases, engineered cells undergo extensive expansion in the subject after administration through multiple rounds of cell division, leading to effector cell differentiation, contraction (cell death), and for the survivors, long-lived memory cell generation that is maintained by more gradual rates of homeostatic proliferation. In many cases, the initial pool of cells for engineering is heterogeneous in terms of the relative abundance of cells at different stages of T cell differentiation, and they are also heterogeneous in their ability to generate progeny to populate the subject with effector cells and cells that maintain high potential to expand upon encountering antigens. The provided embodiments are based on observations that the clonal repertoire is heterogenous at different stages of adoptive cell therapy.

Provided herein are methods in which the native TCR sequence of genetically engineered T cells (e.g. CAR-T cells) that are determined to have a desirable property or feature, such as following administration to a subject, e.g. persistent cells of a responder, is used as a biological barcode to identify an originating cell (sister cell) of such T cell by searching for the same TCR sequence (clonotype) in a starting composition or compositions of that same subject. In some embodiments, the originator or sister T cells in a starting composition can be distinguished from the others cells in the composition via their TCR sequences or clonotype. Once identified, methods can be carried out to determine a feature or property, such as a molecular signature, of the originator T cells in the starting composition. In some embodiments, single cell sequencing and analysis is employed.

In some embodiments, such starting or initial compositions include any T cell composition containing T cells produced as part of an ex vivo process for producing genetically engineered T cells, e.g. CAR-T cells, involving one or more of selection or isolation of T cells from a subject; activation or stimulation of T cells, such as via inducing a primary and/or accessory stimulation signal in the T cell; introduction of sequences encoding a genetically engineered recombinant receptor, such as by transduction with a viral vector; cultivation of the cells under conditions to promote expansion or proliferation and/or the produced genetically engineered T cell composition that is to be administered to a subject from which the T cells were originally isolated. In some embodiments, the starting composition of T cells contains T cells isolated, selected or enriched from a subject prior to engineering the T cells with the recombinant receptor. In some embodiments, the starting composition of T cells contains the engineered drug product that includes the resulting produced genetically engineered T cells that have been engineered with a recombinant receptor (e.g. CAR). In some aspects, it is contemplated that a particular desirable feature or property of genetically engineered T cells (e.g. CAR-T cells) in vivo in a subject, e.g. persistence of cells in responders, are associated with a unique molecular signature of T cells in such starting (ex vivo) T cell compositions.

The provided embodiments offer advantages in identifying and characterizing specific attributes of cells associated with certain cell clones, e.g., T cell clones, that are capable of persisting and expanding in the subject over time. The provided embodiments employ clonotype analysis to identify T cell clones that exhibit one or more desirable features, such as persistance over time in the subject, and to track the characteristics, e.g., phenotype, function, attribute, property or attributes, such as molecular signatures, of such cells sharing the same clonotype at various stages of adoptive cell therapy. Thus, the methods can be used to select and identify characteristics or attributes of a T cell clone, e.g. originator T cell clone, present in a starting or initial composition or drug product prior to administration of a T cell therapy to a subject that tracks to a T cell that shares the same clonotype and/or native TCR sequence, such as a progenitor T cell thereof, and that exhibits higher or greater persistence and expansion or other desired feature following administration to the subject.

Thus, in the provided embodiments, the natural diversity of the T cell receptor is utilized to document the degree of differential expansion of clones in the subject relative to the starting engineered cell population, and to identify originator T cell clones, in a composition of engineered T cells or T cell compositions obtained from the subject for engineering, that share the differentially expanded TCRs. Various properties and parameters, such as gene expression or transcriptome profile or surface phenotypes, of the originator T cell clone can be used to identify characteristics, e.g., phenotype, function, attribute, property or attribute, that are associated with higher persistence and/or expansion or other desired feature or property, and ultimately high efficacy. The provided methods can be carried out or applied to a plurality of subjects (e.g. 2 or more, such as 10, 50, 100 or more subjects) in order to identify common characteristics or attributes of cells, e.g. observed among T cell clones present in an initial cell composition or engineered drug product of a majority of subjects, that is associated with a desired feature of cells, e.g. high potential expansion and/or persistence, following administration of a particular cell therapy or regimen of a cell therapy among a population of subjects so treated by the cell therapy.

In some aspects, single cell analysis of immune sequence to identify a TCR clonotype is coupled to single cell methods for analyzing phenotype and molecular signatures, such as gene expression, in order to identify and/or select features or attributes of T cell clones that are associated with a desired feature or property, such as greater persistence and/or high efficacy, when administered. In some embodiments of the methods provided herein, clonotypes of T cells having a predetermined function, parameter or phenotype, such as high abundance or high expansion in the subject's body after administration, are assessed and evaluated in combination with results from phenotype and molecular signatures analysis at various stages. For example, phenotypes and molecular signatures of cells in the T cell compositions and samples obtained from the subject are analyzed using population-level and single-cell analysis of phenotypes. In some cases, single-cell gene expression analysis, genome-wide RNA expression profiles and/or single cell surface expression analysis can be coupled with single cell TCR sequencing. In some cases, particular clonotypes that are highly abundant in the samples, e.g. peripheral blood, obtained from the subject after administration are identified, and phenotypes and molecular signatures of the particular clone in the T cell composition obtained from the subject prior to engineering or administration (e.g., “originator T cell population”) is determined.

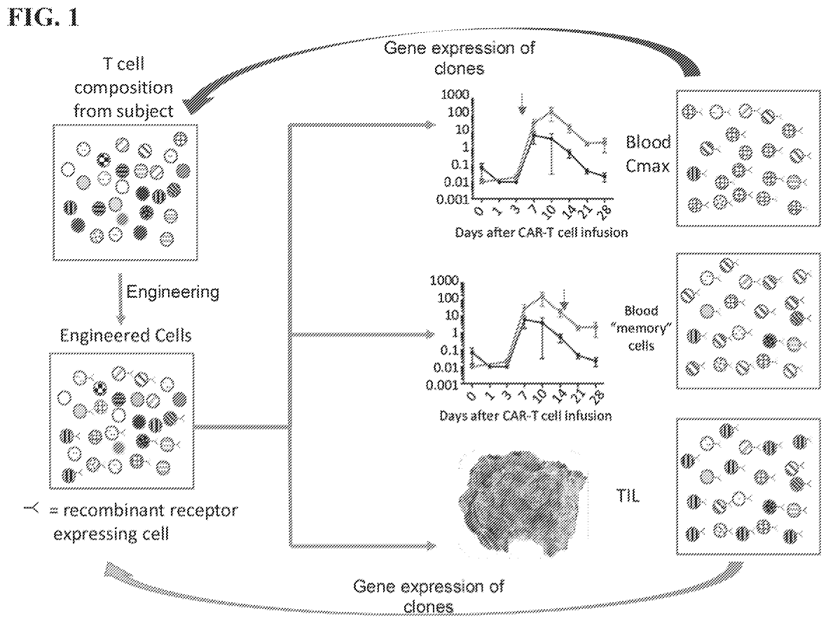

A schematic of an exemplary embodiment of the method is depicted in FIG. 1. In some embodiments, as depicted, T cell compositions from a subject contain various different clonotypes of T cells. In some aspects, a T cell composition is a therapeutic T cell composition that includes T cells that are engineered to express a recombinant receptor, e.g., chimeric antigen receptor (CAR) for use in adoptive cell therapy. In some aspects, following manufacturing and ex vivo generation of a therapeutic T cell composition, represented clonotypes, and associated cell phenotypes or molecular signatures, can change compared to the initial T cell composition obtained or selected from a sample from a subject. Further, after administration of the therapeutic T cell composition or cell therapy to a subject, the abundance or relative abundance of a clonotype represented in the population of cells in the subject can change at various time points after administration, because, in some cases, certain T cell clones may be capable of expanding and persisting at different stages, locations or rates within the subject. With reference to the exemplary depiction in FIG. 1, a clonotype repertoire may be different at the time point of maximum serum concentration of the administered CAR-expressing cells (“Blood Cmax”), at a time point after the Cmax where memory-type cells are common (“Blood ‘memory’ cells”) and/or in tumor infiltrating lymphocytes at the tumor site (“TIL”). In some cases, the clonotype repertoire can be different compared to the initial T cell composition or engineered T cell compositions, as certain clones expand and persist better than others. The provided methods exploit the capability to track T cell clones over time, including at different stages of a cell engineering process, such as from patient material and drug product, based on clonotypic analysis to assess and/or identify attributes in starting material cells that may increase the likelihood of a desired property or outcome of a therapeutic cell product.

In some embodiments, the methods involve identifying the clonotype of at least one T cell from at least one test biological sample from a subject, said test biological sample obtained from the subject following administration of a cell therapy comprising T cells expressing the recombinant receptor, wherein the T cell in the test biological sample is genetically engineered with the recombinant receptor and exhibits a predetermined phenotype, function or parameter; identifying, from a T cell composition, a cell that has the same clonotype as the at least one T cell previously identified, thereby identifying an originator T cell, wherein the T cell composition comprises T cells previously obtained from the subject prior to administering the cell therapy to the subject; and determining at least one property or attribute of the originator T cell.

In some embodiments, the methods involve identifying one or more clonotypes of a T cell genetically engineered with a recombinant receptor in at least one test biological sample from a subject that is present in a T cell composition, thereby identifying an originator T cell, wherein: the test biological sample is obtained from the subject following administration of a cell therapy comprising T cells expressing the recombinant receptor; and the T cell composition comprises T cells previously obtained from the subject prior to administering the cell therapy to the subject; and determining at least one or property or attribute of the originator T cell.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DETERMINING T CELL CLONOTYPES AND CLONAL DIVERSITY

The provided methods in some aspects involve identifying cellular attributes of cells used in connection with a process or method for producing and/or administering to a subject an adoptive cell therapy, e.g., engineered T cells, such as CAR-T cells. In some embodiments, the provided methods involve determining and identifying the phenotype, function, attribute, or property of cells in compositions or populations of cells at various stages of producing a composition of engineered cells for adoptive cell therapy and/or after administering such compositions to a subject, such as by clonal analysis of TCR sequences (for example, native TCR sequences). In some embodiments, clonal diversity of a composition or population of T cells at various stages in a process for producing engineered T cells (e.g. CAR-T cells) and/or after administration of the engineered T cells (e.g. CAR-T cells) to a subject can be determined. In some embodiments, clonotypes of T cells (such as the set of T cells with the same T cell receptor, for example, the same native cell receptor) in a composition of population of T cells at various stages in a process for producing engineered T cells (e.g. CAR-T cells) and/or after administration of the engineered T cells to a subject, can be determined which, in some aspects, can be used in methods for clonotypic tracking of T cells.

Various methods for assessing the TCR repertoire for clonotype identification and TCR repertoire analysis are known (see e.g. Rosati et al. (2017) BMC Biotechnology, 17:61; Friedensohn et al. (2016) Trends in Biotechnology, 35:203-214). In some aspects, the methods involve high-throughput or next-generation sequencing methods. In some embodiments, bulk methods are used to assess clonotypes that are present in a population or composition of cells, such as the frequency and variety of different clones present in the population or composition. In some embodiments, bulk methods can be utilized to assess the clonality, clonal diversity or clonal heterogeneity of a population or composition of cells, for example, based on the determined frequency and/or variety of clonotypes present in the population or composition. In some embodiments, single-cell sequencing methods are carried out to identify a clonotype on a particular cell. In certain aspects, paired αβ TCR sequencing methods are used (see e.g. WO2017053902A1). In some embodiments, sequencing methods are carried out on DNA, such as genomic DNA or complementary DNA. In some embodiments, sequencing methods are carried out on RNA. In some embodiments, high-throughput or next-generation sequencing of TCR sequences or by sequencing the whole genome or transcriptome (e.g., RNAseq). In some aspects, the methods used are RNAseq-based methods.

In some embodiments of the methods provided herein, T cell clonotype assessment and clonality and diversity in various T cell populations or compositions or samples containing T cells, are determined using high-throughput sequencing of all or a portion of the TCR genes or based on sequences obtained from high-throughput whole genome or transcriptome analysis, on the population or composition of cells, and/or in a single cell. In some embodiments, the provided methods can include various features of the methods as described in WO2016/044227, WO2016/176322, WO2012/048340, WO2012/048341, WO2014/144495, WO2017/053902, WO2017/053903 or WO2017/053905, each incorporated by reference in their entirety.

In some embodiments of the methods provided herein, T cell clonotype assessment and clonality and diversity in various T cell populations or compositions or samples containing T cells, are determined using high-throughput and/or single cell-based sequencing methods. In some embodiments of the methods provided herein, the clonotypes of the T cells in various T cell populations or compositions or samples containing T cells, are determined using high-throughput single cell-based sequencing methods. In some embodiments, such methods include single-cell αβ-paired TCR sequencing. Such methods can be used to determine TCR clonotypes present in the population, TCR repertoire, T cell clonality and diversity, and the relative abundance of the identified clones in a cell population, based on barcoded single-cell sequencing of the expressed TCR genes. In some embodiments, exemplary high-throughput single cell-based sequencing methods used herein are described in, for example, WO2016/044227, WO2016/176322 and WO2012/048340, each incorporated by reference in their entirety.

In some embodiments, the methods involve obtaining a sample of cells containing T cells. The sample of cells can be a sample of a composition containing T cells used in connection with producing an engineering cell therapy (e.g. CAR-T cells), see e.g. Section III. In some aspects, the sample is a leukapheresis sample from a subject, a sample from a starting or initial composition of T cells isolated or selected from a subject, e.g. a composition enriched in CD4+, CD8+ or CD4+ and CD8+ T cells, a composition containing T cells transduced with a nucleic acid encoding a recombinant receptor (e.g. CAR), a sample at various times during or following stimulation and/or expansion of T cells, or an engineered composition that is ready for administration to a subject. In some embodiments, the sample is from a population of cells obtained from a subject, e.g. from the blood or tumor of a subject, that had been administered engineered T cells (e.g. CAR-T cells). In some embodiments, nucleic acids, e.g. DNA or RNA, from the sample are amplified and the amplified molecules are sequenced to determine TCR clonotype. In some aspects, nucleic acid barcodes can be utilized to track and/or identify particular sequences.

In particular embodiments of any of the provided methods, the recombinant receptor is a chimeric antigen receptor (CAR) and the clonotypes of T cells in a population of cells, such as from a composition or sample containing T cells, is of the native TCR sequence in cells of the population.

A. Clonotype Identification or Tracking

In some embodiments, determination of clonotype includes determining the sequence of all or a portion of TCR alpha and/or beta variable region, or pair thereof In some embodiments, determination of clonotype includes determining the pair of TCR α and β chains, such as the native TCR α and β chains. In some embodiments, the clonotype is a sequence of all or a portion of a TCR alpha and/or beta variable region, or pair thereof, such as natively expressed in a subject. Thus, for purposes of the provided method, the clonotype is not or does not include the recombinant receptor (e.g., genetically engineered receptors, such as chimeric antigen receptor or recombinant T cell receptor).

In some embodiments, the clonotypes present in a T cell or T cells of composition that are part of a process for engineering T cells with a recombinant receptor (e.g. CAR-T cells), compositions containing engineered T cells (e.g. engineered CAR-T cells) and/or samples containing or suspected or likely to contain engineered T cells, such as obtained from a subject administered engineered cells (e.g. CAR-T cells).

In some embodiments, the clonotypes present in T cell populations are determined over time. In some embodiments, the clonotype determination is performed at various stages of adoptive cell therapy, e.g., before and after engineering of the cells and/or before and after administration of the cells in the subject. In some embodiments, the clonotype determination is performed using various cell compositions and samples obtained from the subject, at various time points and stages of adoptive cell therapy. For example, for autologous cell transfer, a composition of cells, including immune cells, e.g., T cells, is initially obtained from the subject. Certain T cells are isolated from the composition, by immunoaffinity-based enrichment, and subject to genetic engineering, e.g., to express a recombinant receptor. The engineered cells then can be administered to the subject for therapy. In some embodiments, clonotype determination is performed at any one or more points or stages throughout the process, and compared with the clonotype determination performed at other points or stages. Exemplary methods that can be used for adoptive cell therapy, e.g., CAR-expressing T cell therapy, are described below in Section III.B. The methods provided herein can be used in any stages or time points of performing adoptive cell therapy, using any compositions or samples, such as those obtained from the subject or engineered or processed. Particular T cell clones or clonotypes can be traced throughout the generation of cells for therapy and after administration of the cells to subjects.

The clonotypes of a cell or the clonotypes present in a population or composition of cells, in some examples, is determined by targeted sequencing of particular genes or transcripts (e.g., immune sequencing or TCR sequencing). In some embodiments, sequencing methods that can be employed include high-throughput or next-generation sequencing. In some aspects, next-generation sequencing methods can be employed, using genomic DNA or cDNA from T cells, to assess the TCR repertoire, including sequences encoding the complementarity-determining region 3 (CDR3). In some embodiments, whole transcriptome sequencing by RNAseq can be employed. In some aspects, the TCR repertoire information, e.g., TCR sequences and relative frequency, can be constructed or extracted from whole transcriptome sequencing (e.g., by RNAseq). For example, in some aspects, computational methods such as MIXCR (Bolotin et al. Nature Methods 12 (2015) 380-381, Bolotin et al., Nature Biotechnology 35 (2017) 908-911) or IMREP (Mangul et al., bioRxiv (2017) 089235) can be utilized to determine the repertoire TCR sequences or a portion thereof (e.g., CDR3) from whole transcriptome RNAseq results. In some embodiments, single-cell sequencing methods can be used. In some embodiments, clonotypes can be assessed or determined by spectratype analysis (a measure of the TCR Bβ, Vα, Vγ, or Vδ chain hypervariable region repertoire). Clonotypes can also be determined by generation and characterization of antigen-specific clones to an antigen of interest.

In some embodiments of the methods provided herein, T cell clonotype assessment are determined using high-throughput sequencing of all or a portion of the TCR genes or based on sequences obtained from high-throughput whole genome or transcriptome analysis, on the population or composition of cells, and/or in a single cell. In some embodiments, bulk sequencing of targeted sequences (e.g., TCR chains or portion thereof) or bulk whole genome or transcriptome sequencing (e.g., by RNAseq) can be used to determine the clonotypes present in the cells in the population or composition. In some aspects, T cell clonotype assessment can involve sequencing of a portion of the variable region of one or more native TCR chains, such as the complementarity-determining region 3 (CDR3). In some aspects, single cell sequencing can be employed. In some embodiments, the provided methods can include various features of the methods as described in WO2016/044227, WO2016/176322, WO2012/048340, WO2012/048341, WO2014/144495, WO2017/053902, WO2017/053903 or WO2017/053905, each incorporated by reference in their entirety. In some embodiments, such methods can be used to obtain sequence information about a target polynucleotide of interest within a cell, such as nucleic acid sequences encoding a TCR or a chain, domain, region or portion thereof. The target genes can be obtained from genomic DNA or mRNA of a cell from a sample or population of cells. The sample or population of cells can include immune cells. For example, for target TCR molecules, the genes encoding chains of a TCR can be obtained from genomic DNA or mRNA of immune cells or T cells.

In some embodiments, the methods described herein can comprise characterizing cells utilizing single-cell sequencing and/or barcoding (for example, bulk immune sequencing of a population employing nucleic acid barcoding). In some embodiments, the methods include determining the clonal composition, clonal diversity, clonal repertoire and/or clonality of a plurality of cells, e.g., a population of cells, via determining the sequence of the expressed T cell receptor (TCR) on the surface of the cell.

In some aspects, determination of clonotypes involve sequencing of a single chain or a portion thereof of the TCR, e.g., sequencing of one of TCR α, β, γ, or δ chains or a portion thereof. In some embodiments, determination of clonotypes involve sequencing of paired sequencing, such sequencing of all or a portion of TCR α and β chain pair, or TCR γ and δ pair, or a portion thereof.

In some embodiments, the TCR sequence and/or clonal composition of a plurality of cells is determined using a method involving high-throughput or next-generation sequencing of TCR sequences, or via high-throughput or next-generation sequencing of whole genome or transcriptome, and assessing the TCR sequences within the whole genome or whole transcriptome. In some aspects, the sequencing involves bulk sequencing of a population or composition of cells. In some embodiments, the sequencing involves single-cells sequencing.

In some embodiments, the TCR sequence and/or clonal composition of a plurality of cells is determined using a method comprising: forming a plurality of vessels each comprising a single cell from a sample comprising a plurality of cells, a plurality of molecular barcoded polynucleotides, and a vessel barcoded polynucleotide; producing: a first complementary polynucleotide that is complementary to a first cell polynucleotide, e.g. such as one of the alpha and beta (Vα or Vβ) and/or one or the gamma and delta chains (Vγ or Vδ) from the single cell, and a second complementary polynucleotide that is complementary to a second cell polynucleotide, e.g. the other of the alpha and beta and/or the other of the gamma and delta chains, from the single cell; attaching: a first molecular barcoded polynucleotide of the plurality to the first complementary polynucleotide, and a second molecular barcoded polynucleotide to the second complementary polynucleotide, thereby forming a first and a second single cell single-barcoded polynucleotide; and attaching the vessel barcoded polynucleotide, or an amplified product thereof to the first single cell single-barcoded polynucleotide, and the second single cell single-barcoded polynucleotide, thereby forming a first and a second single cell dual-barcoded sequences.

Embodiments of the provided methods can include sampling of a large number of single cells. A polynucleotide harboring a vessel barcode can also be introduced during formation of the vessels. These vessel barcoded polynucleotides can carry degenerate barcodes such that each oligonucleotide containing a vessel barcode contains a unique identity code corresponding to the vessel they are in.

Oligonucleotides can be amplified and amplified products of the reaction can be recovered from the vessels. Amplified products can be PCR enriched to add next-generation sequencing (NGS) tags. The library can be sequenced using a high throughput sequencing platform followed by analysis of vessel barcode sequences. Because each single cell is isolated in its respective vessel, for each vessel barcode observed twice, the amplified oligonucleotide products sequenced originated from the same vessel and therefore from a unique single cell. Because each TCR chain sequence contains a barcode and each single cell is isolated in its respective vessel, for each TCR observed for sequences containing the same vessel barcode, the amplified oligonucleotide products sequenced originated from a particular single cell in the same vessel.

In one aspect, clonotype determination described herein further comprises generating polynucleotide libraries for high-throughput sequencing. The provided disclosure can be applied to multiple different types of paired variable sequences, e.g., T-cell receptor chain pairs, together with single cell characterization of other properties and/or parameters. For example, polynucleotides complementary to cell polynucleotides, such as alpha and/or beta and/or gamma and/or delta chains, e.g., Vα/Vβ and Vγ/Vδ T-cell receptor (TCR) chains (such as those derived from framework portions thereof), can be introduced during formation of (or included within) the vessels. A polynucleotide harboring a vessel barcode can also be introduced during formation of (or included within) a vessel. These vessel barcoded polynucleotides can carry degenerate barcodes such that each cell polynucleotide containing a vessel barcode contains a unique identity code corresponding to the vessel it is in during the reaction(s). Thus in some such embodiments, a plurality of polynucleotides with the same unique identity code are deemed to have originated from the same vessel and in some aspects thus from a single cell. A plurality of polynucleotides harboring a molecular barcode can also be introduced during formation of or included in the vessels. These molecular barcoded polynucleotides can carry degenerate barcodes such that each cell polynucleotide molecule containing a molecular barcode contains a unique identity code corresponding to a single cell polynucleotide molecule from which they came. The millions of single immune cells can be lysed inside the emulsion and cell transcripts, such as Vα/Vβ and/or Vγ/Vδ chain transcripts, can be reverse transcribed or copied using primers, followed by tagging with a vessel barcode and a molecular barcode, and PCR amplification of the barcoded polynucleotides. Each Vα/Vβ and/or Vγ/Vδ chain stemming from a single immune cell (e.g., a T-cell) can be virtually linked to each other with the same vessel barcode identity.

The Vα/Vβ and/or Vγ/Vδ chains can then be recovered from the vessels and PCR enriched in order to add next-generation sequencing (NGS) tags. The library can be sequenced using a high throughput sequencing platform followed by analysis of repertoire diversity, TCR frequency, CDR3 characterization, somatic hypermutation phylogeny analysis, etc. A database of correctly matched Vα/Vβ and/or Vγ/Vδ pairs can be generated by deconvoluting the vessel and molecular barcode sequences. Because each single immune cell are isolated in their respective vessel, for each vessel barcode observed twice, the transcripts sequenced originated from the same emulsion droplets and therefore from a unique single cell. For each different molecular barcode observed, for sequences containing the same vessel barcode, the transcripts sequenced originated from a different transcript molecule from a single cell. For each same molecular barcode observed, for sequences containing the same vessel barcode, the transcripts sequenced originated from a same transcript molecule from a single cell (e.g., PCR duplicates).

In some embodiments, the single-cell immune sequencing allows comprehensive analysis of natively paired TCRs from complex heterogeneous samples using a microfluidic emulsion-based method for parallel isolation and DNA barcoding of large numbers of single cells. Up to a million cells per hour can be isolated in individual ˜65 picoliter emulsion droplets. Within the droplets cells are lysed, target mRNA is reverse transcribed with target-specific primers and a two-step DNA barcoding process attaches both molecule-specific and droplet-specific barcodes to the cDNAs. After subsequent recovery and next generation sequencing, the dual barcoding strategy allows clustering of sequence reads into both their molecules and cells of origin. This allows extensive correction of errors and amplification biases, clone counting at both the mRNA and cellular levels, heavy chain isotype determination, and importantly, recovery of full-length, natively paired TCRs simultaneously at extremely high throughput.

1. Samples

In some embodiments, the clonotype determination is performed in samples from various stages of adoptive cell therapy, e.g., before and after engineering of the cells and/or before and after administration of the cells in the subject. In some embodiments, the clonotypes are determined for samples from one or more stages, time points and/or locations. In some embodiments, the clonotype determination is performed in various cell compositions and samples obtained from the subject, at various time points and stages of adoptive cell therapy. For example, for autologous cell transfer, a composition of cells, including immune cells, e.g., T cells, is initially obtained from the subject. Certain T cells are isolated from the composition, by immunoaffinity-based enrichment, and subject to genetic engineering, e.g., to express a recombinant receptor. The engineered cells then can be administered to the subject for therapy. In some embodiments, clonotype determination is performed at any one or more points or stages throughout the process, and compared with the clonotype determination performed at other points or stages. Exemplary methods that can be used for adoptive cell therapy, e.g., CAR-expressing T cell therapy, are described below in Section III.B. The methods provided herein can be used in any stages or time points of performing adoptive cell therapy, using any compositions or samples, such as those obtained from the subject or engineered or processed. Particular T cell clones or clonotypes can be traced throughout the generation of cells for therapy and after administration of the cells to subjects.

a. T Cell Composition

In some embodiments, clonotype determination and/or cell property determination is performed in a T cell composition comprising T cells that are, or have been derived from, T cells previously obtained from the subject prior to administering the cell therapy to the subject. In some embodiments, the T cell composition contains cells that are obtained from the subject for genetic engineering, such as a whole blood sample, a buffy coat sample, a peripheral blood mononuclear cells (PBMC) sample, an unfractionated T cell sample, a lymphocyte sample, a white blood cell sample, an apheresis product, or a leukapheresis product. In some embodiments, the compositions or samples obtained from the subject are further subject to purification or isolation. In some embodiments, the initial T cell composition contains cells that are derived or isolated is blood or a blood-derived composition, or is derived from an apheresis or leukapheresis product. Exemplary compositions obtained from the subject include whole blood, peripheral blood mononuclear cells (PBMCs), leukocytes, bone marrow, thymus, and/or cells derived therefrom. In some embodiments, cells are derived, isolated, and/or selected from a composition or sample obtained from the subject prior to engineering. In the context of cell therapy, e.g., adoptive cell therapy, the T cells in the T cell composition can be from autologous and allogeneic sources. In some embodiments, the T cell composition contains patient material, such as cells obtained from the subject.

In some embodiments, the T cell composition can contain cells that are derived, isolated and/or selected from a composition or cells obtained from the subject, and is further engineered, e.g., isolated, activated, transduced and/or expanded in vitro. In some embodiments, the T cells in the T cell composition are cells that have been engineered to express a recombinant receptor, e.g., a chimeric antigen receptor (CAR) or a recombinant T cell receptor (TCR). In some embodiments, the T cells in the T cell composition include cells that have been obtained and/or engineered as described in Section III below. In some embodiments, the T cell composition contains the drug product for administration in adoptive cell therapy, e.g., drug product that contains engineered cells.

In some embodiments, the clonotype determination and/or cell property determination is performed in samples from one or more stages of obtaining and engineering cells for adoptive cell therapy, prior to administration. In some embodiments, the clonotype determination and/or cell property determination is performed in T cell composition from subject (before engineering) and/or among the engineered cells (after engineering), or at various intermediate stages of engineering.

b. Test Biological Samples

In some embodiments, clonotype determination and/or cell property determination is performed in one or more test biological samples form a subject. In some embodiments, the clonotype determination and/or cell property determination is performed in at least one T cell from at least one test biological sample from a subject, said test biological sample obtained from the subject following administration of a cell therapy comprising T cells expressing a recombinant receptor.

In some embodiments, the test biological samples from the subject are obtained at one or more time points and/or stages of adoptive cell therapy, or at one or more locations or biological samples from the subject. In some embodiments, the test biological sample is obtained from a subject at or about or within 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, or more following administration of the cell therapy containing engineered cells (e.g. CAR-T cells). In some embodiments, the clonotype is determined on T cells from test biological samples obtained at more than one, such as two, three, four, or five time points after administration of the cell therapy. For example, in some embodiments, the test biological sample containing the administered engineered cell is obtained on various days, such as days 15, 22, 29, after administration of the cells. In some embodiments, the test biological sample is obtained at more than one time point.

In some embodiments, the test biological sample is obtained from different locations in the subject's body, such as a biopsy of a solid tumor (e.g., tumor infiltrating lymphocytes) or form the plasma of the subject. In some embodiments, the test biological sample from a subject is derived from whole blood, peripheral blood mononuclear cells (PBMCs), leukocytes, bone marrow and/or thymus of the subject after administration of the adoptive cell therapy.

In some embodiments, the test biological sample is obtained from a subject at a time when the administered engineered cells of the cell therapy, e.g., adoptively transferred cells, are detectable in the subject. In some aspects, the test biological sample is obtained from the subject at a time in which detectable cells in the subject is indicative or may indicate that engineered cells of the cell therapy persist in the subject. In some embodiments, the test biological sample is obtained from the subject more than 14 days, such as more than 22 days or more than 29 days after administering the cell therapy.

In some embodiments, the test biological sample is a sample obtained from a subject at a time after, such as immediately after, that subject has been identified, known or suspected as exhibited a particular therapeutic response or outcome to administration of a cell therapy containing engineered cells (e.g. CAR-T cells). In some embodiments, a response outcome in a subject to administration of a cell therapy can be monitored and/or assessed. In particular embodiments, subjects administered a cell therapy containing engineered cells (e.g. CAR-T cells) are assessed and/or monitored for a period of time to determine if the subjects experience a response. In certain embodiments, test biological sample is obtained from a subject in which the subject exhibits a reduced disease burden, e.g. reduced tumor size, following administration of the engineered cells. In certain embodiments, test biological sample is obtained from a subject in which the response outcome is a complete response (CR) following administration of the engineered cells. In some embodiments, the test biological sample is from a subject that has minimum residual disease (MRD) following the administration of the engineered cells. In some embodiments, the test biological sample is from a subject that does not have MRD following administration of the engineered cells. In some embodiments, the test biological sample is obtained from a subject in which the response outcome is a partial response following administration of the engineered cells. In some embodiments, the test biological sample is obtained from a subject in which the subject exhibits a durable response for at least 3 months, at least 6 months, at least 9 months, at least 12 months or more following administration of the engineered cells. In some embodiments, the test biological sample is obtained from a subject in which the response outcome is progression free survival following administration of the engineered cells. In some embodiments, the test biological sample is obtained from a subject in which the response outcome is no response or in which the subject exhibits progressive disease following administration of the engineered cells. In some embodiments, response outcome is assessed by monitoring the disease burden in the subject. In some embodiments, the presence of no response, a partial response or a clinical or complete response can be assessed.

In some aspects, response rates in subjects are based on the Lugano criteria. (Cheson et al., (2014) JCO 32(27):3059-3067; Johnson et al., (2015) Radiology 2:323-338; Cheson, B. D. (2015) Chin Clin Oncol 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake ≤mediastinum; 3, uptake>mediastinum but ≤liver; 4, uptake moderately>liver; 5, uptake markedly higher than liver and/or new lesions; X, new areas of uptake unlikely to be related to lymphoma.

In some aspects, a complete response as described using the Lugano criteria involves a complete metabolic response and a complete radiologic response at various measureable sites. In some aspects, these sites include lymph nodes and extralymphatic sites, wherein a CR is described as a score of 1, 2, or 3 with or without a residual mass on the 5-point scale, when PET-CT is used. In some aspects, in Waldeyer's ring or extranodal sites with high physiologic uptake or with activation within spleen or marrow (e.g., with chemotherapy or myeloid colony-stimulating factors), uptake may be greater than normal mediastinum and/or liver. In this circumstance, complete metabolic response may be inferred if uptake at sites of initial involvement is no greater than surrounding normal tissue even if the tissue has high physiologic uptake. In some aspects, response is assessed in the lymph nodes using CT, wherein a CR is described as no extralymphatic sites of disease and target nodes/nodal masses must regress to ≤1.5 cm in longest transverse diameter of a lesion (LDi). Further sites of assessment include the bone marrow wherein PET-CT-based assessment should indicate a lack of evidence of FDG-avid disease in marrow and a CT-based assessment should indicate a normal morphology, which if indeterminate should be IHC negative. Further sites may include assessment of organ enlargement, which should regress to normal. In some aspects, non-measured lesions and new lesions are assessed, which in the case of CR should be absent (Cheson et al., (2014) JCO 32(27):3059-3067; Johnson et al., (2015) Radiology 2:323-338; Cheson, B. D. (2015) Chin Clin Oncol 4(1):5).

In some aspects, a partial response (PR; also known in some cases as partial remission) as described using the Lugano criteria involves a partial metabolic and/or radiological response at various measureable sites. In some aspects, these sites include lymph nodes and extralymphatic sites, wherein a PR is described as a score of 4 or 5 with reduced uptake compared with baseline and residual mass(es) of any size, when PET-CT is used. At interim, such findings can indicate responding disease. At the end of treatment, such findings can indicate residual disease. In some aspects, response is assessed in the lymph nodes using CT, wherein a PR is described as ≥50% decrease in SPD of up to 6 target measureable nodes and extranodal sites. If a lesion is too small to measure on CT, 5 mm×5 mm is assigned as the default value; if the lesion is no longer visible, the value is 0 mm×0 mm; for a node >5 mm×5 mm, but smaller than normal, actual measurements are used for calculation. Further sites of assessment include the bone marrow wherein PET-CT-based assessment should indicate residual uptake higher than uptake in normal marrow but reduced compared with baseline (diffuse uptake compatible with reactive changes from chemotherapy allowed). In some aspects, if there are persistent focal changes in the marrow in the context of a nodal response, consideration should be given to further evaluation with MRI or biopsy, or an interval scan. In some aspects, further sites may include assessment of organ enlargement, where the spleen must have regressed by >50% in length beyond normal. In some aspects, nonmeasured lesions and new lesions are assessed, which in the case of PR should be absent/normal, regressed, but no increase. No response/stable disease (SD) or progressive disease (PD) can also be measured using PET-CT and/or CT based assessments. (Cheson et al., (2014) JCO 32(27):3059-3067; Johnson et al., (2015) Radiology 2:323-338; Cheson, B. D. (2015) Chin Clin Oncol 4(1):5).

In some respects, progression-free survival (PFS) is described as the length of time during and after the treatment of a disease, such as cancer, that a subject lives with the disease but it does not get worse. In some aspects, objective response (OR) is described as a measurable response. In some aspects, objective response rate (ORR; also known in some cases as overall response rate) is described as the proportion of patients who achieved CR or PR. In some aspects, overall survival (OS) is described as the length of time from either the date of diagnosis or the start of treatment for a disease, such as cancer, that subjects diagnosed with the disease are still alive. In some aspects, event-free survival (EFS) is described as the length of time after treatment for a cancer ends that the subject remains free of certain complications or events that the treatment was intended to prevent or delay. These events may include the return of the cancer or the onset of certain symptoms, such as bone pain from cancer that has spread to the bone, or death.

In some embodiments, the measure of duration of response (DOR) includes the time from documentation of tumor response to disease progression. In some embodiments, the parameter for assessing response can include durable response, e.g., response that persists after a period of time from initiation of therapy. In some embodiments, durable response is indicated by the response rate, e.g. CR, at approximately 1, 2, 3, 4, 5, 6, 7, 8, 9, 10, 11, 12, 18 or 24 months after initiation of therapy. In some embodiments, the response is durable for greater than 3 months or greater than 6 months.

In some aspects, the RECIST criteria is used to determine objective tumor response; in some aspects, in solid tumors. (Eisenhauer et al., European Journal of Cancer 45 (2009) 228-247.) In some aspects, the RECIST criteria is used to determine objective tumor response for target lesions. In some respects, a complete response as determined using RECIST criteria is described as the disappearance of all target lesions and any pathological lymph nodes (whether target or non-target) must have reduction in short axis to <10 mm. In other aspects, a partial response as determined using RECIST criteria is described as at least a 30% decrease in the sum of diameters of target lesions, taking as reference the baseline sum diameters. In other aspects, progressive disease (PD) is described as at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm (in some aspects the appearance of one or more new lesions is also considered progression). In other aspects, stable disease (SD) is described as neither sufficient shrinkage to qualify for PR nor sufficient increase to qualify for PD, taking as reference the smallest sum diameters while on study.

In some embodiments, a partial response or complete response is one in which the therapeutic cell composition reduces or prevents the expansion or burden of the disease or condition in the subject. For example, where the disease or condition is a tumor, reduced disease burden exists or is present if there is a reduction in the tumor size, bulk, metastasis, percentage of blasts in the bone marrow or molecularly detectable cancer and/or an improvement prognosis or survival or other symptom associated with tumor burden compared to prior to treatment with the therapeutic cell composition (e.g. CAR T cells).

In some embodiments, the disease or condition is a tumor and a reduction in disease burden is a reduction in tumor size. In some embodiments, the disease burden reduction is indicated by a reduction in one or more factors, such as load or number of disease cells in the subject or fluid or organ or tissue thereof, the mass or volume of a tumor, or the degree or extent of metastases. In some embodiments, disease burden, e.g. tumor burden, can be assessed or monitored for the extent of morphological disease and/or minimal residual disease.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the mass of a solid tumor and/or the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In certain embodiments, the burden or a disease or condition is detected, assessed, and/or measured in subjects in a treatment regimen to determine the efficacy rate of the treatment regimen.

In some embodiments, disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood or bone marrow in the context of certain hematological malignancies. Disease burden can include, in some embodiments, the mass of a tumor, the number or extent of metastases and/or the percentage of blast cells present in the bone marrow.

In some embodiments, a subject has leukemia. The extent of disease burden can be determined by assessment of residual leukemia in blood or bone marrow. In particular embodiments, residual leukemia in blood or bone marrow is detected, assessed, and/or measured in subjects of a treatment regimen to determine the efficacy rate of the treatment regimen.

In some embodiments, a response outcome exists if there is a reduction in the percent of blasts in the bone marrow compared to the percent of blasts in the bone marrow prior to treatment with the therapeutic agent. In some embodiments, reduction of disease burden exists if there is a decrease or reduction of at least or at least about 20%, 30%, 40%, 50%, 60%, 70%, 80%, 90%, 95% or more in the number or percentage of blasts in the bone marrow compared to the number or percent of blasts in the bone marrow prior to treatment. In particular embodiments, the percent of blasts in the bone marrow compared to the percent of blasts in the bone marrow prior to treatment with the therapeutic agent is detected, assessed, and/or measured in subjects in a treatment regimen to determine the efficacy rate of the treatment regimen.

In some embodiments, the subject exhibits a response if the subject does not exhibit morphologic disease (non-morphological disease) or does not exhibit substantial morphologic disease. In some embodiments, a subject exhibits morphologic disease if there are greater than or equal to 5% blasts in the bone marrow, for example, as detected by light microscopy. In some embodiments, a subject exhibits complete or clinical remission if there are less than 5% blasts in the bone marrow. In particular embodiments, morphological disease is detected, assessed, and/or measured in subjects of a treatment regimen to determine the efficacy rate of the regimen.

In some aspects, response rates in subjects, such as subjects with certain leukemias, e.g. CLL, are based on the International Workshop on Chronic Lymphocytic Leukemia (IWCLL) response criteria (Hallek, et al., Blood 2008, Jun. 15; 111(12): 5446-5456). In some aspects, these criteria are described as follows: complete remission (CR; also known in some cases as complete response), which in some aspects requires the absence of peripheral blood clonal lymphocytes by immunophenotyping, absence of lymphadenopathy, absence of hepatomegaly or splenomegaly, absence of constitutional symptoms and satisfactory blood counts; complete remission with incomplete marrow recovery (CRi), which in some aspects is described as CR above, but without normal blood counts; partial remission (PR; also known in some cases as partial response), which in some aspects is described as ≥50% fall in lymphocyte count, ≥50% reduction in lymphadenopathy or ≥50% reduction in liver or spleen, together with improvement in peripheral blood counts; progressive disease (PD), which in some aspects is described as ≥50% rise in lymphocyte count to ≥5×10⁹/L, ≥50% increase in lymphadenopathy, ≥50% increase in liver or spleen size, Richter's transformation, or new cytopenias due to CLL; and stable disease, which in some aspects is described as not meeting criteria for CR, CRi, PR or PD.

In some embodiments, the subjects exhibits a CR or OR if, within 1 month of the administration of the dose of cells, lymph nodes in the subject are less than at or about 20 mm in size, less than at or about 10 mm in size or less than at or about 10 mm in size.

In some embodiments, a subject exhibits morphologic disease if there are greater than or equal to 5% blasts in the bone marrow, for example, as detected by light microscopy, such as greater than or equal to 10% blasts in the bone marrow, greater than or equal to 20% blasts in the bone marrow, greater than or equal to 30% blasts in the bone marrow, greater than or equal to 40% blasts in the bone marrow or greater than or equal to 50% blasts in the bone marrow. In some embodiments, a subject exhibits complete or clinical remission if there are less than 5% blasts in the bone marrow.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable cancer. In some embodiments, molecularly detectable cancer can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify cancer cell based on leukemia-specific immunophenotypes. In some embodiments, molecular detection of cancer can detect as few as 1 leukemia cell in 100,000 normal cells. In some embodiments, a subject exhibits MRD that is molecularly detectable if at least or greater than 1 leukemia cell in 100,000 cells is detected, such as by PCR or flow cytometry. In some embodiments, the disease burden of a subject is molecularly undetectable or MRD⁻, such that, in some cases, no leukemia cells are able to be detected in the subject using PCR or flow cytometry techniques.

In some embodiments, an index clone of the leukemia, e.g. CLL, is not detected in the bone marrow of the subject (or in the bone marrow of greater than 50%, 60%, 70%, 80%, 90% or more of the subjects treated according to the methods. In some embodiments, an index clone of the leukemia, e.g. CLL, is assessed by IGH deep sequencing. In some embodiments, the index clone is not detected at a time that is at or about or at least at or about 1, 2, 3, 4, 5, 6, 12, 18 or 24 months following the administration of the cells.

In some cases, methods can be carried out to detect administered engineered cells, e.g., adoptively transferred cells, in the subject, assess a pharmacokinetic activity of the cells, such as persistence, pharmacokinetics or availability, e.g., bioavailability of the administered cells. In some embodiments, the degree or extent of persistence of administered cells can be detected or quantified after administration to a subject. For example, in some aspects, quantitative PCR (qPCR) is used to assess the quantity of cells expressing the chimeric receptor (e.g., CAR-expressing cells) in the blood or serum or organ or tissue (e.g., disease site) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the recombinant receptor (e.g. CAR-expressing cells) can be used to distinguish the administered cells from endogenous cells in a subject.

Methods for determining the presence or number of adoptively transferred cells may include drawing peripheral blood from subjects that have been administered engineered cells, and determining the number or ratio of the engineered cells in the peripheral blood. Approaches for selecting and/or isolating cells may include use of chimeric antigen receptor (CAR)-specific antibodies (e.g., Brentj ens et al., Sci. Transl. Med. 2013 March; 5(177): 177ra38) Protein L (Zheng et al., J. Transl. Med. 2012 February; 10:29), epitope tags, such as Strep-Tag sequences, introduced directly into specific sites in the CAR, whereby binding reagents for Strep-Tag are used to directly assess the CAR (Liu et al. (2016) Nature Biotechnology, 34:430; international patent application Pub. No. WO2015095895) and monoclonal antibodies that specifically bind to a CAR polypeptide (see international patent application Pub. No. WO2014190273). Extrinsic marker genes may in some cases be utilized in connection with engineered cell therapies to permit detection or selection of cells and, in some cases, also to promote cell suicide. A truncated cell surface receptor, such as a truncated epidermal growth factor receptor (EGFRt), in some cases can be co-expressed with a transgene of interest (a CAR or TCR) in transduced cells (see e.g. U.S. Pat. No. 8,802,374). The truncated cell surface receptor, e.g. EGFRt, may contain an epitope recognized by the antibody cetuximab (Erbitux®) or other therapeutic anti-EGFR antibody or binding molecule, which can be used to identify or select cells that have been engineered with the EGFRt construct and another recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434).

In some embodiments, the number of CAR⁺ T cells in a biological sample obtained from the patient, e.g., blood, can be determined at a period of time after administration of the cell therapy, e.g., to determine the pharmacokinetics of the cells. In some embodiments, a test biological sample is obtained from a subject that has been administered a cell therapy containing engineered CAR+ T cells at a time when the number of CAR⁺ T cells, optionally CAR⁺ CD8⁺ T cells and/or CAR⁺ CD4⁺ T cells, detectable in the blood of the subject is greater than 1 cells per μL, greater than 5 cells per μL or greater than per 10 cells per μL.

In some embodiments, the sample, such as the test biological sample, obtained from the subject is further selected, purified or isolated, such as by selection of particular cells or subsets of cells, prior to clonotype determination and/or cell property determination. In some embodiments, selection of cells expressing the recombinant receptor, e.g., CAR-expressing cells, is carried out prior to clonotype determination and/or cell property determination. In some embodiments, cells from a test biological sample are further selected for cells that have a particular phenotype associated with one or more sub-types or subpopulations of T cells.

In some cases, cells can be selected based on phenotype, such as by expression of one or more markers, e.g. surface markers, using flow-cytometry-based cell sorting. When referring to a surface marker, the term refers to the presence of surface expression as detected by flow cytometry, for example, by staining with an antibody that specifically binds to the marker and detecting or selecting the cells positive or negative for the surface marker. In some cases, a cell is detected or selected if the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker. In some embodiments, a cell is detected or selected as negative or not expressing a surface marker when the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Among the phenotypes are the expression or surface expression of one or more markers generally associated with one or more sub-types or subpopulations of T cells, or phenotypes thereof. T cell subtypes and subpopulations may include CD4+ and/or of CD8+ T cells and subtypes thereof that may include naive T (T_{N}) cells, effector T cells (T_{EFF}), memory T cells and sub-types thereof, such as stem cell memory T (T_{SCM}), central memory T (T_{CM}), effector memory T (T_{EM}), T_{EMRA} cells or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the phenotype is or includes a phenotype of or associated with a memory T cell or memory T cell subset. Memory T cells are antigen-specific T cells that have previously been exposed to their cognate antigen. Memory T cells persist long-term after an infection has resolved. Memory T cells quickly expand to large numbers of effector T cells upon re- exposure to their cognate antigen, thus providing the immune system with “memory” against past infections. Memory T cells comprise three subtypes: central memory T cells (T_{CM} cells) and two types of effector memory T cells (T_{EM} cells and T_{EMRA} cells). In some embodiments the phenotype is or includes a phenotype of a memory T cell (or one or more markers associated therewith), such as a T_{CM} cell, a T_{EM} cell, or a T_{EMRA} cell, a memory stem T cell (T_{SCM}) cell, or a combination thereof. In particular embodiments, the phenotype is or includes the expression of one or more specific molecules that is a marker for memory and/or memory T cells or subtypes thereof.

In particular embodiments, the phenotype is or includes the expression of one or more specific molecules that is a marker for naive-like T cells. Naïve T cells include fresh T cells that have been produced in the bone marrow and are able to respond to newly encountered pathogens containing antigens the immune system has not processed before. After stimulation by their cognate antigen, a portion activated naive T-cells will develop into memory cells.

In some embodiments, the phenotype is or includes a phenotype associated with memory T cell or a naive T cell. In certain embodiments, the phenotype is the positive or negative expression of one or more specific molecules that are markers for memory. In some embodiments, the memory marker is a specific molecule that may be used to define a memory T cell population.

In some embodiments, the phenotype is or includes a phenotype of or one or more marker associated with a non-memory T cell or sub-type thereof; in some aspects, it is or includes a phenotype or marker(s) associated with a naive cell or a naive-like cell. In some embodiments, to identify naive-like T cells, in some embodiments, various signatures of naive-like T cells can be utilized. In some embodiments, expression of particular markers of naive-like T cells can be assessed. For examples, in some cases, the naive-like T cells are surface positive for a marker, including T cell activation markers, selected from the group consisting of CD27, CD28, CD45RA, CD62L, and CCR7. In some aspects, the naive-like T cells are surface negative for CD56 and/or CD45RO. In some aspects, the naive-like T cells are surface negative for CD45RO and cell surface positive for CD27, CD45RA, and CCR7. In some cases, the naive-like T cells are negative for intracellular expression of a cytokine such as IL-2, IFN-γ, IL-4, and/or IL-10. In some further examples, the naive-like T cells are negative for expression of markers CD25 and/or perforin. In some cases, the naive-like T cells are CD95^{lo}. In some embodiments, the phenotype is CCR7⁺/CD27⁺/CD28⁺/CD45RA⁺. In certain embodiments, the phenotype is or includes CCR7⁺/CD45RA⁺.

In some embodiments, phenotype is associated with memory T cells, such as long-lived memory T cells. In some embodiments, the memory T cells are central memory (T_{CM}) T cells. In some embodiments, the T cell subset has a phenotypic characteristic CD45RA−, CD45RO^{low/+}, CCR7+, CD62L+, CD27+, CD28+, CD95+ CD122+ and/or KLGR1^{low}.

In some embodiments, the memory T cells are stem central memory (T_{SCM}) T cells. In some embodiments, the T cell subset has a phenotypic characteristic CD45RA^{low/+}, CD45RO^{low/+}, CCR7+, CD62L+, CD27+, CD28+, CD95+, CD122+ and/or KLGR1−. In some embodiments, the T cell subset has a phenotypic characteristic CD45RA^{low/+}, CD45RO⁻, CCR7+, CD62L+, CD27+, CD28+, CD95+, CD122+ and/or KLGR1−. In some embodiments, the T cell subset has a phenotypic characteristic CD45RO⁻, CCR7⁺, CD45RA⁺, CD62L⁺, CD27⁺, CD28⁺, IL-7Rα⁺, CD95⁺, IL-2Rβ⁺, CXCR3⁺, and/or LFA-1⁺. In some embodiments, the T cell subset has a phenotypic characteristic CD45RA⁺, CCR7⁺, CD62L⁺, and/or CD95⁺. In some embodiments, the T cell subset has a phenotypic characteristic CD45RA⁺, CD45RO⁻, CCR7⁺, CD62L⁺, CD27⁺, CD28⁺, CD95⁺, and/or IL-2Rβ⁺. In some embodiments, the T cell subset has a phenotypic characteristic CD45RO⁻, CD45RA⁺, CCR7⁺, CD62L⁺, CD27⁺, CD28⁺, CD127⁺, and/or CD95⁺. In some embodiments, the T cell subset has a phenotypic characteristic CD45RA⁺, CD44^{+/-}, CD62L⁺, CD127⁺, IL-2Rβ⁺, CD28⁺, CD43⁻, KLRG1⁻, Peforin⁻, and/or GranzymeB⁻. In some embodiments, the T cell subset expresses high levels of CCR7, CD62L, CD27, and/or CD28, intermediate levels of CD95 and/or IL-2Rβ, low levels of CD45RA, and/or does not express CD45RO and/or KLRG-1. In some embodiments, the T cell subset expresses high levels of CD62L, low levels of CD44 and t-bet, and/or is Sca-1⁺. In some embodiments, the T cell subset has a phenotypic characteristic intermediate IL-2 -producing capacity, low IFNγ-producing capacity, low cytotoxicity, and/or high self-renewal capacity.

In some embodiments, the phenotype is or includes a phenotype of or one or more marker associated with the non-naive-like T cells, which, in some aspects, can include effector T (T_{EFF}) cells, memory T cells, central memory T cells (T_{CM}), effector memory T (T_{EM}) cells, and combinations thereof. To identify non-naive-like T cells, in some embodiments, various signatures of non-naive-like T cells can be utilized. In some embodiments, expression of particular markers of non-naïve-like T cells can be assessed. For example, in some cases, the non-naive-like T cells are surface negative for a marker, including T cell activation markers, such as CD27, CD28, CD45RA, and CCR7; and in some cases, the non-naive-like T cells are surface positive for a marker, including CD62L. In some aspects, the non-naive-like T cells are surface positive for CD56 and/or CD45RO. In some aspects, the non-naive-like T cells are surface positive for CD45RO and cell surface negative for CD27, CD45RA, and CCR7. In some cases, the non-naive-like T cells are positive for intracellular expression of a cytokine such as IL-2, IFN-γ, IL-4, and/or IL-10. In some further examples, the non-naive-like T cells are positive for expression of markers CD25 and/or perforin. In some cases, the non-naive-like T cells are CD95^{hi}.

In some embodiments, the phenotype is or includes a phenotype of a central memory T cell. In particular embodiments, the phenotype is or includes CCR7⁺/CD27⁺/CD28⁺/CD45RA⁻. In some embodiments, the phenotype is or includes an effector memory cell. In some embodiments, the phenotype is or includes CCR7⁻/CD27⁺/CD28⁺/CD45RA⁻. In certain embodiments, the phenotype is or includes that of a T_{EMRA} cell or a T_{SCM} cell. In certain embodiments, the phenotype is or includes CD45RA⁺. In particular embodiments, the phenotype is or includes CCR7⁻/CD27⁻/CD28⁻/CD45RA⁺. In some embodiments, the phenotype is or includes one of CD27⁺/CD28⁺, CD27⁻/CD28⁺, CD27⁻/CD28⁻, or CD27⁻/CD28⁻.

In some embodiments the phenotype is or includes any of the foregoing phenotypic properties and further includes the expression of a recombinant receptor, such as phenotype associated with a memory T cell or memory subtype and that expresses a CAR, or a phenotype associated with a naïve-like cell that expresses a CAR. In certain embodiments, the phenotype is or includes that of a central memory T cell or stem central memory T cell that expresses a CAR. In particular embodiments, the phenotype is or includes that of an effector memory cell that expresses a CAR. In some embodiments, the phenotype is or includes that of a T_{EMRA} cell that expresses a CAR. In particular embodiments, the phenotype is or includes CAR⁺/CCR7⁺/CD27⁺/CD28⁺/CD45RA⁻; CAR⁺/CCR7⁻/CD27⁺/CD28⁺/CD45RA⁻; CAR⁺/CCR7⁻/CD27⁻/CD28⁻/CD45RA⁺; CAR⁺/CD27⁺/CD28⁺; CAR⁺/CD27⁻/CD28⁺; CAR⁺/CD27⁺/CD28⁻; or CAR⁺/CD27⁻/CD28⁻.

c. Sample Preparation

Any biological sample, including a sample containing a population of cells, containing polynucleotides can be used in the methods described herein. Any sample containing a cell generally can be used in the methods described herein. For example, a sample can be a biological sample from a subject or from a sample derived therefrom containing RNA or DNA. The polynucleotides can be extracted from the biological sample, or the sample can be directly subjected to the methods without extraction or purification of the polynucleotides. The sample can be extracted or isolated DNA or RNA. A sample can also be total RNA or DNA extracted from a biological specimen, a cDNA library, viral, or genomic DNA. In one embodiment, polynucleotides are isolated from a biological sample containing a variety of other components, such as proteins, lipids and non-template nucleic acids. Nucleic acid template molecules can be obtained from any cellular material, obtained from an animal, plant, bacterium, fungus, or any other cellular organism.

In certain embodiments, the polynucleotides are obtained from a single cell, such as a cell present in a population of cells. In certain embodiments, the polynucleotides are obtained from a population or composition of cells, such as a population or composition containing a plurality of T cells. Polynucleotides can be obtained directly from an organism or from a biological sample obtained from an organism. Any tissue or body fluid specimen may be used as a source for nucleic acid for use in the embodiments. Polynucleotides can also be isolated from cultured cells, such as a primary cell culture or a cell line. In some embodiments the cell can be a blood cell, an immune cell, a tissue cell, or a tumor cell. In some embodiments, the cell is an immune cell, such as a T cell. The cells or tissues from which template nucleic acids are obtained can be infected with a virus or other intracellular pathogen.

In the present embodiments the lymphocyte pool can be enriched for the desired immune cells by any suitable method, such as screening and sorting the cells using fluorescence-activated cell sorting (FACS), magnetic activated cell sorting (MACS), panning or other screening method to generate a plurality of immune cells from a sample, such as an immune cell library, before TCR chains are sequenced, TCRs are made, or an expression library is/are made. In contrast to prior art enrichment methods, which provide only a few subsets of immune cells expressing different TCRs, and therefore only a few naturally occurring combinations of variable domains, the immune cell library of the present embodiments contains at least 2 subsets of or individual immune cells expressing different TCRs. For example, the immune cell library of the present embodiments can contain at least 5, 10, 100, 250, 500, 750, 1,000, 2,500, 5,000, 10,000, 25,000, 50,000, 75,000, 10,000, 250,000, 500,000, 750,000, 1,000,000, 2,500,000, 5,000,000, 7,500,000, or 10,000,000 subsets of or individual immune cells expressing different TCRs. The methods of the present embodiments maximize immune cell recovery, and afford very high diversity.

T cells can be obtained from a number of sources, including peripheral blood mononuclear cells, bone marrow, thymus, tissue biopsy, tumor, lymph node tissue, gut associated lymphoid tissue, mucosa associated lymphoid tissue, spleen tissue, or any other lymphoid tissue, and tumors. T cells can be obtained from T cell lines and from autologous or allogeneic sources. T cells may be obtained from a single individual or a population of individuals, for example, a population of individual who all suffer from the same disease, such as, a cancer or an infectious disease. In some embodiments, cells from the circulating blood of an individual are obtained by apheresis or leukapheresis. The apheresis product typically contains lymphocytes, including T cells, monocytes, granulocytes, B cells, other nucleated while blood cells, red blood cells, and platelets. In one embodiment, the cells collected by apheresis or leukapheresis may be washed to remove the plasma fraction and to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge. After washing, the cells may be resuspended in a variety of biocompatible buffers, such as, for example. Ca++/Mg++ free PBS. Alternatively, the undesirable components of the apheresis sample may be removed and the cells directly resuspended in culture media. In other embodiments, T cells are isolated from peripheral blood lymphocytes by lysing the red blood cells and by centrifugation through a PERCOLL™ gradient. A specific subpopulation of T cells, such as CD28, CD4, CD8, CD45RA, and CD45RO+T cells, can be further isolated by positive or negative selection techniques. For example, CD3, CD28+ T cells can be positively selected using CD3/CD28 conjugated magnetic beads (e.g., DYNABEADS® M-450 CD3/CD28 T Cell Expander).

In some embodiments, enrichment of a T cell population by negative selection can be accomplished with a combination of antibodies directed to surface markers unique to the negatively selected cells. One such method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. Another method for preparing T cells for stimulation is to freeze the cells after the washing step, which does not require the monocyte-removal step. Wishing not to be bound by theory, the freeze and subsequent thaw step provides a more uniform product by removing granulocytes and, to some extent, monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° C. per minute and stored in the vapor phase of a liquid nitrogen storage tank.

In some embodiments, the population of cells is enriched from a sample. In some embodiments, cells are enriched for a particular subset or subtype of cell. In some embodiments, the populations of cells are enriched for or contain T cells. In some embodiments, the populations of cells are enriched for or contain CD4+ or CD8+ cells. In some embodiments, the populations of cells are enriched for or contain central memory T cells, effector memory T cells, naïve T cells, stem central memory T cells, effector T cells and regulatory T cells. In some embodiments, immune cells can be selected based on the affinity of the immune receptors from the cell for a selected target antigen or complex. In some aspects, affinity refers to the equilibrium constant for the reversible binding of two agents and is expressed as KD. Affinity of a binding protein to a ligand such as affinity of an antibody for an epitope or such as affinity for a TCR for a MCH-peptide complex can be, for example, from about 100 nanomolar (nM) to about 0.1 nM, from about 100 nM to about 1 picomolar (pM), or from about 100 nM to about 1 femtomolar (fM). The term “avidity” refers to the resistance of a complex of two or more agents to dissociation after dilution.

In some cases, in order to obtain sufficient nucleic acid for testing, a blood volume of at least 0.001, 0.005, 0.01, 0.05, 0.1, 0.5, 1, 2, 3, 4, 5, 10, 20, 25, 30, 35, 40, 45, or 50 mL is drawn.

In some cases, the sample is peripheral blood. The peripheral blood cells can be enriched for a particular cell type (e.g., mononuclear cells; red blood cells; CD4+ cells; CD8+ cells; immune cells; T cells, NK cells, or the like). The peripheral blood cells can also be selectively depleted of a particular cell type (e.g., mononuclear cells; red blood cells; CD4+ cells; CD8+ cells; immune cells; T cells, NK cells, or the like).

In some cases, the sample can be a tissue sample comprising a solid tissue, with non-limiting examples including brain, liver, lung, kidney, prostate, ovary, spleen, lymph node (including tonsil), thyroid, pancreas, heart, skeletal muscle, intestine, larynx, esophagus, and stomach. In other cases, the starting material can be cells containing nucleic acids, immune cells, and in particular B-cells or T-cells. In some cases, the sample can be a sample containing nucleic acids, from any organism, from which genetic material can be obtained. In some cases, a sample is a fluid, e.g., blood, saliva, lymph, or urine.

In some cases, non-nucleic acid materials can be removed from the sample using enzymatic treatments (such as protease digestion).

In some cases, blood can be collected into an apparatus containing a magnesium chelator including but not limited to EDTA, and is stored at 4° C. Optionally, a calcium chelator, including but not limited to EGTA, can be added. In another case, a cell lysis inhibitor is added to the blood including but not limited to formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfhydryl addition or disulfide reduction, carbohydrate reactive crosslinkers, carboxyl reactive crosslinkers, photoreactive crosslinkers, or cleavable crosslinkers.

In some cases when the extracted material comprises single-stranded RNA, double-stranded RNA, or DNA-RNA hybrid, these molecules can be converted to double-stranded DNA using techniques known in the field. For example, reverse transcriptase can be employed to synthesize DNA from RNA molecules. In some cases, conversion of RNA to DNA can require a prior ligation step, to ligate a linker fragment to the RNA, thereby permitting use of universal primers to initiate reverse transcription. In other cases, the poly-A tail of an mRNA molecule, for example, can be used to initiate reverse transcription. Following conversion to DNA, the methods detailed herein can be used, in some cases, to further capture, select, tag, or isolate a desired sequence.

Nucleic acid molecules include deoxyribonucleic acid (DNA) and/or ribonucleic acid (RNA). Nucleic acid molecules can be synthetic or derived from naturally occurring sources. In one embodiment, nucleic acid molecules are isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. In certain embodiments, the nucleic acid molecules are obtained from a single cell. Biological samples for use in the present embodiments include viral particles or preparations. Nucleic acid molecules can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the embodiments. Nucleic acid molecules can also be isolated from cultured cells, such as a primary cell culture or a cell line. The cells or tissues from which template nucleic acids are obtained can be infected with a virus or other intracellular pathogen.

A sample can also be total RNA extracted from a biological specimen, a cDNA library, viral, or genomic DNA. In certain embodiments, the nucleic acid molecules are bound as to other target molecules such as proteins, enzymes, substrates, antibodies, binding agents, beads, small molecules, peptides, or any other molecule Generally, nucleic acid can be extracted from a biological sample by a variety of techniques such as those described by Sambrook and Russell, Molecular Cloning: A Laboratory Manual, Third Edition, Cold Spring Harbor, N.Y. (2001). Nucleic acid molecules may be single-stranded, double-stranded, or double-stranded with single-stranded regions (for example, stem- and loop-structures).

Methods of DNA extraction are well-known in the art. A classical DNA isolation protocol is based on extraction using organic solvents such as a mixture of phenol and chloroform, followed by precipitation with ethanol (J. Sambrook et al., “Molecular Cloning: A Laboratory Manual,” 1989, 2nd Ed., Cold Spring Harbour Laboratory Press: New York, N.Y.). Other methods include: salting out DNA extraction (P. Sunnucks et al., Genetics, 1996, 144: 747-756; S. M. Aljanabi et al., Nucl. Acids Res. 1997, 25: 4692-4693), trimethylammonium bromide salts DNA extraction (S. Gustincich et al., BioTechniques, 1991, 11: 298-302) and guanidinium thiocyanate DNA extraction (J. B. W. Hammond et al., Biochemistry, 1996, 240: 298-300). A variety of kits are commercially available for extracting DNA from biological samples (e.g., BD Biosciences Clontech (Palo Alto, Calif.): Epicentre Technologies (Madison, Wis.); Gentra Systems, Inc. (Minneapolis, Minn.); MicroProbe Corp. (Bothell, Wash.); Organon Teknika (Durham, N.C.); and Qiagen Inc. (Valencia, Calif.)).

Methods of RNA extraction are also well known in the art (e.g., J. Sambrook et al., “Molecular Cloning: A Laboratory Manual” 1989, 211d Ed., Cold Spring Harbour Laboratory Press: New York) and kits for RNA extraction from bodily fluids are commercially available (e.g., Ambion, Inc. (Austin, Tex.); Amersham Biosciences (Piscataway, N.J.); BD Biosciences Clontech (Palo Alto, Calif.); BioRad Laboratories (Hercules, Calif.); Dynal Biotech Inc. (Lake Success, N.Y.); Epicentre Technologies (Madison, Wis.); Gentra Systems, Inc. (Minneapolis, Minn.); GIBCO BRL (Gaithersburg, Md.); Invitrogen Life Technologies (Carlsbad, Calif.); MicroProbe Corp. (Bothell, Wash.); Organon Teknika (Durham, N.C.); Promega, Inc. (Madison, Wis.); and Qiagen Inc. (Valencia, Calif.)).

One or more samples can be from one or more sources. One or more of samples may be from two or more sources. One or more of samples may be from one or more subjects. One or more of samples may be from two or more subjects. One or more of samples may be from the same subject. One or more subjects may be from the same species. One or more subjects may be from different species. The one or more subjects may be healthy. The one or more subjects may be affected by a disease, disorder or condition.

In some embodiments, a sample is a fluid, such as blood, saliva, lymph, urine, cerebrospinal fluid, seminal fluid, sputum, stool, or tissue homogenates.

In some embodiments, the polynucleotides are bound to other target molecules such as proteins, enzymes, substrates, antibodies, binding agents, beads, small molecules, peptides, or any other molecule. In some embodiments, the polynucleotides are not bound to a solid support. Nucleic acids can be extracted from a biological sample by a variety of techniques (Sambrook et al., Molecular Cloning: A Laboratory Manual, Third Edition, Cold Spring Harbor, N.Y. (2001)).

In some embodiments, the sample is saliva. In some embodiments, the sample is whole blood. In some embodiments, in order to obtain sufficient amount of polynucleotides for testing, a blood volume of at least about 0.001, 0.005, 0.01, 0.05, 0.1, 0.5, 1, 2, 3, 4, 5, 10, 20, 25, 30, 35, 40, 45, or 50 mL is drawn. In some embodiments, blood can be collected into an apparatus containing a magnesium chelator including but not limited to EDTA, and is stored at 4° C. Optionally, a calcium chelator, including but not limited to EGTA, can be added.

In some embodiments, a cell lysis inhibitor is added to the blood including but not limited to formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfhydryl addition or disulfide reduction, carbohydrate reactive crosslinkers, carboxyl reactive crosslinkers, photoreactive crosslinkers, or cleavable crosslinkers. In some embodiments, non-nucleic acid materials can be removed from the starting material using enzymatic treatments (such as protease digestion).

In some embodiments, cell suspensions can be preheated before analysis. In some embodiments, cell suspensions are heated immediately before emulsion generation to a temperature and for a sufficient duration to enhance the activity of the DNA polymerase inside the cell, but minimize undesired effects, such as RNA degradation. Thus, the cells are heated to optimize the yield of the methods provided herein. In some examples, the cells are heated to approximately 30° C. to 70° C., such as 30 to 60, 25 to 60, 30 to 60, 40 to 60, 45 to 55, for a duration of 1, 2 3, 4, 5, 6, 7, 8, 9, 10, 11, 12, 13, 14, 15, 16, 17, 18, 19, or 20 minutes. After heating the cells, the cell suspension can be held at room temperature or placed on ice for 30 seconds to up to 4 hours, such as 30 seconds, 45 seconds, 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 1 hour, 1.5 hours, 2 hours, 2.5 hours, 3 hours, 3.5 hours or 4 hours prior to forming the emulsion.

A plurality of samples may comprise at least 2, 3, 4, 5, 10, 20, 30, 40, 50, 60, 70, 80, 90 or 100 or more samples. The plurality of samples may comprise at least about 100, 200, 300, 400, 500, 600, 700, 800, 900 or 1000 or more samples. The plurality of samples may comprise at least about 1000, 2000, 3000, 4000, 5000, 6000, 7000, 8000 samples, 9000, or 10,000 samples, or 100,000 samples, or 1,000,000 or more samples. The plurality of samples may comprise at least about 10,000 samples.

The one or more polynucleotides in a first sample may be different from one or more polynucleotides in a second sample. The one or more polynucleotides in a first sample may be different from one or more polynucleotides in a plurality of samples. One or more polynucleotides in a sample can comprise at least about 80%, 85%, 90%, 95%, 96%, 97%, 98%, 99%, or 100% sequence identity. In some embodiments, one or more polynucleotides in a sample can differ by less than about 100, 90, 80, 70, 60, 50, 40, 30, 25, 20, 25, 10, 9, 8, 7, 6, 5, 4, 3, 2, or 1 nucleotide or base pair. A plurality of polynucleotides in one or more samples of the plurality of samples can comprise two or more identical sequences. At least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 98%, 99% or 100% of the total polynucleotides in one or more of the plurality of samples can comprise the same sequence. A plurality of polynucleotides in one or more samples of the plurality of samples may comprise at least two different sequences. At least about 5%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% of the total polynucleotides in one or more of the plurality of samples may comprise at least two different sequences. In some embodiments, one or more polynucleotides are variants of each other. For example, one or more polynucleotides may contain single nucleotide polymorphisms or other types of mutations. In another example, one or more polynucleotides are splice variants.

A first sample may comprise one or more cells and the second sample may comprise one or more cells. The one or more cells of the first sample may be of the same cell type as the one or more cells of the second sample. The one or more cells of the first sample may be of a different cell type as one or more different cells of the plurality of samples.

The plurality of samples may be obtained concurrently. A plurality of samples can be obtained at the same time. The plurality of samples can be obtained sequentially. A plurality of samples can be obtained over a course of years, e.g., 100 years, 10 years, 5 years, 4 years, 3 years, 2 years or 1 year of obtaining one or more different samples. One or more samples can be obtained within about one year of obtaining one or more different samples. One or more samples can be obtained within 12 months, 11 months, 10 months, 9 months, 8 months, 7 months, 6 months, 5 months, 4 months, 3 months, 2 months or 1 month of obtaining one or more different samples. One or more samples can be obtained within 30 days, 28 days, 26 days, 24 days, 21 days, 20 days, 18 days, 17 days, 16 days, 15 days, 14 days, 13 days, 12 days, 11 days, 10 days, 9 days, 8 days, 7 days, 6 days, 5 days, 4 days, 3 days, 2 days or 1 day of obtaining one or more different samples. One or more samples can be obtained within about 24 hours, 22 hours, 20 hours, 18 hours, 16 hours, 14 hours, 12 hours, 10 hours, 8 hours, 6 hours, 4 hours, 2 hours or 1 hour of obtaining one or more different samples. One or more samples can be obtained within about 60 seconds, 45 seconds, 30 seconds, 20 seconds, 10 seconds, 5 seconds, 2 seconds or 1 second of obtaining one or more different samples. One or more samples can be obtained within less than one second of obtaining one or more different samples.

The different polynucleotides of a sample can be present in the sample at different concentrations or amounts (e.g., different number of molecules). For example, the concentration or amount of one polynucleotide can be greater than the concentration or amount of another polynucleotide in the sample. In some embodiments, the concentration or amount of at least one polynucleotide in the sample is at least about 1.5, 2, 3, 4, 5, 6, 7, 8, 9, 10, 11, 12, 13, 14, 15, 20, 25, 30, 35, 40, 45, 50, 60, 70, 80, 90, 100, 200, 300, 400, 500, 600, 700, 800, 900, 1000, or more times greater than the concentration or amount of at least one other polynucleotide in the sample. In another example, the concentration or amount of one polynucleotide is less than the concentration or amount of another polynucleotide in the sample. The concentration or amount of at least one polynucleotide in the sample may be at least about 1.5, 2, 3, 4, 5, 6, 7, 8, 9, 10, 11, 12, 13, 14, 15, 20, 25, 30, 35, 40, 45, 50, 60, 70, 80, 90, 100, 200, 300, 400, 500, 600, 700, 800, 900, 1000, or more times less than the concentration or amount of at least one other polynucleotide in the sample.

In some embodiments, two or more samples may contain different amounts or concentrations of the polynucleotides. In some embodiments, the concentration or amount of one polynucleotide in one sample may be greater than the concentration or amount of the same polynucleotide in a different sample. For example, a blood sample might contain a higher amount of a particular polynucleotide than a urine sample. Alternatively, a single sample can divided into two or more subsamples. The subsamples may contain different amounts or concentrations of the same polynucleotide. The concentration or amount of at least one polynucleotide in one sample may be at least about 1.5, 2, 3, 4, 5, 6, 7, 8, 9, 10, 11, 12, 13, 14, 15, 20, 25, 30, 35, 40, 45, 50, 60, 70, 80, 90, 100, 200, 300, 400, 500, 600, 700, 800, 900, 1000, or more times greater than the concentration or amount of the same polynucleotide in another sample. Alternatively, the concentration or amount of one polynucleotide in one sample may be less than the concentration or amount of the same polynucleotide in a different sample. For example, the concentration or amount of at least one polynucleotide in one sample may be at least about 1.5, 2, 3, 4, 5, 6, 7, 8, 9, 10, 11, 12, 13, 14, 15, 20, 25, 30, 35, 40, 45, 50, 60, 70, 80, 90, 100, 200, 300, 400, 500, 600, 700, 800, 900, 1000, or more times less than the concentration or amount of the same polynucleotide in another sample.

2. Polynucleotide Libraries

In some embodiments, methods provided herein are directed to amplification and sequencing of one or more target polynucleotide molecules and amplification and sequencing a collection of polynucleotides, such as one or more target molecules and a collection of polynucleotides from a cell, such as a T cell, or a population or composition of cells, such as a population or composition of cells containing T cells. In some embodiments, the methods and compositions described herein are useful for single cells analysis or bulk analysis on a population or composition of cells, such as, e.g., for the study of genomes, transcriptomes, proteomes, metabolic pathways and the like of complex cell samples. In other embodiments, TCR pairing information and single-cell analysis can be combined to associate cell function or cell status information with a particular T cell clone.

In some embodiments, the methods provided herein involves steps in which nucleic acids are manipulated in order to generate libraries of polynucleotides for sequencing. In a general sense, in some embodiments, amplification of immune cell and/or T cell genetic material, e.g. reverse transcription polymerase chain reaction (reverse transcription-PCR) is employed to generate cDNA amplification of immune cell genetic material.

In some embodiments, the provided methods can include various features of the methods as described in WO2016/044227, WO2016/176322, WO2012/048340, WO2012/048341, WO2014/144495, WO2016/044227, WO2016/176322, or WO2017/053902, each incorporated by reference in their entirety. In some aspects, exemplary methods, including single cell paired TCR sequencing methods, are described herein. In some aspects, other methods, such as bulk immune sequencing methods (e.g., barcoded bulk TCR sequencing), high-throughput whole genome or whole transcriptome sequencing methods, can be utilized for clonotype analysis.

In some embodiments, the methods utilize steps in which nucleic acids are manipulated in order to generate libraries of polynucleotides for sequencing. In some embodiments, the methods utilize steps in which nucleic acids are manipulated in order to produce recombinant monoclonal antibodies. In some embodiments, the methods utilize steps in which nucleic acids are manipulated in order to produce polynucleotides that represent the transcriptome or genome of one or more cells. In a general sense, in some embodiments, amplification of immune cell and/or T cell genetic material, e.g. reverse transcription polymerase chain reaction (reverse transcription-PCR) is employed to generate cDNA amplification of immune cell genetic material.

In some embodiments, in addition to obtaining full-length sequence data of a target polynucleotide of interest, e.g. immune molecule, such as TCR, the provided methods also permit efficient generation of high quality DNA sequencing libraries from both the whole transcriptome product and the full-length target polynucleotide, e.g. TCR, including full-length paired immune receptor product. In some embodiments, such methods include the addition (e.g. ligation) of adaptor DNA sequence to the single-stranded polynucleotide products, which can permit amplification and next-generation sequencing of the transcriptome.

In some embodiments, methods are provided for producing a polynucleotide library, that include the steps of (a) lysing cells within each of a plurality of vessels, wherein each of said vessels comprises a cell from a sample comprising a population of cells, a plurality of molecular barcoded oligonucleotides, and a first adaptor comprising a vessel barcoded oligonucleotide; (b) producing, in each vessel, a plurality of single-stranded polynucleotides comprising (i) one or more target single-stranded polynucleotide(s) that is complementary to one or more target polynucleotide(s) present in the cell; and (ii) a collection of single-stranded polynucleotides that each are complementary to a polynucleotide in the cell; (c) attaching to each single-stranded polynucleotide one of the plurality of molecular barcoded oligonucleotides, thereby generating a plurality of barcoded single-stranded polynucleotides each comprising a unique molecular barcode; (d) attaching the first adaptor comprising the vessel barcoded oligonucleotide, or an amplified product thereof, to each of the barcoded single-stranded polynucleotides, thereby generating a plurality of dual-barcoded single-stranded polynucleotides, wherein each of the dual-barcoded single-stranded polynucleotides in the same vessel comprise the same vessel barcode; and (e) adding a second adaptor to each of the dual-barcoded single-stranded polynucleotides, wherein the first adaptor and second adaptor are present at or near opposite ends of each of the dual-barcoded single-stranded polynucleotides.

In some embodiments, methods are provided for producing a polynucleotide library, whereby an adaptor is added to each of a plurality of previously adaptor-tagged, barcoded single-stranded polynucleotides, such that the adaptors are at opposite ends of the polynucleotides, wherein the plurality of barcoded single-stranded polynucleotides include (i) one or more target single-stranded polynucleotide(s) that is complementary to one or more target polynucleotide(s) present in a cell of a population of cells; and (ii) a collection of single-stranded polynucleotides that each are complementary to a polynucleotide in the cell, and for each of the plurality of barcoded single-stranded polynucleotides, the first vessel barcode that is the same for all complementary polynucleotides from the same cell of the population of cells.

The polynucleotide starting material, such as RNA, can be reverse transcribed into cDNA using one or a pool of polynucleotides. The polynucleotides can comprise a portion complementary to a region of the target RNA, such as in a constant region of the target or to a poly-A tail of the mRNA.

cDNA resulting from reverse transcription can be tagged with one or more barcodes. For example the cDNA can be tagged with a vessel barcode, which can be a stretch of ˜20 degenerate nucleotides with or without a known intercalating base position, such as NNNNWISCNNNWISCNNN (SEQ ID NO: 49), where W means A or T can be used to tag the cDNA molecules processed in the same vessel. In particular examples, the cDNA molecules processed in the same vessel are complementary to RNA molecules from the same cell.

In some examples, the cDNA resulting from reverse transcription can be tagged with a vessel barcode and a molecular barcode. Various oligonucleotides of particular design can be used for tagging. Tagged cDNA resulting from reverse transcription can be amplified one or more times, such as by PCR amplification. Various primers of particular design can be used for the amplification. A product of a first amplification reaction, such as PCR, can be amplified using a second amplification reaction, such as a first or second PCR phase. Various primers can be used for the amplification step. A library of amplified polynucleotides can be generated using the methods described herein. A resulting library can comprise a full or partial TCR sequence with appropriate molecular and vessel barcodes.

In other embodiments, template switching can be used to generate libraries, such as for immune repertoire sequencing. For example, template switching can be employed during reverse transcription to generate a region on the product of the reverse transcription that is complementary to a polynucleotide harboring a barcode, such as a vessel barcoded polynucleotide or a molecular barcoded polynucleotide. Template switching can be employed during reverse transcription to remove issues of PCR bias.

Target polynucleotides can be reverse transcribed into cDNA using one or a pool of polynucleotides. Examples of primers in a pool of polynucleotides for reverse transcribing a target polynucleotide can comprise a portion complementary to a region of the target polynucleotide. In some embodiments, the portion complementary to a region of the target polynucleotide can be complementary to a constant region or to a poly-A tail of the target polynucleotide, such as mRNA. Multiple oligonucleotides, such as primers, can be used to anneal one or more constant regions. A reverse transcriptase can be employed to carry out the reverse transcription reaction. In particular embodiments, a reverse transcriptase can comprise a non-template terminal transferase activity. When a reverse transcriptase comprising non-template terminal transferase activity reaches the end of a template, it can add three or more non-template residues, such as three or more non-template cytosine residues. In some embodiments, Superscript II™ reverse transcriptase is used for this purpose. In some embodiments, Maxima™ reverse transcriptase is used for this purpose. In some embodiments, Protoscript II™ reverse transcriptase is used for this purpose. In some embodiments, Maloney murine leukemia virus reverse transcriptase (MMLV-RT) is used for this purpose. In some embodiments, HighScriber™ Reverse Transcriptase is used for this purpose. In some embodiments a terminal deoxynucleotidyl transferase is used for this purpose. In some embodiments avian myeloblastosis virus (AMV) reverse transcriptase is used for this purpose. Any reverse transcriptase capable of transcribing R A that has non-template terminal transferase activity can be used. Any reverse polymerase capable of transcribing RNA that has non-template terminal transferase activity can be used. Any reverse polymerase capable of transcribing DNA that has non-template terminal transferase activity can be used.

Reverse transcription reactions, such as those described above, can be conducted in the presence of a 3′ tagging polynucleotide. A 3′ tagging polynucleotide can be a polynucleotide used to add nucleic acids to a 3′ end of a target polynucleotide, such as a cDNA. A 3′ tagging polynucleotide can be a polynucleotide used as a template to add nucleic acids to a 3′ end of a target polynucleotide, such as a cDNA. A 3′ tagging polynucleotide can be a polynucleotide that hybridizes to a 3′ end of a target polynucleotide, such as a cDNA. A 3′ tagging polynucleotide can be a polynucleotide that contains a 3′ region, such as a 3′ terminal region, that hybridizes to a 3′ end of a target polynucleotide, such as a cDNA. For example, a 3′ tagging polynucleotide can comprise a segment, such as a segment that anneals to three or more non-template residues. In some embodiments, a 3′ tagging polynucleotide is a molecular barcode polynucleotide. In some embodiments, a 3′ tagging polynucleotide can comprise a molecular barcode. In some embodiments, a 3′ tagging polynucleotide can comprise 3′ riboguanosine residues or analogues thereof on the 3′ end (rGrGrG) (RNA bases) that are complementary to and annealed to the strand produced by the reverse transcription enzyme. In some embodiments, three or more guanine residues can be used instead of riboguanosine (DNA nucleotide instead of RNA nucleotide). In some embodiments, a 3′ tagging polynucleotide can comprise 1 or 2 riboguanosine residues on the 3′ end and a riboguanosine residue or analogue thereof on the 3′ end (rGrGG) that are complementary to and annealed to the strand produced by the reverse transcription enzyme.

Upon annealing of a 3′ tagging polynucleotide to a CCC of the cDNA strand, a reverse transcriptase can continue extending the cDNA into the tagging polynucleotide, thereby attaching a molecular barcode or complement thereof, to a target population of polynucleotides, such as cDNAs, in the reaction. For example, 3′ tagging polynucleotide can be a polynucleotide that contains a region 5′ to the 3′ region that hybridizes to a 3′ end of a target polynucleotide. The region 5′ to the 3′ region that hybridizes to a 3′ end of a target polynucleotide can comprise a region that is not complementary to the target polynucleotide, such as a cDNA. The region 5′ to the 3′ region that hybridizes to a 3′ end of a target polynucleotide can comprise a molecular barcode. The region 5′ to the 3′ region that hybridizes to a 3′ end of a target polynucleotide can comprise a region complementary to a vessel barcoded polynucleotide or complement thereof. In other experiments, template switching can be performed in separate reactions. For example, a 3′ tagging polynucleotide can be added after the reverse transcription reaction, and enzymes such as a reverse transcriptase or polymerase can be used to extend into a tagging polynucleotide. Because a tagging polynucleotide can harbor a unique degenerate molecular barcode on each molecule in a vessel, each cDNA in a vessel can be uniquely tagged with a molecular barcode. In some embodiments, template switching can be performed at the same time as a reverse transcription reaction is conducted.

In some embodiments, a 3′ tagging polynucleotide, such as a molecular barcoded polynucleotide, can further comprise a 5′ region, such as a 5′ terminal region that is complementary to a 3′ tagging polynucleotide or complement thereof containing another barcode, such as a vessel barcode. In some embodiments, a target polynucleotide that contains a molecular barcode or complement thereof, such as a tagged cDNA molecule, can comprise a 3′ region, such as a 3′ terminal region that is complementary to a 3′ tagging polynucleotide or complement thereof containing another barcode, such as a vessel barcode.

In some embodiments, a 3′ tagging polynucleotide is a vessel barcoded polynucleotide. Upon generation of a polynucleotide containing a molecular barcode or complement thereof from a target polynucleotide, a vessel barcode can be added to the molecular barcoded target polynucleotide. A 3′ tagging polynucleotide can be a polynucleotide used to add nucleic acids to a 3′ end of a target polynucleotide, such as a molecular barcoded target polynucleotide. A 3′ tagging polynucleotide can be a polynucleotide used as a template to add nucleic acids to a 3′ end of a target polynucleotide, such as a molecular barcoded target polynucleotide. A 3′ tagging polynucleotide can be a polynucleotide that hybridizes to a 3′ end of a target polynucleotide, such as a molecular barcoded target polynucleotide. A 3′ tagging polynucleotide can be a polynucleotide that contains a 3′ region, such as a 3′ terminal region, that hybridizes to a 3′ end of a target polynucleotide, such as a molecular barcoded target polynucleotide. A vessel barcoded polynucleotide can comprise a 3′ region, such as a 3′ terminal region, that hybridizes to a 3′ end of a molecular barcoded target polynucleotide.

Upon annealing of a 3′ tagging polynucleotide to a molecular barcoded target polynucleotide, a reverse transcriptase can continue extending the cDNA into the 3′ tagging polynucleotide, such as a vessel barcoded polynucleotide, thereby attaching a vessel barcode or complement thereof, to a target population of polynucleotides, such as molecular barcoded target polynucleotides, in the reaction. For example, 3′ tagging polynucleotide can be a polynucleotide that contains a region 5′ to the 3′ region that hybridizes to a 3′ end of a molecular barcoded target polynucleotide. The region 5′ to the 3′ region that hybridizes to a 3′ end of a molecular barcoded target polynucleotide can comprise a region that is not complementary to the target polynucleotide or the molecular barcoded target polynucleotide. The region 5′ to the 3′ region that hybridizes to a 3′ end of a molecular barcoded target polynucleotide can comprise a vessel barcode.

In some embodiments, a 3′ tagging polynucleotide is an amplified product. In some embodiments, a 3′ tagging polynucleotide is an amplified product originating from a single molecule. In some embodiments, a 3′ tagging polynucleotide is an amplified product of a vessel barcoded polynucleotide. In some embodiments, a 3′ tagging polynucleotide is an amplified product originating from a single vessel barcoded polynucleotide. The region 5′ to the 3′ region that hybridizes to a 3′ end of a molecular barcoded target polynucleotide can comprise a region complementary to a primer or complement thereof. The region 5′ to the 3′ region that hybridizes to a 3′ end of a molecular barcoded target polynucleotide can comprise a region complementary to a primer or complement thereof that was used to amplify the vessel barcoded polynucleotide.

A dual barcoded target polynucleotide, such as a cDNA containing a molecular barcode and a vessel barcode can then be amplified, such as by PCR. The PCR can then be conducted, for example, by using a primer set. A product of the aforementioned PCR reaction can then be amplified one or more times, such as by one or more rounds of PCR, or directly sequenced.

A library produced according to the methods described herein can be a library comprising a large or full TCR sequence with appropriate barcodes, such as vessel barcodes and molecular barcodes, which are sequenced. In some embodiments, a library produced according to the methods described herein can contain appropriate clustering segments for sequencing. In some embodiments, many copies of identical molecular barcodes can be generated. In some embodiments, many copies of polynucleotides containing identical molecular barcodes can be generated for each starting unique target polynucleotide molecule. In some embodiments, many copies of polynucleotides containing identical molecular barcodes can be generated for each starting unique target polynucleotide molecule tagged with a vessel barcode.

Upon sequencing, sequences with identical molecular barcodes can be matched or paired. Upon sequencing, sequences with identical vessel barcodes can be matched or paired. Upon sequencing, sequences with identical target sequences can be matched or paired. In some embodiments, sequencing reads can be collapsed into consensus sequences. Collapsing matched or paired sequencing reads into a consensus sequence can thereby reduce or eliminate sequencing and PCR errors. Sequencing can be performed using a first primer site for a first read. Sequencing can be performed using the first primer site for a second read. Sequencing can be performed using a second primer site for a second read.

TCR alpha and beta chains containing the same vessel barcodes, can be paired, and in some embodiments, cloned in a mammalian vector system. The TCR construct can be expressed in other human or mammalian host cell lines. The construct can then be validated by transient transfection assays and Western blot analysis of the expressed antibody of interest.

Methods of amplification of RNA or DNA are well known in the art and can be used according to the present embodiments without undue experimentation, based on the teaching and guidance presented herein. Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; 5,142,033 to Innis; 5,122,464 to Wilson, et al.; 5,091,310 to Innis; 5,066,584 to Gyllensten, et al; 4,889,818 to Gelfand, et al.; 4,994,370 to Silver, et al.; 4,766,067 to Biswas; 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

Conveniently, the method steps described herein, such as amplification, sequencing, and the like, may or may not be carried out in a multiplex assay format employing a solid phase on which a plurality of substrates, e.g., antigens, and the like, are immobilized, such as an array. In some embodiments, the array is a protein biochip. Using protein biochips, hundreds and even thousands of antigens can be screened. As used herein, “array,” “microarray,” or “biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface, and therefore, function as probes. A “protein biochip” refers to a biochip adapted for the capture of polypeptides. Many protein biochips are described in the art. Methods of producing polypeptide arrays are described, e.g., in De Wildt et al, 2000, Nat. Biotechnol. 18:989-994; Lueking et al., 1999, Anal. Biochem. 270: 103-1 11; Ge, 2000, Nucleic Acids Res. 28, e3, 1-VH; MacBeath and Schreiber, 2000, Science 289: 1760-1763; WO 01/40803 and WO 99/51773A1. Use of arrays allows a number of the steps, such as screening, to be performed robotically and/or in a high-throughput manner. Polypeptides for the array can be spotted at high speed, e.g., using a commercially available robotic apparatus, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

Upon capture on a biochip, analytes can be detected by a variety of detection methods selected from, for example, a gas phase ion spectrometry method, an optical method, an electrochemical method, atomic force microscopy and a radio frequency method. Of particular interest is the use of mass spectrometry, and in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods Immunoassays in various formats (e.g., ELISA) are popular methods for detection of analytes captured on a solid phase. Electrochemical methods include voltammetry and amperometry methods. Radio frequency methods include multipolar resonance spectroscopy.

In some embodiments, e.g., the natural diversity approach for preparing monoclonal antibodies, techniques which have been established for working with single cells are employed. One technique incorporates a special accessory which can be used in FACS to deflect single cells into separate containers. Such accessories are commercially available and well-known in the art. Such accessories are useful for dispensing single cells into selected compartments of, for example, standard 96 well microtiter culture plates. Alternatively, cells may be deposited into a microtiter plate at a limiting dilution to ensure single cell deposition.

A second technique is PCR performed on single immune cells to amplify the V_{H} and V L segments. In the natural diversity approach, single cell PCR is used to retain the native pairing of V L and V_{H} in the single cell. The specificity of an antibody is determined by the complementarity determining regions (CDRs) within the V L region and V_{H} region.

Methods for performing single-cell PCR are well known in the art (e.g., Larrick, J. W. et al., Bio/Technology 7:934 (1989)). For example, antibody-producing B-cells from the B cell library or TCR-producing T-cells from the T-cell library may be fixed with a fixative solution or a solution containing a chemical such as formaldehyde, glutaraldehyde or the like. The cells are then permeabilized with a permeabilization solution comprising for example a detergent. The fixing and permeabilization process should provide sufficient porosity to allow entrance of enzymes, nucleotides and other reagents into the cells without undue destruction of cellular compartments or nucleic acids therein. Addition of enzymes and nucleotides may then enter the cells to reverse transcribe cellular V_{H} and V_{L} or Vα and Vβ or Vγ and Vδ mRNA, for example, into the corresponding cDNA sequences.

Reverse transcription may be performed in a single step or optionally together with a PCR procedure, using a reverse transcriptase, sufficient quantities of the four dNTPs, and primers that bind to the mRNA providing a 3′ hydroxyl group for reverse transcriptase to initiate polymerization. Target-specific primers and/or random hexamer oligonucleotide primers can be used to initiate the reverse transcription reaction and generate high quality sequencing libraries.

For target sequences, any primer complementary to the target mRNA may be used, but it is preferred to use primers complementary to a 3′-terminal end of the Vα and Vβ or Vγ and Vδ molecules so as to facilitate selection of variable region mRNA. Numerous studies have indicated that degenerate polynucleotides can be prepared to serve as the 5′-end primers for Vα and Vβ or Vγ and Vδ. The combinatorial library method of making targeting molecules relies on such primers. Furthermore, numerous experiments have shown that PCR can amplify the gene segments of interest, such as V Vα and Vβ or Vγ and Vδ, from a single cell. Because of the ability to work with even a single cell, this PCR approach can generate antibodies even where the immune cells of interest occur at low frequency.

In the high diversity embodiment, after FACS sorting, the cells of immune cell library are pooled and the reverse transcription-PCR is performed on the entire pool of cells. Generation of mRNA for cloning antibodies or TCRs purposes is readily accomplished by well-known procedures for preparation and characterization of antibodies or TCRs (see, e.g., Antibodies: A Laboratory Manual, 1988; incorporated herein by reference). cDNA is then synthesized from the RNA by appropriate methods, e.g. using random hexamer polynucleotides, or C-gene or C-gene family-specific primers, or V-gene or V-gene family-specific primers. Again these are processes known to persons skilled in the art as explained above. Libraries of nucleic acid molecules derived from T-cell libraries, e.g. a library of RNA or cDNA molecules derived from such T lymphocytes, may be cloned into expression vectors to form expression libraries. In some embodiments, only the Vα or Vγ domain derived from the immune cell library is amplified to generate a library of Vα or Vγ domains. A Vβ or Vδ library from another source is used in combination with the Vα or Vγ library to generate TCRs using methods described herein. Libraries of TCR fragments can be constructed by combining Vα and Vβ or Vγ and Vδ libraries together in any number of ways as known to the skilled artisan. For example, each library can be created in different vectors, and the vectors recombined in vitro, or in vivo. Alternatively, the libraries may be cloned sequentially into the same vector, or assembled together by PCR and then cloned. PCR assembly can also be used to join Vα and Vβ or Vγ and Vδ DNAs with DNA encoding a flexible peptide spacer to form single chain Fv (scFv) libraries as described elsewhere herein. In yet another technique, in-cell PCR assembly is used to combine Vα and Vβ or Vγ and Vδ genes within lymphocytes by PCR and then clone repertoires of linked genes.

3. Target Polynucleotides

In embodiments, methods provided herein are directed to amplification and sequencing of a target polynucleotide molecule, such as a polynucleotide molecule from a cell or a population or composition of cells. In some cases, methods provided herein are directed to amplification and sequencing of one or more regions of a target polynucleotide molecule. In some cases, methods provided herein are directed to amplification and sequencing of two or more regions of a target polynucleotide molecule. In some cases, methods provided herein are directed to amplification and sequencing of two or more target polynucleotide molecules, such as two or more naturally paired molecules. In one aspect, target polynucleotides are RNA. In one aspect, target polynucleotides are genomic nucleic acids. DNA derived from the genetic material in the chromosomes of a particular organism can be genomic DNA.

In some embodiments, reference to a “target nucleic acid molecule,” “target polynucleotide,” “target polynucleotide molecule,” refers to any nucleic acid of interest.

In some embodiments, target polynucleotides include sequences comprising variable regions of an immune receptor, such as a TCR produced by an immune cell. In some aspects, target polynucleotides include sequences comprising variable region of a single chain or a portion thereof of a TCR, such as one of TCR α, β, γ or δ chain or a portion thereof.

In some embodiments, target polynucleotides that are naturally paired to generate an immune receptor or binding fragment thereof. In some embodiments, target polynucleotides include sequences comprising a variable region of a heavy chain of a TCR produced by an immune cell. In some embodiments, target polynucleotides include sequences comprising a variable region of an alpha chain of a TCR produced by an immune cell. In some embodiments, target polynucleotides include sequences comprising a variable region of a beta chain and sequences comprising a variable alpha chain of a TCR produced by the same immune cell.

In some embodiments, target polynucleotides include sequences comprising a variable region of an alpha chain of a TCR produced by an immune cell. In some embodiments, target polynucleotides include sequences comprising a variable region of a beta chain of a TCR produced by an immune cell. In some embodiments, target polynucleotides include sequences comprising a variable region of an alpha chain of a TCR and sequences comprising a variable region of a beta chain of a TCR produced by the same immune cell. In some embodiments, target polynucleotides include sequences comprising a variable region of a gamma chain of a TCR produced by an immune cell. In some embodiments, target polynucleotides include sequences comprising a variable region of a delta chain of a TCR produced by an immune cell. In some embodiments, target polynucleotides include sequences comprising a variable region of a gamma chain of a TCR and sequences comprising a variable region of a delta chain of a TCR produced by the same immune cell.

In some embodiments, a TCR encompasses full TCRs as well as antigen-binding portions or antigen-binding fragments (also called MHC-peptide binding fragments) thereof. In some embodiments, the TCR is an intact or full-length TCR. In some embodiments, the TCR is an antigen-binding portion that is less than a full-length TCR but that binds to a specific antigenic peptide bound to (i.e., in the context of) an MHC molecule, i.e., an MHC-peptide complex. In some cases, an antigen-binding portion or fragment of a TCR can contain only a portion of the structural domains of a full-length or intact TCR, but yet is able to bind the epitope (e.g., MHC-peptide complex) to which the full TCR binds. In some cases, an antigen-binding portion or fragment of a TCR contains the variable domains of a TCR, such as variable a chain and variable β chain of a TCR, sufficient to form a binding site for binding to a specific MHC-peptide complex, such as generally where each chain contains three complementarity determining regions. Polypeptides or proteins having a binding domain which is an antigen-binding domain or is homologous to an antigen-binding domain are included. Complementarity determining region (CDR) grafted antibodies and TCRs and other humanized antibodies and TCRs (including CDR modifications and framework region modifications) are also contemplated by these terms. It should be noted that while reference may be made only to immunoglobulin chains (e.g., heavy chains and lights chains), the disclosed embodiments can be applied to multiple other different types of paired sequences, e.g., T-cell receptor chain pairs (TCRα and TCRβ chains and TCRγ and TCRδ chains), and is not limited to immunoglobulins.

The ability of T-cells to recognize antigens associated with various cancers or infectious organisms is conferred by its TCR, which is made up of both an alpha (α) chain and a beta (β) chain or a gamma (γ) and a delta (δ) chain. The proteins which make up these chains are encoded by DNA, which employs a unique mechanism for generating the tremendous diversity of the TCR. This multi-subunit immune recognition receptor associates with the CD3 complex and binds peptides presented by the MEW class I and II proteins on the surface of antigen-presenting cells (APCs). Binding of a TCR to the antigenic peptide on the APC is a central event in T-cell activation, which occurs at an immunological synapse at the point of contact between the T-cell and the APC.

Each TCR comprises variable complementarity determining regions (CDRs), as well as framework regions (FRs). The amino acid sequence of the third complementarity-determining region (CDR3) loops of the α and β chain variable domains largely determines the sequence diversity of αβ T-cells arising from recombination between variable (Vβ), diversity (Dβ), and joining (Jβ) gene segments in the β chain locus, and between analogous Vα and Jα gene segments in the a chain locus, respectively. The existence of multiple such gene segments in the TCR α and β chain loci allows for a large number of distinct CDR3 sequences to be encoded. Independent addition and deletion of nucleotides at the Vβ-Dβ, Dβ-Jβ, and Vα-Jα junctions during the process of TCR gene rearrangement further increases CDR3 sequence diversity. In this respect, immunocompetence is reflected in the diversity of TCRs.

A “germline sequence” refers to a genetic sequence from the germline (the haploid gametes and those diploid cells from which they are formed). Germline DNA contains multiple gene segments that encode a single TCRα or TCRβ chain, or a single TCRγ or TCRδ chain. These gene segments are carried in the germ cells but cannot be transcribed and translated until they are arranged into functional genes. During T-cell differentiation in the bone marrow, these gene segments are randomly shuffled by a dynamic genetic system capable of generating more than 108 specificities. Most of these gene segments are published and collected by the germline database.

In some embodiments, the sample, such as a population of cells or a single cell can contain an immune repertoire, e.g. TCR repertoire, and such can be elucidated by the provided methods. In some embodiments, aTCR repertoire refers to a collection of antibodies, TCRs, or fragments thereof. In some embodiments, an antibody repertoire can, for example, be used to select a particular antibody or screen for a particular property, such as binding ability, binding specificity, ability of gastrointestinal transport, stability, affinity, and the like. The term specifically includes antibody and TCR libraries, including all forms of combinatorial libraries, such as, for example, antibody phage display libraries, including, without limitation, single-chain Fv (scFv) and Fab antibody phage display libraries from any source, including naïve, synthetic and semi-synthetic libraries.

Target polynucleotides can be obtained from virtually any source and can be prepared using methods known in the art. For example, target polynucleotides can be directly isolated without amplification using methods known in the art, including without limitation extracting a fragment of genomic DNA or mRNA from an organism or a cell (e.g., an immune cell) to obtain target polynucleotides. A target polynucleotide can also encompass cDNA generated from RNA (such as mRNA) through reverse transcription-PCR. In some cases, a target polynucleotide is an RNA molecule. In some cases, a target polynucleotide is an mRNA molecule, or a cDNA produced from the mRNA molecule. In some cases, a target polynucleotide is an mRNA molecule, or cDNA molecule produced from the mRNA molecule, from a single immune cell. In some cases, target polynucleotides are mRNA molecules, or cDNA molecules produced from the mRNA molecules, from individual immune cells. In some cases, target polynucleotides are mRNA molecules encoding an antibody sequence from a single immune cell. In some cases, target polynucleotides are mRNA molecules encoding heavy chain antibody sequences from individual immune cells. In some cases, target polynucleotides are mRNA molecules encoding a heavy chain antibody sequence from a single immune cell. In some cases, target polynucleotides are mRNA molecules encoding light chain antibody sequences from individual immune cells. In some cases, target polynucleotides are mRNA molecules encoding a light chain antibody sequence from a single immune cell. In some cases, target polynucleotides are mRNA molecules encoding antibody variable sequences from individual immune cells. In some cases, target polynucleotides are mRNA molecules encoding a variable antibody sequence from a single immune cell. In some cases, target polynucleotides are mRNA molecules encoding variable light chain antibody sequences from individual immune cells. In some cases, target polynucleotides are mRNA molecules encoding a variable light chain antibody sequence from a single immune cell. In some cases, target polynucleotides are mRNA molecules encoding variable heavy chain antibody sequences from individual immune cells. In some cases, target polynucleotides are mRNA molecules encoding a variable heavy chain antibody sequence from a single immune cell. In some cases, a target polynucleotide can be a cell-free nucleic acid, e.g., DNA or RNA. In some cases, target polynucleotides are mRNA molecules encoding variable alpha, beta, gamma, and/or delta chain TCR sequences from individual immune cells.

The methods described herein can be used to generate a library of polynucleotides from one or more target polynucleotides for sequencing. Target polynucleotides include any polynucleotides of interest that are not products of an amplification reaction. For example, a target polynucleotide can include a polynucleotide in a biological sample. For example, target polynucleotides do not include products of a PCR reaction. For example, target polynucleotides may include a polynucleotide template used to generate products of an amplification reaction, but do not include the amplification products themselves. For example, target polynucleotides may include a polynucleotide template used to generate products of a reverse transcription reaction or primer extension reaction, and also include the reverse transcription reaction or primer extension reaction products themselves. For example, target polynucleotides include polynucleotides of interest that can be subjected to a reverse transcription reaction or a primer extension reaction. For example, target polynucleotides include RNA or DNA. For example, target polynucleotides include cDNA. In some embodiments, target RNA polynucleotides are mRNA. In some embodiments, target RNA polynucleotides are polyadenylated. In some embodiments, the RNA polynucleotides are not polyadenylated. In some embodiments, the target polynucleotides are DNA polynucleotides. The DNA polynucleotides may be genomic DNA. The DNA polynucleotides may comprise exons, introns, untranslated regions, or any combination thereof.

In some embodiments, libraries can be generated from two or more regions of a target polynucleotide. In some embodiments, methods libraries can be generated from two or more target polynucleotides. In some embodiments, target polynucleotides are genomic nucleic acids or DNA derived from chromosomes. In some embodiments, target polynucleotides include sequences comprising a variant, such as a polymorphism or mutation. In some embodiments, target polynucleotides include DNA and not RNA. In some embodiments, target polynucleotides include RNA and not DNA. In some embodiments, target polynucleotides include DNA and RNA. In some embodiments, a target polynucleotide is an mRNA molecule. In some embodiments, a target polynucleotide is a DNA molecule. In some embodiments, a target polynucleotide is a single stranded polynucleotide. In some embodiments, a target polynucleotide is a double stranded polynucleotide. In some embodiments, a target polynucleotide is a single strand of a double stranded polynucleotide.

Target polynucleotides can be obtained from any biological sample and prepared using methods known in the art. In some embodiments, target polynucleotides are directly isolated without amplification. Methods for direct isolation are known in the art. Non-limiting examples include extracting genomic DNA or mRNA from a biological sample, organism or, cell.

In some embodiments, one or more target polynucleotides are purified from a biological sample. In some embodiments, a target polynucleotide is not purified from the biological sample in which it is contained. In some embodiments, a target polynucleotide is isolated from a biological sample. In some embodiments, a target polynucleotide is not isolated from the biological sample in which it is contained. In some embodiments, a target polynucleotide can be a cell-free nucleic acid. In some embodiments, a target polynucleotide can be a fragmented nucleic acid. In some embodiments, a target polynucleotide can be a transcribed nucleic acid. In some embodiments, a target polynucleotide is a modified polynucleotide. In some embodiments, a target polynucleotide is a non-modified polynucleotide.

In some embodiments, a target polynucleotide is polynucleotide from a single cell. In some embodiments, target polynucleotides are from individual cells. In some embodiments, a target polynucleotide is polynucleotide from a sample containing a plurality of cells.

In some embodiments, a target polynucleotide encodes a biomarker sequence. In some embodiments, a target polynucleotide encodes two or more biomarker sequences. In some embodiments, a plurality of target polynucleotides encodes a biomarker sequence. In some embodiments, a plurality of target polynucleotides encodes two or more biomarker sequences. In some embodiments, a plurality of target polynucleotides encodes 3, 4, 5, 6, 7, 8, 9, 10, 20, 30, 40, 50, 60, 70, 80, 90, or 100 or more biomarker sequences.

In some embodiments, a plurality of target polynucleotides comprises a panel of TCR sequences. In some embodiments, a panel of TCR sequences contains 1, 2, 3, 4, 5, 6, 7, 8, 9, or 10 TCR sequences. In some embodiments, a panel of TCR sequences contains at least about 10, 20, 30, 40, 50, 60, 70, 80, 90, 100, 150, 200, 250, 300, 350, 400, 3000, 4000, 5000, 6000, 7000, 450, 500, 550, 600, 650, 700, 750, 800, 850, 900, 1000, 1500, 2000, 8000, 9000, 10,000, 11,000, 12,000, 13,000, 14,000, 15,000, 16,000, 17,000, 18,000, 19,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁷, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁷, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰ , 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², or 9×10¹² TCR sequences. In some embodiments, a panel of TCR sequences contains at most about 10, 20, 30, 40, 50, 60, 70, 80, 90, 100, 150, 200, 250, 300, 350, 400, 450, 500, 550, 600, 650, 700, 750, 800, 850, 900, 1000, 1500, 2000, 3000, 4000, 5000, 6000, 7000, 8000, 9000, 10,000, 11,000, 12,000, 13,000, 14,000, 15,000, 16,000, 17,000, 18,000, 19,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², or 9×10¹² TCR sequences. In some embodiments, a panel of TCR sequences contains from about 10-20, 10-30, 10-40, 10-30, 10-40, 10-50, 10-60, 10-70, 10-80, 10-90, 10-100, 50-60, 50-70, 50-80, 50-90, 50-100, 100-200, 100-300, 100-400, 100-300, 100-400, 100-500, 100-600, 100-700, 100-800, 100-900, 100-1000, 500-600, 500-700, 500-800, 500-900, 500-1000, 1000-2000, 1000-3000, 1000-4000, 1000-3000, 1000-4000, 1000- 5000, 1000-6000, 1000-7000, 1000-8000, 1000-9000, 1000-10000, 5000-6000, 5000-7000, 5000- 8000, 5000-9000, 5000-10000, 1-1×10⁵, 1-2×10⁵, 1-3×10⁵, 1-4×10⁵, 1-5×10⁵, 1-6×10⁵, 1-7×10⁵, 1- 8×10⁵, 9×10⁵, 1-1×10⁶, 1-2×10⁶, 1-3×10⁶, 1-4×10⁶, 1-5×10⁶, 1-6×10⁶, 1-7×10⁶, 1-8×10⁶, 9×10⁶, 1- 1×10⁷, 1-2×10⁷, 1-3×10⁷, 1-4×10⁷, 1-5×10⁷, 1-6×10⁷, 1-7×10⁷, 1-8×10⁷, 1-9×10⁷, 1-1×10⁸, 1-2×10⁸, 1-3×10⁸, 1-4×10⁸, 1-5×10⁸, 1-6×10⁸, 1-7×10⁸, 1-8×10⁸, 1-9×10⁸, 1-1×10⁹, 1-2×10⁹, 1-3×10⁹, 1-4×10⁹, 1-5×10⁹, 1-6×10⁹, 1-7×10⁹, 1-8×10⁹, 1-9×10⁹,1-1×10¹⁰, 1-2×10¹⁰, 1-3×10¹⁰, 1-4×10¹⁰ , 1-5×10¹⁰, 1-6×10¹⁰, 1-7×10¹⁰, 1-8×10¹⁰, 1-9×10¹⁰, 1-1×10¹¹, 1-2×10¹¹, 1-3×10¹¹, 1-4×10¹¹, 1-5×10¹¹, 1-6×10¹¹, 1-7×10¹¹, 1-8×10¹¹, 1-9×10¹¹, 1-1×10¹², 1-2×10¹², 1-3×10¹², 1-4×10¹², 1-5×10¹², 1-6×10¹², 1-7×10¹², 1-8×10¹², or 1-9×10¹² TCR sequences.

In some embodiments, a target polynucleotide is about 10, 20, 30, 40, 50, 60, 70, 80, 90, 100, 150, 200, 250, 300, 350, 400, 450, 500, 550, 600, 650, 700, 750, 800, 850, 900, 1000, 1500, 2000, 3000, 4000, 5000, 6000, 7000, 8000, 9000, 10,000, 11,000, 12,000, 13,000, 14,000, 15,000, 16,000, 17,000, 18,000, 19,000, or 20,000 bases or base-pairs in length. In some embodiments, a target polynucleotide is at least about 10, 20, 30, 40, 50, 60, 70, 80, 90, 100, 150, 200, 250, 300, 350, 400, 450, 500, 550, 600, 650, 700, 750, 800, 850, 900, 1000, 1500, 2000, 3000, 4000, 5000, 6000, 7000, 8000, 9000, 10,000, 11,000, 12,000, 13,000, 14,000, 15,000, 16,000, 17,000, 18,000, 19,000, or 20,000 bases or base-pairs in length. In some embodiments, a target polynucleotide is at most about 10, 20, 30, 40, 50, 60, 70, 80, 90, 100, 150, 200, 250, 300, 350, 400, 450, 500, 550, 600, 650, 700, 750, 800, 850, 900, 1000, 1500, 2000, 3000, 4000, 5000, 6000, 7000, 8000, 9000, 10,000, 11,000, 12,000, 13,000, 14,000, 15,000, 16,000, 17,000, 18,000, 19,000, or 20,000 bases or base-pairs in length. In some embodiments, a target polynucleotide is from about 10-20, 10-30, 10-40, 10- 30, 10-40, 10-50, 10-60, 10-70, 10-80, 10-90, 10-100, 50-60, 50-70, 50-80, 50-90, 50-100, 100-200, 100-300, 100-400, 100-300, 100-400, 100-500, 100-600, 100-700, 100-800, 100-900, 100-1000, 500-600, 500-700, 500-800, 500-900, 500-1000, 1000-2000, 1000-3000, 1000-4000, 1000-3000, 1000-4000, 1000-5000, 1000-6000, 1000-7000, 1000-8000, 1000-9000, 1000-10000, 5000-6000, 5000-7000, 5000-8000, 5000-9000, or 5000-10000 bases or base-pairs in length. In some embodiments, the average length of the target polynucleotides, or fragments thereof, can be less than about 100, 200, 300, 400, 500, or 800 base pairs, or less than about 5, 10, 20, 30, 40, 50, 60, 70, 80, 90, 100, 110, 120, 130, 140, 150, 160, 170, 180, 190, or 200 nucleotides, or less than about 1, 2, 5, 10, 20, 30, 40, 50, 60, 70, 80, 90, 100 kilobases. In some embodiments, a target sequence from a relative short template, such as a sample containing a target polynucleotide, is about 40, 45, 50, 55, 60, 65, 70, 75, 80, 85, 90, 95, or 100 bases. In certain embodiments, sequencing data are aligned against known or expected sequences using a database containing sequences or immunoglobulin or TCR sequences associated with a disease or condition.

4. Single Cell Barcoding and Sequence Determination

In some aspects, the provided methods can employ single-cell based sequencing methods to determine the clonotype of one cell, such as one T cell from a biological sample or in a composition comprising T cells. In some embodiments, the aspect of determining TCR sequence or clonotypes of a plurality of cells involves a method comprising: (a) forming a plurality of vessels each comprising a single cell from a sample comprising a plurality of cells, a plurality of molecular barcoded polynucleotides, and a vessel barcoded polynucleotide; (b) producing: a first complementary polynucleotide that is complementary to a first cell polynucleotide from the single cell, and a second complementary polynucleotide that is complementary to a second cell polynucleotide from the single cell; (c) attaching: a first molecular barcoded polynucleotide of the plurality to the first complementary polynucleotide, and a second molecular barcoded polynucleotide to the second complementary polynucleotide, thereby forming a first and a second single cell single- barcoded polynucleotide; and (d) attaching the vessel barcoded polynucleotide, or an amplified product thereof to the first single cell single-barcoded polynucleotide or an amplified product thereof, and the second single cell single-barcoded polynucleotide or an amplified product thereof, thereby forming a first and a second single cell dual-barcoded sequences.

In some embodiments, a composition can be used to determine the TCR sequence and/or clonal composition, such as a composition comprising: a plurality of vessels each comprising a single cell from a sample comprising a plurality of cells, a plurality of molecular barcoded polynucleotides, a vessel barcoded polynucleotide; a first complementary polynucleotide that is complementary to a first cell polynucleotide from the single cell, and a second complementary polynucleotide that is complementary to a second cell polynucleotide from the single cell; wherein the first complementary polynucleotide comprises a first molecular barcode of the plurality of molecular barcoded polynucleotides and the vessel barcode of the vessel barcoded polynucleotide or an amplified product of the vessel barcoded polynucleotide, and wherein the second complementary polynucleotide comprises a second molecular barcode of the plurality of molecular barcoded polynucleotides and the vessel barcode of the vessel barcoded polynucleotide or an amplified product of the vessel barcoded polynucleotide.

For single cell barcoding with a vessel barcode and molecular barcode, vessels, such as water in oil emulsions, can be created in such way that resulting vessels contain 1 cell or less per vessel. The vessels can be created in such way that resulting vessels also contain 1 vessel barcode per vessel. The vessels can be created in such way that resulting vessels also contain 1 molecular barcoded polynucleotide per vessel. The vessels can be created in such way that resulting vessels also contain two or more, or a plurality of, molecular barcoded polynucleotides per vessel. The cells/vessels can be subject to an RNA or DNA single barcoding protocol as described herein, and the vessel barcode and one or more molecular barcode of each vessel can be fused with a target of interest, such as a cell polynucleotide. In some embodiments, matching vessel barcoded polynucleotides can be fused to cell components present in the same vessel as the one or more molecular barcoded polynucleotides. Following sequencing, vessel barcode and molecular barcode deconvolution can be used to identify which RNA (or DNA) originated from which cell. In some embodiments, vessels, such as water in oil emulsions, can be created in such way that resulting emulsions contained 1 cell or more per emulsion. In some embodiments, water in oil emulsions can be created in such way that resulting emulsions contain lvessel barcoded polynucleotide and two or more molecular barcoded polynucleotides per vessel. In some embodiments, vessels can be created in such way that resulting vessels contain more than 1 vessel barcoded polynucleotide and two or more molecular barcoded polynucleotides per vessel. In some embodiments, a vessel barcode and molecular barcode can be introduced into vessels when in solution. In some embodiments, a vessel barcode and molecular barcode can be introduced into vessels when not attached to a solid support, such as a bead.

In some aspects, single cells can be isolated inside an emulsion, which can act as a compartment. The cells can be lysed and transcripts from the cell can be barcoded. Each of the transcripts can be fused with a molecular barcode or vessel barcode, in such way that when two or more RNA transcripts are detected with the same vessel barcode, they can be determined to have originated from the same starting cell. This can be applied to many different types of sequences. One particular application can be linking VH and VL or Vα and Vβ or Vγ and Vδ chains of antibody and TCR sequences.

One or more single cells can be isolated in one or more emulsions, in the presence of a vessel barcode and molecular barcodes, so that one droplet of the one or more emulsions can contain a maximum of 1 cell or less. Cells can be lysed chemically by a buffer contained in an emulsion or by freeze thaw, thereby releasing the contents of a cell in an emulsion.

RNAs of a single cell can be reverse transcribed into cDNA. A reverse transcription reaction can be done with a reverse transcriptase that possesses non-template terminal transferase activity which adds about 3 cytosine residues as described above. All reverse transcription buffers, enzymes, and nucleotides can be present when forming an emulsion. In some embodiments, a primer can be generalized (such as polynucleotide comprising a poly dT sequence) to target all mRNA. In some embodiments, DNA can be used. In some embodiments, more than 2 RNAs can be targeted.

In some embodiments, a vessel barcode can be linked to an RNA during reverse transcription. In some embodiments, a molecular barcode can be linked to an RNA during reverse transcription. In some embodiments, a vessel barcode and molecular barcode can be linked to a RNA during reverse transcription.

A reverse transcription reaction can be conducted in a presence of a 3′ tagging polynucleotide. A 3′ tagging polynucleotide can comprise a P7 segment which can be used for annealing a sequencing primer. A 3′ tagging polynucleotide can comprise a vessel barcode or a molecular barcode. A 3′ tagging polynucleotide can comprise 3′ riboguanosine residues on a 3′ end (rGrGrG) (RNA bases) that can be complementary to and annealed to a strand produced by a reverse transcription enzyme. Thus, a vessel barcode and molecular barcode can be added to a terminal end of a cDNA in this same emulsion by reverse transcription enzymes. In some embodiments, guanine residues can be used instead of riboguanosine (DNA nucleotide instead of RNA nucleotide). Upon annealing of a 3′ tagging polynucleotide to a CCC of a cDNA strand, a reverse transcriptase continues extending a cDNA into a 3′ tagging polynucleotide, thereby creating a molecular barcoded tag to all cDNAs in a reaction. Upon annealing of a 3′ tagging polynucleotide to a region of a molecular barcoded cDNA, a reverse transcriptase or polymerase continues extending a molecular barcoded cDNA into another 3′ tagging polynucleotide, thereby creating a vessel barcoded tag to all cDNAs in a reaction.

In some embodiments, template switching can be done in a separate reaction instead of being done at the same time a reverse transcription reaction can be conducted. In some embodiments, a 3′ tagging polynucleotide can be added after a reverse transcription reaction, and enzymes such as a reverse transcriptase or polymerase can be used to extend into a tagging polynucleotide in a similar fashion. Because a 3′ tagging polynucleotide can harbor a unique degenerate molecular barcode on each single molecule, each cDNA can be uniquely tagged with a molecular barcode. Because a 3′ tagging polynucleotide can harbor a same degenerate vessel barcode on each single molecule from a single vessel, each cDNA can be tagged with a vessel barcode unique to the vessel.

In some embodiments, a template switching molecule, such as a template switch oligonucleotide containing a barcode (e.g., a molecular barcode) can incorporate modified bases to minimize artifact formation. In some examples, a template-switch oligonucleotide can contain 2′deoxy uridine, which can be reverse transcribed, but cannot be copied by DNA polymerase. In some embodiments, riboguanosine can be incorporated in the template-switch oligonucleotide. In some embodiments, the template-switch oligonucleotide can modified at the 3′ end to prevent extension by reverse transcriptase or DNA polymerase. Such modifications include 3′deoxy, 3′phosphate, 3′amino, and 3′alkyl modification to effect blockage of primer extension.

a. Cloning and Expression of Genetic Material

“Antibody expression library” or “TCR expression library” or “expression library” as used herein can refer to a collection of molecules (i.e. two or more molecules) at either the nucleic acid or protein level. Thus, this term can refer to a collection of expression vectors which encode a plurality of TCR molecules (i.e. at the nucleic acid level) or can refer to a collection of TCR molecules after they have been expressed in an appropriate expression system (i.e. at the protein level). Alternatively the expression vectors/expression library may be contained in suitable host cells in which they can be expressed. The antibody molecules which are encoded or expressed in the expression libraries of the embodiments can be in any appropriate format, e.g., may be whole TCR molecules or may be TCR fragments, e.g., single chain antibodies (e.g. scFv antibodies), Fv antibodies, Fab′ antibodies, (Fab′)2 fragments, diabodies, etc. The terms “encoding” and “coding for” as is nucleic acid sequence “encoding ‘V’ coding for” or a DNA coding sequence of or a nucleotide sequence “encoding ‘V’ coding for” a particular enzyme, as well as other synonymous terms, refer to a DNA sequence which is transcribed and translated into an enzyme when placed under the control of appropriate regulatory sequences. A “promoter sequence” is a DNA regulatory region capable of binding RNA polymerase in a cell and initiating transcription of a downstream (3′ direction) coding sequence. The promoter is part of the DNA sequence. This sequence region has a start codon at its 3′ terminus. The promoter sequence includes the minimum number of bases with elements necessary to initiate transcription at levels detectable above background. However, after the RNA polymerase binds the sequence and transcription is initiated at the start codon (3′ terminus with a promoter), transcription proceeds downstream in the 3′ direction. Within the promotor sequence will be found a transcription initiation site (conveniently defined by mapping with nuclease SI) as well as protein binding domains (consensus sequences) responsible for the binding of RNA polymerase.

TCR molecules identified by, derived from, selected from, or obtainable from the TCR expression libraries of the embodiments form a yet further aspect of the embodiments. Again these TCR molecules may be proteins or nucleic acids encoding TCR molecules, which nucleic acids may in turn be incorporated into an appropriate expression vector and/or be contained in a suitable host cell.

The cDNA pool can be subjected to a PCR reaction with polynucleotides that hybridize to a constant region of the heavy chain of antibody genes and polynucleotides that hybridize to the 5′ end of the V_{H} or Vα or Vγ chain region of TCR genes. The cDNA pool can be subjected to a PCR reaction with polynucleotides that hybridize to a constant region of the heavy chain or alpha or gamma chain of TCR genes and polynucleotides that hybridize to region 5′ to the 5′ end of the V_{H} or Vα or Vγ chain region of a barcoded polynucleotide comprising a TCR sequence. A PCR reaction can also setup for the amplification of the V_{L} or Vβ or Vγ chain pool of e.g., kappa and lambda classes. The cDNA pool can be subjected to a PCR reaction with polynucleotides that hybridize to a constant region of the light chain of antibody genes and polynucleotides that hybridize to the 5′ end of the V_{L} or Vβ or Vγ chain region of TCR genes. The cDNA pool can be subjected to a PCR reaction with polynucleotides that hybridize to a constant region of the light chain of antibody genes and polynucleotides that hybridize to region 5′ to the 5′ end of the V_{L} or Vβ or Vγ chain region of a barcoded polynucleotide comprising a TCR sequence. Such oligonucleotides or primers may be designed based on known and publicly available immunoglobulin or TCR gene sequence database information.

In some embodiments, V_{H} and V_{L} or Vα and Vβ or Vγ and Vδ sequences can be conveniently obtained from a library of V_{H} and V_{L} or Vα and Vβ or Vγ and Vδ sequences produced by PCR amplification using one or more primers that are not specific for heavy or light chain genes and, in particular, for one or both the terminal regions of the V_{H} and V_{L} or Vα and Vβ or Vγ and Vδ polynucleotides. In some embodiments, V_{H} and V_{L} sequences can be conveniently obtained from a library of V_{H} and V_{L} or Vα and Vβ or Vγ and Vδ sequences produced by PCR amplification using primers specific to a region of the vessel barcoded polynucleotide. In some embodiments, V_{H} and V_{L} sequences can be conveniently obtained from a library of V_{H} and V_{L} or Vα and Vβ or Vγ and Vδ sequences produced by PCR amplification using C-gene family-specific primers or C-gene-specific primers. In some embodiments, V_{H} and V_{L} sequences can be conveniently obtained from a library of V_{H} and V_{L} or Vα and Vβ or Vγ and Vδ sequences produced by PCR amplification using a primer set with a first primer specific to a region of the vessel barcoded polynucleotide and a second primer or plurality of second primers that are C-gene family-specific primers or C-gene-specific primers. In some embodiments, V_{H} and V_{L} or Vα and Vβ or Vγ and Vδ sequences can be conveniently obtained from a library of V_{H} and V_{L} or Vα and Vβ or Vγ and Vδ sequences produced by PCR amplification using a primer set with a first primer specific to a region of the vessel barcoded polynucleotide and a second primer specific to a universal sequence.

In some embodiments, upon reverse transcription, the resulting cDNA sequences may be amplified by PCR using one or more primers specific for immunoglobulin genes and, in particular, for one or both the terminal regions of the VH and VL or Vα and Vβ or Vγ and Vδ polynucleotides. In some embodiments, VH and VL sequences can be obtained from a library of VH and VL or Vα and Vβ or Vγ and Vδ sequences produced by PCR amplification using V-gene family-specific primers or V gene-specific primers (Nicholls et al, J. Immunol. Meth., 1993, 165:81; WO93/12227) or are designed according to standard art-known methods based on available sequence information. (The VH and VL or Vα and Vβ or Vγ and Vδ sequences can be ligated, usually with an intervening spacer sequence (e.g., encoding an in-frame flexible peptide spacer), forming a cassette encoding a single-chain antibody). V region sequences can be conveniently cloned as cDNAs or PCR amplification products for immunoglobulin-express sing cells. The VH and VL or Vα and Vβ or Vγ and Vδ regions are sequenced, optionally, in the methods described herein and particularly after certain steps as noted (e.g., after single cell PCR; after mammalian or other cell surface display, after FACS screening, and the like). Sequencing can be used, among other reasons, to verify that the level of diversity is at an acceptable level. Sequencing can include high-throughput sequencing, deep sequencing (in which the same gene is sequenced from a plurality of individual samples to identify differences in the sequences), or combinations of the two.

In some embodiments, it is unnecessary to physically link the natural VH and VL or Vα and Vβ or Vγ and Vδ combinations using the methods described herein. In some embodiments, cDNAs, barcoded polynucleotides, or PCR amplified barcoded cDNAs are not physically linked. In some embodiments, cDNAs, barcoded polynucleotides, or PCR amplified barcoded cDNAs are not physically linked in the same reaction or vessel.

In some embodiments, the natural VH and VL or Vα and Vβ or Vγ and Vδ combinations are physically linked, using, in addition to the cDNA primers, one primer or plurality of primers for the 5′ end of the VH or Vα or Vγ gene and another primer or plurality of primers for the 5′ end of the VL or Vβ or Vδ gene. These primers also contain complementary tails of extra sequence, to allow the self-assembly of the VH and VL or Vα and Vβ or Vγ and Vδ genes. After PCR amplification and linking, the chance of getting mixed products, in other words, mixed variable regions, is minimal because the amplification and linking reactions were performed within each cell. The risk of mixing can be further decreased by utilizing bulky reagents such as digoxigenin-labeled nucleotides to further ensure that V region cDNA pairs do not leave the cellular compartment and intermix, but remain within the cell for PCR amplification and linking. The amplified sequences are linked by hybridization of complementary terminal sequences. After linking, sequences may be recovered from cells for use in further method steps described herein. For example, the recovered DNA can be PCR amplified using terminal primers, if necessary, and cloned into vectors which may be plasmids, phages, cosmids, phagemids, viral vectors or combinations thereof as detailed below. Convenient restriction enzyme sites may be incorporated into the hybridized sequences to facilitate cloning. These vectors may also be saved as a library of linked variable regions for later use.

In some embodiments in which it is desired to provide additional VH and VL or Vα and Vβ or Vγ and Vδ combinations, an expression system is chosen to facilitate this. For example, bacteriophage expression systems allow for the random recombination of heavy- and light-chain sequences. Other suitable expression systems are known to those skilled in the art.

It should be noted that in the case of VH and V L or Vα and Vβ or Vγ and Vδ sequences derived from nonhumans, in some embodiments, it can be preferable to chimerize these sequences with a fully human Fc. As used herein “chimerized” refers to an immunoglobulin or TCR, wherein the heavy and light chain variable regions or Vα and Vβ or Vγ and Vδ regions are not of human origin and wherein the constant regions of the heavy and light chains or Vα and Vβ or Vγ and Vδ chains are of human origin. This is affected by amplifying and cloning the variable domains into a human Fc. The human Fc can be part of the vector, or in a separate molecule, and library of Fc's could also be used. In a preferred embodiment the chimerized molecules grown in mammalian cells such as CHO cells, screened with FACS twice to enrich the cell population for cells expressing the antibody of interest. The chimerized TCRs are characterized, by either sequencing followed by functional characterization, or direct functional characterization or kinetics. Growth, screening and characterization are described in detail below.

It is important to note that the above described PCR reactions are described for cloning the antibodies in the IgG form. These are preferred as they are generally associated with a more mature immune response and generally exhibit higher affinity than IgM antibodies, thereby making them more desirable for certain therapeutic and diagnostic applications. Clearly, however, polynucleotides can be designed which will allow the cloning of one or more of the other forms of immunoglobulin molecules, e.g., IgM, IgA, IgE and IgD if desired or appropriate.

Once a TCR has been identified and the appropriate population of said cells have been isolated at an appropriate time and optionally enriched as described above, the TCR expression libraries need not be generated immediately, providing the genetic material contained in the cells can be kept intact thereby enabling the library to be made at a later date. Thus, for example the cells, a cell lysate, or nucleic acid, e.g., RNA or DNA derived therefrom, can be stored until a later date by appropriate methods, e.g., by freezing, and the expression libraries generated at a later date when desired.

Once the library of expression vectors has been generated, the encoded antibody molecules can then be expressed in an appropriate expression system and screened using appropriate techniques which are well known and documented in the art. Thus the above defined method of the embodiments may comprise the further steps of expressing the library of expression vectors in an appropriate expression system and screening the expressed library for antibodies with desired properties, as explained in further detail below.

As indicated herein, polynucleotides prepared by the methods of the disclosure which comprise a polynucleotide encoding TCR sequences can include, but are not limited to, those encoding the amino acid sequence of a TCR fragment, by itself, the noncoding sequence for the entire TCR or a portion thereof, the coding sequence for a TCR, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example-ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused TCR comprising a TCR fragment or portion.

The primary PCR products can then optionally be subjected to a secondary PCR reaction with new polynucleotide sets that hybridize to the 5′ and 3′ ends of the TCR variable domains V_{H}, V_{L} kappa and V_{L} lambda or Vα and v or Vγ and Vδ (as appropriate depending on whether the primary PCR reaction with which the new polynucleotide sets are used was designed to amplify portions of the heavy or light chain antibody genes or Vα or Vβ TCR genes or Vγ or Vδ TCR genes). These polynucleotides advantageously include DNA sequences specific for a defined set of restriction enzymes (i.e. restriction enzyme sites) for subsequent cloning. The selected restriction enzymes must be selected so as not to cut within human TCR V-gene segments. Such polynucleotides may be designed based on known and publicly available immunoglobulin or TCR gene sequence and restriction enzyme database information. However, preferred restriction enzyme sites to be included are NcoI, Hind III, M and NotI. The products of such secondary PCR reactions are repertoires of various V-heavy, V-light kappa and V-light lambda antibody fragments/domains. This type of secondary PCR reaction is therefore generally carried out when the expression library format of interest is a scFv or Fv format, wherein only the V_{H} and V_{L} or Vα and V or Vγ and Vδ domains of a TCR are present.

PCR products can also be subjected to a PCR reaction with new primer sets that hybridize to the 5′ and 3′ ends of the barcoded polynucleotides. These polynucleotides can advantageously include DNA sequences specific for a defined set of restriction enzymes (i.e. restriction enzyme sites) for subsequent cloning. The selected restriction enzymes must be selected so as not to cut within human TCR V-gene segments. Such polynucleotides may be designed based on known and publicly available immunoglobulin or TCR gene sequence and restriction enzyme database information. However, preferred restriction enzyme sites to be included are NcoI, Hind III, MluI and NotI. The products of such secondary PCR reactions are repertoires of various V_{H}, V_{L} kappa and VL lambda antibody fragments/domains or Vα and Vβ or Vγ and Vδ TCR fragments/domains.

One of skill in the art will recognize that heavy or light chain or Vα or Vβ chain or Vγ or Vδ chain Fv or Fab fragments, or single-chain TCRs may also be used with this system. A heavy or light chain or Vα or v chain or Vy or V chain can be mutagenized followed by the addition of the complementary chain to the solution. The two chains are then allowed to combine and form a functional antibody fragment. Addition of random non-specific light or heavy chain or Vα or i chain or Vy or V chain sequences allows for the production of a combinatorial system to generate a library of diverse members.

Libraries of such repertoires of cloned fragments comprising the variable heavy chain or Vα chain or Vy chain regions, or fragments thereof, and/or variable light chain or v β chain or V chain regions, or fragments thereof, of TCR genes derived from the B or T lymphocytes of immuno-challenged hosts as defined herein form further aspects of the embodiments. These libraries comprising cloned variable regions may optionally be inserted into expression vectors to form expression libraries.

In some embodiments, the PCR reactions can be set up so as to retain all or part of the constant regions of the various TCR chains contained in the isolated immune cell population. This is desirable when the expression library format is a Fab format, wherein the heavy or alpha or gamma chain component comprises V_{H} or Vα or Vy and CH or Ca or Cy domains and the light chain or v β chain or V chain component comprises V L or v β or V chain and CL or β or C domains. Again, libraries of such cloned fragments comprising all or part of the constant regions of TCR chains form further aspects of the embodiments.

These nucleic acids can conveniently comprise sequences in addition to a polynucleotide of the present embodiments. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present embodiments. For example, a hexa-histidine marker sequence provides a convenient means to purify the proteins of the present embodiments. The nucleic acid of the present embodiments, excluding the coding sequence, is optionally a vector, adaptor, or linker for cloning and/or expression of a polynucleotide of the present embodiments.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adaptors, and linkers is well known in the art. (See, e.g., Ausubel, supra; or Sambrook, supra).

The libraries disclosed herein may be used in a variety of applications. As used herein, a library comprises a plurality of molecules. In some embodiments, a library comprises a plurality of polynucleotides. In some embodiments, a library comprises a plurality of primers. In some embodiments, a library comprises a plurality of sequence reads from one or more polynucleotides, amplicons, or amplicon sets. A library can be stored and used multiple times to generate samples for analysis. Some applications include, for example, genotyping polymorphisms, studying RNA processing, and selecting clonal representatives to do sequencing according to the methods provided herein. Libraries comprising a plurality of polynucleotides, such as primers or libraries for sequencing or amplification, can be generated, wherein a plurality of polynucleotides comprises at least about 2, 3, 4, 5, 6, 7, 8, 9, 10, 11, 12, 13, 14, 15, 20, 25, 30, 35, 40, 45, 50, 60, 70, 90, 100, 200, 300, 400, 500, 600, 700, 800, 900, 1000, 15000, 2000, 3000, 4000, 5000, 6000, 7000, 8000, 9000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 50,000,000, 100,000,000 or more molecular barcodes or vessel barcodes. In some embodiments, libraries of polynucleotides comprise a plurality of at least about 2, 3, 4, 5, 6, 7, 8, 9, 10, 11, 12, 13, 14, 15, 20, 25, 30, 35, 40, 45, 50, 60, 70, 80, 90, 100, 200, 300, 400, 500, 600, 700, 800, 900, 1000, 1500, 2000, 3000, 4000, 5000, 6000, 7000, 8000, 9000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 50,000,000, 100,000,000 or more unique polynucleotides, wherein each unique polynucleotide comprises one or more molecular barcodes and vessel barcodes.

b. Barcodes

A barcode can be a molecular barcode or a vessel barcode. In some embodiments, a barcode, such as a molecular barcode or a vessel barcode, can each have a length within a range of from 2 to 36 nucleotides, 4 to 36 nucleotides, or from 6 to 30 nucleotides, or from 8 to 20 nucleotides, 2 to 20 nucleotides, 4 to 20 nucleotides, or from 6 to 20 nucleotides. In certain aspects, the melting temperatures of barcodes within a set are within 10° C. of one another, within 5° C. of one another, or within 2° C. of one another. In certain aspects, the melting temperatures of barcodes within a set are not within 10° C. of one another, within 5° C. of one another, or within 2° C. of one another. In other aspects, barcodes are members of a minimally cross-hybridizing set. For example, the nucleotide sequence of each member of such a set can be sufficiently different from that of every other member of the set that no member can form a stable duplex with the complement of any other member under stringent hybridization conditions. In some embodiments, the nucleotide sequence of each member of a minimally cross-hybridizing set differs from those of every other member by at least two nucleotides. Barcode technologies are described in Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101:1 1046; and Brenner (2004) Genome Biol. 5:240.

As used herein, a molecular barcode comprises information that is unique to a single molecule from a single cell or from a single vessel, or two or more molecules of a plurality or library of molecules from two or more single cells or from two or more single vessels. As used herein, a vessel barcode comprises information that is unique to polynucleotides from a single cell or from a single vessel, compared to polynucleotides from a different single cell or from a different single vessel. In some embodiments the unique information comprises a unique sequence of nucleotides. For example, the sequence of the molecular barcode or a vessel barcode can be determined by determining the identity and order of the unique or random sequence of nucleotides comprising the molecular barcode or a vessel barcode. In some embodiments the unique information cannot be used to identify the sequence of a target polynucleotide. For example, a molecular barcode may be attached to one target polynucleotide, but the molecular barcode cannot be used to determine the target polynucleotide to which it is attached. In some embodiments the unique information is not a known sequence linked to the identity of the sequence of a target polynucleotide. For example, a vessel barcode may be attached to one or more target polynucleotides, but the vessel barcode cannot be used to determine which of the one or more target polynucleotides to which it is attached. In some embodiments, the unique information comprises a random sequence of nucleotides. In some embodiments the unique information comprises one or more unique sequences of nucleotides on a polynucleotide. In some embodiments the unique information comprises a degenerate nucleotide sequence or degenerate barcode. A degenerate barcode can comprise a variable nucleotide base composition or sequence. For example, a degenerate bar code can be a random sequence. In some embodiments, a complement sequence of a molecular barcode or a vessel barcode is also a molecular barcode or a vessel barcode sequence.

A molecular barcode or vessel barcode can comprise any length of nucleotides. For example a molecular barcode or a vessel barcode can comprise at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides. For example a molecular barcode or a vessel barcode can comprise at most about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides. In some embodiments, a molecular barcode or a vessel barcode has a particular length of nucleotides. For example, a molecular barcode or a vessel barcode can be about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides in length.

In some embodiments, each molecular barcode or a vessel barcode in a plurality of molecular barcodes or vessel barcodes has at least about 2 nucleotides. For example, each molecular barcode or a vessel barcode in a plurality of molecular barcodes or vessel barcodes can be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides in length. In some embodiments, each molecular barcode or a vessel barcode in a plurality of molecular barcodes or vessel barcodes has at most about 1000 nucleotides. For example, each molecular barcode or a vessel barcode in a plurality of molecular barcodes or vessel barcodes can be at most about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides in length. In some embodiments, each molecular barcode or a vessel barcode in a plurality of molecular barcodes or vessel barcodes has the same length of nucleotides. For example, each molecular barcode or a vessel barcode in a plurality of molecular barcodes or vessel barcodes can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides in length. In some embodiments, one or more molecular barcodes or vessel barcodes in a plurality of molecular barcodes or vessel barcodes have a different length of nucleotides. For example one or more first molecular barcodes or vessel barcodes in a plurality of molecular barcodes or vessel barcodes can have about, or at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides and one or more second molecular barcodes or vessel barcodes in a plurality of molecular barcodes or vessel barcodes can have about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 500, or 1000 nucleotides, wherein the number of nucleotides of the one or more first molecular barcodes or vessel barcodes is different than the one or more second molecular barcodes or vessel barcodes.

The number of molecular barcodes can be in excess of the total number of molecules to be labeled in a plurality of vessels. The number of vessel barcodes can be in excess of the total number of molecules to be labeled in a plurality of vessels. For example, the number of molecular barcodes or vessel barcodes can be at least about 2, 3, 4, 5, 6, 7, 8, 9, 10, 15, 20, 30, 40, 50, 60, 70, 80, 90, or 100 times greater than the total number of molecules to be labeled in a plurality of vessels.

The number of different molecular barcodes can be in excess of the total number of molecules to be labeled in a plurality of vessels. In some embodiments, the number of different molecular barcodes is at least about 1, 1.5, 2, 2.5, 3, 3.5, 4, 4.5, 5, 6, 7, 8, 9, 10, 15, 20, 30, 40, 50, 60, 70, 80, 90, or 100 times greater than the total number of molecules to be labeled in a plurality of vessels.

The number of different molecular barcodes in a single vessel can be in excess of the number of different molecules to be labeled in the single vessel. In some embodiments, the number of different molecular barcodes in a single vessel is at least about 1, 1.5, 2, 2.5, 3, 3.5, 4, 4.5, 5, 6, 7, 8, 9, 10, 15, 20, 30, 40, 50, 60, 70, 80, 90, or 100 times greater than the number of different molecules to be labeled in the single vessel.

The number of different vessel barcodes can be less than the total number of molecules to be labeled in a plurality of vessels. In some embodiments, the number of different vessel barcodes is at least about 1, 1.5, 2, 2.5, 3, 3.5, 4, 4.5, 5, 6, 7, 8, 9, 10, 15, 20, 30, 40, 50, 60, 70, 80, 90, or 100 times less than the total number of molecules to be labeled in a plurality of vessels.

The number of amplified product molecules from a vessel barcoded polynucleotide molecule in a single vessel can be in excess of the number of different molecules to be labeled in the single vessel. In some embodiments, the number of amplified product molecules from a vessel barcoded polynucleotide molecule in a single vessel is at least about 1, 1.5, 2, 2.5, 3, 3.5, 4, 4.5, 5, 6, 7, 8, 9, 10, 15, 20, 30, 40, 50, 60, 70, 80, 90, or 100 times greater than the number of different molecules to be labeled in the single vessel.

The number of vessel barcoded polynucleotide molecules in a single vessel can be less than the number of different molecules to be labeled in the single vessel. In some embodiments, the number of vessel barcoded polynucleotide molecules in a single vessel is at least about 1, 1.5, 2, 2.5, 3, 3.5, 4, 4.5, 5, 6, 7, 8, 9, 10, 15, 20, 30, 40, 50, 60, 70, 80, 90, or 100 times less than the number of different molecules to be labeled in the single vessel.

The number of vessel barcoded polynucleotide molecules in a single vessel can be one molecule. The number of unamplified vessel barcoded polynucleotide molecules in a single vessel can be one molecule.

In some embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the different molecular barcodes have the same concentration. In some embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the different vessel barcodes have the same concentration.

In some embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the different molecular barcodes have a different concentration. In some embodiments, at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the different vessel barcodes have a different concentration.

The molecular barcodes or vessel barcodes in a population of molecular barcodes or vessel barcodes can have at least 10, 15, 20, 25, 30, 35, 40, 45, 50, 60, 70, 80, 90, 100, 200, 300, 400, 500, 600, 700, 800, 900, 1000 or more different sequences. For example, the molecular barcodes or vessel barcodes in a population can have at least 2,000, 3,000, 4,000, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1,000,000 or more different sequences. Thus, a plurality of molecular barcodes or vessel barcodes can be used to generate at least 10, 15, 20, 25, 30, 35, 40, 45, 50, 60, 70, 80, 90, 100, 200, 300, 400, 500, 600, 700, 800, 900, 1000 or more different sequences from one or more polynucleotides, such as target polynucleotides. For example, a plurality of molecular barcodes or vessel barcodes can be used to generate at least 2,000, 3,000, 4,000, 5,000, 6,000, 7,000, 8,000, 9,000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹² or more different sequences from one or more polynucleotides, such as target polynucleotides. For example, a plurality of molecular barcodes or vessel barcodes can be used to generate at least about 10, 15, 20, 25, 30, 35, 40,45, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁷, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹² or more different sequences from at least about 10, 15, 20, 25, 30, 35, 40, 45, 50, 60, 70, 80, 90, 100, 200, 300, 400, 500, 600, 700, 800, 900, 1000, 2000, 3000, 4000, 5000, 6000, 7000, 8000, 9000, 10,000, 15,000, 20,000, 25,000, 30,000, 35,000, 40,000, 45,000, 50,000, 60,000, 70,000, 80,000, 90,000, 100,000, 200,000, 300,000, 400,000, 500,000, 600,000, 700,000, 800,000, 900,000, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁻, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹² or more target polynucleotides.

In some embodiments, one or more molecular barcodes are used to group or bin sequences. In some embodiments, one or more molecular barcodes are used to group or bin sequences, wherein the sequences in each bin contain the same molecular barcode. In some embodiments, one or more molecular barcodes or vessel barcodes are used to group or bin sequences, wherein the sequences in each bin comprise an amplicon set. In some embodiments, one or more molecular barcodes are used to group or bin sequences, wherein the sequences in each bin comprise a plurality of sequences wherein the polynucleotides from which the plurality of sequences were generated were derived from the same polynucleotide molecule in an amplification reaction.

In some embodiments, one or more vessel barcodes are used to group or bin sequences. In some embodiments, one or more vessel barcodes are used to group or bin sequences, wherein the sequences in each bin contain the same vessel barcode. In some embodiments, one or more vessel barcodes are used to group or bin sequences, wherein the sequences in each bin comprise one or more amplicon sets. In some embodiments, one or more vessel barcodes are used to group or bin sequences, wherein the sequences in each bin comprise a plurality of sequences wherein the polynucleotides from which the plurality of sequences were generated were derived from the polynucleotides from a single vessel or single cell.

In some embodiments, one or more molecular barcodes and vessel barcodes are used to group or bin sequences. In some embodiments, one or more molecular barcodes and vessel barcodes are used to group or bin sequences, wherein the sequences in each bin contain the same molecular barcode and same vessel barcode. In some embodiments, one or more molecular barcodes and vessel barcodes are used to group or bin sequences, wherein the sequences in each bin comprise one or more amplicon sets. In some embodiments, one or more molecular barcodes and vessel barcodes are used to group or bin sequences, wherein the sequences in each bin comprise a plurality of sequences wherein the polynucleotides from which the plurality of sequences were generated were derived from the same polynucleotide in an amplification reaction and from the same single cell or vessel. In some embodiments, one or more molecular barcodes and vessel barcodes are not used to align sequences.

In some embodiments, one or more molecular barcodes are not used to align sequences. In some embodiments, one or more molecular barcodes are used to align sequences. In some embodiments, one or more molecular barcodes are used to group or bin sequences, and a target specific region is used to align sequences. In some embodiments, one or more vessel barcodes are not used to align sequences. In some embodiments, one or more vessel barcodes are used to align sequences. In some embodiments, one or more vessel barcodes are used to group or bin sequences, and a target specific region is used to align sequences. In some embodiments, one or more molecular barcodes and vessel barcodes are used to align sequences. In some embodiments, one or more molecular barcodes and vessel barcodes are used to group or bin sequences, and a target specific region is used to align sequences.

In some embodiments, the aligned sequences contain the same molecular barcode. In some embodiments, the aligned sequences contain the same vessel barcode. In some embodiments, the aligned sequences contain the same molecular barcode and vessel barcode. In some embodiments, one or more molecular barcodes or vessel barcodes are used align sequences, wherein the aligned sequences comprise two or more sequences from an amplicon set. In some embodiments, one or more molecular barcodes or vessel barcodes are used to align sequences, wherein the aligned sequences comprise a plurality of sequences wherein the polynucleotides from which the plurality of sequences were generated were derived from the same polynucleotide molecule in an amplification reaction. In some embodiments, one or more molecular barcodes or vessel barcodes are used to align sequences, wherein the aligned sequences comprise a plurality of sequences wherein the polynucleotides from which the plurality of sequences were generated were derived from a single cell or single vessel.

c. Droplet Generation

Splitting a sample of a plurality of cells into small reaction volumes, coupled with molecular and vessel barcoding of polynucleotides from, or derived from, an individual cell from the plurality of cells can enable high throughput sequencing of a repertoire of sequences, such as biomarker sequences.

Splitting a sample of a plurality of cells into small reaction volumes, coupled with molecular and vessel barcoding of polynucleotides from, or derived from, an individual cell from the plurality of cells can enable high throughput sequencing of a repertoire of sequences, such as sequences representing a percentage of the transcriptome of an organism. For example, a repertoire of sequences can comprise a plurality of sequences representing at least about 0.00001%, 0.00005%, 0.00010%, 0.00050%, 0.001%, 0.005%, 0.01%, 0.05%, 0.1%, 0.5%, 1%, 2%, 2.5%, 3%, 3.5%, 4%, 4.5%,5%,6%,7%,8%,9%, 10%, 15%, 20%, 30%, 35%, 40%, 45, 50%, 55%, 60%, 65%, 70%, 75%, 80%, 85%, 90%, 95%, 98%, 99%, or 100% of the transcriptome of an organism.

Splitting a sample of immune cells into small reaction volumes, coupled with molecular and vessel barcoding of polynucleotides from, or derived from, an individual immune cell from the plurality of immune cells can enable high throughput sequencing of a repertoire of heavy and light chain sequences. These methods can also allow for pairing of the heavy and light chains after sequencing based on the barcoded sequences. Splitting a sample into small reaction volumes as described herein can also enable the use of reduced amounts of reagents, thereby lowering the material cost of the analysis.

In some cases, the reverse transcription reaction and/or the amplification reaction (e.g., PCR) are carried out in droplets, such as in droplet digital PCR. In certain aspects, the embodiments provide fluidic compartments to contain all or a portion of a target material. In some embodiments, a compartment is droplet. While reference is made to “droplets” throughout the specification, that term is used interchangeably with fluid compartment and fluid partition unless otherwise indicated. Except where indicated otherwise, “droplet” is used for convenience and any fluid partition or compartment may be used. The droplets used herein can include emulsion compositions (or mixtures of two or more immiscible fluids), such as described in U.S. Pat. No. 7,622,280. The droplets can be generated by devices described in WO/2010/036352. The term emulsion, as used herein, can refer to a mixture of immiscible liquids (such as oil and water). Oil-phase and/or water-in-oil emulsions allow for the compartmentalization of reaction mixtures within aqueous droplets. The emulsions can comprise aqueous droplets within a continuous oil phase. The emulsions provided herein can be oil-in-water emulsions, wherein the droplets are oil droplets within a continuous aqueous phase. The droplets provided herein are designed to prevent mixing between compartments, with each compartment protecting its contents from evaporation and coalescing with the contents of other compartments.

The mixtures or emulsions described herein can be stable or unstable. The emulsions can be relatively stable and have minimal coalescence. Coalescence occurs when small droplets combine to form progressively larger ones. In some cases, less than 0.00001%, 0.00005%, 0.00010%, 0.00050%, 0.001%, 0.005%, 0.01%, 0.05%, 0.1%, 0.5%, 1%, 2%, 2.5%, 3%, 3.5%, 4%, 4.5%, 5%, 6%, 7%, 8%, 9%, or 10% of droplets generated from a droplet generator coalesce with other droplets. The emulsions can also have limited flocculation, a process by which the dispersed phase comes out of suspension in flakes.

Droplets can be generated having an average diameter of about, less than about, or more than about, or at least about 0.001, 0.01, 0.05, 0.1, 1, 5, 10, 20, 30, 40, 50, 60, 70, 80, 100, 120, 130, 140, 150, 160, 180, 200, 300, 400, or 500 microns. Droplets can have an average diameter of about 0.001 to about 500, about 0.01 to about 500, about 0.1 to about 500, about 0.1 to about 100, about 0.01 to about 100, or about 1 to about 100 microns. Microfluidic methods of producing emulsion droplets using microchannel cross-flow focusing or physical agitation are known to produce either monodisperse or polydisperse emulsions. The droplets can be monodisperse droplets. The droplets can be generated such that the size of the droplets does not vary by more than plus or minus 5% of the average size of the droplets. In some cases, the droplets are generated such that the size of the droplets does not vary by more than plus or minus 2% of the average size of the droplets. A droplet generator can generate a population of droplets from a single sample, wherein none of the droplets vary in size by more than plus or minus about 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% of the average size of the total population of droplets.

Higher mechanical stability can be useful for microfluidic manipulations and higher-shear fluidic processing (e.g., in microfluidic capillaries or through 90 degree turns, such as valves, in fluidic path). Pre- and post-thermally treated droplets or capsules can be mechanically stable to standard pipet manipulations and centrifugation.

A droplet can be formed by flowing an oil phase through an aqueous sample. The aqueous phase can comprise a buffered solution and reagents for performing an amplification reaction, including cells, nucleotides, nucleotide analogues, molecular barcoded polynucleotides, vessel barcoded polynucleotides primers, template nucleic acids, and enzymes, such as a DNA polymerase, R A polymerase, and/or reverse transcriptase.

The aqueous phase can comprise a buffered solution and reagents for performing an amplification reaction with or without a solid surface, such as a bead. The buffered solution can comprise about, more than about, or less than about 1, 5, 10, 15, 20, 30, 50, 100, or 200 mM Tris. In some cases, the concentration of potassium chloride can be about, more than about, or less than about 10, 20, 30, 40, 50, 60, 80, 100, 200 mM. The buffered solution can comprise about 15 mM Tris and 50 mM KC1. The nucleotides can comprise deoxyribonucleotide triphosphate molecules, including dATP, dCTP, dGTP, and dTTP, in concentrations of about, more than about, or less than about 50, 100, 200, 300, 400, 500, 600, or 700 μM each. In some cases dUTP is added within the aqueous phase to a concentration of about, more than about, or less than about 50, 100, 200, 300, 400, 500, 600, or 700, 800, 900, or 1000 μM. In some cases, magnesium chloride or magnesium acetate (MgCl is added to the aqueous phase at a concentration of about, more than about, or less than about 1.0, 2.0, 3.0, 4.0, or 5.0 mM). The concentration of MgCl can be about 3.2 mM. In some cases, magnesium acetate or magnesium is used. In some cases, magnesium sulfate is used.

A non-specific blocking agent such as BSA or gelatin from bovine skin can be used, wherein the gelatin or BSA is present in a concentration range of approximately 0.1-0.9% w/v. Other possible blocking agents can include betalactoglobulin, casein, dry milk, or other common blocking agents. In some cases, preferred concentrations of BSA and gelatin are about 0.1% w/v.

Primers for amplification within the aqueous phase can have a concentration of about, more than about, or less than about 0.05, 0.1, 0.2, 0.3, 0.4, 0.5, 0.6, 0.7, 0.8, 0.9, 1.0, 1.2, 1.5, 1.7, or 2.0 μM. Primer concentration within the aqueous phase can be about 0.05 to about 2, about 0.1 to about 1.0, about 0.2 to about 1.0, about 0.3 to about 1.0, about 0.4 to about 1.0, or about 0.5 to about 1.0 μM. The concentration of primers can be about 0.5 μM. Amenable ranges for target nucleic acid concentrations in PCR include, but are not limited to between about 1 pg and about 500 ng.

In some cases, the aqueous phase can also comprise additives including, but not limited to, non-specific background/blocking nucleic acids (e.g., salmon sperm DNA), biopreservatives (e.g. sodium azide), PCR enhancers (e.g. Betaine, Trehalose, etc.), and inhibitors (e.g. RNAse inhibitors). Other additives can include, e.g., dimethyl sulfoxide (DMSO), glycerol, betaine (mono)hydrate (N,N,N-trimethylglycine=[carboxymethyl] trimethylammonium), trehalose, 7-Deaza-2′-deoxyguanosine triphosphate (dC7GTP or 7-deaza-2′-dGTP), BSA (bovine serum albumin), formamide (methanamide), tetramethylammonium chloride (TMAC), other tetraalkylammonium derivatives (e.g., tetraethyammonium chloride (TEA-C1) and tetrapropylammonium chloride (TPrACl), non-ionic detergent (e.g., Triton X-100, Tween 20, Nonidet P-40 (NP-40)), or PREXCEL-Q. In some cases, the aqueous phase can comprise 0, 1, 2, 3, 4, 5, 6, 7, 8, 9, or 10 different additives. In other cases, the aqueous phase can comprise at least 0, 1, 2, 3, 4, 5, 6, 7, 8, 9, or 10 different additives.

In some cases, a non-ionic Ethylene Oxide/Propylene Oxide block copolymer can be added to the aqueous phase in a concentration of about 0.1%, 0.2%, 0.3%, 0.4%, 0.5%, 0.6%, 0.7%, 0.8%, 0.9%, or 1.0%. Common biosurfactants include non-ionic surfactants such as Pluronic F-68, Tetronics, and Zonyl FSN. Pluronic F-68 can be present at a concentration of about 0.5% w/v.

In some cases magnesium sulfate can be substituted for magnesium chloride, at similar concentrations. A wide range of common, commercial PCR buffers from varied vendors can be substituted for the buffered solution.

The emulsion can be formulated to produce highly monodisperse droplets having a liquid like interfacial film that can be converted by heating into microcapsules having a solid-like interfacial film; such microcapsules can behave as bioreactors able to retain their contents through a reaction process such as PCR amplification. The conversion to microcapsule form can occur upon heating. For example, such conversion can occur at a temperature of greater than about 50° C., 60° C., 70° C., 80° C., 90° C., or 95° C. In some cases this heating occurs using a thermocycler. During the heating process, a fluid or mineral oil overlay can be used to prevent evaporation. Excess continuous phase oil can or cannot be removed prior to heating. The biocompatible capsules can be resistant to coalescence and/or flocculation across a wide range of thermal and mechanical processing. Following conversion, the capsules can be stored at about, more than about, or less than about 3° C., 4° C., 5° C., 6° C., 7° C., 8° C., 9° C. 10° C., 15° C., 20° C., 25° C., 30° C., 35° C., or 40° C. These capsules can be useful in biomedical applications, such as stable, digitized encapsulation of macromolecules, particularly aqueous biological fluids containing a mix of nucleic acids or protein, or both together; drug and vaccine delivery; biomolecular libraries; clinical imaging applications, and others.

The microcapsules can contain one or more polynucleotides and can resist coalescence, particularly at high temperatures. Accordingly, PCR amplification reactions can occur at a very high density (e.g., number of reactions per unit volume). In some cases, greater than 100,000, 500,000, 1,000,000, 1,500,000, 2,000,000, 2,500,000, 5,000,000, or 10,000,000 separate reactions can occur per ml. In some cases, the reactions occur in a single well, e.g., a well of a microtiter plate, without inter-mixing between reaction volumes. The microcapsules can also contain other components necessary to enable a reverse transcription, primer extension, and/or PCR reaction to occur, e.g., primers, probes, dNTPs, DNA or RNA polymerases, etc. These capsules exhibit resistance to coalescence and flocculation across a wide range of thermal and mechanical processing.

In some cases, the amplifying step is carried out by performing digital PCR, such as microfluidic -based digital PCR or droplet digital PCR.

Droplets can be generated using microfluidic systems or devices. As used herein, the “micro-” prefix (for example, as “microchannel” or “microfluidic”), generally refers to elements or articles having widths or diameters of less than about 1 mm, and less than about 100 microns (micrometers) in some cases. In some cases, the element or article includes a channel through which a fluid can flow. Additionally, “microfluidic”, as used herein, refers to a device, apparatus or system that includes at least one microscale channel.

Microfluidic systems and devices have been described in a variety of contexts, typically in the context of miniaturized laboratory (e.g., clinical) analysis. Other uses have been described as well. For example, International Patent Application Publication Nos. WO 01/89788; WO 2006/040551; WO 2006/040554; WO 2004/002627; WO 2008/063227; WO 2004/091763; WO 2005/021 151; WO 2006/096571; WO 2007/089541; WO 2007/081385 and WO 2008/063227.

A droplet generally includes an amount of a first sample fluid in a second carrier fluid. Any technique known in the art for forming droplets may be used with methods of the embodiments. An exemplary method involves flowing a stream of the sample fluid containing the target material (e.g., immune cell) such that it intersects two opposing streams of flowing carrier fluid. The carrier fluid is immiscible with the sample fluid. Intersection of the sample fluid with the two opposing streams of flowing carrier fluid results in partitioning of the sample fluid into individual sample droplets containing the target material.

The carrier fluid may be any fluid that is immiscible with the sample fluid. An exemplary carrier fluid is oil. In certain embodiments, the carrier fluid includes a surfactant.

The same method may be applied to create individual droplets that contain other reagents such as reagents for an amplification reaction such as a polymerase chain reaction (PCR), or a non-PCR based amplification reaction such as multi-strand displacement amplification, or other methods known to one of ordinary skill in the art. Suitable reagents for conducting PCR-based amplification reactions are known to those of ordinary skill in the art and include, but are not limited to, DNA polymerases, forward and reverse primers, deoxynucleotide triphosphates (dNTPs), and one or more buffers.

In certain embodiments, fluidic compartments are formed by providing a first fluid partition (e.g., a droplet) comprising a target material (e.g., an immune cell and/or a solid support such as a bead) and a second fluid (e.g., as a fluid stream or within droplets). The first and second fluids are merged to form a droplet. Merging can be accomplished by application of an electric field to the two fluids. In certain embodiments, the second fluid contains reagents for conducting an amplification reaction, such as a polymerase chain reaction or an amplification reaction.

In certain aspects, the embodiments provides a method of making a library of uniquely barcoded heavy and light chain antibody sequences and/or alpha and beta chain TCR sequences and/or gamma and delta chain TCR sequences including obtaining a plurality of nucleic acid constructs in which each construct includes a unique N-mer and a functional N-mer. The functional N-mer can be a random N-mer, a PCR primer, a universal primer, an antibody, a sticky end, or any other sequence. The method can include making M sets of a number N of fluid compartments each containing one or more copies of a unique construct. The method can create barcode libraries of higher complexity by adding an additional construct to each compartment in a set, and repeating that for each set to produce M compartments each containing a unique pair of constructs. The pairs can be hybridized or ligated to produce new constructs. In each construct in a barcode library, each unique N-mer can be adapted for identification by sequencing, probe hybridization, other methods, or a combination of methods.

d. Droplet Libraries

In general, a droplet library is made up of a number of library elements that are pooled together in a single collection. Libraries may vary in complexity from a single library element to 1×10¹⁵ library elements or more. Each library element is one or more given components at a fixed concentration. The element may be, but is not limited to, cells, beads, amino acids, proteins, polypeptides, nucleic acids, polynucleotides or small molecule chemical compounds. The element may contain an identifier such as a molecular barcode, a vessel barcode, or both.

A cell library element can include, but is not limited to, hybridomas, B-cells, T-cells, primary cells, cultured cell lines, cancer cells, stem cells, or any other cell type. Cellular library elements are prepared by encapsulating a number of cells from one to tens of thousands in individual droplets. The number of cells encapsulated is usually given by Poisson statistics from the number density of cells and volume of the droplet. However, in some cases the number deviates from Poisson statistics as described in Edd et al., “Controlled encapsulation of single-cells into monodisperse picoliter drops.” Lab Chip, 8(8): 1262-1264, 2008. The discreet nature of cells allows for libraries to be prepared in mass with a plurality of cell variants, such as immune cells producing one TCR each, all present in a single starting media and then that media is broken up into individual droplet capsules that contain at most one cell. The cells within the individual droplets capsules are then lysed, heavy chain and light chain polynucleotides and/or alpha and beta chain polynucleotides and/or gamma and delta chain polynucleotides from the lysed cells are barcoded with molecular barcodes and vessel barcodes and amplified and then combined or pooled to form a library consisting of heavy and light chain and/or alpha and beta chain and/or gamma and delta chain library elements.

A bead based library element contains one or more beads, and may also contain other reagents, such as antibodies, enzymes or other proteins. In the case where all library elements contain different types of beads, but the same surrounding media, the library elements can all be prepared from a single starting fluid or have a variety of starting fluids. In the case of cellular libraries prepared in mass from a collection of variants, the library elements will be prepared from a variety of starting fluids. It is desirable to have exactly one cell per droplet with only a few droplets containing more than one cell when starting with a plurality of cells. In some cases, variations from Poisson statistics can be achieved to provide an enhanced loading of droplets such that there are more droplets with exactly one cell per droplet and few exceptions of empty droplets or droplets containing more than one cell.

In some embodiments, it is desirable to have exactly one vessel barcoded polynucleotide per droplet with only a few droplets containing more than one vessel barcoded polynucleotide when starting with a plurality of vessel barcoded polynucleotide. In some cases, variations from Poisson statistics can be achieved to provide an enhanced loading of droplets such that there are more droplets with exactly one vessel barcoded polynucleotide per droplet and few exceptions of empty droplets or droplets containing more than one vessel barcoded polynucleotide.

Examples of droplet libraries are collections of droplets that have different contents, ranging from beads, cells, small molecules, DNA, primers, antibodies, and barcoded polynucleotides. The droplets range in size from roughly 0.5 micron to 500 micron in diameter, which corresponds to about 1 picoliter to 1 nanoliter. However, droplets can be as small as 5 microns and as large as 500 microns. Preferably, the droplets are at less than 100 microns, about 1 micron to about 100 microns in diameter. The most preferred size is about 20 to 40 microns in diameter (10 to 100 picoliters). The preferred properties examined of droplet libraries include osmotic pressure balance, uniform size, and size ranges.

The droplets comprised within the droplet library provided by the instant embodiments are preferably uniform in size. That is, the diameter of any droplet within the library will vary less than 5%, 4%, 3%, 2%, 1% or 0.5% when compared to the diameter of other droplets within the same library. The uniform size of the droplets in the library may be critical to maintain the stability and integrity of the droplets and also may be essential for the subsequent use of the droplets within the library for the various biological and chemical assays described herein.

The embodiments provides a droplet library comprising a plurality of aqueous droplets within an immiscible fluid, wherein each droplet is preferably substantially uniform in size and comprises a different library element. The embodiments provides a method for forming the droplet library comprising providing a single aqueous fluid comprising different library elements, encapsulating each library element into an aqueous droplet within an immiscible fluid.

In certain embodiments, different types of elements (e.g., cells or beads), are pooled in a single source contained in the same medium. After the initial pooling, the elements are then encapsulated in droplets to generate a library of droplets wherein each droplet with a different type of bead or cell is a different library element. The dilution of the initial solution enables the encapsulation process. In some embodiments, the droplets formed will either contain a single element or will not contain anything, i.e., be empty. In other embodiments, the droplets formed will contain multiple copies of a library element. The elements being encapsulated are generally variants of a type. In one example, elements are immune cells of a blood sample, and each immune cell is encapsulated to amplify and barcode the antibody sequences of the nucleotides in the immune cells.

For example, in one type of emulsion library, there are library elements that have different particles, i.e., cells or barcoded polynucleotides in a different medium and are encapsulated prior to pooling. In one example, a specified number of library elements, i.e., n number of different cells, or barcoded polynucleotides, is contained within different mediums. Each of the library elements are separately emulsified and pooled, at which point each of the n number of pooled different library elements are combined and pooled into a single pool. The resultant pool contains a plurality of water-in-oil emulsion droplets each containing a different type of particle.

In some embodiments, the droplets formed will either contain a single library element or will not contain anything, i.e., be empty. In other embodiments, the droplets formed will contain multiple copies of a library element. The contents of the beads follow a Poisson distribution, where there is a discrete probability distribution that expresses the probability of a number of events occurring in a fixed period of time if these events occur with a known average rate and independently of the time since the last event. The oils and surfactants used to create the libraries prevent the exchange of the contents of the library between droplets.

e. Reverse Transcription and Polymerase Chain Reaction (PCR)

In some cases, the target polynucleotides are prepared from RNA, such as mRNA, by reverse transcription. In some cases, the target polynucleotides are prepared from a DNA by primer extension, such as using a polymerase.

The methods described herein can be used in coupled reverse transcription-PCR (reverse transcription-PCR). For example, reverse transcription and PCR can be carried out in two distinct steps. First a cDNA copy of the sample mRNA can be synthesized using either a polynucleotide dT primer, a sequence specific primer, a universal primer, a mixture of random hexamer oligonucleotide primers, or any primer described herein. In some examples, a cDNA copy of the RNA can be generated using a mixture of primers, such as a sequence specific primer and a mixture of random hexamer oligonucleotide primers, for example, to capture specific target RNA molecules of a cell in addition to a collection of polynucleotides that are substantially corresponds to the transcriptome of the same cell.

Reverse transcription and PCR can be carried out in a single closed vessel reaction. For example, a multitude of primers can be employed, one or more primers for reverse transcription and two or more primers for PCR in the same closed vessel. The primer(s) for reverse transcription can bind to the mRNA 3′ to the position of the first PCR amplicon. In some embodiments, the conditions of the PCR can be modified to substantially restrict amplification to the first adaptor, or pool of first adaptors, using primers specific thereto, and limit amplification of the larger molecular-barcoded cDNA. Although not essential, the reverse transcription primer(s) can include RNA residues or modified analogs such as 2′-O-methyl RNA bases, which will not form a substrate for RNase H when hybridized to the mRNA.

The temperature to carry out the reverse transcription reaction depends on the reverse transcriptase being used. In some cases, a thermostable reverse transcriptase is used and the reverse transcription reaction is carried out at about 37° C. to about 75° C., at about 37° C. to about 50° C., at about 37° C. to about 55° C., at about 37° C. to about 60° C., at about 55° C. to about 75° C., at about 55° C. to about 60° C., at about 37° C., or at about 60° C. In some cases, a reverse transcriptase that transfers 3 or more non-template terminal nucleotides to an end of the transcribed product is used.

A reverse transcription reaction and the PCR reaction described herein can be carried out in various formats known in the art, such as in tubes, microtiter plates, microfluidic devices, or, preferably, droplets.

A reverse transcription reaction can be carried out in volumes ranging from 5μL to 100 μL, or in 10 μL to 20 μL reaction volumes. In droplets, reaction volumes can range from 1 pL to 100 nL, or 10 pL to 1 nL. In some cases, the reverse transcription reaction is carried out in a droplet having a volume that is about or less than 1 nL. In some cases, a PCR reaction is in a droplet having a reaction volume ranges from 1 pL to 100 nL preferably 10 pL to 1 nL. In some cases, the PCR reaction is carried out in a droplet having a volume that is about or less than 1 nL. In some cases, a reverse transcription reaction and a PCR reaction are carried out in the same droplet having a reaction volume ranges from 1 pL to 100 nL or 10 pL to 1 nL. In some cases, the reverse transcription reaction and the PCR reaction are carried out in a droplet having a volume that is about or less than 1 nL or a volume that is about or less than 1 pL. In some cases, a reverse transcription reaction and a PCR reaction are carried out in a different droplet. In some cases, a reverse transcription reaction and a PCR reaction are carried out in a plurality of droplets each having a reaction volume ranges from 1 pL to 100 nL or 10 pL to 1 nL. In some cases, the reverse transcription reaction and the PCR reaction are carried out in a plurality of droplets each having a volume that is about or less than 1 nL.

In some cases, a first PCR reaction is in a first droplet having a reaction volume ranges from 1 pL to 100 nL preferably 10 pL to 1 nL and a second PCR reaction is in a second droplet having a reaction volume ranges from 1 pL to 100 nL preferably 10 pL to 1 nL. In some cases, a first PCR reaction is in a first droplet having a volume that is about or less than 1 nL, and a second PCR reaction is in a second droplet having a volume that is about or less than 1 nL.

In some cases, a first PCR reaction and a second PCR reaction are carried out in a plurality of droplets each having a reaction volume ranges from 1 pL to 100 nL or 10 pL to 1 nL. In some cases, a first PCR reaction and a second PCR reaction are carried out in a plurality of droplets each having a volume that is about or less than 1 nL.

Target polynucleotides, such as RNA, can be reverse transcribed into cDNA using one or more reverse transcription primers. The one or more reverse transcription primers can comprise a region complementary to a region of the RNA, such as a constant region (e.g., a heavy or light chain constant region or a poly-A tail of mRNA). In some embodiments, the reverse transcription primers can comprise a first reverse transcription primer with a region complementary to a constant region of a first RNA, and a second reverse transcription primer with a region complementary to a constant region of a second RNA. In some embodiments, the reverse transcription primers can comprise a first reverse transcription primer with a region complementary to a constant region of a first RNA, and one or more reverse transcription primers with a region complementary to a constant region of one or more RNAs, respectively.

In some embodiments, the reverse transcription primers can be modified to minimize artifact formation by exponential amplification of primer-dimer or primer-template switch products in the reaction. In some embodiments, the reverse transcription primers are modified by the addition of a 2′-O-methylation of one or more bases of the primer. In some embodiments, the one or more 2′-O-methylated bases are located near the center of the primer sequence. Such modified primers are typically used in reactions containing a DNA polymerase that cannot incorporate a base opposite the 2′O-methyl-modified residue. Exemplary 2′O-methyl-modified primers are set forth in SEQ ID NOS: 27-48).

In some embodiments the reverse transcription primers are a mixture of random hexamer oligonucleotides. Such primers can bind RNA at random locations, thereby priming the reverse transcription reaction of unknown sequences. In such examples, sufficient supplies of random hexamer primers are used to effect reverse transcription of essentially the transcriptome of the cell. Thus, in such embodiments, a collection of polynucleotides is generated, such as a collection of cDNA polynucleotides that corresponds to the transcriptome of the cell.

In some embodiments, reverse transcription primers do not comprise a barcode.

Reverse transcription primers can further comprise a region that is not complementary to a region of the RNA. In some embodiments, the region that is not complementary to a region of the RNA is 5′ to a region of the primers that is complementary to the RNA. In some embodiments, the region that is not complementary to a region of the RNA is 3′ to a region of the primers that is complementary to the RNA. In some embodiments, the region that is not complementary to a region of the RNA is a 5′ overhang region. In some embodiments, the region that is not complementary to a region of the RNA comprises a priming site for amplification and/or a sequencing reaction. Using the one or more primers described herein, the RNA molecules are reverse transcribed using suitable reagents known in the art.

After performing the reverse transcription reactions of the RNA molecules, the resulting cDNA molecules can be barcoded with a molecular barcode and a vessel barcode and amplified by one or more PCR reactions as described below.

In some embodiments of the methods provided herein, the conditions of the reactions can be modified to effect amplification of selected sequences and minimize the amplification of other sequences. Such modifications can include altering the temperature, such as the melting temperature during PCR thermocycling. For example, “cold” cycles of PCR can be used to selectively amplify shorter oligonucleotides. “Cold” PCR cycles, differ from “hot” PCR cycles in their denaturation temperature. “Cold” cycles of PCR effect denaturation at a lower temperature to preferably amplify shorter sequences which are more readily denatured than longer, double-stranded sequences. Thus “cold” cycles of PCR are used to amplify shorter sequences, while limiting amplification of longer sequences. In some examples, a combination of “cold” cycles and “hot cycles” are used to generate desired amplicons.

In some embodiments, the duration of the denaturing, priming and/or elongation steps of the PCR can be modified to selectively or preferably amplify particular sequences in the reaction volume. In some examples, the primers used for the PCR amplification can be selected or modified to reduce or enhance PCR amplification under particular conditions. In some examples, heat labile accessory groups can be added to the 3′ end of most bases via phophotriester linkages to render the oligonucleotide primers inactive a lower temperatures, but active once exposed to warmer temperatures. In some examples, oligonucleotides, linked to a heat-labile accessory group at the 3′ end are used in the provided methods, such that the oligonucleotides are incapable of primer extension at lower temperatures, such as temperatures at which reverse transcriptase reactions occur, but are rendered active upon exposure to a higher temperature, such as prior to PCR.

After performing the reverse transcription reactions of the RNA molecules or primer extension of genomic molecules, the oligonucleotide containing the vessel barcode is amplified by polymerase chain reaction to generate multiple copies to be appended to molecular barcoded polynucleotides. In some examples, the oligonucleotides containing the vessel barcode are amplified using primers that have been modified by the addition of a heat-labile accessory group at the 3′ end to prevent primer extension during the reverse transcriptase reaction, but enable primer extension and subsequent amplification during PCR. In some embodiments, amplification of the oligonucleotide containing the vessel barcode was carried out using “cold” thermocycling as described herein.

After performing the reverse transcription reactions of the RNA molecules, the resulting cDNA molecules can be barcoded with a molecular barcode and a vessel barcode and amplified by one or more PCR reactions, such as a first and/or a second PCR reaction. The first and/or second PCR reaction can utilize a pair of primers or a plurality of primer pairs. The first and/or second PCR reaction can utilize a plurality of forward/reverse primers and a reverse primer. The first and/or second PCR reaction can utilize a plurality of forward/reverse primers and a forward primer. A first and/or second primer of a plurality of forward/reverse primers can be a forward/reverse primer containing a region complementary to the cDNA molecules or barcoded cDNA molecules. A first and/or second primer of a plurality of forward/reverse primers can be a forward/reverse primer containing a region complementary to the barcoded cDNA molecules.

In some embodiments, a plurality of forward/reverse primers comprises one or more forward/reverse primers wherein each of the forward/reverse primers in the plurality of forward/reverse primers comprises a region complementary to one or more upstream or downstream regions to a V segment of the cDNAs or barcoded cDNAs. For example, a plurality of forward/reverse primers comprises a forward/reverse primer comprising a region complementary to a upstream or downstream region to a V segment of the cDNAs or barcoded cDNAs and one or more other forward/reverse primers comprising a region complementary to one or more other upstream or downstream regions to a V segment of the cDNAs or barcoded cDNAs. For example, a plurality of forward/reverse primers comprises a first and/or second forward/reverse primer comprising a region complementary to a first and/or second upstream or downstream region to a V segment of the cDNAs or barcoded cDNAs and a second forward/reverse primer comprising a region complementary to a second upstream or downstream region to a V segment of the cDNAs or barcoded cDNAs. For example, a plurality of forward/reverse primers comprises a first and/or second forward/reverse primer comprising a region complementary to a first and/or second upstream or downstream region to a V segment of the cDNAs or barcoded cDNAs, a second forward/reverse primer comprising a region complementary to a second upstream or downstream region to a V segment of the cDNAs or barcoded cDNAs, and a third forward/reverse primer comprising a region complementary to a third upstream or downstream region to a V segment of the cDNAs or barcoded cDNAs, etc. The primers in the plurality of forward/reverse primers can be used to anneal to all possible upstream or downstream regions of all V segments expressed by the cells, such as immune B-cells or T-cells, in the sample.

In some embodiments, a plurality of forward/reverse primers comprises one or more forward/reverse primers wherein each of the forward/reverse primers in the plurality of forward/reverse primers comprises a region complementary to one or more upstream or downstream regions to a C segment of the cDNAs or barcoded cDNAs. For example, a plurality of forward/reverse primers comprises a forward/reverse primer comprising a region complementary to a upstream or downstream region to a C segment of the cDNAs or barcoded cDNAs and one or more other forward/reverse primers comprising a region complementary to one or more other upstream or downstream regions to a C segment of the cDNAs or barcoded cDNAs. For example, a plurality of forward/reverse primers comprises a first and/or second forward/reverse primer comprising a region complementary to a first and/or second upstream or downstream region to a C segment of the cDNAs or barcoded cDNAs and a second forward/reverse primer comprising a region complementary to a second upstream or downstream region to a C segment of the cDNAs or barcoded cDNAs. For example, a plurality of forward/reverse primers comprises a first and/or second forward/reverse primer comprising a region complementary to a first and/or second upstream or downstream region to a C segment of the cDNAs or barcoded cDNAs, a second forward/reverse primer comprising a region complementary to a second upstream or downstream region to a C segment of the cDNAs or barcoded cDNAs, and a third forward/reverse primer comprising a region complementary to a third upstream or downstream region to a C segment of the cDNAs or barcoded cDNAs, etc. The primers in the plurality of forward/reverse primers can be used to anneal to all possible upstream or downstream regions of all C segments expressed by the cells, such as immune B-cells or T-cells, in the sample.

In some embodiments, a plurality of forward/reverse primers comprises one or more forward/reverse primers wherein each of the forward/reverse primers in the plurality of forward/reverse primers comprises a region complementary to one or more upstream or downstream regions to a molecular barcode of the barcoded cDNAs. For example, a plurality of forward/reverse primers comprises a forward/reverse primer comprising a region complementary to a upstream or downstream region to a molecular barcode of the barcoded cDNAs and one or more other forward/reverse primers comprising a region complementary to one or more other upstream or downstream regions to a molecular barcode of the barcoded cDNAs. For example, a plurality of forward/reverse primers comprises a first and/or second forward/reverse primer comprising a region complementary to a first and/or second upstream or downstream region to a molecular barcode of the barcoded cDNAs and a second forward/reverse primer comprising a region complementary to a second upstream or downstream region to a molecular barcode of the barcoded cDNAs. For example, a plurality of forward/reverse primers comprises a first and/or second forward/reverse primer comprising a region complementary to a first and/or second upstream or downstream region to a molecular barcode of the barcoded cDNAs, a second forward/reverse primer comprising a region complementary to a second upstream or downstream region to a molecular barcode of the barcoded cDNAs, and a third forward/reverse primer comprising a region complementary to a third upstream or downstream region to a molecular barcode of the barcoded cDNAs, etc. The plurality of forward/reverse primers can be used to anneal to all possible upstream or downstream regions of all molecular barcodes expressed by the cells, such as immune B-cells or T-cells, in the sample.

In some embodiments, a plurality of forward/reverse primers comprises one or more forward/reverse primers wherein each of the forward/reverse primers in the plurality of forward/reverse primers comprises a region complementary to one or more upstream or downstream regions to a vessel barcode of the barcoded cDNAs. For example, a plurality of forward/reverse primers comprises a forward/reverse primer comprising a region complementary to a upstream or downstream region to a vessel barcode of the barcoded cDNAs and one or more other forward/reverse primers comprising a region complementary to one or more other upstream or downstream regions to a vessel barcode of the barcoded cDNAs. For example, a plurality of forward/reverse primers comprises a first and/or second forward/reverse primer comprising a region complementary to a first and/or second upstream or downstream region to a vessel barcode of the barcoded cDNAs and a second forward/reverse primer comprising a region complementary to a second upstream or downstream region to a vessel barcode of the barcoded cDNAs. For example, a plurality of forward/reverse primers comprises a first and/or second forward/reverse primer comprising a region complementary to a first and/or second upstream or downstream region to a vessel barcode of the barcoded cDNAs, a second forward/reverse primer comprising a region complementary to a second upstream or downstream region to a vessel barcode of the barcoded cDNAs, and a third forward/reverse primer comprising a region complementary to a third upstream or downstream region to a vessel barcode of the barcoded cDNAs, etc. The primers in the plurality of forward/reverse primers can be used to anneal to all possible upstream or downstream regions of all vessel barcodes expressed by the cells, such as immune B-cells or T-cells, in the sample.

The forward/reverse primers in the plurality of forward/reverse primers further comprise a region that is not complementary to a region of the RNA. In some embodiments, the region that is not complementary to a region of the RNA is 5′ to a region of the forward/re verse primers that is complementary to the RNA (i.e. upstream or downstream regions of a V segment). In some embodiments, the region that is not complementary to a region of the RNA is 3′ to a region of the forward/reverse primers that is complementary to the RNA. In some embodiments, the region that is not complementary to a region of the RNA is a 5′ overhang region. In some embodiments, the region that is not complementary to a region of the RNA comprises a priming site for amplification and/or a second sequencing reaction. In some embodiments, the region that is not complementary to a region of the RNA comprises a priming site for amplification and/or a third sequencing reaction. In some embodiments, the region that is not complementary to a region of the RNA comprises a priming site for a second and a third sequencing reaction. In some embodiments, the sequence of the priming site for the second and the third sequencing reaction are the same. Using the one or more forward/reverse primers and a reverse primer as described herein, the cDNA molecules are amplified using suitable reagents known in the art. In some embodiments, a region is complementary to a region of the RNA, such as the constant region or a poly-A tail of mRNA.

f. Adaptor Ligation

Prior to adaptor ligation, the dual barcoded polynucleotides can be purified and/or selected for size. The size of the dual-barcoded polynucleotides can be selected to optimize the selected method for sequencing. The desired polynucleotide size is determined by the limitations of the sequencing instrumentation and by the specific sequencing application. In some examples the desired polynucleotide size is 0 base pairs (bp) to 100,000 bp (100 kilobases (kb)), 50 bp to 50 kb, 100bp to 25 kb. In some embodiments, a short-read sequencer is used to sequence the polynucleotides generated herein. Generally, optimal polynucleotide sizes for short-read sequencers range in length from about 20 base pairs (bp) to 2000 bp, 50 bp to 1500 bp, 50 bp to 1250 bp, 50 bp to 1000 bp, 50 bp to 750 bp, 50 bp to 500 bp, 100 bp to 1500 bp, 100 bp to 1250 bp, 100 bp to 1000 bp, 100 bp to 750 bp, 100 bp to 500 bp, 200 bp to 1500 bp, 200 bp to 1250 bp, 200 bp to 1000 bp, 200 bp to 750 bp or 250 bp to 500 bp.

In some embodiments, a long-read sequencer is used to sequence the polynucleotides generated herein. Generally, optimal polynucleotide sizes for short-read sequencers range in length from about 1 kilobase (kb) to 100 kb, such as 1 kb to 50 kb, 5 kb to 25 kb, 5 kb to 20 kb, or approximately 1 kb, 5 kb, 10 kb, 15 kb, or 20 kb.

To generate a collection of polynucleotides of the desired size, the collection of polynucleotides can be sized by modifying the conditions of the reverse transcription or primer extension reactions, such as modifying the time of the extension step of the reactions. In some embodiments, the collection of polynucleotides can be fragmented or sized to a desired length by physical methods (i.e., acoustic shearing and sonication) or enzymatic methods (i.e., non-specific endonuclease cocktails and transposase tagmentation reactions). Polynucleotides of the desired size can be isolated by agarose gel electrophoresis, such as denaturing gel electrophoresis, size exclusion methods, or automated methods or commercial kits (Quail et al, Electrophoresis (2012) 33(23):3521-3528; Duhaime et al., Environ Microbiol (2012) 14(9):2526-2537).

In some embodiments, double stranded dual-barcoded polynucleotides are purified prior to size selection, such as by affinity purification. In some embodiments, the double stranded dual-barcoded polynucleotides are denatured prior to size selection. In some embodiments, the double-stranded dual-barcoded polynucleotides are denatured by disrupting the hydrogen bonds between complementary strands of DNA. In some embodiments, denaturation of double stranded DNA is effected by application of acid or base, a concentrated inorganic sale, an organic solvent, (e.g., alcohol or chloroform), radiation or heat. In some embodiments, denaturation of double stranded DNA is effected by exposure to chemical agents such as formamide, guanidine, sodium salicylate, dimethyl sulfoxide (DMS), propylene glycol, urea, or NaOH. In some embodiments, double stranded DNA molecules are treated with NaOH, such as 0.1 M NaOH to generate single stranded molecules.

Following size selection and/or purification, a second adaptor can be added to the adaptor-tagged, dual barcoded polynucleotides. The adaptor contains a universal priming sequence, which can be used for amplification or sequencing of the adaptor-tagged dual barcoded polynucleotides. The adaptor can contain any known universal priming sequence or fragment thereof. Exemplary universal priming sequences include P7, C7, P5 or C5 priming sequences.

Addition of the second adaptor can be effected using any known method. The adaptor can be added to a single-stranded polynucleotide or a double-stranded polynucleotide. In some examples, the adaptor is added to a single-stranded polynucleotide. In other examples, an adaptor, such as a double-stranded adaptor is added to a double-stranded polynucleotide. In some embodiments, a ligase is used to ligate a single-stranded adaptor. For example, a Thermostable App ligase (NEB) or CircLigase II (Epicentre) can be used to ligate a second adaptor to a single-stranded adaptor to a single-stranded, adaptor-tagged, dual-barcoded polynucleotide.

In some embodiments a second adaptor can be added to single-stranded, adaptor-tagged dual-barcoded polynucleotides by annealing a degenerate splint adaptor. For example a second adaptor can be added by adding a splint adaptor duplex an end of the single-stranded, adaptor-tagged dual-barcoded polynucleotide. Such splint adaptor duplexes contain a paired double stranded oligonucleotide that has a degenerate overhang at one end of the molecule. The degenerate overhang can be 1, 2, 3, 4, 5, 6, 7, 8, 9, 10, 11, 12, 13, 14, 15, 16, 17, 18, 19, or 20 bases. The degenerate nucleotides of the overhang portion of the molecule are annealed to the end of the single-stranded, adaptor-tagged dual-barcoded polynucleotide opposite the end of the first adaptor sequence. In some embodiments of the method, a splint adaptor duplex with a 3′overhang is annealed to the 3′ end of the single-stranded, adaptor-tagged dual-barcoded polynucleotide, opposite the first adaptor. In some embodiments of the method, a splint adaptor duplex with a 5′overhang is annealed to the 5′ end of the single-stranded, adaptor-tagged dual-barcoded polynucleotide, opposite the first adaptor. A ligase, such as a blunt/TA ligase can facilitate annealing a splint adaptor duplex with the single-stranded, adaptor-tagged dual-barcoded polynucleotides.

In some embodiments of the method, enzymatic addition of non-templated nucleotides can be added to an end of single-stranded, adaptor-tagged dual-barcoded polynucleotides, to which an adaptor is annealed. In some embodiments, a second adaptor is annealed directly to the non-templated nucleotides using complementary base pairing. In some embodiments, a splint adaptor duplex can be annealed to the non-templated nucleotides to effect addition of the adaptor to the end of the molecule. In some embodiments, the adaptor is added to the 3′ end of the single-stranded, adaptor-tagged dual-barcoded polynucleotides. In some embodiments, the adaptor is added to the 5′ end of the single-stranded, adaptor-tagged dual-barcoded polynucleotides. A ligase, such as a blunt/TA ligase can facilitate annealing the second adaptor or splint adaptor duplex with the single-stranded, adaptor-tagged dual-barcoded polynucleotides.

The second adaptor contains a universal priming sequence or a universal priming site or a contiguous portion of a universal priming sequence or universal priming site sufficient to anneal to a complementary sequence. Universal priming sequences or universal priming sites contain oligonucleotide sequences that are complementary to universal primers or a contiguous portion thereof. Exemplary universal primers are listed in Section I.4.g.

g. Amplification

The sample containing the target polynucleotide can comprise mRNA, or fragments thereof, which can be amplified. In some cases, the average length of the mRNA, or fragments thereof, can be less than about 100, 200, 300, 400, 500, or 800 base pairs, or less than about 5, 10, 20, 30, 40, 50, 60, 70, 80, 90, 100, 110, 120, 130, 140, 150, 160, 170, 180, 190, or 200 nucleotides, or less than about 1, 2, 5, 10, 20, 30, 40, 50, 60, 70, 80, 90, 100 kilobases. In some cases, a target sequence from a relatively short template, such as a sample containing a template that is about 40, 45, 50, 55, 60, 65, 70, 75, 80, 85, 90, 95, or 100 bases, is amplified.

An amplification reaction can comprise one or more additives. In some cases, the one or more additives are dimethyl sulfoxide (DMSO), glycerol, betaine (mono)hydrate N,N,N-trimethylglycine=[carboxymethyl] trimethylammonium), trehalose, 7-Deaza-2′-deoxyguanosine triphosphate (dC7GTP or 7-deaza-2′-dGTP), BSA (bovine serum albumin), formamide (methanamide), tetramethylammonium chloride (TMAC), other tetraalkylammonium derivatives (e.g., tetraethyammonium chloride (TEA-C1) and tetrapropylammonium chloride (TPrA-Cl), nonionic detergent (e.g., Triton X-100, Tween 20, Nonidet P-40 (NP-40)), or PREXCEL-Q. In some cases, an amplification reaction comprises 0, 1, 2, 3, 4, 5, 6, 7, 8, 9, or 10 different additives. In other cases, an amplification reaction comprises at least 0, 1, 2, 3, 4, 5, 6, 7, 8, 9, or 10 different additives.

Thermocycling reactions can be performed on samples contained in reaction volumes (e.g., droplets). Droplets can be polydisperse or preferably monodisperse, generated through agitation, sonication or microfluidically through a T-channel junction or other means by those familiar with the art. Densities can exceed 20,000 droplets/400 μL (1 nL droplets), 200,000 droplets/40 ul (100 pL droplets). The droplets can remain intact during thermocycling. Droplets can remain intact during thermocycling at densities of greater than about 10,000 droplets/μL, 100,000 droplets/μL, 200,000 droplets/μL, 300,000 droplets/μL, 400,000 droplets/μL, 500,000 droplets/μL, 600,000 droplets/μL, 700,000 droplets/μL, 800,000 droplets/μL, 900,000 droplets/μL, or 1,000,000 droplets/μL. In other cases, two or more droplets do not coalesce during thermocycling. In other cases, greater than 100 or greater than 1,000 droplets do not coalesce during thermocycling.

Any DNA polymerase that catalyzes primer extension can be used, including but not limited to E. coli DNA polymerase, Klenow fragment of E. coli DNA polymerase 1, T7 DNA polymerase, T4 DNA polymerase, Taq polymerase, Pfu DNA polymerase, Vent DNA polymerase, bacteriophage 29, REDTaqTm, Genomic DNA polymerase, or sequenase. In some cases, a thermostable DNA polymerase is used. A hot start PCR can also be performed wherein the reaction is heated to 95° C. for two minutes prior to addition of the polymerase or the polymerase can be kept inactive until the first heating step in cycle 1. Hot start PCR can be used to minimize nonspecific amplification.

Any number of PCR cycles can be used to amplify the DNA, e.g., about, more than about, or less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 cycles. The number of amplification cycles can be about 1-45, 10-45, 20-45, 30-45, 35-45, 10-40, 10-30, 10-25, 10-20, 10-15, 20-35, 25-35, 30-35, or 35-40.

Amplification of target nucleic acids can be performed by any means known in the art. Target nucleic acids can be amplified by polymerase chain reaction (PCR) or isothermal DNA amplification. Examples of PCR techniques that can be used include, but are not limited to, quantitative PCR, quantitative fluorescent PCR (QF-PCR), multiplex fluorescent PCR (MF-PCR), real time PCR (reverse transcription-PCR), single cell PCR, restriction fragment length polymorphism PCR (PCR-RFLP), PCR-RFLP/reverse transcription-PCR-RFLP, hot start PCR, nested PCR, in situ polony PCR, in situ rolling circle amplification (RCA), digital PCR (dPCR), droplet digital PCR (ddPCR), bridge PCR, picoliter PCR and emulsion PCR. Other suitable amplification methods include the ligase chain reaction (LCR), transcription amplification, molecular inversion probe (MIP) PCR, self-sustained sequence replication, selective amplification of target polynucleotide sequences, consensus sequence primed polymerase chain reaction (CP-PCR), arbitrarily primed polymerase chain reaction (AP-PCR), degenerate polynucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938, as well as include Q beta replicase mediated RNA amplification. Amplification can be isothermal amplification, e.g., isothermal linear amplification.

In some embodiments, amplification does not occur on a solid support. In some embodiments, amplification does not occur on a solid support in a droplet. In some embodiments, amplification does occur on a solid support when the amplification is not in a droplet.

An amplification reaction can comprise one or more additives. In some embodiments, the one or more additives are dimethyl sulfoxide (DMSO), glycerol, betaine (mono)hydrate (N,N,N-trimethylglycine=[carboxymethyl] trimethylammonium), trehalose, 7-Deaza-2′-deoxyguanosine triphosphate (dC7GTP or 7-deaza-2′-dGTP), BSA (bovine serum albumin), formamide (methanamide), tetramethylammonium chloride (TMAC), other tetraalkylammonium derivatives (e.g., tetraethyammonium chloride (TEA-C1) and tetrapropylammonium chloride (TPrA-Cl), nonionic detergent (e.g., Triton X-100, Tween 20, Nonidet P-40 (NP-40)), or PREXCEL-Q. In some embodiments, an amplification reaction can comprise 0, 1, 2, 3, 4, 5, 6, 7, 8, 9, or 10 different additives. In other cases, an amplification reaction can comprise at least 0, 1, 2, 3, 4, 5, 6, 7, 8, 9, or 10 different additives.

Generally, one or more pairs of primers can be used in an amplification reaction; one primer of a primer pair can be a forward primer and one primer of a primer pair can be a reverse primer.

In some cases, a first pair of primers can be used in the amplification reaction; one primer of the first pair can be a forward primer complementary to a sequence of a first target polynucleotide molecule and one primer of the first pair can be reverse primer can be complementary to a second sequence of the first target polynucleotide molecule, and a first target locus can reside between the first sequence and the second sequence. In some embodiments, the first target locus comprises a V_{H} or Vα or Vγ sequence.

In some cases, a second pair of primers can be used in the amplification reaction; one primer of the second pair can be a forward primer complementary to a first sequence of a second target polynucleotide molecule and one primer of the second pair can be a reverse primer complementary to a second sequence of the second target polynucleotide molecule, and a second target locus can reside between the first sequence and the second sequence. In some embodiments, the second target locus comprises a V_{L} or Vβ or Vδ sequence.

In some cases, a third pair of primers can be used in the amplification reaction; one primer of the third pair can be a forward primer complementary to a first sequence of a third target polynucleotide molecule and one primer of the third pair can be a reverse primer complementary to a second sequence of the third target polynucleotide molecule, and a third target locus can reside between the first sequence and the second sequence. In some embodiments, the third target locus comprises a barcode, such as a molecular barcode or vessel barcode.

The length of the forward primer and the reverse primer can depend on the sequence of the target polynucleotide and the target locus. For example, the length and/or T of the forward primer and reverse primer can be optimized. In some case, a primer can be about, more than about, or less than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length. In some cases, a primer is about 15 to about 20, about 15 to about 25, about 15 to about 30, about 15 to about 40, about 15 to about 45, about 15 to about 50, about 15 to about 55, about 15 to about 60, about 20 to about 25, about 20 to about 30, about 20 to about 35, about 20 to about 40, about 20 to about 45, about 20 to about 50, about 20 to about 55, or about 20 to about 60 nucleotides in length.

A primer can be a single-stranded DNA prior to binding a template polynucleotide. In some cases, the primer initially comprises double-stranded sequence. The appropriate length of a primer can depend on the intended use of the primer but can range from about 6 to about 50 nucleotides, or from about 15 to about 3 5 nucleotides. Short primer molecules can generally require cooler temperatures to form sufficiently stable hybrid complexes with a template. In some embodiments, a primer need not reflect the exact sequence of the template nucleic acid, but can be sufficiently complementary to hybridize with a template. In some cases, a primer can be partially double-stranded before binding to a template polynucleotide. A primer with double-stranded sequence can have a hairpin loop of about, more than about, or less than about 3, 4, 5, 6, 7, 8, 9, 10, 11, 12, 13, 14, 15, 16, 17, 18, 19, or 20 bases. A double stranded portion of a primer can be about, more than about, less than about, or at least about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base-pairs. The design of suitable primers for the amplification of a given target sequence is well known in the art.

Primers can incorporate additional features that allow for the detection or immobilization of the primer but do not alter a basic property of the primer (e.g., acting as a point of initiation of DNA synthesis). For example, primers can contain an additional nucleic acid sequence at the 5′ end which does not hybridize to a target nucleic acid, but which facilitates cloning or further amplification, or sequencing of an amplified product. For example, the additional sequence can comprise a primer binding site, such as a universal primer binding site. A region of the primer which is sufficiently complementary to a template to hybridize can be referred to herein as a hybridizing region.

In another case, a primer utilized in methods and compositions described herein can comprise one or more universal nucleosides. Non-limiting examples of universal nucleosides are 5-nitroindole and inosine, as described in U.S. Appl. Pub. Nos. 2009/0325169 and 2010/0167353.

Primers can be designed according to known parameters for avoiding secondary structures and self-hybridization. Different primer pairs can anneal and melt at about the same temperatures, for example, within 1° C., 2° C., 3° C., 4° C., 5° C., 6° C., 7° C., 8° C., 9° C. or 10° C. of another primer pair. In some cases, greater than 1, 2, 3, 4, 5, 6, 7, 8, 9, 10, 15, 20, 25, 30, 35, 40, 45, 50, 100, 200, 500, 1000, 5000, 10,000 or more primers are initially used. Such primers can hybridize to target polynucleotides described herein.

Primers can be prepared by a variety of methods including but not limited to cloning of appropriate sequences and direct chemical synthesis using methods well known in the art (Narang et al, Methods Enzymol. 68:90 (1979); Brown et al, Methods Enzymol. 68:109 (1979)). Primers can also be obtained from commercial sources. The primers can have an identical melting temperature. The primers can have non-identical melting temperatures. The lengths of the primers can be extended or shortened at the 5′ end or the 3′ end to produce primers with desired melting temperatures. One of the primers of a primer pair can be longer than the other primer. The 3′ annealing lengths of the primers, within a primer pair, can differ. Also, the annealing position of each primer pair can be designed such that the sequence and length of the primer pairs yield the desired melting temperature. An equation for determining the melting temperature of primers smaller than 25 base pairs is the Wallace Rule (T=2(A+T)+4(G+C)). Computer programs can also be used to design primers. The T_{M} (melting or annealing temperature) of each primer can be calculated using software programs. The annealing temperature of the primers can be recalculated and increased after any cycle of amplification, including but not limited to cycle 1, 2, 3, 4, 5, cycles 6-10, cycles 10-15, cycles 15-20, cycles 20-25, cycles 25-30, cycles 30-35, or cycles 35-40. After the initial cycles of amplification, the 5′ half of the primers can be incorporated into the products from each loci of interest; thus the T_{M} can be recalculated based on both the sequences of the 5′ half and the 3′ half of each primer.

Conducting the one or more reactions of the methods disclosed herein can comprise the use of one or more primers. As used herein, a primer comprises a double-stranded, single-stranded, or partially single-stranded polynucleotide that is sufficiently complementary to hybridize to a template polynucleotide. A primer can be a single-stranded DNA prior to binding a template polynucleotide. In some embodiments, the primer initially comprises double-stranded sequence. A primer site includes the area of the template to which a primer hybridizes. In some embodiments, primers are capable of acting as a point of initiation for template-directed nucleic acid synthesis. For example, primers can initiate template-directed nucleic acid synthesis when four different nucleotides and a polymerization agent or enzyme, such as DNA or RNA polymerase or reverse transcriptase.

A primer pair includes 2 primers: a first primer with a 5′ upstream region that hybridizes with a 5′ end of a template sequence, and a second primer with a 3′ downstream region that hybridizes with the complement of the 3′ end of the template sequence. A primer set includes two or more primers: a first primer or first plurality of primers with a 5′ upstream region that hybridizes with a 5′ end of a template sequence or plurality of template sequences, and a second primer or second plurality of primers with a 3′ downstream region that hybridizes with the complement of the 3′ end of the template sequence or plurality of template sequences.

In some embodiments, a primer comprises a target specific sequence. In some embodiments, a primer comprises a sample barcode sequence. In some embodiments, a primer comprises a universal priming sequence. In some embodiments, a primer comprises a PCR priming sequence. In some embodiments, a primer comprises a PCR priming sequence used to initiate amplification of a polynucleotide. (Dieffenbach, PCR Primer: A Laboratory Manual, 2nd Edition (Cold Spring Harbor Press, New York (2003)). The universal primer binding site or sequence allows the attachment of a universal primer to a polynucleotide and/or amplicon. Universal primers are well known in the art and include, but are not limited to, −47F (M13F), alfaMF, AOX3′, AOX5′, BGHr, CMV-30, CMV-50, CVMf, LACrmt, lambda gt 10F, lambda gt 10R, lambda gt 11F, lambda gt 11R, M13 rev, M13Forward(−20), M13Reverse, male, pQEproseq, pQE, pA-120, pet4, pGAP Forward, pGLRVpr3, pGLpr2R, pKLAC14, pQEFS, pQERS, pucU1, pucU2, reversA, seqIREStam, seqIRESzpet, seqori, seqPCR, seqpIRES−, seqpIRES, seqpSecTag, seqpSecTag, seqretro+PSI, SP6, T3-prom, T7-prom, and T7-termInv.

As used herein, attach can refer to both or either covalent interactions and noncovalent interactions. Attachment of the universal primer to the universal primer binding site may be used for amplification, detection, and/or sequencing of the polynucleotide and/or amplicon. The universal primer binding site may comprise at least about 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, or 1000 nucleotides or base pairs. In another example, the universal primer binding site comprises at least about 1500, 2000, 2500, 3000, 3500, 4000, 4500, 5000, 5500, 6000, 6500, 7000, 7500, 8000, 8500, 9000, 9500, or 10000 nucleotides or base pairs. In some embodiments, the universal primer binding site comprises 1-10, 10-20, 10-30 or 10-100 nucleotides or base pairs. In some embodiments, the universal primer binding site comprises from about 1-90, 1-80, 1-70, 1-60, 1-50, 1-40, 1-30, 1-20, 1-10, 2-90, 2-80, 2- 70, 2-60, 2-50, 2-40, 2-30, 2-20, 2-10, 1-900, 1-800, 1-700, 1-600, 1-500, 1-400, 1-300, 1-200, 1- 100, 2-900, 2-800, 2-700, 2-600, 2-500, 2-400, 2-300, 2-200, 2-100, 5-90, 5-80, 5-70, 5-60, 5-50, 5- 40, 5-30, 5-20, 5-10, 10-90, 10-80, 10-70, 10-60, 10-50, 10-40, 10-30, 10-20, 10-10, 5-900, 5-800, 5- 700, 5-600, 5-500, 5-400, 5-300, 5-200, 5-100, 10-900, 10-800, 10-700, 10-600, 10-500, 10-400, 10- 300, 10-200, 10-100, 25-900, 25-800, 25-700, 25-600, 25-500, 25-400, 25-300, 25-200, 25-100, 100- 1000, 100-900, 100-800, 100-700, 100-600, 100-500, 100-400, 100-300, 100-200, 200-1000, 200- 900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-800, 600-700, 700-1000, 700-900, 700-800, 800-1000, 800-900, or 900-1000 nucleotides or base pairs.

Primers can have a length compatible with its use in synthesis of primer extension products. A primer can be a polynucleotide that is 8 to 200 nucleotides in length. The length of a primer can depend on the sequence of the template polynucleotide and the template locus. For example, the length and/or melting temperature (T_{M}) of a primer or primer set can be optimized. In some case, a primer can be about, more than about, or less than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length. In some embodiments, primers are about 8-100 nucleotides in length, for example, 10-7 5, 15-60, 15-40, 18-30, 20-40, 21-50, 22-45, 25-40, 7-9, 12-15, 15-20, 15-25, 15-30, 15-45, 15-50, 15-55, 15-60, 20-25, 20-30, 20-35, 20-45, 20-50, 20-55, or 20-60 nucleotides in length and any length there between. In some embodiments, primers are at most about 10, 12, 15, 20, 21, 22, 23, 24, 25, 26, 27, 28, 29, 30, 35, 40, 45, 50, 55, 60, 65, 70, 75, 80, 85, 90, 95 or 100 nucleotides in length.

Generally, one or more pairs of primers can be used in an exponential amplification reaction; one primer of a primer pair can be a forward primer and one primer of a primer pair can be a reverse primer. In some embodiments, a first pair of primers can be used in the exponential amplification reaction; one primer of the first pair can be a forward primer complementary to a sequence of a first template polynucleotide molecule and one primer of the first pair can be a reverse primer complementary to a second sequence of the first template polynucleotide molecule, and a first template locus can reside between the first sequence and the second sequence. In some embodiments, a second pair of primers can be used in the amplification reaction; one primer of the second pair can be a forward primer complementary to a first sequence of a second target polynucleotide molecule and one primer of the second pair can be a reverse primer complementary to a second sequence of the second target polynucleotide molecule, and a second target locus can reside between the first sequence and the second sequence. In some embodiments, the second target locus comprises a variable light chain antibody sequence. In some embodiments, a third pair of primers can be used in the amplification reaction; one primer of the third pair can be a forward primer complementary to a first sequence of a third template polynucleotide molecule and one primer of the third pair can be a reverse primer complementary to a second sequence of the third template polynucleotide molecule, and a third template locus can reside between the first sequence and the second sequence.

The one or more primers can anneal to at least a portion of a plurality of template polynucleotides. The one or more primers can anneal to the 3′ end and/or 5′ end of the plurality of template polynucleotides. The one or more primers can anneal to an internal region of the plurality of template polynucleotides. The internal region can be at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends or 5′ ends the plurality of template polynucleotides. The one or more primers can comprise a fixed panel of primers. The one or more primers can comprise at least one or more custom primers. The one or more primers can comprise at least one or more control primers. The one or more primers can comprise at least one or more housekeeping gene primers. The one or more primers can comprise a universal primer. The universal primer can anneal to a universal primer binding site. In some embodiments, the one or more custom primers anneal to an SBC, a target specific region, complements thereof, or any combination thereof. The one or more primers can comprise a universal primer. The one or more primers primer can be designed to amplify or perform primer extension, reverse transcription, linear extension, non-exponential amplification, exponential amplification, PCR, or any other amplification method of one or more target or template polynucleotides

The target specific region can comprise at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39,40, 41, 42, 43, 44, 45, 46, 47, 48, 49, 50, 100, 150, 200, 220, 230, 240, 250, 260, 270,280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides or base pairs. In another example, the target specific region comprises at least about 1500, 2000, 2500, 3000, 3500, 4000, 4500, 5000, 5500, 6000, 6500, 7000, 7500, 8000, 8500, 9000, 9500, or 10000 nucleotides or base pairs in some embodiments, the target specific region comprises from about 5-10, 10-15, 10-20, 10-30, 15-30, 10-75, 15-60, 15-40, 18-30, 20-40, 21-50, 22-45, 25-40, 7-9, 12-15, 15-20, 15-25, 15-30, 15-45, 15-50, 15-55, 15-60, 20-25, 20-30, 20-35, 20-45, 20-50, 20-55, 20-60, 2-900, 2-800, 2-700, 2-600, 2-500, 2-400, 2-300, 2-200, 2-100, 25-900, 25-800, 25-700, 25- 600, 25-500, 25-400, 25-300, 25-200, 25-100, 100-1000, 100-900, 100-800, 100-700, 100-600, 100- 500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-800, 600-700, 700-1000, 700-900, 700-800, 800-1000, 800-900, or 900-1000 nucleotides or base pairs.

Primers can be designed according to known parameters for avoiding secondary structures and self-hybridization. In some embodiments, different primer pairs can anneal and melt at about the same temperatures, for example, within 1° C., 2° C., 3° C., 4° C., 5° C., 6° C., 7° C., 8° C., 9° C. or 10° C. of another primer pair. In some embodiments, one or more primers in a plurality of primers can anneal and melt at about the same temperatures, for example, within 1, 2, 3, 4, 5, 6, 7, 8, 9 or 10° C. of another primer in the plurality of primers. In some embodiments, one or more primers in a plurality can anneal and melt at different temperatures than another primer in the plurality of primers.

A plurality of primers for one or more steps of the methods described herein can comprise a plurality of primers comprising about, at most about, or at least about 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000, 1500, 2000, 3000, 4000, 5000, 6000, 7000, 8000, 9000, 10,000, 11,000, 12,000, 13,000, 14,000, 15,000, 16,000, 17,000, 18,000, 19,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 50,000,000, 100,000,000 different primers. For example, each primer in a plurality of primers can comprise a different target or template specific region or sequence.

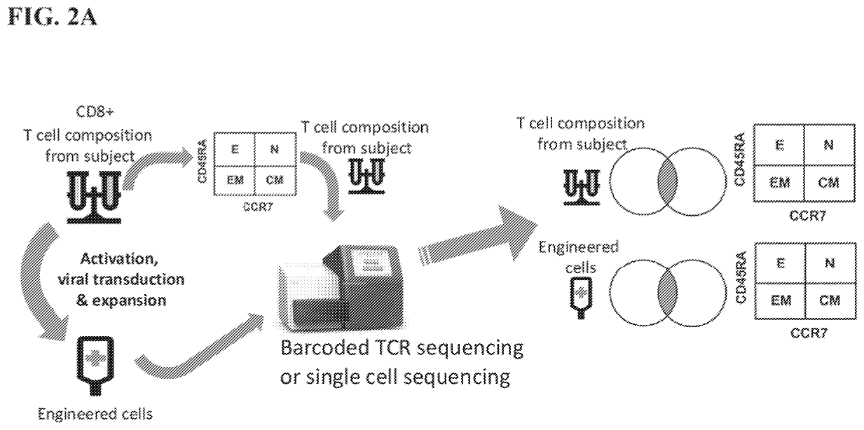

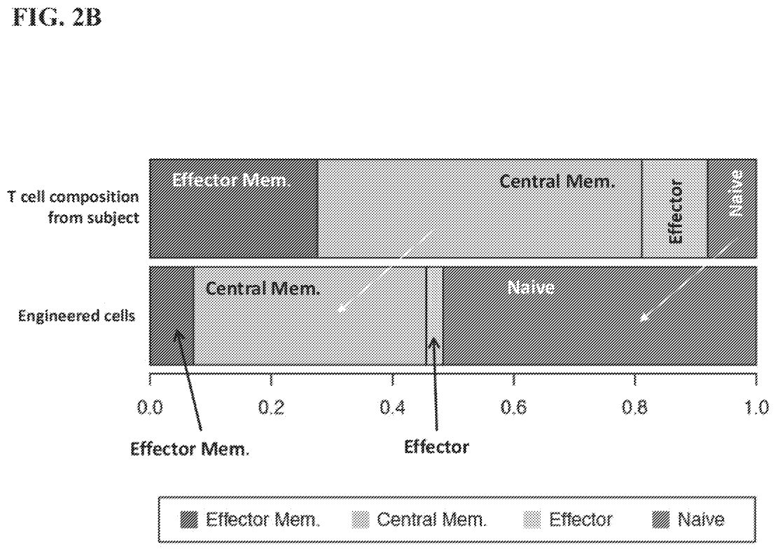

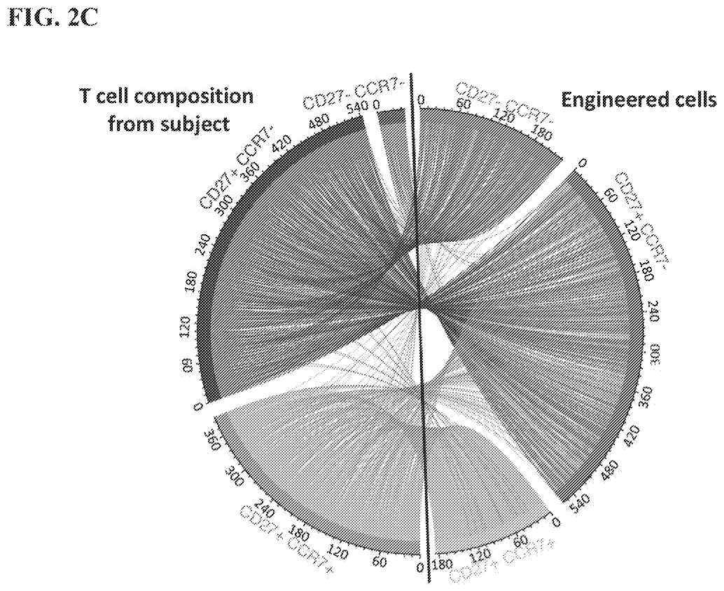

FIG. 2 depicts exemplary amplification reactions for amplification of the polynucleotide library provided herein, generated by the methods provided herein. In the exemplary methods provided herein, amplification of the library for the purposes of sequencing use primers linked to a sequencing adapter to be used for sequencing, such as next-generation sequencing. Such primers are known and described herein. Sequencing adapter-tagged primers are used in the exemplary applications provided below.

In some embodiments, a target gene is amplified for sequencing. In some embodiments, the target gene is amplified using a primer directed to an adapter sequence at one end of the polynucleotide and a target specific primer positioned to sequence the full-length target polypeptide or a selected portion thereof. In some examples the target sequence can be present in the library from a single cell or a plurality of cells. In some embodiments, one or more target sequences is amplified using primers specific to a universal priming sequence of the polynucleotide and one or more target-specific primers. In some embodiments, two or more target sequences are amplified, each with universal sequence and target-specific primers as described. In some embodiments the two or more target sequences are linked, such as two target sequences that are co-expressed in a cell, for example, target sequences that are expressed as a dimer (e.g., a heterodimer). Thus, using the provided embodiment, paired sequence information, such as full-length paired sequence information can be obtained using the provided methods.

In some embodiments the entire prepared library of polynucleotides can be amplified for sequencing using primers specific to the universal priming sequence of the first adapter and the universal priming sequence of the second adapter. Amplification of the polynucleotide libraries provided herein using primers specific to the universal priming sequences at the two ends of the polynucleotides of the library, can provide the transcriptome or genome, or portion thereof, of all cells used to make the library. Such transcriptomic information can be used for mining at later time points and/or used to evaluate expression (at the transcript level) of several genes within the population of cells from which the sample was prepared. In some embodiments, the transcriptomic information of all cells can be analyzed and used to generate clusters of cells with similar transcript expression profiles from the total population of cells from which the library was produced.

In some embodiments, the polynucleotides from a single cell can be specifically amplified and sequenced using a primer specific to the vessel barcode sequence and a primer specific to a universal priming site present in the second adapter. In such embodiments, one or more target sequence(s) is/are amplified as described above, and the vessel barcode(s) is/are identified in the target sequence(s) that are identified as of interest. As all polynucleotides from the same cell are barcoded with the same vessel barcode, this application of the method yields sequence information of all polynucleotides from the selected cell or cells. The amplification of all the polynucleotides in the library from the selected cell or cells can then provide expression profiles or genetic profiles of the cell or cells that express the one or more particular target sequences.

h. Sequencing

After performing one or more of the methods or method steps described herein, a library of polynucleotides generated can be sequenced.

Sequencing can be performed by any sequencing method known in the art. In some embodiments, sequencing can be performed in high throughput. Suitable next generation sequencing technologies include the 454 Life Sciences platform (Roche, Branford, Conn.) (Margulies et al., Nature, 437, 376-380 (2005)); Illumina's Genome Analyzer, GoldenGate Methylation Assay, or Infinium Methylation Assays, i.e., Infinium HumanMethylation 27K BeadArray or VeraCode GoldenGate methylation array (Illumina, San Diego, Calif.; Bibkova et al, Genome Res. 16, 383-393 (2006); and U.S. Pat. Nos. 6,306,597, 7,598,035, 7,232,656), or DNA Sequencing by Ligation, SOLiD System (Applied Biosystems/Life Technologies; U.S. Pat. Nos. 6,797,470, 7,083,917, 7,166,434, 7,320,865, 7,332,285, 7,364,858, and 7,429,453); or the Helicos True Single Molecule DNA sequencing technology (Harris et al, Science, 320, 106-109 (2008); and U.S. Pat. Nos. 7,037,687, 7,645,596, 7,169,560, and 7,769,400), the single molecule, real-time (SMRTTm) technology of Pacific Biosciences, and sequencing (Soni et al, Clin. Chem. 53, 1996-2001 (2007)). These systems allow multiplexed parallel sequencing of many polynucleotides isolated from a sample (Dear, Brief Funct. Genomic Proteomic, 1(4), 397-416 (2003) and McCaughan et al, J. Pathol, 220, 297-306 (2010)). In some embodiments, polynucleotides are sequenced by sequencing by ligation of dye-modified probes, pyrosequencing, or single-molecule sequencing. Determining the sequence of a polynucleotide may be performed by sequencing methods such as Helioscope™ single molecule sequencing, Nanopore DNA sequencing, Lynx Therapeutics' Massively Parallel Signature Sequencing (MPSS), 454 pyrosequencing, Single Molecule real time (RNAP) sequencing, Illumina (Solexa) sequencing, SOLiD sequencing, Ion Torrent™, Ion semiconductor sequencing, Single Molecule SMRT(TM) sequencing, Polony sequencing, DNA nanoball sequencing, and VisiGen Biotechnologies approach. Alternatively, determining the sequence of polynucleotides may use sequencing platforms, including, but not limited to, Genome Analyzer IIx, HiSeq, and MiSeq offered by Illumina, Single Molecule Real Time (SMRTTM) technology, such as the PacBio RS system offered by Pacific Biosciences (California) and the Solexa Sequencer, True Single Molecule Sequencing (tSMSTM) technology such as the HeliScope™ Sequencer offered by Helicos Inc. (Cambridge, Mass.). Sequencing can comprise MiSeq sequencing. Sequencing can comprise HiSeq sequencing. In some embodiments, determining the sequence of a polynucleotide comprises paired-end sequencing, nanopore sequencing, high-throughput sequencing, shotgun sequencing, dye-terminator sequencing, multiple-primer DNA sequencing, primer walking, Sanger dideoxy sequencing, Maxim-Gilbert sequencing, pyrosequencing, true single molecule sequencing, or any combination thereof Alternatively, the sequence of a polynucleotide can be determined by electron microscopy or a chemical-sensitive field effect transistor (chemFET) array.

A method can further comprise sequencing one or more polynucleotides in the library. A method can further comprise aligning one or more polynucleotide sequences, sequence reads, amplicon sequences, or amplicon set sequences in the library to each other.

As used herein, aligning comprises comparing a test sequence, such as a sequence read, to one or more other test sequences, reference sequences, or a combination thereof. In some embodiments, aligning can be used to determine a consensus sequence from a plurality of sequences or aligned sequences. In some embodiments, aligning comprises determining a consensus sequence from a plurality of sequences that each has an identical molecular barcode or vessel barcode. In some embodiments, the length of a sequence aligned for comparison purposes is at least 30%, at least 40%, at least 50%, at least 60%, at least 70%, at least 80%, at least 90%, or at least 95%, of the length of a reference sequence. The actual comparison of the two or more sequences can be accomplished by well-known methods, for example, using a mathematical algorithm. A non-limiting example of such a mathematical algorithm is described in Karlin, S. and Altschul, S., Proc. Natl. Acad. Sci. USA, 90- 5873-5877 (1993). Such an algorithm is incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In some embodiments, the percent identity between two amino acid sequences can be accomplished using, for example, the GAP program in the GCG software package (Accelrys, Cambridge, UK).

Sequencing can comprise sequencing at least about 10, 20, 30, 40, 50, 60, 70, 80, 90, 100 or more nucleotides or base pairs of the polynucleotides. In some embodiments, sequencing comprises sequencing at least about 200, 300, 400, 500, 600, 700, 800, 900, 1000, or more nucleotides or base pairs of the polynucleotides. In other instances, sequencing comprises sequencing at least about 1500, 2000, 3000, 4000, 5000, 6000, 7000, 8000, 9000, 10,000, or more nucleotides or base pairs of the polynucleotides.

Sequencing can comprise at least about 200, 300, 400, 500, 600, 700, 800, 900, 1000 or more sequencing reads per run. As used herein, a sequence read comprises a sequence of nucleotides determined from a sequence or stream of data generated by a sequencing technique. In some embodiments, sequencing comprises sequencing at least about 1500, 2000, 3000, 4000, 5000, 6000, 7000, 8000, 9000, 10,000, or more sequencing reads per run. Sequencing can comprise more than, less than, or equal to about 1,000,000,000 sequencing reads per run. Sequencing can comprise more than, less than, or equal to about 200,000,000 reads per run.

In some embodiments, the number of sequence reads used to determine a consensus sequence is from about 2-1000 sequence reads. For example, the number of sequence reads used to determine a consensus sequence can be from about 2-900, 2-800, 2-700, 2-600, 2-500, 2-400, 2-300, 2-200, 2-100, 25-900, 25-800, 25-700, 25-600, 25-500, 25-400, 25-300, 25-200, 25-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-800, 600-700, 700-1000, 700-900, 700-800, 800-1000, 800-900, or 900-1000 sequence reads. In some embodiments, the number of sequence reads used to determine a consensus sequence is at least about 1000, 1500, 2000, 3000, 4000, 5000, 6000, 7000, 8000, 9000, 10,000, 11,000, 12,000, 13,000, 14,000, 15,000, 16,000, 17,000, 18,000, 19,000, 20,000, 25,000, 30,000,35,000, 40,000, 45,000, 50,000, 55,000, 60,000, 65,000, 70,000, 75,000, 80,000, 85,000, 90,000, 95000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 50,000,000, or 100,000,000 reads. In some embodiments, the number of sequence reads used to determine a consensus sequence is at most about 1000, 1500, 2000, 3000, 4000, 5000, 6000, 7000, 8000, 9000, 10,000, 11,000, 12,000, 13,000, 14,000, 15,000, 16,000, 17,000, 18,000, 19,000, 20,000, 25,000, 30,000,35,000, 40,000, 45,000, 50,000, 55,000, 60,000, 65,000, 70,000, 75,000, 80,000, 85,000, 90,000, 95000, 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, 1,000,000, 50,000,000, or 100,000,000 reads.

A method can comprise sequencing misreads. A method can comprise determining the number of misreads, such as for determining a reaction condition or designing primer sequences. Comparing the number of misreads generated under one or more first conditions or sets of conditions can be used to determine a preferred condition or condition set. For example, a first method can be carried out at a high salt concentration during a PCR reaction, and a second method can be carried out at a low salt concentration during a PCR reaction, wherein the first and second method are carried out substantially the same aside from the salt concentration difference. If the first method results in a higher number of misreads, such as a higher number of misreads for a particular target polynucleotide sequence or primer, a lower salt reaction condition can be determined to be preferred for that particular target polynucleotide sequence or primer.

i. Enzymes

The methods and kits disclosed herein may comprise one or more enzymes. Examples of enzymes include, but are not limited to ligases, reverse transcriptases, polymerases, and restriction nucleases.

In some embodiments, attachment of an adaptor to polynucleotides comprises the use of one or more ligases. Examples of ligases include, but are not limited to, DNA ligases such as DNA ligase I, DNA ligase III, DNA ligase IV, and T4 DNA ligase, and RNA ligases such as T4 RNA ligase I and T4 RNA ligase II.

The methods and kits disclosed herein may further comprise the use of one or more reverse transcriptases. In some embodiments, the reverse transcriptase is a HIV-1 reverse transcriptase, MMLV reverse transcriptase, AMV reverse transcriptase, and telomerase reverse transcriptase. In some embodiments, the reverse transcriptase is M-MLV reverse transcriptase.

In some embodiments, the methods and kits disclosed herein comprise the use of one or more proteases

In some embodiments, the methods and kits disclosed herein comprise the use of one or more polymerases. Examples of polymerases include, but are not limited to, DNA polymerases and RNA polymerases. In some embodiments, the DNA polymerase is a DNA polymerase I, DNA polymerase II, DNA polymerase III holoenzyme, and DNA polymerase IV. Commercially available DNA polymerases include, but are not limited to, Bst 2.0 DNA Polymerase, Bst 2.0 WarmStart™0 DNA Polymerase, Bst DNA Polymerase, Sulfolobus DNA Polymerase IV, Taq DNA Polymerase, 9° NTMm DNA Polymerase, Deep VentR™ (exo-) DNA Polymerase, Deep VentR™ DNA Polymerase, Hemo KlenTaq™, LongAmp® Taq DNA Polymerase, OneTaq® DNA Polymerase, Phusion® DNA Polymerase, Q5TM High-Fidelity DNA Polymerase, Therminator™ γ DNA Polymerase, Therminator™ DNA Polymerase, Therminator™ II DNA Polymerase, Therminator™ III DNA Polymerase, VentR® DNA Polymerase, VentR® (exo-) DNA Polymerase, Bsu DNA Polymerase, phi29 DNA Polymerase, T4 DNA Polymerase, T7 DNA Polymerase, Terminal Transferase, Titanium® Taq Polymerase, KAPA Taq DNA Polymerase and KAPA Taq Hot Start DNA Polymerase.

In some embodiments, the polymerase is an RNA polymerases such as RNA polymerase I, RNA polymerase II, RNA polymerase III, E. coli Poly(A) polymerase, phi6 RNA polymerase (RdRP), Poly(U) polymerase, SP6 RNA polymerase, and T7 RNA polymerase.

j. Additional Reagents

The methods and kits disclosed herein may comprise the use of one or more reagents. Examples of reagents include, but are not limited to, PCR reagents, ligation reagents, reverse transcription reagents, enzyme reagents, hybridization reagents, sample preparation reagents, affinity capture reagents, solid supports such as beads, and reagents for nucleic acid purification and/or isolation.

A solid support can comprise virtually any insoluble or solid material, and often a solid support composition is selected that is insoluble in water. For example, a solid support can comprise or consist essentially of silica gel, glass (e.g. controlled-pore glass (CPG)), nylon, Sephadex®, Sepharose®, cellulose, a metal surface (e.g. steel, gold, silver, aluminum, silicon and copper), a magnetic material, a plastic material (e.g., polyethylene, polypropylene, polyamide, polyester, polyvinylidene difluoride (PVDF)) and the like. Examples of beads for use according to the embodiments can include an affinity moiety that allows the bead to interact with a nucleic acid molecule. A solid phase (e.g. a bead) can comprise a member of a binding pair (e.g. avidin, streptavidin or derivative thereof). For instance, the bead may be a streptavidin-coated bead and a nucleic acid molecule for immobilization on the bead can include a biotin moiety. In some cases, each polynucleotide molecule can include two affinity moieties, such as biotin, to further stabilize the polynucleotide. Beads can include additional features for use in immobilizing nucleic acids or that can be used in a downstream screening or selection processes. For example, the bead may include a binding moiety, a fluorescent label or a fluorescent quencher. In some cases, the bead can be magnetic. In some instances, the solid support is a bead. Examples of beads include, but are not limited to, streptavidin beads, agarose beads, magnetic beads, Dynabeads®, MACS® microbeads, antibody conjugated beads (e.g., anti-immunoglobulin microbead), protein A conjugated beads, protein G conjugated beads, protein A/G conjugated beads, protein L conjugated beads, polynucleotide-dT conjugated beads, silica beads, silica-like beads, anti-biotin microbead, anti-fluorochrome microbead, and BcMag™ Carboxy-Terminated Magnetic Beads. Beads or particles may be swellable (e.g., polymeric beads such as Wang resin) or non-swellable (e.g., CPG). In some embodiments a solid phase is substantially hydrophilic. In some embodiments a solid phase (e.g. a bead) is substantially hydrophobic. In some embodiments a solid phase comprises a member of a binding pair (e.g. avidin, streptavidin or derivative thereof) and is substantially hydrophobic or substantially hydrophilic. In some embodiments, a solid phase comprises a member of a binding pair (e.g. avidin, streptavidin or derivative thereof) and has a binding capacity greater than about 1350 picomoles of free capture agent (e.g. free biotin) per mg solid support. In some embodiments the binding capacity of solid phase comprising a member of a binding pair is greater than 800, 900, 1000, 1100, 1200, 1250, 1300, 1350, 1400, 1450, 1500, 1600, 1800, 2000 picomoles of free capture agent per mg solid support. Other examples of beads that are suitable for the embodiments are gold colloids or beads such as polystyrene beads or silica beads. Substantially any bead radii may be used. Examples of beads may include beads having a radius ranging from 150 nanometers to 10 microns. Other sizes may also be used.

The methods and kits disclosed herein may comprise the use of one or more buffers. Examples of buffers include, but are not limited to, wash buffers, ligation buffers, hybridization buffers, amplification buffers, and reverse transcription buffers. In some embodiments, the hybridization buffer is a commercially available buffer, such as TMAC Hyb solution, SSPE hybridization solution, and ECONO™ hybridization buffer. The buffers disclosed herein may comprise one or more detergents.

The methods and kits disclosed herein may comprise the use of one or more carriers. Carriers may enhance or improve the efficiency of one or more reactions disclosed herein (e.g., ligation reaction, reverse transcription, amplification, hybridization). Carriers may decrease or prevent non-specific loss of the molecules or any products thereof (e.g., a polynucleotide and/or amplicon). For example, the carrier may decrease non-specific loss of a polynucleotide through absorption to surfaces. The carrier may decrease the affinity of a polynucleotide to a surface or substrate (e.g., container, Eppendorf tube, pipet tip). Alternatively, the carrier may increase the affinity of a polynucleotide to a surface or substrate (e.g., bead, array, glass, slide, or chip). Carriers may protect the polynucleotide from degradation. For example, carriers may protect an RNA molecule from ribonucleases. Alternatively, carriers may protect a DNA molecule from a DNase. Examples of carriers include, but are not limited to, polynucleotides such as DNA and/or RNA, or polypeptides. Examples of DNA carriers include plasmids, vectors, polyadenylated DNA, and DNA polynucleotides. Examples of RNA carriers include polyadenylated RNA, phage RNA, phage MS2 RNA, E. coli RNA, yeast RNA, yeast tRNA, mammalian RNA, mammalian tRNA, short polyadenylated synthetic ribonucleotides and RNA polynucleotides. The RNA carrier may be a polyadenylated RNA. Alternatively, the RNA carrier may be a non-polyadenylated RNA. In some embodiments, the carrier is from a bacteria, yeast, or virus. For example, the carrier may be a polynucleotide or a polypeptide derived from a bacteria, yeast or virus. For example, the carrier is a protein from Bacillus subtilis. In another example, the carrier is a polynucleotide from Escherichia coli. Alternatively, the carrier is a polynucleotide or peptide from a mammal (e.g., human, mouse, goat, rat, cow, sheep, pig, dog, or rabbit), avian, amphibian, or reptile.

The methods and kits disclosed herein may comprise the use of one or more control agents. Control agents may include control polynucleotides, inactive enzymes, and/or non-specific competitors. Alternatively, the control agents comprise bright hybridization, bright probe controls, nucleic acid templates, spike-in controls, PCR amplification controls. The PCR amplification controls may be positive controls. In other instances, the PCR amplification controls are negative controls. The nucleic acid template controls may be of known concentrations. The control agents may comprise one or more labels.

Spike-in controls may be templates that are added to a reaction or sample. For example, a spike-in template may be added to an amplification reaction. The spike-in template may be added to the amplification reaction any time after the first amplification cycle. In some embodiments, the spike-in template is added to an amplification reaction after cycle number 2, 3, 4, 5, 6, 7, 8, 9, 10, 11, 12, 13, 14, 15, 20, 25, 30, 35, 40, 45, or 50. The spike-in template may be added to the amplification reaction any time before the last amplification cycle. The spike-in template may comprise one or more nucleotides or nucleic acid base pairs. The spike-in template may comprise DNA, RNA, or any combination thereof. The spike-in template may comprise one or more labels.

Disclosed herein are molecules, materials, compositions, and components that can be used for, can be used in conjunction with, can be used in preparation for, or are products of methods and compositions disclosed herein. It is understood that when combinations, subsets, interactions, groups, etc. of these materials are disclosed and while specific reference of each various individual and collective combinations and permutation of these molecules and compounds cannot be explicitly disclosed, each is specifically contemplated and described herein. For example, if a nucleotide or nucleic acid is disclosed and discussed and a number of modifications that can be made to a number of molecules including the nucleotide or nucleic acid are discussed, each and every combination and permutation of nucleotide or nucleic acid and the modifications that are possible are specifically contemplated unless specifically indicated to the contrary. This concept applies to all aspects of this application including, but not limited to, steps in methods of making and using the disclosed methods and compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

While some embodiments described herein have been shown and described herein, such embodiments are provided by way of example only. Numerous variations, changes, and substitutions will now occur to those skilled in the art without departing from the disclosure provided herein. It should be understood that various alternatives to the embodiments described herein can be employed in practicing the methods described herein.

Unless otherwise explained, all technical and scientific terms used herein have the same meaning as commonly understood by one of ordinary skill in the art to which this disclosure belongs. The following references contain embodiments of the methods and compositions that can be used herein: The Merck Manual of Diagnosis and Therapy, 18th Edition, published by Merck Research Laboratories, 2006 (ISBN 0-91 19102); Benjamin Lewin, Genes IX, published by Jones & Bartlett Publishing, 2007 (ISBN-13: 9780763740634); Kendrew et al. (eds.), The Encyclopedia of Mol. Biology, published by Blackwell Science Ltd., 1994 (ISBN 0-632-02182-9); and Robert A. Meyers (ed.), Mol. Biology and Biotechnology: a Comprehensive Desk Reference, published by VCH Publishers, Inc., 1995 (ISBN 1-56081-569-8).

Standard procedures of the present disclosure are described, e.g., in Maniatis et al, Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al, Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl (eds.), Academic Press Inc., San Diego, USA (1987)). Current Protocols in Molecular Biology (CPMB) (Fred M. Ausubel, et al. ed., John Wiley and Sons, Inc.), Current Protocols in Protein Science (CPPS) (John E. Coligan, et. al., ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), and Animal Cell Culture Methods (Methods in Cell Biology, Vol. 57, Jennie P. Mather and David Barnes editors, Academic Press, 1st edition, 1998).

B. T Cell Clonality and Diversity

In some embodiments, cells of T cell composition that are part of a process for engineering T cells with a recombinant receptor (e.g. CAR-T cells), compositions containing engineered T cells (e.g. engineered CAR-T cells) and/or samples containing or suspected or likely to contain engineered T cells, such as obtained from a subject administered engineered cells (e.g. CAR-T cells, can be assessed by determining the clonality, clonal diversity or clonal heterogeneity of the T cell population or composition, based on the determined clonotypes. In some aspects, clonality of cells in a population or composition of cells from the same subject prior to or during the process of genetically engineering the cells with a recombinant receptor (e.g. CAR) and following the administration of resulting autologous genetically engineered cells to a subject, are assessed as a read-out or characteristic of the T cells in the population or composition.

In some embodiments, assessing the clonality of the population of T cells is an assessment of clonal diversity of the population of T cells. In some embodiments, a population of T cells may be polyclonal or multiclonal, which indicates the population exhibits diversity among clonotypes of TCR in T cells of the population. In some cases, polyclonality can be measured by the variety and breadth and relative frequency of clonotypes present in a cell population or a cell composition, for example, based on the clonotype determination described herein. In some cases, polyclonality can be measured by the breadth of the response of the population to a given antigen. In some aspects, response to antigen can be assessed by measuring the number of different epitopes recognized by antigen-specific cells. This can be carried out using standard techniques for generating and cloning antigen-specific T cells in vitro. In some embodiments, the T cells of a population may be polyclonal (or multiclonal) with no single clonotypic population predominating in the population.

In the context of a population or composition of T cells, in some aspects, the signature of polyclonality refers to a population of T cells that has multiple and broad TCR sequences and/or antigen specificity. In some embodiments, polyclonality relates to a population of T cells that exhibits high diversity in the TCR repertoire, e.g., high diversity of clonotypes present in the population or composition. In some cases, diversity of the TCR repertoire is due to V(D)J recombination events that, in some respects, are triggered by selection events to self and foreign antigens. In some embodiments, a population of T cells that is diverse or polyclonal is a population of T cells in which analysis indicates the presence of a plurality of varied or different TCR transcripts or products, e.g. native TCR transcripts or products or clonotypes, present in the population (for example, as assessed based on clonotype determination of cells present in the population described herein). In some embodiments, a population of T cells that exhibits high or relatively high clonality is a population of T cells in which the TCR repertoire is less diverse. In some embodiments, T cells are oligoclonal, if analysis indicates the presence of several, such as two,three or four, predominant TCR transcripts or products or clonotypes in a population of T cells. In some embodiments, T cells are monoclonal if analysis indicates the presence of a single TCR transcript or product or clonotype in a population of T cells. In the methods herein, it is understood that clonality analysis of a population containing T cells is based on the TCR repertoire of native TCRs on such T cells in the population.

The clonality of the cells, such as T cells in a population or composition of cells, in some examples, is determined by clonal sequencing, such as any sequencing-based clonotype determination methods described herein, optionally high-throughput or next-generation sequencing, or spectratype analysis. In some aspects, high-throughput or next-generation sequencing methods can be employed, using genomic DNA or cDNA from T cells, to assess the TCR repertoire. In some embodiments, targeted sequencing of particular sequence, e.g., sequence of all or a portion of one or more TCR chains, can be employed. In some aspects, such sequencing can be carried out in a high-throughput manner. In some embodiments, whole transcriptome sequencing by RNA-seq can be employed. In some embodiments, single-cell sequencing methods can be used. In other embodiments, bulk sequencing of targeted sequences (e.g., TCR chains or portion thereof) or bulk whole genome or transcriptome sequencing (e.g., by RNAseq) can be used to determine the clonality of the cells in the population or composition.

In some embodiments, clonality can be assessed or determined by spectratype analysis (a measure of the TCR Vβ, Vα, V≢5, or Vδ chain hypervariable region repertoire). A population of T cells is considered polyclonal when the Vβ spectratype profile for a given TCR Vβ, Vα, Vγ, or Vδ family has multiple peaks, typically 5 or more predominant peaks and in most cases with Gaussian distribution. Clonality can also be assessed by generation and characterization of antigen-specific clones to an antigen of interest.

In some embodiments, the methods for assessing clonality can be based on or include various features of the methods as described in International Publication Nos. WO2012/048341, WO 2019/051335 PCT/US2018/050114 WO2014/144495, WO2017/053902, WO2016044227, WO2016176322 and WO2012048340 each incorporated by reference in their entirety. In some embodiments, such methods, or any clonotype determination methods described herein, can be used to obtain sequence information about a target polynucleotide of interest within a cell, such as a TCR. The target genes can be obtained from genomic DNA or mRNA of a cell from a sample or population of cells. The sample or population of cells can include immune cells. For example, for target TCR molecules, the genes encoding chains of a TCR can be obtained from genomic DNA or mRNA of immune cells or T cells. In some embodiments, the starting material is RNA from T cells composed of genes that encode for a chain of a TCR.

In some aspects, clonality of a population or composition of cells, such as population or composition comprising T cells, can be expressed as a numerical value. For example, in some aspects, the clonality can be represented as a numerical value calculated based on the formula: Clonality=1−Entropy/log2(N), of the various clonotypes in the population. In some aspects, the numerical clonality can range from 0, being a polyclonal or multiclonal population (low clonality, high diversity), to 1, being a monoclonal population (high clonality, low diversity).

In some aspects, a raw clonality value can be calculated for a population or composition. In some embodiments, the Shannon index is applied to the clonality as a threshold to filter clones to determine the clonality value (“Shannon- adjusted clonality”), see, Chaara et al. (2018) Front Immunol 9:1038). In some embodiments, a fixed threshold value, such as the top 25 clones, can be used to filter clones to determine the clonality value.

### II. DETERMINING T CELL PROPERTIES

In some embodiments, the methods provided herein involve determination and/or assessment of one or more properties, parameters, phenotypes, attributes or functions of the T cells, in addition to the clonotype determination.

In some embodiments, the determination and/or assessment of one or more properties, parameters, phenotypes, attributes or functions is performed in samples from various stages of adoptive cell therapy, e.g., before and after engineering of the cells and/or before and after administration of the cells in the subject. In some embodiments, the clonotypes are determined for samples from one or more stages, time points and/or locations. In some embodiments, the determination and/or assessment of one or more properties, parameters, phenotypes, attributes or functions is performed in various cell compositions and samples obtained from the subject, at various time points and stages of adoptive cell therapy. For example, for autologous cell transfer, a composition of cells, including immune cells, e.g., T cells, is initially obtained from the subject. Certain T cells are isolated from the composition, by immunoaffinity-based enrichment, and subject to genetic engineering, e.g., to express a recombinant receptor. The engineered cells then can be administered to the subject for therapy. In some embodiments, determination and/or assessment of one or more properties, parameters, phenotypes, attributes or functions is performed at any one or more points or stages throughout the process, and compared with the properties, parameters, phenotypes, attributes or functions performed at other points or stages. Exemplary methods that can be used for adoptive cell therapy, e.g., CAR-expressing T cell therapy, are described below in Section III.B. The methods provided herein can be used in any stages or time points of performing adoptive cell therapy, using any compositions or samples, such as those obtained from the subject or engineered or processed. Particular T cell clones or clonotypes can be traced throughout the generation of cells for therapy and after administration of the cells to subjects.

In some embodiments, the methods involve assessing one or more properties or parameters of the originator T cell population, e.g., T cell population from a T cell composition obtained from a subject prior to administering the cell therapy to the subject, wherein the cell has the same clonotype as one or more clonotypes identified from a biological sample from a subject obtained after administration of engineered cells, e.g., CAR-expressing cells, to the subject. In some embodiments, the methods involve determination of phenotypes, functions and/or parameters of particular samples and/or cell compositions obtained from the subject, e.g., following administration of a cell therapy comprising T cells expressing a recombinant receptor. In some embodiments, the determination of phenotypes, functions and/or parameters are determined at more than one, such as two, three, four, or five time points after administration of the cell therapy. In some embodiments, the determination and/or assessment of one or more properties, parameters, phenotypes, attributes or functions of the T cells can be performed on any of the test biological samples, cell compositions or engineered cells provided herein, at any stage or time point in the adoptive cell therapy.

In some embodiments, the determination and/or assessment of one or more properties, parameters, phenotypes, attributes or functions of the T cells involve a single cell based analysis and/or a high-throughput analysis. In some embodiments, the determination and/or assessment of one or more properties, parameters, phenotypes, attributes or functions of the T cells involve population-level analysis or analysis of a plurality of cells.

In some embodiments, exemplary determination and/or assessment of one or more properties, parameters, phenotypes, attributes or functions of the T cells involve assessment of gene expression analysis, transcriptome profiling, surface cell phenotype, epigenetic profiles, cell surface protein expression, activation phenotype or effector function. In some embodiments, the one or more properties or features is assessed by single cell analysis. In some embodiments, the determination and/or assessment of one or more properties, parameters, phenotypes, attributes or functions of the T cells involve single cell surface phenotyping and/or single cell gene expression profiling. In some embodiments, the determination and/or assessment of one or more properties, parameters, phenotypes, attributes or functions of the T cells can include various features of the methods as described in WO2016/044227, WO2016/176322, WO2012/048340, WO2012/048341, WO2014/144495, WO2017/053902, WO2017/053903 or WO2017/053905, each incorporated by reference in their entirety. In some aspects, when coupled with high-throughput sequencing technology, such methods allow analyzing a large number of single cells and achieving the analysis in one single reaction assay.

In some embodiments, the assessment of one or more properties, parameters, phenotypes, attributes or functions of the T cells is performed simultaneously with or concurrently with determination of the clonotype. In some embodiments, the assessment of one or more properties, parameters, phenotypes, attributes or functions of the T cells is performed before the determination of the clonotype. In some embodiments, the assessment of one or more properties, parameters, phenotypes, attributes or functions of the T cells is performed after identification of particular clones or clonotypes. In some embodiments, the determination and/or assessment of one or more properties, parameters, phenotypes, attributes or functions of the T cells, is performed at any stage and time point in the adoptive cell therapy. In some embodiments, the determination and/or assessment of one or more properties, parameters, phenotypes, attributes or functions of the T cells is performed before, after, simultaneously with and/or concurrently with the determination of the clonotype and/or the identification of particular clonotypes. In some embodiments, the results or data obtained from the determination and/or assessment of one or more properties, parameters, phenotypes, attributes or functions of the T cells that were previously performed or obtained, is analyzed or assessed after identification of particular clones or clonotypes of interest, employing methods provided herein. For example, additional DNA or mRNA analysis, phenotypic measurements, functional testing, cell-sorting or other analyses can be carried out prior to, concurrently with, or after assessment of the clonotypes.

In some embodiments, determination or assessment of one or more properties or parameters is coupled with clonotype determination at the single cell level. In such embodiments, the property or attribute of cells with a particular identified clonotype, can be readily identified. For example, in some embodiments, determination or assessment of one or more properties or parameters and clonotype determination is performed with samples and compositions obtained at different stages of adoptive cell therapy, including with a T cell composition comprising T cells previously obtained from the subject prior to administering the cell therapy to the subject, and a test biological sample from a subject obtained from the subject following administration of a cell therapy comprising T cells expressing the recombinant receptor. In some embodiments, a particular clonotype from a test biological sample can be selected for identification, such as the clonotypes of cells that exhibit a predetermined phenotype, function or parameter. Then, the properties or parameters of the cells in the T cell composition can be determined based on the coupled single-cell determination of properties or parameters and clonotype determination. In some embodiments, such methods can be used to assess one or more properties or parameters of the originator T cell population, which includes a cell that has the same clonotype as identified cells in the test biological samples that exhibit a predetermined phenotype, function or parameter.

In some embodiments, any single-cell based analysis can be used, in conjunction with T cell clonotype determination, to assess properties, parameters and/or features of a particular cell population and individual clones, e.g., the originator cell population. In some embodiments, the one or more properties, parameters, phenotypes, attributes or functions of the T cells include assessment of the properties, parameters, phenotypes, attributes or functions on a population level before or concurrently with clonotype determination.

In some embodiments, the properties, parameters, phenotypes, attributes and/or functions are compared between clones that have been identified to share the same clonotype, but at different stages of adoptive cell therapy.

In some embodiments, the methods involve determining a phenotype, function or parameter of the one or more cells in the plurality of sample, prior to the identifying. In some embodiments, the genetically engineered T cell in the test biological sample exhibits a predetermined phenotype, function or parameter. In some embodiments, the predetermined phenotype, function or attribute is an effector function associated with T cell activation state, is a cell surface phenotype or is a pharmacokinetic property. In some embodiments, the predetermined phenotype, function or attribute is a pharmacokinetic property and the pharmacokinetic property comprises the number or relative number of recombinant receptor-expressing T cells in the sample. In some embodiments, the predetermined phenotype, function or attribute is a cell surface phenotype and the cell surface phenotype is a naive phenotype or a long-lived memory phenotype. In some embodiments, the cell surface phenotype is determined based on surface expression of one or more of CD45RA, CCR7, CD27, CD4 and CD8, such as one or both of CD27 and CCR7.

1. Single Cell Gene Expression Analysis

In some embodiments, the one or more properties, parameters, phenotypes, attributes or functions of the T cells include determination of gene expression profiles of the T cell. In some embodiments, the gene expression profile is determined at the single cell level. In some embodiments, the gene expression profile is determined for one or more genes, e.g., one or more genes involved in immune cell function or genes indicative of immune cell phenotypes. In some embodiments, gene expression is determined at the genome-wide level, e.g., by transcriptome analysis. In some embodiments, the gene expression profile determination is performed using high-throughput methods. In some embodiments, gene expression profiling is coupled with clonotype determination at the single cell level. In such embodiments, the gene expression profile and the cell clonotype can be readily associated. In some embodiments, the gene expression profiling include various features of the methods as described in WO2016/044227, WO2016/176322, WO2012/048340, WO2012/048341, WO2014/144495, WO2017/053902, WO2017/053903 or WO2017/053905, each incorporated by reference in their entirety

In some embodiments, genome-wide level expression analysis, e.g., transcriptome analysis, is performed at a single cell level. In some embodiments, the transcriptome analysis includes determining gene expression in single cells or in a plurality of single cells. In some embodiments, exemplary methods for single cell transcriptome analysis can involve efficient generation of high quality polynucleotide (e.g. DNA) sequencing libraries from both the whole-transcriptome product or a portion thereof, and the full-length of a target gene of interest. In some embodiments, each of the plurality of polynucleotides in the library contain adapter sequences that allow for next-generation sequencing of the total recovered products, as opposed to specific genes that must be decided upon performing the experiment. In some embodiments, subsequent PCR amplification can be carried out using primers specific for these adaptor sequences. In some embodiments, the transcriptome analysis permits processing of tens or hundreds of thousands of cells in a single experiment, thereby yielding single-cell sequencing data, e.g. RNA-seq data, such as mRNA counts, combined with full-length gene sequences, in an efficient and high-throughput manner. In some embodiments, clonotype determination, e.g., paired TCRαβ sequencing, and the genome or transcriptome sequences of a plurality of cells are produced in one simultaneous reaction, and provide a mechanism for linking sequence information of sequences derived from the same cell. In some embodiments, the single cell transcriptome analysis can couple clonotype determination, e.g., by sequencing of TCR genes, and single cell barcoding in conjunction with analysis of gene expression in single cells.

In some embodiments, single cell transcriptome analysis includes analysis of analyze the genomic or mRNA content of a selected cell, such as a cell that expresses a particular gene or genes of interest, such as a particular TCR gene or a particular TCR clonotype. In some instances, it is desirable to obtain the genomic or transcriptomic content of a selected cell while also obtaining the full-length sequence of a particular gene, such as an immune receptor gene (e.g., TCR). Existing tools for single-cell transcriptome sequencing include microarrays, 96-well based methods, such as traditional FACS sorting into wells, and microfluidic instruments, such as the Fluidigm C1. These tools can be used to prepare whole transcriptome and target libraries, but their throughput is limited, because they are limited in the number of cells that can be analyzed (e.g., hundreds to thousands of cells).

Ultra high throughput methods using microwell arrays or emulsions have been described to allow whole single-cell transcriptome sequencing (see e.g., Klein et al., Cell (2015) 161(5):1187-1201; Macosko et al., Cell (2015) 161(5): 1202-1214; WO/2015/164212; WO/2016/040476), but efficient capture of full-length target sequences, such as full-length immune receptor sequences, is not possible with the existing technology. These methods are also limited to smaller numbers of cells, typically in the low thousands, due to limitations imposed by the bead-based approach, which requires larger droplets and larger reaction volumes per cell.

In some embodiments, single cell transcriptome analysis can involve assessing target sequences, such as target immune molecules (e.g. TCR, indicating T cell clonotype), and the genome or transcriptome sequences of a plurality of cells are produced in one simultaneous reaction, and provide a mechanism for linking sequence information of sequences derived from the same cell. In some aspects, the presently disclosed methods, when coupled with high-throughput sequencing technology allows analyzing a large number of single cells and achieving the analysis in one single reaction assay. Using these methods, one can sequence any number of cells and any number of targeted regions per cell. In some aspects, the number of single cells that can be processed is limited only by practical constraints, such as the speed of high throughput sequencing. In some embodiments, the methods disclosed herein are adaptable for use with beads. In other embodiments, the methods disclosed herein do not include a bead-based sequencing or amplification step.

In some aspects, the single cell transcriptome analysis can overcome, or reduce, the problems of existing methods by providing a method of preparing cDNA libraries which can be used to analyze gene expression in a plurality of single cells. In some embodiments, the single cell transcriptome analysis result in the production of a polynucleotide library, for ultra-high throughput sequencing, that allows the recovery of synthesis-ready, full-length target sequences, including sequences of paired heterodimeric or multimeric targets, while simultaneously capturing complementary quantitative genomic or transcriptomic information of the cells identified as expressing the target sequence(s).

In particular, the single cell transcriptome analyses include for preparing a polynucleotide library, e.g. cDNA, library from a plurality of single cells. The methods are based on determining gene expression levels from a population of individual cells, which can be used to identity natural variations in gene expression on a cell by cell level. The methods can also be used to identify and characterize the cellular composition of a population of cells, including in the absence of suitable cell-surface marker. The methods described herein also provide the advantage of generating a cDNA library representative of RNA content in a cell population using single cells, whereas cDNA libraries prepared by classical methods typically require total RNA isolated from a large population. Thus, in some aspects, a cDNA library produced using the single cell transcriptome analyses permit at least equivalent representation of RNA content in a population of cells by utilizing a smaller subpopulation of individual cells along with additional advantages as described herein.

Embodiments of the single cell transcriptome analyses also permit sampling of a large number of single cells. Using similarity of expression patterns, a map of cells can be built showing how the cells relate. This map can be used to distinguish cell types in silico, by detecting clusters of closely related cells. By sampling not just a few, but large numbers of single cells, similarity of expression patterns can be used to build a map of cells and how they are related. This method permits access to undiluted expression data from every distinct type of cell present in a population, without the need for prior purification of those cell types, In addition, where known markers are available, these can be used in silico to delineate cells of interest.

Among the provided embodiments is a method of preparing a polynucleotide library, e.g. cDNA library, from a plurality of single cells by releasing mRNA from each single cell to provide a plurality of individual samples, wherein the mRNA in each individual mRNA sample is from a single cell, synthesizing a first strand of cDNA from the mRNA in each individual mRNA sample and incorporating a tag into the cDNA to provide a plurality of tagged cDNA samples, wherein the cDNA in each tagged cDNA sample is complementary to mRNA from a single cell pooling the tagged cDNA samples and amplifying the pooled cDNA samples to generate a cDNA library comprising double-stranded cDNA. By utilizing the above method, it is feasible to prepare samples for sequencing from several hundred single cells in a short time. Traditional methods for preparing a fragment library from RNA for sequencing include gel excision steps that are laborious. In some aspects of the methods described herein, a set of about 96 cells is prepared as a single sample (after cDNA synthesis), which makes it feasible to prepare several hundred cells for sequencing. Additionally, technical variation can be minimized because each set of about −96 cells is prepared together (in a single tube).

In some aspects of the embodiments, each cDNA sample obtained from a single cell is tagged, which allows gene expression to be analyzed at the level of a single cell. This allows dynamic processes, such as the cell cycle, to be studied and distinct cell types in a complex tissue (e.g. the brain) to be analyzed. In some aspects, the cDNA samples can be pooled prior to analysis. Pooling the samples simplifies handling of the samples from each single cell and reduces the time required to analyze gene expression in the single cells, which allows for high throughput analysis of gene expression. Pooling of the cDNA samples prior to amplification also provides the advantage that technical variation between samples is virtually eliminated, In addition, as the cDNA samples are pooled before amplification, less amplification is required to generate sufficient amounts of cDNA for subsequent analysis compared to amplifying and treating cDNA samples from each single ceil separately. This reduces amplification bias, and also means that any bias will be similar across all the cells used to provide pooled cDNA samples. RNA purification, storage and handling are also not required, which helps to eliminate problems caused by the unstable nature of RNA.

T cell receptor chain pairs are types of immune receptors contemplated to be sequenced using the presently disclosed methods. In some embodiments, the single cell transcriptome analyses allow the generation of polynucleotide libraries for high-throughput sequencing and gene expression analysis that and can be coupled to sequencing of one or more target sequences, such as one or more sequences of a TCR, e.g., for the purposes of determining clonotypes, and sequences that can be combined to provide genomic and/or transcriptomic sequencing information. In some embodiments, a polynucleotide library can be developed that is a human derived library panel for TCR discovery from patient or cohorts with specific common attributes, or for determination of clonotypes present in a population of cells, e.g., T cells. In some embodiments of the provided method, the starting material can be any source that contains a population of cells of interest that does or is likely to contain the target polynucleotide of interest, such as the immune molecule or receptor, e.g. TCR. In some embodiments, starting material can be peripheral blood or from a tissue biopsy, from which immune cells are globally isolated or sub-sorted for naive, memory and/or antibody secreting cells (ASC) if desired. In some embodiments, the provided method can be applied to multiple different types of singular or paired variable sequences, e.g., T-cell receptor chain pairs and antibody immunoglobulin chain pairs.

In some aspects, the cDNA libraries produced by the single cell transcriptome analyses are suitable for the analysis of gene expression profiles of single cells by direct sequencing, and it is possible to use these libraries to study the expression of genes, including expression of genes associated with or cells bearing a particular target polynucleotide of interest, such TCR. In some embodiments, gene expression profiles which were not previously known can be analyzed. In some embodiments, the single cell transcriptome analyses can be used to characterize or compare each of a plurality of cells from a sample for their transcriptional cell state, e.g. activated, exhausted, proliferating or other desired parameter or attribute of cells. In some embodiments, the single cell transcriptome analyses can be used to facilitate the discovery of therapeutic candidates, such as TCRs, by looking at the response of particular cells bearing a particular TCR specific to an antigen of interest.

In some embodiments, the provided methods can be used to possible to identify cells expressing T cell clonotype that is associated with a desired response, e.g. nature or degree of T cell activation. In some embodiments, the single cell transcriptome analyses coupled with T cell clonotype determination make it possible to capture a richer data set by analysis of the whole transcriptome.

In some embodiments, single cell gene expression profiling involves determining the gene expression of one or more selected genes, e.g., one or more genes involved in immune cell function or genes indicative of immune cell phenotypes. In some embodiments, a panel of genes can be assessed. In some embodiments, the gene expression analysis of one or a panel of genes can be coupled to clonotype determination at the single cell level. In some embodiments, both gene expression analysis and clonotype determination can be achieved in one simultaneous reaction, and provide a mechanism for linking sequence information of sequences derived from the same cell. For example, in some embodiments, the TCRaP genes can be sequenced among other panel of genes in the single cell, thereby associating the T cell clonotype and gene expression profile of genes of interest, of a single cell.

In some embodiments, exemplary panel of genes for expression profile analysis includes genes involved in function of immune cells, e.g., T cells, or phenotypic markers of an immune cell, e.g., T cells. In some embodiments, such panel of genes include genes that are associated with function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Exemplary genes in the panel can include genes encoding proteins such as CD4, ICOS, FOXP3, FOXP3V1, PMCH, CD80, FOXP3Y, CD86, CD70, CD40, IL-6, CD2, CD3D, GPR171, CXCL13, PD-1 (CD279), IL-2, IL-4, IL-10, CD8B, KLRK1, CCL4, RUNX3V1, RUNX3, NKG7, CD45RA, CD45RO, CD62L, CD69, CD25, CCR7, CD27, CD28, CD56, CD122, CD127, CD95, CXCR3, LFA-1, KLRG1, T-bet, CD8, IL-7Rα, IL-2Rβ, CD3, CD14, ROR1, granzyme B, granzyme H, CD20, CD11b, CD16, HLA-DR, PD-L1, IFNγ, KIRK1, caspase 2, caspase 3, caspase 6, caspase 7, caspase 8, caspase 9, caspase 10, Bcl-2, Bax, Bad, Bid, CD196 (CCR6), CTLA-4 (CD152), TIGIT (VSIG9, VSTM3), LAG-3 (CD223), 2B4 (CD244), BTLA (CD272), TIM3 (HAVCR2), VISTA (PD1-H) or CD96. One or more of these genes or other genes can be selected in a panel for gene expression profile analysis of the single cell, together with clonotype determination.

2. Single Cell Phenotype Determination

In some embodiments, the one or more properties, parameters, phenotypes, attributes or functions of the T cells include determination of the phenotype of the T cell. In some embodiments, the cell phenotype includes cell surface expression of a marker, such as a cell surface protein, that are associated with function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation of the cell. In some embodiments, the cell phenotype is determined at the single cell level. In some embodiments, the cell phenotype determination, e.g., cell surface protein expression, is determined for one or more phenotypes, e.g., expression of one or more cell surface markers. In some embodiments, the cell phenotype determination is performed using high-throughput methods. In some embodiments, cell phenotype is coupled with clonotype determination at the single cell level. In such embodiments, the cell phenotype and cell clonotype can be readily associated. In some embodiments, the cell phenotype analysis can include various features of the methods as described in WO2017/053905, which is incorporated by reference in its entirety.

In some embodiments, clonotype determination, e.g., paired TCRαβ sequencing, and the cell phenotype determination of a plurality of cells are produced in one simultaneous reaction, and provide a mechanism for linking sequence information of sequences derived from the same cell. In some embodiments, the single cell phenotype determination can be coupled to clonotype determination, e.g., by sequencing of TCR genes, and single cell barcoding. In some embodiments, both cell phenotype determination and clonotype determination can be achieved in one simultaneous reaction, and provide a mechanism for linking sequence information of sequences derived from the same cell.

In some embodiments, cell phenotype is determined on a single cell surface phenotyping or single cell immunophenotyping. In some embodiments, the single cell surface phenotyping involves emulsion-based single cell separation and analysis. In some embodiments, the methods involve using oligonucleotide barcoding conjugated to an affinity agent, e.g., an antibody or antigen-binding fragment thereof or an MHC-peptide tetramer. The oligonucleotide barcodes can be used to assess binding of the affinity agent on the surface marker, thereby assessing the surface marker expression at a single cell level without the requirement of fluorophores. In some embodiments, in single cell surface phenotyping, surface protein-specific antibodies are conjugated to oligonucleotides. In some embodiments, the oligonucleotides are designed to contain a sequence motif which is unique to the target-specificity of the conjugated antibody. The oligonucleotide can be conjugated to the affinity agent portion of the affinity agent-oligonucleotide conjugate (e.g., an antibody) covalently or non-covalently (e.g., biotin-oligonucleotide to streptavidin-antibody).

In some embodiments, the single cell surface phenotyping involves incubating cells in a mixture or a solution with one or more affinity agent-oligonucleotide conjugates. The cells can be washed to remove unbound affinity agent-oligonucleotide conjugates. Cells are then encapsulated in vessels, e.g., an emulsion. The cells can be present in the vessels at a single cell per vessel density. Thus, the affinity agent-oligonucleotide conjugates within a vessel, e.g., droplet, are bound to the cell surface, e.g., through a specific antibody-surface protein interaction. The method can comprise attaching a vessel-specific DNA sequence (e.g., a unique vessel barcode) to the affinity agent-conjugated oligonucleotides. Additional cellular DNA or mRNA analysis, phenotypic measurements, functional testing, cell-sorting or other analyses can be carried out prior to, concurrently with, or after barcoding the affinity agent-conjugated oligonucleotide, (e.g., with a DNA barcode).

In some embodiments, exemplary phenotypes for assessment include expression of markers, e.g., cell surface markers, or other factors, e.g., cytokines or other factors, involved in function of immune cells, e.g., T cells. In some embodiments, such phenotypes include expression of markers that are associated with function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation in T cells. Exemplary markers or factors for phenotypic determination include one or more of CD28, CD62L, CCR7, CD27, CD127, CD4, CD8, CD45RA, CD45RO, CD3, CD14, ROR1, granzyme B, granzyme H, CD20, CD11b, CD16, HLA-DR, ICOS, FOXP3, PMCH, CD80, CD86, CD40, CD70, GPR171, PD-L1, CD2, CD3d, IFNγ, KIRK1, CCL4, RUNX3, NKG7, IL-6, CD56, KLRG1, CD95, CD25, IL-2, IFN-γ, IL-4, IL-10, caspase 2, caspase 3, caspase 6, caspase 7, caspase 8, caspase 9, caspase 10, Bcl-2, Bax, Bad, Bid, CD196 (CCR6), CTLA-4 (CD152), PD-1 (CD279), TIGIT (VSIG9, VSTM3), LAG-3 (CD223), 2B4 (CD244), BTLA (CD272), TIM3 (HAVCR2), VISTA (PD1-H) and CD96. One or more of these phenotypes can be selected for phenotypic analysis of a single cell, together with clonotype determination.

3. Other Assessment

In some embodiments, the one or more properties, parameters, phenotypes, attributes or functions of the T cells include assessment of the properties, parameters, phenotypes, attributes or functions on a population level before or concurrently with clonotype determination. For example, in some embodiments, clonotype determination can be performed on a population of cells, e.g., a particular subset of cells obtained from the subject in a biological test sample or a T cell composition. In some embodiments, population level assessment can be used to select particular cells for analysis. In some embodiments, particular population of cells or cells can be selected based on population level analysis prior to or simultaneously with clonotype determination and identification. The information obtained from such population level determination of properties, parameters, phenotypes, attributes or functions of the T cells can also be coupled with the clonotype determination to identify specific clones of interest. In some embodiments, the population level assessment can include assessment of gene expression analysis, transcriptome profiling, surface cell phenotype, epigenetic profiles, cell surface protein expression, activation phenotype or effector function.

In some embodiments, flow cytometry can be used on the population level to sort specific subsets of cells for clonotype analysis. In some embodiments, cell surface phenotype determination and cell sorting based on cell surface markers, can be used to select specific populations of cell of interest. For example, surface expression of markers that are associated with function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation in T cells, can be used to select cells exhibiting particular phenotypes or attributes.

### III. T CELL COMPOSITIONS FOR ADOPTIVE T CELL THERAPY

The provided methods can be carried out on T cell compositions containing T cells that are for use in producing a therapeutic T cell composition, such as a T cell therapy containing T cell engineered with a recombinant receptor (e.g. CAR), including T cell compositions at various stages of adoptive cell therapy, e.g., before and after engineering of the cells and/or before and after administration of the cells in the subject. In some embodiments, the provided methods involve determining characteristics, such as clonotypes present and/or clonal diversity, of populations of T cells, such as T cells that are genetically engineered to express a recombinant receptor or precursor thereof.

In some embodiments, the process of generating the cell therapy includes analyzing the cells or identifying cellular attributes of cells used in adoptive cell therapy, e.g., engineered T cells, such as using the methods provided herein. In some embodiments, the provided methods involve determining and identifying the phenotype, function, attribute, or property of cells at various stages of adoptive cell therapy, such as cells identified by clonotypic tracking of T cells. In some embodiments, the methods can be used to identify features or attributes of T cells, such as T cells obtained from a subject and/or cells used in connection with manufacturing or formulating a drug product, that are predicted to or likely to result in one or more advantageous or desired features associated with cell therapy upon administration of the therapeutic T cell drug product. In certain embodiments, the T cell therapy contains one or more cells that express a recombinant receptor, e.g., a CAR.

A. Recombinant Receptors

Among the recombinant receptors are antigen receptors and receptors containing one or more component thereof. The recombinant receptors may include chimeric receptors, such as those containing ligand-binding domains or binding fragments thereof and intracellular signaling domains, functional non-TCR antigen receptors, chimeric antigen receptors (CARs), and T cell receptors (TCRs), such as transgenic TCRs, and components of any of the foregoing. In some embodiments, the chimeric receptor, such as a CAR, generally includes the extracellular antigen (or ligand) binding domain linked to one or more intracellular signaling components, in some aspects via linkers and/or transmembrane domain(s).

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentj ens et al., Sci Transl Med. 2013 5(177). See also WO2014031687, U.S. Pat. No. 8,339,645, 7,446,179, US 2013/0149337, U.S. Pat. Nos. 7,446,190, and 8,389,282.

1. Chimeric Antigen Receptors (CAR)

In some embodiments, a CAR is generally a genetically engineered receptor with an extracellular ligand binding domain, such as an extracellular portion containing an antibody or fragment thereof, linked to one or more intracellular signaling components. In some embodiments, the chimeric antigen receptor includes a transmembrane domain and/or intracellular domain linking the extracellular domain and the intracellular signaling domain. Such molecules typically mimic or approximate a signal through a natural antigen receptor and/or signal through such a receptor in combination with a costimulatory receptor.

In particular embodiments, the recombinant receptor, such as chimeric receptor, contains an intracellular signaling region, which includes a cytoplasmic signaling domain (also interchangeably called an intracellular signaling domain), such as a cytoplasmic (intracellular) region capable of inducing a primary activation signal in a T cell, for example, a cytoplasmic signaling domain of a T cell receptor (TCR) component (e.g. a cytoplasmic signaling domain of a zeta chain of a CD3-zeta (CD3ζ) chain or a functional variant or signaling portion thereof) and/or that comprises an immunoreceptor tyrosine-based activation motif (ITAM).

In some embodiments, the chimeric receptor further contains an extracellular ligand-binding domain that specifically binds to a ligand (e.g. antigen) antigen. In some embodiments, the chimeric receptor is a CAR that contains an extracellular antigen-recognition domain that specifically binds to an antigen.

In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

In some embodiments, engineered cells, such as T cells, are provided that express a CAR with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). In some embodiments, the CAR contains an antibody or an antigen-binding fragment (e.g. scFv) that specifically recognizes an antigen, such as an intact antigen, expressed on the surface of a cell.

Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g. CAR), includes a ligand-binding domain that binds, such as specifically binds, to an antigen (or a ligand). In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the antigen (or a ligand) is a tumor antigen or cancer marker. In some embodiments, the antigen (or a ligand) is or includes orphan tyrosine kinase receptor ROR1, B cell maturation antigen (BCMA), carbonic anhydrase 9 (CAIX), Her2/neu (receptor tyrosine kinase erbB2), CD19, CD20, CD22, and hepatitis B surface antigen, anti-folate receptor, CD23, CD24, CD30, CD33, CD38, CD44, chondroitin sulfate proteoglycan 4 (CSPG4), EGFR, epithelial glycoprotein 2 (EPG-2), epithelial glycoprotein 40 (EPG-40), ephrin receptor A2 (EPHa2), Her2/neu (receptor tyrosine kinase erb-B2), Her3 (erb-B3), Her4 (erb-B4), erbB dimers, EGFR vIII, folate binding protein (FBP), Fc receptor like 5 (FCRL5, also known as Fc receptor homolog 5 or FCRH5), fetal acetylcholine receptor (fetal AchR), ganglioside GD3, Human high molecular weight-melanoma-associated antigen (HMW-MAA), IL-22 receptor alpha(IL-22R-alpha), IL-13 receptor alpha 2 (IL-13R-alpha2), kinase insert domain receptor (kdr), kappa light chain, Leucine Rich Repeat Containing 8 Family Member A (LRRC8A), Lewis Y, L1-cell adhesion molecule, (L1-CAM), Melanoma-associated antigen (MAGE)-A1, MAGE-A3, MAGE-A6, MAGE-A10, Preferentially expressed antigen of melanoma (PRAME), survivin, TAG72, B7-H3, B7-H6, IL-13 receptor alpha 2 (IL-13Ra2), carbonic anhydrase 9 (CA9, also known as CAIX or G250), CD171, Human leukocyte antigen A1 (HLA-AI), MAGE A1, Human leukocyte antigen A2 (HLA-A2), cancer/testis antigen 1B (CTAG, also known as NY-ESO-1), folate receptor-alpha, CD44v6, CD44v7/8, αvβ6 integrin (avb6 integrin), 8H9, neural cell adhesion molecule (NCAM), vascular endothelial growth factor receptor (VEGF receptors), Trophoblast glycoprotein (TPBG also known as 5T4), NKG2D ligands, CD44v6, dual antigen, a cancer-testes antigen, mesothelin (MSLN), murine cytomegalovirus (CMV), mucin 1 (MUC1), MUC16, a prostate specific antigen, prostate stem cell antigen (PSCA), prostate specific membrane antigen (PSMA), natural killer group 2 member D (NKG2D) ligands, NY-ESO-1, melan A (MART-1), glycoprotein 100 (gp100), glypican-3 (GPC3), G Protein Coupled Receptor 5D (GPRC5D), oncofetal antigen, Receptor Tyrosine Kinase Like Orphan Receptor 1 (ROR1), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor 2 (VEGF-R2), carcinoembryonic antigen (CEA), Her2/neu, estrogen receptor, progesterone receptor, ephrinB2, CD123, CD133, c-Met, ganglioside GD-2, O-acetylated GD2 (OGD2), CE7 epitope of L1-CAM, Wilms Tumor 1 (WT-1), a cyclin, cyclin A2, C-C Motif Chemokine Ligand 1 (CCL-1), CD138, a pathogen-specific antigen and an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the antigen (or a ligand) is a tumor antigen or cancer marker. In some embodiments, the antigen (or a ligand) is or includes orphan tyrosine kinase receptor ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the antigen is a pathogen-specific antigen. In some embodiments, the antigen is a viral antigen (such as a viral antigen from HIV, HCV, HBV, etc.), bacterial antigens, and/or parasitic antigens.

In some embodiments, the antigen or antigen binding domain is CD19. In some embodiments, the scFv contains a VH and a VL derived from an antibody or an antibody fragment specific to CD19. In some embodiments, the antibody or antibody fragment that binds CD19 is a mouse derived antibody such as FMC63 and SJ25C1. In some embodiments, the antibody or antibody fragment is a human antibody, e.g., as described in U.S. Patent Publication No. US 2016/0152723.

In some embodiments, the scFv is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). The FMC63 antibody comprises CDRH1 and H2 set forth in SEQ ID NOS: 54, 55 respectively, and CDRH3 set forth in SEQ ID NOS:56, 70 and CDRL1 set forth in SEQ ID NO: 51 and CDR L2 set forth in SEQ ID NOS: 52 or 71 and CDR L3 set forth in SEQ ID NOS: 53 or 70. The FMC63 antibody comprises the heavy chain variable region (V_{H}) comprising the amino acid sequence of SEQ ID NO: 57 and the light chain variable region (V_{L}) comprising the amino acid sequence of SEQ ID NO: 58. In some embodiments, the svFv comprises a variable light chain containing the CDRL1 sequence of SEQ ID NO:51, a CDRL2 sequence of SEQ ID NO:52, and a CDRL3 sequence of SEQ ID NO:53 and/or a variable heavy chain containing a CDRH1 sequence of SEQ ID NO: 54, a CDRH2 sequence of SEQ ID NO:55, and a CDRH3 sequence of SEQ ID NO:56. In some embodiments, the scFv comprises a variable heavy chain region of FMC63 set forth in SEQ ID NO:57 and a variable light chain region of FMC63 set forth in SEQ ID NO:58. In some embodiments, the variable heavy and variable light chain are connected by a linker. In some embodiments, the linker is set forth in SEQ ID NO:72. In some embodiments, the scFv comprises, in order, a V_{H}, a linker, and a V_{L}. In some embodiments, the scFv comprises, in order, a V_{L}, a linker, and a V_{H}. In some embodiments, the svFc is encoded by a sequence of nucleotides set forth in SEQ ID NO:73 or a sequence that exhibits at least 85%, 86%, 87%, 88%, 89%, 90%, 91%, 92%, 93%, 94%, 95%, 96%, 97%, 98%, or 99% sequence identity to SEQ ID NO:73. In some embodiments, the scFv comprises the sequence of amino acids set forth in SEQ ID NO:59 or a sequence that exhibits at least 85%, 86%, 87%, 88%, 89%, 90%, 91%, 92%, 93%, 94%, 95%, 96%, 97%, 98%, or 99% sequence identity to SEQ ID NO:59.

In some embodiments the scFv is derived from SJ25C1. SJ25C1 is a mouse monoclonal IgG1 antibody raised against Nalm-1 and -16 cells expressing CD19 of human origin (Ling, N. R., et al. (1987). Leucocyte typing III. 302). The SJ25C1 antibody comprises CDRH1, H2 and H3 set forth in SEQ ID NOS:63-65, respectively, and CDRL1, L2 and L3 sequences set forth in SEQ ID NOS: 60-62, respectively. The SJ25C1 antibody comprises the heavy chain variable region (V_{H}) comprising the amino acid sequence of SEQ ID NO: 66 and the light chain variable region (V_{L}) comprising the amino acid sequence of SEQ ID NO: 67. In some embodiments, the svFv comprises a variable light chain containing the CDRL1 sequence of SEQ ID NO:60, a CDRL2 sequence of SEQ ID NO: 61, and a CDRL3 sequence of SEQ ID NO:62 and/or a variable heavy chain containing a CDRH1 sequence of SEQ ID NO:63, a CDRH2 sequence of SEQ ID NO:64, and a CDRH3 sequence of SEQ ID NO:65. In some embodiments, the scFv comprises a variable heavy chain region of SJ25C1 set forth in SEQ ID NO:66 and a variable light chain region of SJ25C1 set forth in SEQ ID NO:67. In some embodiments, the variable heavy and variable light chain are connected by a linker. In some embodiments, the linker is set forth in SEQ ID NO:68. In some embodiments, the scFv comprises, in order, a V_{H}, a linker, and a V_{L}. In some embodiments, the scFv comprises, in order, a V_{L}, a linker, and a V_{H}. In some embodiments, the scFv comprises the sequence of amino acids set forth in SEQ ID NO:69 or a sequence that exhibits at least 85%, 86%, 87%, 88%, 89%, 90%, 91%, 92%, 93%, 94%, 95%, 96%, 97%, 98%, or 99% sequence identity to SEQ ID NO:69.

In some embodiments, the antigen is CD20. In some embodiments, the scFv contains a VH and a VL derived from an antibody or an antibody fragment specific to CD20. In some embodiments, the antibody or antibody fragment that binds CD20 is an antibody that is or is derived from Rituximab, such as is Rituximab scFv.

In some embodiments, the antigen is CD22. In some embodiments, the scFv contains a VH and a VL derived from an antibody or an antibody fragment specific to CD22. In some embodiments, the antibody or antibody fragment that binds CD22 is an antibody that is or is derived from m971, such as is m971 scFv.

In some embodiments, the antigen or antigen binding domain is BCMA. In some embodiments, the scFv contains a VH and a VL derived from an antibody or an antibody fragment specific to BCMA. In some embodiments, the antibody or antibody fragment that binds BCMA is or contains a VH and a VL from an antibody or antibody fragment set forth in International Patent Applications, Publication Number WO 2016/090327 and WO 2016/090320.

In some embodiments, the antigen or antigen binding domain is GPRC5D. In some embodiments, the scFv contains a VH and a VL derived from an antibody or an antibody fragment specific to GPRC5D. In some embodiments, the antibody or antibody fragment that binds GPRC5D is or contains a VH and a VL from an antibody or antibody fragment set forth in International Patent Applications, Publication Number WO 2016/090329 and WO 2016/090312.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, WIC class I molecules are heterodimers having a membrane spanning a chain, in some cases with three a domains, and a non-covalently associated β2 microglobulin. Generally, MHC class II molecules are composed of two transmembrane glycoproteins, α and β, both of which typically span the membrane. An WIC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human WIC can also be referred to as human leukocyte antigen (HLA).

The term “MHC-peptide complex” or “peptide-WIC complex” or variations thereof, refers to a complex or association of a peptide antigen and an WIC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by methods known in the art (see e.g. US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US published application No. US20020150914, US2014/0294841; and Cohen C J. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4, more particularly, IgG1 (e.g., human IgG1). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known in the art.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the recombinant receptor such as the CAR, such as the antibody portion thereof, further includes a spacer, such as a spacer region between the antigen-binding molecules, such as one or more antigen-binding fragment, e.g. scFv, and a transmembrane domain. In some embodiments, the spacer may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014031687. In some embodiments, the spacer has the sequence set forth in SEQ ID NO: 1, and is encoded by the sequence set forth in SEQ ID NO: 2. In some embodiments, the spacer has the sequence set forth in SEQ ID NO: 3. In some embodiments, the spacer has the sequence set forth in SEQ ID NO: 4.

In some embodiments, the spacer includes at least a portion of an immunoglobulin constant region or variant or modified version thereof. In some embodiments, the constant region or portion is of IgD. In some embodiments, the spacer has the sequence set forth in SEQ ID NO: 5.

In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 1, 3, 4 and 5.

In some embodiments, the spacer has a sequence of amino acids that exhibits at least 85%, 86%, 87%, 88%, 89%, 90%, 91%, 92%, 93%, 94%, 95%, 96%, 97%, 98%, 99% or more sequence identity to any of SEQ ID NOS: 76-82, 74, 75.

The antigen recognition domain generally is linked to one or more intracellular signaling components, such as signaling components that mimic activation through an antigen receptor complex, such as a TCR complex, in the case of a CAR, and/or signal via another cell surface receptor. Thus, in some embodiments, the antigen binding component (e.g., antibody) is linked to one or more transmembrane and intracellular signaling regions. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CDS, CD8, CD9, CD16, CD22, CD33, CD37, CD64, CD80, CD86, CD 134, CD137, CD 154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). The receptor, e.g., the CAR, generally includes at least one intracellular signaling component or components. Among the intracellular signaling region are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The recombinant receptor, e.g., the CAR, generally includes at least one intracellular signaling component or components. In some embodiments, the receptor, e.g. CAR, includes an intracellular component of a TCR complex, such as a TCR CD3 chain that mediates T-cell activation and cytotoxicity, e.g., CD3 zeta chain. Thus, in some aspects, the antigen- binding molecules, such as one or more antigen-binding fragment, e.g. scFv, is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor y, CD8, CD4, CD25, or CD16. For example, in some aspects, the CAR includes a chimeric molecule between CD3-zeta (CD3ζ) or Fc receptor γ and CD8, CD4, CD25 or CD16.

In some embodiments, upon ligation of the CAR or other chimeric receptor, the cytoplasmic domain or intracellular signaling region of the CAR activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling region of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CD8, CD22, CD79a, CD79b, and CD66d. In some aspects, examples of ITAM containing primary cytoplasmic signaling sequences include those derived from TCR or CD3 zeta, FcR gamma or FcR beta. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, CD27, DAP10, and ICOS. In some embodiments, the CAR includes a signaling region and/or transmembrane portion of a costimulatory receptor, such as CD28, 4-1BB, OX40, DAP10, and ICOS. In some aspects, the same CAR includes both the signaling region and costimulatory components.

In some embodiments, the signaling region is included within one CAR, whereas the costimulatory component is provided by another CAR recognizing another antigen. In some embodiments, the CARs include activating or stimulatory CARs, and costimulatory CARs, both expressed on the same cell (see WO2014/055668). In some aspects, the CAR is the stimulatory or activating CAR; in other aspects, it is the costimulatory CAR. In some embodiments, the cells further include inhibitory CARs (iCARs, see Fedorov et al., Sci. Transl. Medicine, 5(215) (December, 2013), such as a CAR recognizing a different antigen, whereby an activating signal delivered through a CAR recognizing a first antigen is diminished or inhibited by binding of the inhibitory CAR to its ligand, e.g., to reduce off-target effects.

In some embodiments, the intracellular signaling domain of the CD8+ cytotoxic T cells is the same as the intracellular signaling domain of the CD4+ helper T cells. In some embodiments, the intracellular signaling domain of the CD8+ cytotoxic T cells is different than the intracellular signaling domain of the CD4+ helper T cells.

In certain embodiments, the intracellular signaling region comprises a CD28 transmembrane and signaling domain linked to a CD3 (e.g., CD3-zeta) intracellular domain. In some embodiments, the intracellular signaling region comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular portion, such as an antigen-binding portion, containing the antibody or fragment described herein. In some aspects, the chimeric antigen receptor includes an extracellular portion, such as an antigen-binding portion, containing the antibody or fragment described herein and an intracellular signaling domain. In some embodiments, the antibody or fragment includes an scFv or a single-domain V_{H} antibody and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain disposed between the extracellular domain and the intracellular signaling region.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the CAR contains an antibody, e.g., an antibody fragment, such as any described herein, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, such as any described herein, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 8 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO:8in some embodiments, the transmembrane-domain containing portion of the recombinant receptor comprises the sequence of amino acids set forth in SEQ ID NO: 9 or a sequence of amino acids having at least at or about 85%, 86%, 87%, 88%, 89%, 90%, 91%, 92%, 93%, 94%, 95%, 96%, 97%, 98%, 99% or more sequence identity thereto.

In some embodiments, the chimeric antigen receptor contains an intracellular domain of a T cell costimulatory molecule. In some aspects, the T cell costimulatory molecule is CD28 or 4-1BB.

In some embodiments, the intracellular signaling region comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 10 or 11 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 10 or 11. In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 12 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 12.

In some embodiments, the intracellular signaling region comprises a human CD3 chain, optionally a CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 13, 14 or 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 13, 14 or 15.

In some aspects, the spacer contains only a hinge region of an IgG, such as only a hinge of IgG4 or IgG1, such as the hinge only spacer set forth in SEQ ID NO:1. In other embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO:3. In some embodiments, the spacer is an Ig hinge, e.g., an IgG4 hinge, linked to a C_{H}3 domain only, such as set forth in SEQ ID NO:4. In some embodiments, the spacer is or comprises a glycine-serine rich sequence or other flexible linker such as known flexible linkers.

For example, in some embodiments, the CAR includes: an extracellular ligand-binding portion, such as an antigen-binding portion, such as an antibody or fragment thereof, including sdAbs and scFvs, that specifically binds an antigen, e.g. an antigen described herein; a spacer such as any of the Ig-hinge containing spacers; a transmembrane domain that is a portion of CD28 or a variant thereof an intracellular signaling domain containing a signaling portion of CD28 or functional variant thereof; and a signaling portion of CD3 zeta signaling domain or functional variant thereof. In some embodiments, the CAR includes: an extracellular ligand-binding portion, such as an antigen-binding portion, such as an antibody or fragment thereof, including sdAbs and scFvs, that specifically binds an antigen, e.g. an antigen described herein; a spacer such as any of the Ig-hinge containing spacers; a transmembrane domain that is a portion of CD28 or a variant thereof an intracellular signaling domain containing a signaling portion of 4-1BB or functional variant thereof; and a signaling portion of CD3 zeta signaling domain or functional variant thereof

2. T Cell Receptors

In some embodiments, the recombinant protein is or includes a recombinant T cell receptor (TCR). In some embodiments, the recombinant TCR is specific for an antigen, generally an antigen present on a target cell, such as a tumor-specific antigen, an antigen expressed on a particular cell type associated with an autoimmune or inflammatory disease, or an antigen derived from a viral pathogen or a bacterial pathogen.

In some embodiments, the genetically engineered antigen receptors include recombinant T cell receptors (TCRs) and/or TCRs cloned from naturally occurring T cells. In some embodiments, the recombinant TCR is different from the native TCR (e.g., sequenced for clonotype determination) of the T cell obtained from a subject or to be engineered.

In some embodiments, a “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MEW receptor. In some embodiments, the TCR is in the αβ form. Typically, TCRs that exist αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MEW) molecules. In some embodiments, a TCR also can contain a constant domain, a transmembrane domain and/or a short cytoplasmic tail (see, e.g., Janeway et al., Immunobiology: The Immune System in Health and Disease, 3^{rd} Ed., Current Biology Publications, p. 4:33, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable α chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., V_{α} or V_{β}; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or C_{α}, typically amino acids 117 to 259 based on Kabat, β-chain constant domain or C_{β}, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, a such as a high-affinity T cell clone can be isolated from a patient, and the TCR isolated. In some embodiments, the T-cells can be a cultured T-cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354. In some embodiments, the TCR or antigen-binding portion thereof can be synthetically generated from knowledge of the sequence of the TCR.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources can be used. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T- cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Va and VP from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPredl (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models can be used. For predicting MHC class I binding sites, such models include, but are not limited to, ProPredl (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007)

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO 2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR a chain variable region sequence is fused to the N terminus of a sequence corresponding to a TCR a chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR a chain containing a variable a domain, a constant a domain and a first dimerization motif attached to the C-terminus of the constant a domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR a chain and TCR β chain together.

In some embodiments, the TCR is a scTCR. Typically, a scTCR can be generated using methods known to those of skill in the art, See e.g., Soo Hoo, W. F. et al. PNAS (USA) 89, 4759 (1992); Willfing, C. and Pliickthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR a chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an α chain variable region sequence fused to the N terminus of an a chain extracellular constant domain sequence, and a second segment constituted by a β chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a β chain extracellular constant domain sequence, and a second segment constituted by an α chain variable region sequence fused to the N terminus of a sequence a chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula -P-AA-P- wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula -PGGG-(SGGGG)5-P- wherein P is proline, G is glycine and S is serine (SEQ ID NO: 22). In some embodiments, the linker has the sequence GSADDAKKDAAKKDGKS (SEQ ID NO: 23).

In some embodiments, the scTCR contains a covalent disulfide bond linking a residue of the immunoglobulin region of the constant domain of the a chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10-5 and 10-12 M and all individual values and ranges therein. In some embodiments, the target antigen is an MHC-peptide complex or ligand.

In some embodiments, after the T-cell clone is obtained, the TCR alpha and beta chains are isolated and cloned into a gene expression vector. In some embodiments, nucleic acid or nucleic acids encoding a TCR, such as α and β chains, can be amplified by PCR, cloning or other suitable means and cloned into a suitable expression vector or vectors. In some embodiments, the TCR alpha and beta genes are linked via a picornavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; Hackett et al. (2010) Molecular Therapy: The Journal of the American Society of Gene Therapy. 18:674-683. The expression vector can be any suitable recombinant expression vector, and can be used to transform or transfect any suitable host. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif), the pET series (Novagen, Madison, Wis.), the pGEX series (Pharmacia Biotech, Uppsala, Sweden), or the pEX series (Clontech, Palo Alto, Calif). In some cases, bacteriophage vectors, such as λG10, λGT11, λZapII (Stratagene), λEMBL4, and λNM1149, also can be used. In some embodiments, plant expression vectors can be used and include pBI01, pBI101.2, pBI101.3, pBI121 and pBIN19 (Clontech). In some embodiments, animal expression vectors include pEUK-Cl, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

B. Methods for Producing Engineered Cells

In some embodiments, the T cell compositions are used in connection with manufacturing, generating or producing a cell therapy, which can be carried out via a process that includes one or more processing steps, such as steps for the isolation, separation, selection, activation or stimulation, transduction, cultivation, expansion, washing, suspension, dilution, concentration, and/or formulation of the cells. In some embodiments, the methods of generating or producing a cell therapy include isolating cells from a subject, preparing, processing, culturing under one or stimulating conditions. In some embodiments, the method includes processing steps carried out in an order in which: cells, e.g. primary cells, are first isolated, such as selected or separated, from a biological sample; selected cells are incubated with viral vector particles for transduction, optionally subsequent to a step of stimulating the isolated cells in the presence of a stimulation reagent; culturing the transduced cells, such as to expand the cells; formulating the transduced cells in a composition for administration to a subject. In some embodiments, the generated engineered cells are re-introduced into the same subject, before or after cryopreservation.

In some embodiments, the one or more processing steps can include one or more of (a) washing a biological sample containing cells (e.g., a whole blood sample, a buffy coat sample, a peripheral blood mononuclear cells (PBMC) sample, an unfractionated T cell sample, a lymphocyte sample, a white blood cell sample, an apheresis product, or a leukapheresis product), (b) isolating, e.g. selecting, from the sample a desired subset or population of cells (e.g., CD4+ and/or CD8+ T cells), for example, by incubation of cells with a selection or immunoaffinity reagent for immunoaffinity-based separation; c) incubating the isolated, such as selected cells, with viral vector particles, (d) culturing, cultivating or expanding the cells such using methods as described and (e) formulating the transduced cells, such as in a pharmaceutically acceptable buffer, cryopreservative or other suitable medium. In some embodiments, the methods can further include (e) stimulating cells by exposing cells to stimulating conditions, which can be performed prior to, during and/or subsequent to the incubation of cells with viral vector particles. In some embodiments, one or more further step of washing or suspending step, such as for dilution, concentration and/or buffer exchange of cells, can also be carried out prior to or subsequent to any of the above steps. In some aspects, the resulting engineered cell composition is introduced into one or more provided biomedical culture vessel.

In some embodiments, the provided methods are carried out such that one, more, or all steps in the preparation of cells for clinical use, e.g., in adoptive cell therapy, are carried out without exposing the cells to non-sterile conditions and without the need to use a sterile room or cabinet. In some embodiments of such a process, the cells are isolated, separated or selected, transduced, washed, optionally activated or stimulated and formulated, all within a closed system. In some aspects of such a process, the cells are expressed from a closed system and introduced into one or more of the biomaterial vessels. In some embodiments, the methods are carried out in an automated fashion. In some embodiments, one or more of the steps is carried out apart from the closed system or device.

In some embodiments, a closed system is used for carrying out one or more of the other processing steps of a method for manufacturing, generating or producing a cell therapy. In some embodiments, one or more or all of the processing steps, e.g., isolation, selection and/or enrichment, processing, incubation in connection with transduction and engineering, and formulation steps is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps. In one example, the system is a system as described in International Patent Application, Publication Number WO2009/072003, or US 20110003380 A1. In one example, the system is a system as described in International Publication Number WO2016/073602.

In some embodiments, cells can be formulated into the vials in an amount for dosage administration, such as for a single unit dosage administration or multiple dosage administration.

In some embodiments, the methods provided herein are methods that involve assessing and determining clonotypes and other properties and parameters of cells, such as engineered cells that express a recombinant receptor. In some embodiments, the assessment and determination of clonotype and other properties and parameters is performed at different stages of engineering and administering the cells for adoptive cell therapy. In some embodiments, assessment and determination of clonotype and other properties and parameters is performed on T cells obtained from a subject prior to administration of the cell therapy and/or prior to engineering the cells. In some embodiments, the assessment and determination of clonotype and other properties and parameters is performed on a biological sample obtained from a subject administered a cell therapy comprising T cells expressing a recombinant receptor.

In some embodiments, the cells in the method are engineered cells that contain a recombinant receptor. In some embodiments, the cells in the method are populations of cells, such as populations of immune cells obtained from a subject for adoptive cell therapy. Also provided are populations of such cells, compositions containing such cells and/or enriched for such cells, such as in which cells expressing the recombinant receptor, e.g. chimeric receptor, make up at least 50, 60, 70, 80, 90, 91, 92, 93, 94, 95, 96, 97, 98, 99, or more percent of the total cells in the composition or cells of a certain type such as T cells or CD8+ or CD4+ cells. Among the compositions are pharmaceutical compositions and formulations for administration, such as for adoptive cell therapy. Also provided are methods for assessing, therapeutic methods for administering the cells and compositions to subjects, e.g., patients, and methods for detecting, selecting, isolating or separating such cells.

1. Isolation or Selection of Cells from Samples

In some embodiments, the processing steps include isolation of cells or compositions thereof from biological samples, such as those obtained from or derived from a subject, such as one having a particular disease or condition or in need of a cell therapy or to which cell therapy will be administered. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. In some embodiments, the cells comprise CD4+ and CD8+ T cells. In some embodiments, the cells comprise CD4+ or CD8+ T cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In some embodiments, the cell is a regulatory T cell (Treg). In some embodiments, the cell further comprises a recombinant FOXP3 or variant thereof.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, preparation of the engineered cells includes one or more culture and/or preparation steps. The cells for engineering may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contain cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished in a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the preparation methods include steps for freezing, e.g., cryopreserving, the cells, either before or after isolation, selection and/or enrichment and/or incubation for transduction and engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, isolation of the cells or populations includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include density-based cell separation methods, such as the preparation of white blood cells from peripheral blood by lysing the red blood cells and centrifugation through a Percoll or Ficoll gradient.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with a selection regent, such as an antibody or binding partner that specifically binds to such markers, followed generally by washing steps and separation of cells having bound the antibody or binding partner, from those cells having not bound to the antibody or binding partner.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. In some embodiments, the selection and/or other aspects of the process is as described in International Patent Application Publication Number WO/2015/164675.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g. magnetic beads, that are coated with a selection agent (e.g. antibody) specific to the marker of the cells. The particles (e.g. beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, by conducting such selection steps or portions thereof (e.g., incubation with antibody-coated particles, e.g., magnetic beads) in the cavity of a centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g. bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g. is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least or about or 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from or from about 5 minutes to 6 hours, such as 30 minutes to 3 hours, for example, at least or about at least 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, the process steps further include negative and/or positive selection of the incubated and cells, such as using a system or apparatus that can perform an affinity-based selection. In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker_{high}) on the positively or negatively selected cells, respectively.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28′ CD62L′ CCR7′ CD27′ CD127′ CD4′ CD8′ CD45RA′ and/or CD45RO⁺ T cells, are isolated by positive or negative selection techniques. In some embodiments, such cells are selected by incubation with one or more antibody or binding partner that specifically binds to such markers. In some embodiments, the antibody or binding partner can be conjugated, such as directly or indirectly, to a solid support or matrix to effect selection, such as a magnetic bead or paramagnetic bead.

For example, CD3′ CD28⁺ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood.1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or ROR1, and positive selection based on a marker characteristic of central memory T cells, such as CD62L or CCR7, where the positive and negative selections are carried out in either order.

CD4⁺ T helper cells are sorted into naive, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA′ CD62L′ CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher© Humana Press Inc., Totowa, N.J.).

In some aspects, the sample or composition of cells to be separated is incubated with a selection reagent, such as containing small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some aspects, separation is achieved in a procedure in which the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain aspects, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International PCT Publication No. WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood may be automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood.1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10:1567-1573; and Godin et al. (2008) J Biophoton. 1(5):355-376). In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

2. Activation and Stimulation of T Cells

In some embodiments, the one or more processing steps include a step of stimulating the isolated cells, such as selected cell populations. The incubation may be prior to or in connection with genetic engineering, such as prior to or in connection of transducing cells with a nucleic acid or vector encoding the recombinant receptor (e.g. CAR). In some embodiments, the stimulation results in activation and/or proliferation of the cells, for example, prior to transduction.

In some embodiments, the provided methods for producing engineered cell include cultivation, incubation, culture, and/or genetic engineering steps. For example, in some embodiments, provided are methods for incubating and/or engineering the depleted cell populations and culture-initiating compositions. In some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant receptor, e.g., CAR.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling region of a TCR complex. In some aspects, the agent turns on or initiates TCR/CD3 intracellular signaling cascade in a T cell. Such agents can include antibodies, such as those specific for a TCR, e.g. anti-CD3. In some embodiments, the stimulating conditions include one or more agent, e.g. ligand, which is capable of stimulating a costimulatory receptor, e.g., anti-CD28. In some embodiments, such agents and/or ligands may be, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2, IL-15 and/or IL-7. In some aspects, the IL-2 concentration is at least about 10 units/mL. In some aspects, the IL-2 concentration is at least about 50 units/mL, at least about 100 units/mL or at least about 200 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood.1:72-82, and/or Wang et al. (2012) J Immunother. 35(9):689-701.

In some embodiments, at least a portion of the incubation in the presence of one or more stimulating conditions or stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g. is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g. in a tube or bag with periodic shaking or rotation. In some embodiments, the incubation is performed with the addition of an incubation buffer to the cells and stimulating agent to achieve a target volume with incubation of the reagent of, for example, 10 mL to 200 mL, such as at least or about at least or about or 10 mL, 20 mL, 30 mL, 40 mL, 50 mL, 60 mL, 70 mL, 80 mL, 90 mL, 100 mL, 150 mL, or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80g to 100g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

3. Methods for Genetic Engineering

In some embodiments, the processing steps include introduction of a nucleic acid molecule encoding a recombinant protein. Various methods for the introduction of genetically engineered components, e.g., recombinant receptors, e.g., CARs or TCRs, are well known and may be used with the provided methods and compositions. Exemplary methods include those for transfer of nucleic acids encoding the polypeptides or receptors, including via viral vectors, e.g., retroviral or lentiviral, non-viral vectors or transposons, e.g. Sleeping Beauty transposon system. Methods of gene transfer can include transduction, electroporation or other method that results into gene transfer into the cell.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, it may be desired to safeguard against the potential that overexpression of a stimulatory factor (for example, a lymphokine or a cytokine) could potentially result in an unwanted outcome or lower efficacy in a subject, such as a factor associated with toxicity in a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, bacterial cytosine deaminase (Mullen et al., Proc. Natl. Acad. Sci. USA. 89:33 (1992)).

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion, e.g. with nucleic acids encoding a recombinant receptor, e.g., a T cell receptor (TCR) or a chimeric antigen receptor (CAR). This transfection for the introduction of the gene of the desired polypeptide or receptor can be carried out with any suitable retroviral vector, for example. The genetically modified cell population can then be liberated from the initial stimulus (the CD3/CD28 stimulus, for example) and subsequently be stimulated with a second type of stimulus (e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

As described above, in some embodiments, the cells are incubated and/or cultured prior to or in connection with genetic engineering. The incubation steps can include culture, cultivation, stimulation, activation, propagation and/or freezing for preservation, e.g. cryopreservation.

In some embodiments, the introducing is carried out by contacting one or more cells of a composition with a nucleic acid molecule encoding the recombinant protein, e.g. recombinant receptor. In some embodiments, the contacting can be effected with centrifugation, such as spinoculation (e.g. centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the system is included with and/or placed into association with other instrumentation, including instrumentation to operate, automate, control and/or monitor aspects of the transduction step and one or more various other processing steps performed in the system, e.g. one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, the composition containing cells, viral particles and reagent can be rotated, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm). In some embodiments, the rotation is carried at a force, e.g., a relative centrifugal force, of from or from about 100 g to 3200 g (e.g. at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, during at least a part of the genetic engineering, e.g. transduction, and/or subsequent to the genetic engineering the cells are transferred to a container such as a bag for culture of the genetically engineered cells, such as for cultivation or expansion of the cells, as described above. In some embodiments, the container for cultivation or expansion of the cells is a bioreactor bag, such as a perfusion bag.

a. Nucleic Acids and Vectors

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, the recombinant receptors are encoded by one or more polynucleotides (e.g., nucleic acid molecules). In some embodiments, the cells that are used in adoptive cell therapy are engineered using, vectors for genetic engineering.

In some aspects, the polynucleotide contains a single coding sequence. In other instances, the polynucleotide contains at least two different coding sequences. In some aspects, the recombinant receptor is or contains a chimeric antigen receptor (CAR). In some aspects, the recombinant receptor is or contains a T cell receptor (TCR), e.g., a transgenic TCR. In some embodiments, the polynucleotides and vectors are used for expression in cells the recombinant receptor.

In some cases, the nucleic acid sequence encoding the recombinant receptor contains a signal sequence that encodes a signal peptide. In other aspects, the signal sequence may encode a heterologous or non-native signal peptide, such as the exemplary signal peptide of the GMCSFR alpha chain set forth in SEQ ID NO: 25 and encoded by the nucleotide sequence set forth in SEQ ID NO:24. In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 25 and encoded by the nucleotide sequence set forth in SEQ ID NO:24, or the CD8 alpha signal peptide set forth in SEQ ID NO:26.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor.

In certain cases where nucleic acid molecules encode two or more different polypeptide chains, each of the polypeptide chains can be encoded by a separate nucleic acid molecule. For example, two separate nucleic acids are provided, and each can be individually transferred or introduced into the cell for expression in the cell.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U .S. Pat. No. 6,060,273). In some embodiments, transcription units can be engineered as a bicistronic unit containing an IRES (internal ribosome entry site), which allows coexpression of gene products ((e.g. encoding a cell surface marker or modified form thereof and encoding the recombinant receptor) by a message from a single promoter. Alternatively, in some cases, a single promoter may direct expression of an RNA that contains, in a single open reading frame (ORF), two or three genes (e.g. encoding a cell surface marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 21), equine rhinitis A virus (E2A, e.g., SEQ ID NO: 20), Thosea asigna virus (T2A, e.g., SEQ ID NO: 6 or 17), and porcine teschovirus-1 (P2A, e.g., SEQ ID NO: 18 or 19) as described in U.S. Patent Publication No. 20070116690.

In some embodiments, the nucleic acid encoding a cell surface marker and the nucleic acid encoding the recombinant receptor are operably linked to the same promoter and are optionally separated by an internal ribosome entry site (IRES), or a nucleic acid encoding a self-cleaving peptide or a peptide that causes ribosome skipping, which optionally is a T2A, a P2A, a E2A or a F2A. In some embodiments, the nucleic acid encoding a cell surface marker and the nucleic acid encoding the recombinant receptor are operably linked to two different promoters. In some embodiments, the nucleic acid encoding a cell surface marker and the nucleic acid encoding the recombinant receptor are present or inserted at different locations within the genome of the cell.

In some embodiments, the vector contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

Exemplary surrogate markers can include truncated forms of cell surface polypeptides, such as truncated forms that are non-functional and to not transduce or are not capable of transducing a signal or a signal ordinarily transduced by the full-length form of the cell surface polypeptide, and/or do not or are not capable of internalizing. Exemplary truncated cell surface polypeptides including truncated forms of growth factors or other receptors such as a truncated human epidermal growth factor receptor 2 (tHER2), a truncated epidermal growth factor receptor (tEGFR, exemplary tEGFR sequence set forth in SEQ ID NO: 7 or 16) or a prostate-specific membrane antigen (PSMA) or modified form thereof. tEGFR may contain an epitope recognized by the antibody cetuximab (Erbitux®) or other therapeutic anti-EGFR antibody or binding molecule, which can be used to identify or select cells that have been engineered with the tEGFR construct and an encoded exogenous protein, and/or to eliminate or separate cells expressing the encoded exogenous protein. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, a CD19 or a truncated CD19, e.g., a truncated non-human CD19, or epidermal growth factor receptor (e.g., tEGFR). In some embodiments, the marker is or comprises a fluorescent protein, such as green fluorescent protein (GFP), enhanced green fluorescent protein (EGFP), such as super-fold GFP (sfGFP), red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), β-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

In some embodiments, the nucleic acid encoding the marker is operably linked to a polynucleotide encoding for a linker sequence, such as a cleavable linker sequence, e.g., a T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 7 or 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 7 or 16.

Any of the recombinant receptors described herein can be encoded by polynucleotides containing one or more nucleic acid sequences encoding a cell surface marker and/or recombinant receptors, in any combinations or arrangements. For example, one, two, three or more polynucleotides can encode one, two, three or more different polypeptides, e.g., a cell surface marker and/or recombinant receptors. In some embodiments, one vector or construct contains a nucleic acid sequence encoding a cell surface marker, and a separate vector or construct contains a nucleic acid sequence encoding a recombinant receptor, e.g., CAR. In some embodiments, the nucleic acid encoding the a cell surface marker and the nucleic acid encoding the recombinant receptor are operably linked to two different promoters. In some embodiments, the nucleic acid encoding the recombinant receptor is present downstream of the nucleic acid encoding the a cell surface marker.

b. Viral Vectors and Preparation of Viral Vectors

In some embodiments, the polynucleotide encoding the recombinant receptor is introduced into a composition containing cultured cells, such as by retroviral transduction, transfection, or transformation.

Also provided are vectors or constructs containing such nucleic acids and/or polynucleotides. In some embodiments, the vectors or constructs contain one or more promoters operatively linked to the nucleic acid encoding the recombinant receptor to drive expression thereof. In some embodiments, the promoter is operatively linked to one or more than one nucleic acid molecules or polynucleotides. Thus, also provided are vectors, such as those that contain any of the polynucleotides provided herein. In some embodiments, the vector includes a first polynucleotide encoding a cell surface marker and a second polynucleotide encoding a recombinant receptor, e.g., CAR.

In some cases, the vector is a viral vector, such as a retroviral vector, e.g., a lentiviral vector or a gammaretroviral vector. Also provided a set or combination of vectors. In some embodiments, the set or combination of vectors comprises a first vector and a second vector, wherein the first vector comprises the first polynucleotide, e.g., a first polynucleotide encoding a cell surface marker, and the second vector comprises the second polynucleotide encoding a recombinant receptor, e.g., CAR. Also provided are compositions containing such set or combination of vectors. In some embodiments, the set or combination of vectors, are used together for engineering of cells. In some embodiments, the first and the second vectors in the set are introduced simultaneously or sequentially, in any order into a cell for engineering.

In some embodiments, the vectors include viral vectors, e.g., retroviral or lentiviral, non-viral vectors or transposons, e.g. Sleeping Beauty transposon system, vectors derived from simian virus 40 (SV40), adenoviruses, adeno-associated virus (AAV), lentiviral vectors or retroviral vectors, such as gamma-retroviral vectors, retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV) or adeno-associated virus (AAV).

The viral vector genome is typically constructed in a plasmid form that can be transfected into a packaging or producer cell line. In any of such examples, the nucleic acid encoding a recombinant protein, such as a recombinant receptor, is inserted or located in a region of the viral vector, such as generally in a non-essential region of the viral genome. In some embodiments, the nucleic acid is inserted into the viral genome in the place of certain viral sequences to produce a virus that is replication defective.

Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g. vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g. antigen receptor, such as CAR, can be detected.

4. Cultivating and/or Expansion

In some embodiments, the provided methods include one or more steps for cultivating engineered cells, e.g., cultivating cells under conditions that promote proliferation and/or expansion. In some embodiments, engineered cells are cultivated under conditions that promote proliferation and/or expansion subsequent to a step of genetically engineering, e.g., introducing a recombinant polypeptide to the cells by transduction or transfection. In particular embodiments, the cells are cultivated after the cells have been incubated under stimulating conditions and transduced or transfected with a recombinant polynucleotide, e.g., a polynucleotide encoding a recombinant receptor. In some embodiments, the cultivation produces an output composition containing a composition of enriched T cells that express the recombinant receptor (e.g. CAR).

In some embodiments, the engineered cells are cultured in a container that can be filled, e.g. via the feed port, with cell media and/or cells for culturing of the added cells. The cells can be from any cell source for which culture of the cells is desired, for example, for expansion and/or proliferation of the cells.

In some aspects, the culture media is an adapted culture medium that supports that growth, cultivation, expansion or proliferation of the cells, such as T cells. In some aspects, the medium can be a liquid containing a mixture of salts, amino acids, vitamins, sugars or any combination thereof. In some embodiments, the culture media further contains one or more stimulating conditions or agents, such as to stimulate the cultivation, expansion or proliferation of cells during the incubation. In some embodiments, the stimulating condition is or includes one or more cytokine selected from IL-2, IL-7 or IL-15. In some embodiments, the cytokine is a recombinant cytokine. In some embodiments, the concentration of the one or more cytokine in the culture media during the culturing or incubation, independently, is from or from about 1 IU/mL to 1500 IU/mL, such as from or from about 1 IU/mL to 100 IU/mL, 2 IU/mL to 50 IU/mL, 5 IU/mL to 10 IU/mL, 10 IU/mL to 500 IU/mL, 50 IU/mL to 250 IU/mL or 100 IU/mL to 200 IU/mL, 50 IU/mL to 1500 IU/mL, 100 IU/mL to 1000 IU/mL or 200 IU/mL to 600 IU/mL. In some embodiments, the concentration of the one or more cytokine, independently, is at least or at least about 1 IU/mL, 5 IU/mL, 10 IU/mL, 50 IU/mL, 100 IU/mL, 200 IU/mL, 500 IU/mL, 1000 IU/mL or 1500 IU/mL.

In some aspects, the cells are incubated for at least a portion of time after transfer of the engineered cells and culture media. In some embodiments, the stimulating conditions generally include a temperature suitable for the growth of primary immune cells, such as human T lymphocytes, for example, at least about 25 degrees Celsius, generally at least about 30 degrees, and generally at or about 37 degrees Celsius. In some embodiments, the cells are incubated at a temperature of 25 to 38 degrees Celsius, such as 30 to 37 degrees Celsius, for example at or about 37 degrees Celsius±2 degrees Celsius. In some embodiments, the incubation is carried out for a time period until the culture, e.g. cultivation or expansion, results in a desired or threshold density, number or dose of cells. In some embodiments, the incubation is greater than or greater than about or is for about or 24 hours, 48 hours, 72 hours, 96 hours, 5 days, 6 days, 7 days, 8 days, 9 days or more.

In some embodiments, the cells are incubated under conditions to maintain a target amount of carbon dioxide in the cell culture. In some aspects, this ensures optimal cultivation, expansion and proliferation of the cells during the growth. In some aspects, the amount of carbon dioxide (CO₂) is between 10% and 0% (v/v) of said gas, such as between 8% and 2% (v/v) of said gas, for example an amount of or about 5% (v/v) CO₂.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, cells are incubated using containers, e.g., bags, which are used in connection with a bioreactor. In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is or is about 20°, 19°, 18°, 17°, 16°, 15°, 14°, 13°, 12°, 11°, 10°, 9°, 8°, 7°, 6°, 5°, 4°, 3+, 2° or 1°. In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 1 12, 13, 14 15, 16, 17, 18, 19, 20, 21, 22, 23, 24, 25, 26, 27, 28, 29, 30, 31, 32, 33, 34, 35, 36, 37, 38, 39, 40 rpm. In some embodiments, the rock rate is between 4 and 12 rpm, such as between 4 and 6 rpm, inclusive. At least a portion of the cell culture expansion is performed with a rocking motion, such as at an angle of between 5° and 10°, such as 6°, at a constant rocking speed, such as a speed of between 5 and 15 RPM, such as 6 RMP or 10 RPM. The CD4+ and CD8+ cells are each separately expanded until they each reach a threshold amount or cell density.

In some embodiments, at least a portion of the incubation is carried out under static conditions. In some embodiments, at least a portion of the incubation is carried out with perfusion, such as to perfuse out spent media and perfuse in fresh media during the culture. In some embodiments, the method includes a step of perfusing fresh culture medium into the cell culture, such as through a feed port. In some embodiments, the culture media added during perfusion contains the one or more stimulating agents, e.g. one or more recombinant cytokine, such as IL-2, IL-7 and/or IL-15. In some embodiments, the culture media added during perfusion is the same culture media used during a static incubation.

In some embodiments, subsequent to the incubation, the container, e.g., bag, is re-connected to a system for carrying out the one or more other processing steps of for manufacturing, generating or producing the cell therapy, such as is re-connected to the system containing the centrifugal chamber. In some aspects, cultured cells are transferred from the bag to the internal cavity of the chamber for formulation of the cultured cells.

C. Compositions and Formulations

In some cases, one or more steps (e.g. carried out in the centrifugal chamber and/or closed system) for manufacturing, generating or producing a cell therapy and/or engineered cells may include formulation of cells, such as formulation of genetically engineered cells resulting from the provided transduction processing steps prior to or after the culturing, e.g. cultivation and expansion, and/or one or more other processing steps as described. In some cases, the cells can be formulated in an amount for dosage administration, such as for a single unit dosage administration or multiple dosage administration. In some embodiments, the provided methods associated with formulation of cells include processing transduced cells, such as cells transduced and/or expanded using the processing steps described above, in a closed system.

In some embodiments, T cells, such as CD4+ and/or CD8+ T cells, generated by one or more of the processing steps are formulated. In some aspects, a plurality of compositions are separately manufactured, produced or generated, each containing a different population and/or sub-types of cells from the subject, such as for administration separately or independently, optionally within a certain period of time. For example, separate formulations of engineered cells containing different populations or sub-types of cells can include CD8+ and CD4+ T cells, respectively, and/or CD8+- and CD4+-enriched populations, respectively, e.g., CD4+ and/or CD8+ T cells each individually including cells genetically engineered to express the recombinant receptor. In some embodiments, at least one composition is formulated with CD4+ T cells genetically engineered to express the recombinant receptor. In some embodiments, at least one composition is formulated with CD8+ T cells genetically engineered to express the recombinant receptor.

In some embodiments, the cells produced using any of the methods of incubating (e.g., stimulating) described herein, such as cells genetically engineered with a recombinant receptor (e.g., CAR-T cells) are provided as compositions, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent. In some embodiments, a composition of cells is generated or manufactured for the purposes of a cell therapy. In some embodiments, the cell composition is a pharmaceutical composition or formulation. Such compositions can be used in accord with the provided methods, for example, for clonotype assessment and/or to assess clonal diversity. Such compositions can be used in accord with adoptive cell therapy methods, including methods for the prevention or treatment of diseases, conditions, and disorders, or in detection, diagnostic, and prognostic methods.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains a dose of the engineered cells in amounts effective to treat or prevent the disease or condition, such as a therapeutically effective or prophylactically effective amount.

In some embodiments, the administration of the dose comprises administration of a first composition comprising a dose of CD8+ T cells or a dose of CD4+ T cells and administration of a second composition comprising the other of the dose of CD4+ T cells and the CD8+ T cells. In some embodiments, a first composition comprising a dose of CD8+ T cells or a dose of CD4+ T cells is administered prior to the second composition comprising the other of the dose of CD4+ T cells and the CD8+ T cells. In some embodiments, the administration of the dose comprises administration of a composition comprising both of a dose of CD8+ T cells and a dose of CD4+ T cells.

The term “pharmaceutical formulation” refers to a preparation which is in such form as to permit the biological activity of an active ingredient contained therein to be effective, and which contains no additional components which are unacceptably toxic to a subject to which the formulation would be administered. In some embodiments, methods provided herein may be used to compare surface glycan expression of cell compositions composed of the same engineered cells, but with different pharmaceutical formulations.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In particular embodiments, methods provided herein may be used to compare surface glycan expression of cell compositions composed of the same engineered cells, but with different pharmaceutically acceptable carriers.

In some embodiments, the T cell therapy, such as engineered T cells (e.g., CAR T cells), are formulated with a pharmaceutically acceptable carrier. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being prevented or treated with the cells, including one or more active ingredients where the activities are complementary to the cells and/or the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

The pharmaceutical composition in some embodiments contains cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The cells may be formulated for administration using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

IV. Methods of Treatment and Uses

The engineered cells and compositions can be used as a cell-based therapy to treat or prevent diseases, conditions, and disorders, including cancers. In some embodiments, the cell-based therapy is or comprises administration of cells, such as immune cells, for example T cell, that target a molecule expressed on the surface of a lesion, such as a tumor or a cancer, such as via the engineered recombinant receptor (e.g. CAR). The methods and uses include methods and uses for adoptive cell therapy. In some embodiments, the methods include administration of the engineered cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition, ameliorate one or more symptom of the disease or condition, such as by lessening tumor burden in a cancer expressing an antigen recognized by an engineered T cell.

The disease or condition that is treated in some aspects can be any in which expression of an antigen is associated with, specific to, and/or expressed on a cell or tissue of a disease, disorder or condition and/or involved in the etiology of a disease, condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the immunomodulatory polypeptide and/or recombinant receptor, e.g., the chimeric antigen receptor or TCR, specifically binds to an antigen associated with the disease or condition. In some embodiments, the subject has a disease, disorder or condition, optionally a cancer, a tumor, an autoimmune disease, disorder or condition, or an infectious disease.

In some embodiments, the disease, disorder or condition includes tumors associated with various cancers. The cancer can in some embodiments be any cancer located in the body of a subject, such as, but not limited to, cancers located at the head and neck, breast, liver, colon, ovary, prostate, pancreas, brain, cervix, bone, skin, eye, bladder, stomach, esophagus, peritoneum, or lung. For example, the anti-cancer agent can be used for the treatment of colon cancer, cervical cancer, cancer of the central nervous system, breast cancer, bladder cancer, anal carcinoma, head and neck cancer, ovarian cancer, endometrial cancer, small cell lung cancer, non-small cell lung carcinoma, neuroendocrine cancer, soft tissue carcinoma, penile cancer, prostate cancer, pancreatic cancer, gastric cancer, gall bladder cancer or espohageal cancer. In some cases, the cancer can be a cancer of the blood. In some embodiments, the disease, disorder or condition is a tumor, such as a solid tumor, lymphoma, leukemia, blood tumor, metastatic tumor, or other cancer or tumor type. In some embodiments, the disease, disorder or condition is selected from among cancers of the colon, lung, liver, breast, prostate, ovarian, skin, melanoma, bone, brain cancer, ovarian cancer, epithelial cancers, renal cell carcinoma, pancreatic adenocarcinoma, cervical carcinoma, colorectal cancer, glioblastoma, neuroblastoma, Ewing sarcoma, medulloblastoma, osteosarcoma, synovial sarcoma, and/or mesothelioma.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease, disorder or condition is a tumor, cancer, malignancy, neoplasm, or other proliferative disease or disorder. Such diseases include but are not limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma,diffuse large B-cell lymphoma (DLBCL) and multiple myeloma (MM), a B cell malignancy is selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is selected from the group consisting of or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen or ligand is a tumor antigen or cancer marker. In some embodiments, the antigen or ligand the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

In some embodiments, the disease or condition is a B cell malignancy. In some embodiments, the B cell malignancy is a leukemia or a lymphoma. In some aspects, the disease or condition is acute lymphoblastic leukemia (ALL), adult ALL, chronic lymphoblastic leukemia (CLL), non-Hodgkin lymphoma (NHL), or Diffuse Large B-Cell Lymphoma (DLBCL). In some cases, the disease or condition is an NHL, such as or including an NHL that is an aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B). In some aspects, the recombinant receptor, such as a CAR, specifically binds to an antigen associated with the disease or condition or expressed in cells of the environment of a lesion associated with the B cell malignancy. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the disease or condition is a myeloma, such as a multiple myeloma. In some aspects, the recombinant receptor, such as a CAR, specifically binds to an antigen associated with the disease or condition or expressed in cells of the environment of a lesion associated with the multiple myeloma. Antigens targeted by the receptors in some embodiments include antigens associated with multiple myeloma, such as GPRC5D or BCMA.

In some embodiments, the antigen is a pathogen-specific or pathogen-expressed antigen. In some embodiments, the antigen is a viral antigen (such as a viral antigen from HIV, HCV, HBV, etc.), bacterial antigens, and/or parasitic antigens.

In some embodiments, the immune cells express a T cell receptor (TCR) or other antigen-binding receptor. In some embodiments, the immune cells express a recombinant receptor, such as a transgenic TCR or a chimeric antigen receptor (CAR). In some embodiments, the cells are autologous to the subject. In some embodiments, the cells are allogeneic to the subject.

Methods for administration of engineered cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al., (2013) Nat Biotechnol. 31(10): 928-933; Tsukahara et al., (2013) Biochem Biophys Res Commun 438(1): 84-9; Davila et al., (2013) PLoS ONE 8(4): e61338.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 5×10⁸ total recombinant receptor (e.g., CAR)-expressing cells, T cells, or peripheral blood mononuclear cells (PBMCs). In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the dose of genetically engineered cells comprises from or from about 1×10⁵ to 5×10⁸ total CAR-expressing T cells, 1×10⁵ to 2.5×10⁸ total CAR-expressing T cells, 1×10⁵ to 1×10⁸ total CAR-expressing T cells, 1×10⁵ to 5×10⁷ total CAR-expressing T cells, 1×10⁵ to 2.5×10⁷ total CAR-expressing T cells, 1×10⁵ to 1×10⁷ total CAR-expressing T cells, 1×10⁵ to 5×10⁶ total CAR-expressing T cells, 1×10⁵ to 2.5×10⁶ total CAR-expressing T cells, 1×10⁵ to 1×10⁶ total CAR-expressing T cells, 1×10⁶ to 5×10⁸ total CAR-expressing T cells, 1×10⁶ to 2.5×10⁸ total CAR-expressing T cells, 1×10⁶ to 1×10⁸ total CAR-expressing T cells, 1×10⁶ to 5×10⁷ total CAR-expressing T cells, 1×10⁶ to 2.5×10⁷ total CAR-expressing T cells, 1×10⁶ to 1×10⁷ total CAR-expressing T cells, 1×10⁶ to 5×10⁶ total CAR-expressing T cells, 1×10⁶ to 2.5×10⁶ total CAR-expressing T cells, 2.5×10⁶ to 5×10⁸ total CAR-expressing T cells, 2.5×10⁶ to 2.5×10⁸ total CAR-expressing T cells, 2.5×10⁶ to 1×10⁸ total CAR-expressing T cells, 2.5×10⁶ to 5×10⁷ total CAR-expressing T cells, 2.5×10⁶ to 2.5×10⁷ total CAR-expressing T cells, 2.5×10⁶ to 1×10⁷ total CAR-expressing T cells, 2.5×10⁶ to 5×10⁶ total CAR-expressing T cells, 5×10⁶ to 5×10⁸ total CAR-expressing T cells, 5×10⁶ to 2.5×10⁸ total CAR-expressing T cells, 5×10⁶ to 1×10⁸ total CAR-expressing T cells, 5×10⁶ to 5×10⁷ total CAR-expressing T cells, 5×10⁶ to 2.5×10⁷ total CAR-expressing T cells, 5×10⁶ to 1×10⁷ total CAR-expressing T cells, 1×10⁷ to 5×10⁸ total CAR-expressing T cells, 1×10⁷ to 2.5×10⁸ total CAR-expressing T cells, 1×10⁷ to 1×10⁸ total CAR-expressing T cells, 1×10⁷ to 5×10⁷ total CAR-expressing T cells, 1×10⁷ to 2.5×10⁷ total CAR-expressing T cells, 2.5×10⁷ to 5×10⁸ total CAR-expressing T cells, 2.5×10⁷ to 2.5×10⁸ total CAR-expressing T cells, 2.5×10⁷ to 1×10⁸ total CAR-expressing T cells, 2.5×10⁷ to 5×10⁷ total CAR-expressing T cells, 5×10⁷ to 5×10⁸ total CAR-expressing T cells, 5×10⁷ to 2.5×10⁸ total CAR-expressing T cells, 5×10⁷ to 1×10⁸ total CAR-expressing T cells, 1×10⁸ to 5×10⁸ total CAR-expressing T cells, 1×10⁸ to 2.5×10⁸ total CAR-expressing T cells, or 2.5×10⁸ to 5×10⁸ total CAR-expressing T cells.

In some embodiments, the dose of genetically engineered cells comprises at least or at least about 1×10⁵ CAR-expressing cells, at least or at least about 2.5×10⁵ CAR-expressing cells, at least or at least about 5×10⁵ CAR-expressing cells, at least or at least about 1×10⁶ CAR-expressing cells, at least or at least about 2.5×10⁶ CAR-expressing cells, at least or at least about 5×10⁶ CAR-expressing cells, at least or at least about 1×10⁷ CAR-expressing cells, at least or at least about 2.5×10⁷ CAR-expressing cells, at least or at least about 5×10⁷ CAR-expressing cells, at least or at least about 1×10⁸ CAR-expressing cells, at least or at least about 2.5×10⁸ CAR-expressing cells, or at least or at least about 5×10⁸ CAR-expressing cells.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷, 1×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the cells are administered as part of a further combination treatment, such as simultaneously with or sequentially with, in any order, another therapeutic intervention, such as an antibody or engineered cell or receptor or agent, such as a cytotoxic or therapeutic agent. For example, in some embodiments, an anti-cancer agent or immunomodulatory agent can be used in combination therapy with adoptive cell therapy with engineered cell expressing a recombinant receptor, e.g. a CAR. In some contexts, the cells are co-administered with another therapy sufficiently close in time such that the cell populations enhance the effect of the P one or more additional therapeutic agents, or vice versa. In some embodiments, the cells are administered prior to the one or more additional therapeutic agents. In some embodiments, the cells are administered after the one or more additional therapeutic agents.

In some embodiments, the one or more additional therapeutic agents include a cytokine, such as IL-2, for example, to enhance persistence. In some embodiments, the methods comprise administration of a chemotherapeutic agent. In some embodiments, the one or more additional therapeutic agents include one or more lymphodepleting therapies, such as prior to or simultaneous with initiation of administration of the engineered cells. In some embodiments, the lymphodepleting therapy comprises administration of a phosphamide, such as cyclophosphamide. In some embodiments, the lymphodepleting therapy can include administration of fludarabine. In some embodiments, fludarabine is excluded in the lymphodepleting therapy. In some embodiments, a lymphodepleting therapy is not administered.

In some embodiments, the methods include administering a preconditioning agent, such as a lymphodepleting or chemotherapeutic agent, such as cyclophosphamide, fludarabine, or combinations thereof, to a subject prior to the initiation of the cell therapy. For example, the subject may be administered a preconditioning agent at least 2 days prior, such as at least 3, 4, 5, 6, or 7 days prior, to the initiation of the cell therapy. In some embodiments, the subject is administered a preconditioning agent no more than 7 days prior, such as no more than 6, 5, 4, 3, or 2 days prior, to the initiation of the cell therapy.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 40 mg/m², or 24 mg/m² and 35 mg/m², inclusive. In some instances, the subject is administered about 30 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the first or subsequent dose.

The cells can be administered by any suitable means. The cells are administered in a dosing regimen to achieve a therapeutic effect, such as a reduction in tumor burden. Dosing and administration may depend in part on the schedule of administration of the immunomodulatory compound, which can be administered prior to, subsequent to and/or simultaneously with initiation of administration of the T cell therapy. Various dosing schedules of the T cell therapy include but are not limited to single or multiple administrations over various time-points, bolus administration, and pulse infusion.

V. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, a “subject” is a mammal, such as a human or other animal, and typically is human. In some embodiments, the subject, e.g., patient, to whom the immunomodulatory polypeptides, engineered cells, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or engineered cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the immunomodulatory polypeptides or engineered cells administered. In some embodiments, the provided methods involve administering the immunomodulatory polypeptides, engineered cells, or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, one can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A.M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”. In certain embodiments, “about” a stated value refers to a value within ±25%, ±20%, ±10%, ±5%, ±1%, ±0.1%, or ±0.01% of the stated value.

As used herein, a composition refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

Several aspects are described below with reference to example applications for illustration. It should be understood that numerous specific details, relationships, and methods are set forth to provide a full understanding of the features described herein. In some cases, the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

### VI. EXEMPLARY EMBODIMENTS

Among the provided embodiments are:
1. A method for identifying a property or attribute of a cell, the method comprising:
(a) identifying the clonotype and/or a TCR sequence of all of a portion of a native TCR alpha and/or beta variable region or pair thereof, of at least one T cell from at least one test biological sample from a subject, said test biological sample obtained from the subject following administration of a cell therapy comprising T cells expressing a recombinant receptor, wherein the T cell in the test biological sample is genetically engineered with and/or expresses the recombinant receptor;
(b) identifying, from a T cell composition, a cell that has the same clonotype or the same TCR sequence as the at least one T cell identified in (a), thereby identifying an originator T cell, wherein the T cell composition comprises T cells that are, or have been derived from, T cells previously obtained from the subject prior to administering the cell therapy to the subject; and
(c) determining at least one property or attribute of the originator T cell.
2. A method for identifying a property or attribute of a cell, the method comprising:
(a) identifying the clonotype and/or a TCR sequence of all of a portion of a native TCR alpha and/or beta variable region or pair thereof, of at least one T cell from at least one test biological sample from a subject, said test biological sample obtained from the subject following administration of a cell therapy comprising T cells expressing a recombinant receptor, wherein the T cell in the test biological sample is genetically engineered with and/or expresses the recombinant receptor;
(b) determining at least one property or attribute of a cell, from a T cell composition, that has the same clonotype or the same TCR sequence as the at least one T cell identified in (a), wherein the T cell composition comprises T cells that are, or have been derived from, T cells previously obtained from the subject prior to administering the cell therapy to the subject.
3. The method of embodiment 1, wherein the genetically engineered T cell in the test biological sample exhibits a predetermined phenotype, function or parameter.
4. The method of any of embodiments 1-3, wherein the predetermined phenotype, function or attribute is an effector function associated with T cell activation state, is a cell surface phenotype or is a pharmacokinetic activity.
5. The method of embodiment 4, wherein the predetermined phenotype, function or attribute is a pharmacokinetic activity and the pharmacokinetic activity comprises determining the number or relative number of recombinant receptor-expressing T cells in the sample.
6. The method of embodiment 4, wherein the predetermined phenotype, function or attribute is a cell surface phenotype and the cell surface phenotype is a naive phenotype or a long-lived memory phenotype.
7. A method for identifying a property or attribute of a cell, the method comprising:
(a) identifying the clonotype and/or a TCR sequence of all or a portion of a native TCR alpha and/or beta variable region or pair thereof of one or more T cell genetically engineered with a recombinant receptor in at least one test biological sample from a subject, wherein said clonotype is known to be, determined to be, or suspected of being present in a cell in a T cell composition, thereby identifying one or more originator T cell, wherein:
   the at least one test biological sample is obtained from the subject following administration of a cell therapy comprising T cells expressing the recombinant receptor; and
   the T cell composition comprises T cells that are or are derived from cells of a sample obtained from the subject prior to administering the cell therapy to the subject; and
(b) determining at least one or property or attribute of the one or more originator T cell.
8. The method of embodiment 7, wherein the one or more clonotype and/or TCR sequence that is identified is present in the test biological sample at the same or increased frequency or relative frequency as in the T cell composition.
9. A method for identifying a property or attribute of a cell, the method comprising:
(a) identifying one or more clonotypes and/or one or more TCR sequences of all or a portion of a native TCR alpha and/or beta variable region or pair thereof that are the same in a plurality of samples at different stages of a cell engineering process for generating a T cell therapy and/or following administration of the T cell therapy to a subject, said T cell therapy comprising T cells expressing the recombinant receptor, thereby identifying an originator T cell; and
(b) determining at least one property or attribute of the originator T cell.
10. The method of embodiment 9, wherein at least one of the plurality of samples is a T cell composition at a stage of a cell engineering process, said T cell composition comprising T cells that are, or have been derived from, T cells previously obtained from the subject prior to administering the cell therapy to the subject.
11. The method of embodiment 9 or embodiment 10, wherein at least one of the plurality of samples is test biological sample, said test biological sample obtained from the subject following administration of a cell therapy comprising T cells expressing a recombinant receptor.
12. The method of any of embodiments 1-11 that is repeated for a plurality of subjects.
13. The method of embodiment 12, further comprising identifying the at least one property or parameter of originator T cells that is present in a T cell composition from a majority of subjects.
14. The method of any of embodiments 1-13, wherein the at least one property or parameter is identified as an attribute of a T cell composition that is predicted to increase likelihood or a desired property or outcome of a cell therapy following administration to a subject.
15. The method of any of embodiments 1-14, wherein the T cell composition is an input composition that does not comprise T cells genetically engineered with the recombinant receptor.
16. The method of embodiment 15, wherein the input composition is obtained by isolating a population of cells comprising the T cells from a biological sample.
17. The method of any of embodiments 1-16, wherein the T cell composition is an output composition comprising T cells genetically engineered with the recombinant receptor.
18. The method of embodiment 17, wherein the output composition is the cell therapy administered to the subject in (a).
19. The method of embodiment 17 or embodiment 18, wherein the output composition is produced by a process comprising:
(i) incubating an input composition comprising T cells with an agent comprising a nucleic acid molecule encoding the recombinant receptor under conditions to introduce the nucleic acid encoding the recombinant receptor into cells in the population; and
(ii) stimulating the cells, prior to, during and/or subsequent to said incubation, wherein stimulating comprises incubating the cells in the presence of a stimulating condition that induces a primary signal, signaling, stimulation, activation and/or expansion of the cells.
20. The method of embodiment 19, wherein the process further comprises, prior to (i), isolating the population of cells from a biological sample.
21. The method of embodiment 16 or embodiment 20, wherein the isolating comprises, selecting cells based on surface expression of CD3 or based on surface expression of one or both of CD4 and CD8, optionally by positive or negative selection.
22. The method of embodiment 16, 20 or 21, wherein the isolating comprises carrying out immunoaffinity-based selection.
23. The method of any of embodiments 16 and 20-22, wherein the biological sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product.
24. The method of any of embodiments 19-23, wherein the stimulating condition comprises incubation with a stimulatory reagent capable of activating one or more intracellular signaling domains of one or more components of a TCR complex and/or one or more intracellular signaling domains of one or more costimulatory molecules.
25. The method of embodiment 24, wherein the stimulatory reagent comprises a primary agent that specifically binds to a member of a TCR complex and a secondary agent that specifically binds to a T cell costimulatory molecule.
26. The method of embodiment 24 or embodiment 25, wherein the primary agent specifically binds to CD3 and/or the costimulatory molecule is selected from the group consisting of CD28, CD137 (4-1-BB), OX40, or ICOS.
27. The method of embodiment 25 or embodiment 26, wherein the primary and secondary agents comprise antibodies and/or are present on the surface of a solid support, optionally a bead.
28. The method of any of embodiments 19-27, wherein the stimulating the cells is carried out or is initiated prior to the incubating, optionally for 18-24 hours at or about 37 deg.
29. The method of any of embodiments 19-28, wherein the stimulating condition comprises a cytokine selected from among IL-2, IL-15 and IL-7.
30. The method of any of embodiments 19-29, wherein the stimulating cells is carried out subsequent to the incubating, optionally for a period of time to achieve a threshold concentration.
31. The method of any of embodiments 19-30, wherein the agent comprising a nucleic acid molecule encoding the recombinant receptor is a viral vector, optionally a lentiviral vector or a gamma retroviral vector.
32. The method of any of embodiments 19-31, wherein:
the incubating and/or stimulating is carried out in the presence of one or more test agents or conditions; or
the process further comprises culturing the input composition and/or stimulated cells in the presence of one or more test agents or conditions.
33. The method of embodiment 32, wherein the one or more test agents or conditions comprises presence or concentration of serum; time in culture; presence or amount of a stimulating agent; the type or extent of a stimulating agent; presence or amount of amino acids; temperature; the source or cell types of the input composition; the ratio or percentage of cell types in the input composition, optionally the CD4+/CD8+ cell ratio; the presence or amount of beads; cell density; static culture; rocking culture; perfusion; the type of viral vector; the vector copy number; the presence of a transduction adjuvant; cell density of the input composition in cryopreservation; the extent of expression of the recombinant receptor; or the presence of a compound to modulate cell phenotype.
34. The method of embodiment 32 or embodiment 33, wherein the one or more test agents or conditions comprises one or more compounds from a library of test compounds.
35. The method of any of embodiments 1-34, wherein the test biological sample is a serum, blood or plasma sample.
36. The method of any of embodiments 1-35, wherein the test biological sample is or comprises a tumor sample.
37. The method of any of embodiments 1-36, wherein the test biological sample is obtained from the subject greater than or greater than about 7 days, 10 days, 14 days, 21 days, 28 days, 2 months, 3 months, 4 months, 5 months, 6 months, 1 year or 2 years or more after initiation of administration of the cell therapy.
38. The method of any of embodiments 1-37, wherein the test biological sample is obtained from the subject greater than or greater than about 28 days after initiation of administration of the cell therapy, optionally at or about at day 29 or greater after initiation of administration of the cell therapy.
39. The method of any of embodiments 1-38, wherein the at least one test biological sample comprises a plurality of test biological samples, optionally at least 2, 3, 4, 5, 6, 7, 8, 9, 10 or more test biological samples.
40. The method of embodiment 39, wherein each of the plurality of test biological samples is obtained from the subject on different days after initiation of administration of the cell therapy, optionally on consecutive days, every other day, every third day, or once a week for a predetermined time after initiation of administration of the cell therapy.
41. The method of embodiment 39 or embodiment 40, wherein the identified clonotype is one whose frequency or relative frequency is retained or increased among the plurality of assessed tested biological samples over the predetermined period of time.
42. The method of any of embodiments 1-41, wherein the clonotype in a) and/or b) is determined by high-throughput single cell immune sequencing of nucleic acid encoding natively paired TCR chains.
43. The method of embodiment 42, wherein the natively paired TCR chains comprise TCR α-β or TCR γ-δ pairs.
44. The method of any of embodiments 1-43, wherein the test biological sample comprises a plurality of T cells and the one or more clonotype is identified simultaneously or from a single reaction.
45. The method of any of embodiments 1-44, wherein the T cell composition comprises a plurality of T cells and the one or more clonotype is identified from a single reaction.
46. The method of any of embodiments 1-45, wherein the at least one property or parameter is determined by single cell gene expression profiling and/or single cell surface phenotyping.
47. The method of embodiment 46, wherein the at least one property or parameter is determined by single cell gene expression profiling, wherein the single cell gene expression profiling is of at least one gene product or is of the whole-transcriptome or a portion thereof.
48. The method of embodiment 47, wherein the at least one gene product is selected from CD4, ICOS, FOXP3, FOXP3V1, PMCH, CD80, FOXP3Y, CD86, CD70, CD40, IL-6, CD2, CD3D, GPR171, CXCL13, PD-1 (CD279), IL-2, IL-4, IL-10, CD8B, KLRK1, CCL4, RUNX3V1, RUNX3, NKG7, CD45RA, CD45RO, CD62L, CD69, CD25, CCR7, CD27, CD28, CD56, CD122, CD127, CD95, CXCR3, LFA-1, KLRG1, T-bet, CD8, IL-7Rα, IL-2Rβ, CD3, CD14, ROR1, granzyme B, granzyme H, CD20, CD11b, CD16, HLA-DR, PD-L1, IFNγ, KIRK1, caspase 2, caspase 3, caspase 6, caspase 7, caspase 8, caspase 9, caspase 10, Bcl-2, Bax, Bad, Bid, CD196 (CCR6), CTLA-4 (CD152), TIGIT (VSIG9, VSTM3), LAG-3 (CD223), 2B4 (CD244), BTLA (CD272), TIM3 (HAVCR2), VISTA (PD1-H) and CD96.
49. The method of embodiment 48, wherein the at least one property or parameter is determined by single cell surface phenotyping of at least one T cell surface marker.
50. The method of embodiment 49, wherein the at least one T cell surface marker is selected from CD4, CD8, CD45RA, CD45RO, CD62L, CD69, CD25, CCR7, CD27, CD28, CD56, CD122, CD127, T-bet, IL-7Ra, CD95, CXCR3, LFA-1 or KLRG1.
51. The method of any of embodiments 42-50, wherein the single cell gene expression profiling or single cell surface phenotyping is coupled to or carried out in the same reaction as the single cell immune sequencing.
52. The method of any of embodiments 1-51, wherein the recombinant receptor is or comprises a chimeric receptor.
53. The method of any of embodiments 1-52, wherein the chimeric receptor is capable of binding to a target antigen that is associated with, specific to, and/or expressed on a cell or tissue of a disease, disorder or condition.
54. The method of embodiment 53, wherein the disease, disorder or condition is an infectious disease or disorder, an autoimmune disease, an inflammatory disease, or a tumor or a cancer.
55. The method of embodiment 53 or embodiment 54, wherein the target antigen is a tumor antigen.
56. The method of any of embodiments 53-55, wherein the target antigen is selected from among ROR1, B cell maturation antigen (BCMA), carbonic anhydrase 9 (CAIX), Her2/neu (receptor tyrosine kinase erbB2), CD19, CD20, CD22, and hepatitis B surface antigen, anti-folate receptor, CD23, CD24, CD30, CD33, CD38, CD44, chondroitin sulfate proteoglycan 4 (CSPG4), EGFR, epithelial glycoprotein 2 (EPG-2), epithelial glycoprotein 40 (EPG-40), ephrin receptor A2 (EPHa2), Her2/neu (receptor tyrosine kinase erb-B2), Her3 (erb-B3), Her4 (erb-B4), erbB dimers, EGFR vIII, folate binding protein (FBP), Fc receptor like 5 (FCRL5, also known as Fc receptor homolog 5 or FCRH5), fetal acetylcholine receptor (fetal AchR), ganglioside GD3, Human high molecular weight-melanoma-associated antigen (HMW-MAA), IL-22 receptor alpha(IL-22R-alpha), IL-13 receptor alpha 2 (IL-13R-alpha2), kinase insert domain receptor (kdr), kappa light chain, Leucine Rich Repeat Containing 8 Family Member A (LRRC8A), Lewis Y, L1-cell adhesion molecule, (L1-CAM), Melanoma-associated antigen (MAGE)-A1, MAGE-A3, MAGE-A6, MAGE-A10, Preferentially expressed antigen of melanoma (PRAME), survivin, TAG72, B7-H3, B7-H6, IL-13 receptor alpha 2 (IL-13Ra2), carbonic anhydrase 9 (CA9, also known as CAIX or G250), CD171, Human leukocyte antigen A1 (HLA-AI), MAGE A1, Human leukocyte antigen A2 (HLA-A2), cancer/testis antigen 1B (CTAG, also known as NY-ESO-1), folate receptor-alpha, CD44v6, CD44v7/8, αvβ6 integrin (avb6 integrin), 8H9, neural cell adhesion molecule (NCAM), vascular endothelial growth factor receptor (VEGF receptors), Trophoblast glycoprotein (TPBG also known as 5T4), NKG2D ligands, CD44v6, dual antigen, a cancer-testes antigen, mesothelin (MSLN), murine cytomegalovirus (CMV), mucin 1 (MUC1), MUC16, a prostate specific antigen, prostate stem cell antigen (PSCA), prostate specific membrane antigen (PSMA), natural killer group 2 member D (NKG2D) ligands, NY-ESO-1, melan A (MART-1), glycoprotein 100 (gp100), glypican-3 (GPC3), G Protein Coupled Receptor 5D (GPRCSD), oncofetal antigen, Receptor Tyrosine Kinase Like Orphan Receptor 1 (ROR1), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor 2 (VEGF-R2), carcinoembryonic antigen (CEA), Her2/neu, estrogen receptor, progesterone receptor, ephrinB2, CD123, CD133, c-Met, ganglioside GD-2, O-acetylated GD2 (OGD2), CE7 epitope of L1-CAM, Wilms Tumor 1 (WT-1), a cyclin, cyclin A2, C-C Motif Chemokine Ligand 1 (CCL-1), CD138, a pathogen-specific antigen and an antigen associated with a universal tag and/or biotinylated molecules, and/or molecules expressed by HIV, HCV, HBV or other pathogens.
57. The method of any of embodiments 52-57, wherein chimeric receptor is a chimeric antigen receptor (CAR).
58. The method of any of embodiments 52-57, wherein chimeric receptor comprises an extracellular domain comprising an antigen-binding domain.
59. The method of embodiment 58, wherein the antigen-binding domain is or comprises an antibody or an antibody fragment thereof, which optionally is a single chain fragment.
60. The method of embodiment 59, wherein the fragment comprises antibody variable regions joined by a flexible linker.
61. The method of embodiment 59 or embodiment 60, wherein the fragment comprises an scFv.
62. The method of any of embodiments 52-618, wherein the chimeric receptor further comprises a spacer and/or a hinge region.
63. The method of any of embodiments 52-62, wherein chimeric receptor comprises an intracellular signaling region.
64. The method of embodiment 63, wherein the intracellular signaling region comprises an intracellular signaling domain.
65. The method of embodiment 64, wherein the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).
66. The method of embodiment 65, wherein the intracellular signaling domain is or comprises an intracellular signaling domain of a CD3 chain, optionally a CD3-zeta (CD3) chain, or a signaling portion thereof.
67. The method of any of embodiments 63-66, wherein chimeric receptor further comprises a transmembrane domain disposed between the extracellular domain and the intracellular signaling region.
68. The method of any of embodiments 63-67, wherein the intracellular signaling region further comprises a costimulatory signaling region.
69. The method of embodiment 68, wherein the costimulatory signaling region comprises an intracellular signaling domain of a T cell costimulatory molecule or a signaling portion thereof.
70. The method of embodiment 68 or embodiment 69, wherein the costimulatory signaling region comprises an intracellular signaling domain of a CD28, a 4-1BB or an ICOS or a signaling portion thereof.
71. The method of any of embodiments 68-70, wherein the costimulatory signaling region is between the transmembrane domain and the intracellular signaling region.
72. The method of any of embodiments 1-71, wherein the T cell composition and/or cell therapy comprises CD4 and/or CD8 T cells.
73. The method of any of embodiments 1-71, wherein the clonotype comprises the TCR sequences of all or a portion of a native TCR alpha and/or beta variable region or pair thereof.
74. The method of any of embodiments 1-73, wherein the clonotype and/or TCR sequence is of a T cell genetically engineered with or expressing the recombinant receptor.
75. The method of any of embodiments 1-74, wherein the clonotype and/or TCR sequence is of a CD8+ T cell.

### VII. EXAMPLES

The following examples are included for illustrative purposes only and are not intended to limit the scope of the invention.

### Example 1—Assessment of Abundance and Phenotype of T Cell Clones Before and After Production of Therapeutic Engineered T Cell Compositions Generated From T Cells Derived from Human Subjects

Compositions of T cells genetically engineered to express a chimeric antigen receptor were assessed for phenotype and T cell clonal abundance, using flow cytometry and sequencing of a portion of the T cell receptor (TCR) transcript, before and after genetic engineering to express a chimeric antigen receptor (CAR).

T cells were isolated by immunoaffinity-based enrichment from leukapheresis of human peripheral blood mononuclear cells (PBMC) from a plurality of subjects. A composition of such isolated T cells (before genetic engineering, designated “T cell composition from subject”) were sorted by flow cytometry based on expression of CD45RA, CCR7, CD27, CD4 and CD8, markers indicative of different subtypes in the isolated T cell composition (CD45RA⁺/CCR7⁺ cells were designated as having phenotype typical of naive T cells (T_{N}); CD45RA7⁻/CCR7⁻ cells were designated as having phenotype typical of central memory T cells (T_{CM}); CD45RA⁻/CCR7⁺ cells were designated as having phenotype typical of effector memory T cells (T_{EM}); and CD45RA⁺/CCR7⁻ cells were designated as having phenotype typical of effector T cells (T_{E})). Sorted populations of cells were subjected to barcoded TCR sequencing, generally as described in WO2016044227, WO2016176322 and WO2012048340. In some cases, single-cell αβ-paired TCR sequencing can be used to determine TCR clonotypes present in a given population, TCR repertoire, T cell clonality and diversity, and the relative abundance of the identified clones in a cell population, based on barcoded single-cell sequencing of TCR genes (see, e.g., WO2016044227, WO2016176322 and WO2012048340).

A subset of the isolated T cell composition from the subject was subject to genetic engineering to express a chimeric antigen receptor (CAR). To generate a CAR+ T cell composition, the isolated T cells were activated by incubation with anti-CD3 and anti-CD28 antibody-coated beads in the presence of cytokines, and then were transduced with a lentiviral vector encoding an anti-CD19 CAR. The CAR contained an scFv antigen-binding domain specific for CD19, a spacer, a CD28 transmembrane region, a 4-1BB costimulatory signaling region, and a CD3-zeta derived intracellular signaling domain. After transduction, cells were expanded and then frozen by cryopreservation. The cells in the frozen composition (after genetic engineering, designated “engineered cells”) were assessed by flow cytometry for surface expression of CD45RA, CCR7, CD4 and CD8 and were subject to barcoded TCR sequencing. FIG. 2A depicts a general schematic representation of the experimental design.

FIG. 2B shows, for CD8+ TCR clones determined by barcoded TCR sequencing to be present among cells in the “T cell composition from subject” (before engineering) and among the “engineered cells” (after engineering), the relative percentages of different T cell subtypes, based on flow cytometry sorting based on cell surface staining of CD45RA and CCR7. As shown, among the CD8+ clones that were detected both before and after genetic engineering, the proportion exhibiting the naive phenotype increased and the proportion exhibiting the effector memory phenotype decreased after engineering. FIG. 2C shows a trace diagram indicating changes in CD27/CCR7 based phenotype of individual CD8+ clones determined to be present in both populations, as assessed by barcoded TCR sequencing. The results are consistent with the utility of such an approach in tracking phenotypic changes and attributes of a plurality of individual T cell clones at different stages of a cell engineering process, including patient material and drug product, for example, to assess and/or identify attributes in starting material cells that may increase the likelihood of a desired property or outcome of a therapeutic cell product.

### Example 2—Determination of T Cell Clonotypes and Clonal Abundance at Various Stages of Adoptive Cell Therapy

Clonotype repertoire of T cells was determined at different stages of engineering and adoptive cell therapy.

CD4+ and CD8+ T cells obtained from human subjects who had relapsed/refractory Diffuse large B-cell lymphoma (DLBCL) were engineered to express an anti-CD19 CAR, generally as described in Example 1 above. Subjects were intravenously administered autologous T cells expressing an anti-CD19 chimeric antigen receptor (CAR). On days 15, 22 and 29 after administration of the CAR-expressing cells, peripheral blood mononuclear cells (PBMC) samples were obtained from the subject. Samples derived from individual T cell compositions before and after engineering, and the CAR+ cells in blood samples from subjects obtained at indicated days following initiation of administration of the therapeutic cell composition, were individually subject to flow cytometry-based sorting and barcoded TCR sequencing, generally as described above in Example 1.

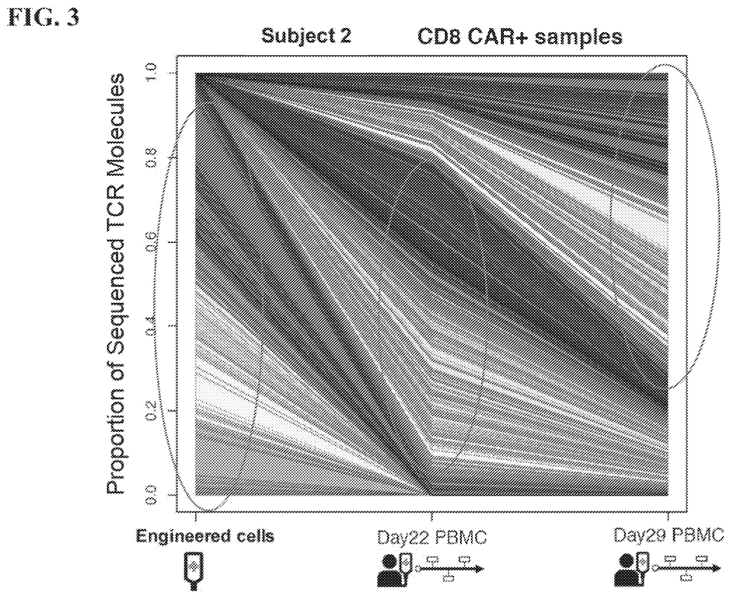

The changes in T cell clonotype repertoire and the relative abundance of identified clones in an exemplary subject (for clonotypes that were detected in 10 or more sequenced TCR molecules in each sample, and that were detected in each of the indicated compositions) for Subject 2, who was observed to exhibit a complete response (CR) following administration, is depicted in FIG. 3.

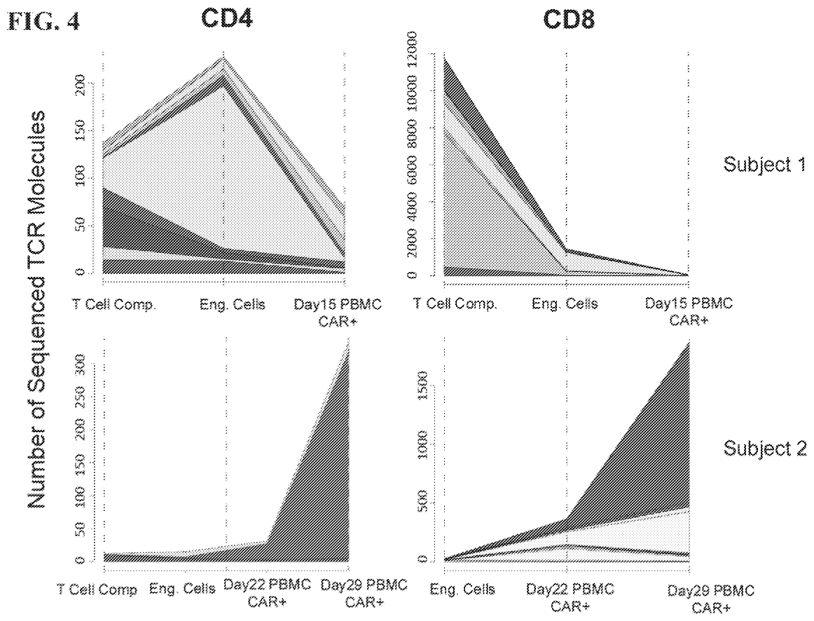

FIG. 4 shows clonal abundance of TCR clones detected at a threshold level across samples in CD4+ and CD8+ cell compositions and post-administration samples obtained at different stages, in 2 exemplary subjects. The results showed that in Subject 1, who exhibited a partial response (PR) following administration, clonal expansion was observed over the course of the engineering process, followed by a decrease in abundance in patient blood, at day 15 post-administration. In Subject 2, who was observed to exhibit a complete response (CR) following administration, clonal expansion was observed in the subject's blood after administration, with certain clones observed as expanded or continuing to expand at day 29, after peak levels of CAR-T cells generally were observed in patient blood.

### Example 3—Analysis of Phenotypes to Determine Molecular Signature of Clones Exhibiting a Predetermined Attribute

The clonotypes of T cells having a predetermined parameter or attribute, such as high abundance or high expansion in the subject's body after administration, are assessed and evaluated in combination with results from phenotype and molecular signatures analysis at various stages, including in the T cell composition obtained from the subject prior to engineering or administration, and/or cells from a test biological sample obtained from subjects who had been administered engineered cells, on various time points.

For example, phenotypes and molecular signatures of cells in the T cell compositions and samples obtained from the subject are analyzed using population-level and single-cell analysis of phenotypes. In some cases, single-cell gene expression analysis, genome-wide RNA expression profiles and/or single cell surface expression analysis can be coupled with single cell TCR sequencing. In some cases, particular clonotypes that are highly abundant in the PBMC samples obtained from the subject after administration are identified, and phenotypes and molecular signatures of the particular clone in the T cell composition obtained from the subject prior to engineering or administration (e.g., “originator T cell population”) is determined.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | ESKYGPPCPPCP | spacer (IgG4hinge) |
| | | (aa) |
| | | Homo sapiens |
| 2 | GAATCTAAGTACGGACCGCCCTGCCCCCCTTGCCCT | spacer (IgG4hinge) |
| | | (nt) |
| | | Homo sapiens |
| 3 | ESKYGPPCPPCPGQPREPQVYTLPPSQEEMTKNQVSLTCLVKG | Hinge-CH3 spacer |
| | FYPSDIAVEWESNGQPENNYKTTPPVLDSDGSFFLYSRLTVDK | Homo sapiens |
| | SRWQEGNVFSCSVMHEALHNHYTQKSLSLSLGK | |
| 4 | ESKYGPPCPPCPAPEFLGGPSVFLFPPKPKDTLMISRTPEVTC | Hinge-CH2-CH3 |
| | VVVDVSQEDPEVQFNWYVDGVEVHNAKTKPREEQFNSTYRVVS | spacer Homo sapiens |
| | VLTVLHQDWLNGKEYKCKVSNKGLPSSIEKTISKAKGQPREPQ | |
| | VYTLPPSQEEMTKNQVSLTCLVKGFYPSDIAVEWESNGQPENN | |
| | YKTTPPVLDSDGSFFLYSRLTVDKSRWQEGNVFSCSVMHEALH | |
| | NHYTQKSLSLSLGK | |
| 5 | RWPESPKAQASSVPTAQPQAEGSLAKATTAPATTRNTGRGGEE | IgD-hinge-Fc Homo |
| | KKKEKEKEEQEERETKTPECPSHTQPLGVYLLTPAVQDLWLRD | sapiens |
| | KATFTCFVVGSDLKDAHLTWEVAGKVPTGGVEEGLLERHSNGS | |
| | QSQHSRLTLPRSLWNAGTSVTCTLNHPSLPPQRLMALREPAAQ | |
| | APVKLSLNLLASSDPPEAASWLLCEVSGFSPPNILLMWLEDQR | |
| | EVNTSGFAPARPPPQPGSTTFWAWSVLRVPAPPSPQPATYTCV | |
| | VSHEDSRTLLNASRSLEVSYVTDH | |
| 6 | LEGGGEGRGSLLTCGDVEENPGPR | T2A artificial |
| 7 | MLLLVTSLLLCELPHPAFLLIPRKVCNGIGIGEFKDSLSINAT | tEGFR artificial |
| | NIKHFKNCTSISGDLHILPVAFRGDSFTHTPPLDPQELDILKT | |
| | VKEITGFLLIQAWPENRTDLHAFENLEIIRGRTKQHGQFSLAV | |
| | VSLNITSLGLRSLKEISDGDVIISGNKNLCYANTINWKKLFGT | |
| | SGQKTKIISNRGENSCKATGQVCHALCSPEGCWGPEPRDCVSC | |
| | RNVSRGRECVDKCNLLEGEPREFVENSECIQCHPECLPQAMNI | |
| | TCTGRGPDNCIQCAHYIDGPHCVKTCPAGVMGENNTLVWKYAD | |
| | AGHVCHLCHPNCTYGCTGPGLEGCPTNGPKIPSIATGMVGALL | |
| | LLLVVALGIGLFM | |
| 8 | FWVLVVVGGVLACYSLLVTVAFIIFWV | CD28 (amino acids |
| | | 153-179 of Accession |
| | | No. P10747) Homo |
| | | sapiens |
| 9 | IEVMYPPPYLDNEKSNGTIIHVKGKHLCPSPLFPGPSKP | CD28 (amino acids |
| | FWVLVVVGGVLACYSLLVTVAFIIFWV | 114-179 of Accession |
| | | No. P10747) Homo |
| | | sapiens |
| 10 | RSKRSRLLHSDYMNMTPRRPGPTRKHYQPYAPPRDFAAYRS | CD28 (amino acids |
| | | 180-220 of P10747) |
| | | Homo sapiens |
| 11 | RSKRSRGGHSDYMNMTPRRPGPTRKHYQPYAPPRDFAAYRS | CD28 (LL to GG) |
| | | Homo sapiens |
| 12 | KRGRKKLLYIFKQPFMRPVQTTQEEDGCSCRFPEEEEGGCEL | 4-1BB (amino acids |
| | | 214-255 of |
| | | Q07011.1) Homo |
| | | sapiens |
| 13 | RVKFSRSADAPAYQQGQNQLYNELNLGRREEYDVLDKRRGRDP | CD3 zeta Homo |
| | EMGGKPRRKNPQEGLYNELQKDKMAEAYSEIGMKGERRRGKGH | sapiens |
| | DGLYQGLSTATKDTYDALHMQALPPR | |
| 14 | RVKFSRSAEPPAYQQGQNQLYNELNLGRREEYDVLDKRRGRDP | CD3 zeta Homo |
| | EMGGKPRRKNPQEGLYNELQKDKMAEAYSEIGMKGERRRGKGH | sapiens |
| | DGLYQGLSTATKDTYDALHMQALPPR | |
| 15 | RVKFSRSADAPAYKQGQNQLYNELNLGRREEYDVLDKRRGRDP | CD3 zeta Homo |
| | EMGGKPRRKNPQEGLYNELQKDKMAEAYSEIGMKGERRRGKGH | sapiens |
| | DGLYQGLSTATKDTYDALHMQALPPR | |
| 16 | RKVCNGIGIGEFKDSLSINATNIKHFKNCTSISGDLHILPVAF | tEGFR artificial |
| | RGDSFTHTPPLDPQELDILKTVKEITGFLLIQAWPENRTDLHA | |
| | FENLEIIRGRTKQHGQFSLAVVSLNITSLGLRSLKEISDGDVI | |
| | ISGNKNLCYANTINWKKLFGTSGQKTKIISNRGENSCKATGQV | |
| | CHALCSPEGCWGPEPRDCVSCRNVSRGRECVDKCNLLEGEPRE | |
| | FVENSECIQCHPECLPQAMNITCTGRGPDNCIQCAHYIDGPHC | |
| | VKTCPAGVMGENNTLVWKYADAGHVCHLCHPNCTYGCTGPGLE | |
| | GCPTNGPKIPSIATGMVGALLLLLVVALGIGLFM | |
| 17 | EGRGSLLTCGDVEENPGP | T2A artificial |
| 18 | GSGATNFSLLKQAGDVEENPGP | P2A |
| 19 | ATNFSLLKQAGDVEENPGP | P2A |
| 20 | QCTNYALLKLAGDVESNPGP | E2A |
| 21 | VKQTLNFDLLKLAGDVESNPGP | F2A |
| 22 | PGGG-(SGGGG)5-P- wherein P is proline, | linker |
| | G is glycine and S is serine | |
| 23 | GSADDAKKDAAKKDGKS | linker |
| 24 | atgcttctcctggtgacaagccttctgctctgtgagttaccac | GMC SFR alpha |
| | acccagcattcctcctgatccca | chain signal sequence |
| 25 | MLLLVTSLLLCELPHPAFLLIP | GMCSFR alpha |
| | | chain signal sequence |
| 26 | MALPVTALLLPLALLLHA | CD8 alpha signal |
| | | peptide |
| 27 | AGGACAGCC mGmGmG AAGGTGT | IgG constant RT |
| | | primer |
| 28 | GCTCCCGG mG T mAmG AAGTCA | IgL constant RT |
| | | primer |
| 29 | GGCCTCTCTG mGmGmA TAGAAGT | IgK constant RT |
| | | primer |
| 30 | TGTGAGGTGGCT mGmCmG TACTTG | IgM constant RT |
| | | primer |
| 31 | CTGGCTRGGTG mGmGmA AGTTTCT | IgA constant RT |
| | | primer |
| 32 | CACGCATTTGT mAmC T mC GCCTTG | IgD constant RT |
| | | primer |
| 33 | GATGGTGGC mA T mAmG TGACCAG | IgE constant RT |
| | | primer |
| 34 | TGTTTGAGAATCAA mAmA T mC GGTGAA | TRA constant RT |
| | | primer |
| 35 | ACGTGGTC mGmGmG GAAGAAG | TRB constant RT |
| | | primer |
| 36 | CAAGAAGACAAA mGmG T mA TGTTCC | TRG constant RT |
| | | primer |
| 37 | TCTTCTTGGAT mGmAmC ACGAGA | TRD constant RT |
| | | primer |
| 38 | AGGACAGCC mGmGmG AAGGTGT | IgG constant RT |
| | | primer |
| 39 | GCTCCCGG mG T mAmG AAGTCA | IgL constant RT |
| | | primer |
| 40 | GGCCTCTCTG mGmGmA TAGAAGT | IgK constant RT |
| | | primer |
| 41 | TGTGAGGTGGCT mGmCmG TACTTG | IgM constant RT |
| | | primer |
| 42 | CTGGCTRGGTG mGmGmA AGTTTCT | IgA constant RT |
| | | primer |
| 43 | CACGCATTTGT mAmC T mC GCCTTG | IgD constant RT |
| | | primer |
| 44 | GATGGTGGC mA T mAmG TGACCAG | IgE constant RT |
| | | primer |
| 45 | TGTTTGAGAATCAA mAmA T mC GGTGAA | TRA constant RT |
| | | primer |
| 46 | ACGTGGTC mGmGmG GAAGAAG | TRB constant RT |
| | | primer |
| 47 | CAAGAAGACAAA mGmG T mA TGTTCC | TRG constant RT |
| | | primer |
| 48 | TCTTCTTGGAT mGmAmC ACGAGA | TRD constant RT |
| | | primer |
| 49 | NNNNWISCNNNWISCNNN | Exemplary vessel |
| | | barcode |
| 50 | QQGNTLPYT | FMC63 LC-CDR3 |
| 51 | RASQDISKYLN | FMC63 CDR Ll |
| 52 | SRLHSGV | FMC63 CDR L2 |
| 53 | GNTLPYTFG | FMC63 CDR L3 |
| 54 | DYGVS | FMC63 CDR H1 |
| 55 | VIWGSETTYYNSALKS | FMC63 CDR H2 |
| 56 | YAMDYWG | FMC63 CDR H3 |
| 57 | EVKLQESGPGLVAPSQSLSVTCTVSGVSLPDYGVSWIRQPPRK | FMC63 VH |
| | GLEWLGVIWGSETTYYNSALKSRLTIIKDNSKSQVFLKMNSLQ | |
| | TDDTAIYYCAKHYYYGGSYAMDYWGQGTSVTVSS | |
| 58 | DIQMTQTTSSLSASLGDRVTISCRASQDISKYLNWYQQKPDGT | FMC63 VL |
| | VKLLIYHTSRLHSGVPSRFSGSGSGTDYSLTISNLEQEDIATY | |
| | FCQQGNTLPYTFGGGTKLEIT | |
| 59 | DIQMTQTTSSLSASLGDRVTISCRASQDISKYLNWYQQKPDGT | FMC63 scFy |
| | VKLLIYHTSRLHSGVPSRFSGSGSGTDYSLTISNLEQEDIATY | |
| | FCQQGNTLPYTFGGGTKLEITGSTSGSGKPGSGEGSTKGEVKL | |
| | QESGPGLVAPSQSLSVTCTVSGVSLPDYGVSWIRQPPRKGLEW | |
| | LGVIWGSETTYYNSALKSRLTIIKDNSKSQVFLKMNSLQTDDT | |
| | AIYYCAKHYYYGGSYAMDYWGQGTSVTVSS | |
| 60 | KASQNVGTNVA | SJ25C1 CDR L1 |
| 61 | SATYRNS | SJ25C1 CDR L2 |
| 62 | QQYNRYPYT | SJ25C1 CDR L3 |
| 63 | SYWMN | SJ25C1 CDR H1 |
| 64 | QIYPGDGDTNYNGKFKG | SJ25C1 CDR H2 |
| 65 | KTISSVVDFYFDY | SJ25C1 CDR H3 |
| 66 | EVKLQQSGAELVRPGSSVKISCKASGYAFSSYWMNWVKQRPGQ | SJ25C1 VH |
| | GLEWIGQIYPGDGDTNYNGKFKGQATLTADKSSSTAYMQLSGL | |
| | TSEDSAVYFCARKTISSVVDFYFDYWGQGTTVTVSS | |
| 67 | DIELTQSPKFMSTSVGDRVSVTCKASQNVGTNVAWYQQKPGQS | SJ25C1 VL |
| | PKPLIYSATYRNSGVPDRFTGSGSGTDFTLTITNVQSKDLADY | |
| | FCQQYNRYPYTSGGGTKLEIKR | |
| 68 | GGGGSGGGGSGGGGS | Linker |
| 69 | EVKLQQSGAELVRPGSSVKISCKASGYAFSSYWMNWVKQRPGQ | SJ25C1 scFv |
| | GLEWIGQIYPGDGDTNYNGKFKGQATLTADKSSSTAYMQLSGL | |
| | TSEDSAVYFCARKTISSVVDFYFDYWGQGTTVTVSSGGGGSGG | |
| | GGSGGGGSDIELTQSPKFMSTSVGDRVSVTCKASQNVGTNVAW | |
| | YQQKPGQSPKPLIYSATYRNSGVPDRFTGSGSGTDFTLTITNV | |
| | QSKDLADYFCQQYNRYPYTSGGGTKLEIKR | |
| 70 | HYYYGGSYAMDY | FMC63 HC-CDR3 |
| 71 | HTSRLHS | FMC63 LC-CDR2 |
| 72 | GSTSGSGKPGSGEGSTKG | Linker |
| 73 | gacatccagatgacccagaccacctccagcctgagcgccagcctgggcgaccgggtgaccatcag | Sequence encoding |
| | ctgccgggccagccaggacatcagcaagtacctgaactggtatcagcagaagcccgacggcaccg | scFv |
| | tcaagctgctgatctaccacaccagccggctgcacagcggcgtgcccagccggtttagcggcagc | |
| | ggctccggcaccgactacagcctgaccatctccaacctggaacaggaagatatcgccacctactt | |
| | ttgccagcagggcaacacactgccctacacctttggcggcggaacaaagctggaaatcaccggca | |
| | gcacctccggcagcggcaagcctggcagcggcgagggcagcaccaagggcgaggtgaagctgcag | |
| | gaaagcggccctggcctggtggcccccagccagagcctgagcgtgacctgcaccgtgagcggcgt | |
| | gagcctgcccgactacggcgtgagctggatccggcagccccccaggaagggcctggaatggctgg | |
| | gcgtgatctggggcagcgagaccacctactacaacagcgccctgaagagccggctgaccatcatc | |
| | aaggacaacagcaagagccaggtgttcctgaagatgaacagcctgcagaccgacgacaccgccat | |
| | ctactactgcgccaagcactactactacggcggcagctacgccatggactactggggccagggca | |
| | ccagcgtgaccgtgagcagc | |
| 74 | X1PPX2P | Hinge |
| | X1 is glycine, cysteine or arginine | |
| | X2 is cysteine or threonine | |
| 75 | Glu Pro Lys Ser Cys Asp Lys Thr His Thr Cys Pro Pro Cys Pro | Hinge |
| 76 | Glu Arg Lys Cys Cys Val Glu Cys Pro Pro Cys Pro | Hinge |
| 77 | ELKTPLGDTHTCPRCPEPKSCDTPPPCPRCPEPKSCDTPPPCPRCP | Hinge |
| | EPKSCDTPPPCPRCP | |
| 78 | Glu Ser Lys Tyr Gly Pro Pro Cys Pro Ser Cys Pro | Hinge |
| 79 | Glu Ser Lys Tyr Gly Pro Pro Cys Pro Pro Cys Pro | Hinge |
| 80 | Tyr Gly Pro Pro Cys Pro Pro Cys Pro | Hinge |
| 81 | Lys Tyr Gly Pro Pro Cys Pro Pro Cys Pro | Hinge |
| 82 | Glu Val Val Val Lys Tyr Gly Pro Pro Cys Pro Pro Cys Pro | Hinge |

1. A method for identifying a property or attribute of a cell, the method comprising:
(a) identifying the clonotype and/or a TCR sequence of all of a portion of a native TCR alpha and/or beta variable region or pair thereof, of at least one T cell from at least one test biological sample from a subject, said test biological sample obtained from the subject following administration of a cell therapy comprising T cells expressing a recombinant receptor, wherein the T cell in the test biological sample is genetically engineered with and/or expresses the recombinant receptor;
(b) identifying, from a T cell composition, a cell that has the same clonotype or the same TCR sequence as the at least one T cell identified in (a), thereby identifying an originator T cell, wherein the T cell composition comprises T cells that are, or have been derived from, T cells previously obtained from the subject prior to administering the cell therapy to the subject; and
(c) determining at least one property or attribute of the originator T cell.

2. A method for identifying a property or attribute of a cell, the method comprising:
(a) identifying the clonotype and/or a TCR sequence of all of a portion of a native TCR alpha and/or beta variable region or pair thereof, of at least one T cell from at least one test biological sample from a subject, said test biological sample obtained from the subject following administration of a cell therapy comprising T cells expressing a recombinant receptor, wherein the T cell in the test biological sample is genetically engineered with and/or expresses the recombinant receptor;
(b) determining at least one property or attribute of a cell, from a T cell composition, that has the same clonotype or the same TCR sequence as the at least one T cell identified in (a), wherein the T cell composition comprises T cells that are, or have been derived from, T cells previously obtained from the subject prior to administering the cell therapy to the subject.

3. A method for identifying a property or attribute of a cell, the method comprising:
(a) identifying the clonotype and/or a TCR sequence of all or a portion of a native TCR alpha and/or beta variable region or pair thereof of one or more T cell genetically engineered with a recombinant receptor in at least one test biological sample from a subject, wherein said clonotype is known to be, determined to be, or suspected of being present in a cell in a T cell composition, thereby identifying one or more originator T cell, wherein:
the at least one test biological sample is obtained from the subject following administration of a cell therapy comprising T cells expressing the recombinant receptor; and
the T cell composition comprises T cells that are or are derived from cells of a sample obtained from the subject prior to administering the cell therapy to the subject; and
(b) determining at least one or property or attribute of the one or more originator T cell.

4. The method of any of claims 1-3, wherein the one or more clonotype and/or TCR sequence that is identified is present in the test biological sample at the same or increased frequency or relative frequency as in the T cell composition.

5. A method for identifying a property or attribute of a cell, the method comprising:
(a) identifying one or more clonotypes and/or one or more TCR sequences of all or a portion of a native TCR alpha and/or beta variable region or pair thereof that are the same in a plurality of samples, said plurality of samples selected from one or more compositions at different stages of a cell engineering process for generating a T cell therapy and/or a biological sample from a subject following administration of the T cell therapy to the subject, said T cell therapy comprising T cells expressing the recombinant receptor, thereby identifying an originator T cell; and
(b) determining at least one property or attribute of the originator T cell.

6. The method of claim 5, wherein at least one of the plurality of samples is a T cell composition at a stage of a cell engineering process, said T cell composition comprising T cells that are, or have been derived from, T cells previously obtained from the subject prior to administering the cell therapy to the subject.

7. The method of claim 5 or claim 6, wherein at least one of the plurality of samples is a test biological sample, said test biological sample obtained from the subject following administration of a cell therapy comprising T cells expressing a recombinant receptor.

8. The method of any of claims 1-4 and 7, where the test biological sample is obtained from the subject at or about or within 1 days, 3 days, 6 days, 9 days, 12 days, 15 days, 18 days, 21 days, 24 days, 27 days or 30 days, optionally at or about 12 days, 12 days, 14 days, 15 days, 16 days, 17 days, 18 days, 19 days, 20 days, 21 days, 22 days, 23 days, 24 days, 25 days, 26 days, 27 days, 28 days or 29 days following the administration of the cell therapy comprising the T cells expressing the recombinant receptor.

9. The method of any of claims 1-4, 7 and 8, wherein the test biological sample is obtained from a subject at a time after the subject exhibits a response to the cell therapy following the administration, said response selected from a complete response (CR), progression free survival (PFS) or a partial response (PR).

10. The method of claim 9, wherein the response is durable in the subject for at least at least 3 months, at least 6 months, at least 9 months or at least 12 months, and the test biological sample is obtained from the subject at a time when the response is still durable in the subject.

11. The method of 1-10, wherein the genetically engineered T cell in the at least one test biological sample or the plurality of samples exhibits a predetermined phenotype, function or parameter.

12. The method of claim 11, wherein the predetermined phenotype, function or attribute is an effector function associated with T cell activation state, is a cell surface phenotype or is a pharmacokinetic activity.

13. The method of claim 12, wherein the predetermined phenotype, function or attribute is a pharmacokinetic activity and the pharmacokinetic activity comprises the number or relative number of recombinant receptor-expressing T cells in the sample.

14. The method of claim 13, wherein the test biological sample is obtained from the subject at a time at or immediately after a peak T cells expressing the recombinant receptor are detectable in the blood of the subject.

15. The method of claim 14, wherein the predetermined phenotype, function or attribute is a cell surface phenotype and the cell surface phenotype is a naive phenotype or a long-lived memory phenotype.

16. The method of claim 15, wherein the cell surface phenotype comprises a phenotype surface negative for CD56 or CD45RO and/or a surface positive for CD27, CD45RA, or CCR7.

17. The method of claim 15 or claim 16, wherein the cell surface phenotype is of one or both of CD27 and CCR7.

18. The method of any of claims 1-17, wherein the at least one T cell from the at least one test biological sample or the plurality of samples is selected or isolated from a biological sample from the subject based on the predetermined phenotype, function, or attribute.

19. The method of any of claims 1-18, wherein the at least one T cell from the at least one test biological sample or the plurality of samples is positive for or expresses the recombinant receptor, optionally is surface positive for the recombinant receptor.

20. The method of claim 19, wherein the at least one T cell that is positive for or expresses the recombinant receptor is selected or isolated from a biological sample from a subject.

21. The method of any of claims 1-20 that is repeated for a plurality of subjects.

22. The method of claim 21, further comprising identifying the at least one property or parameter of originator T cells that is present in a T cell composition from a majority of subjects.

23. The method of any of claims 1-22, wherein the at least one property or parameter is identified as an attribute of a T cell composition that is predicted to increase likelihood or a desired phenotype, property or attribute of a cell therapy following administration to a subject.

24. The method of any of claims 1-23, wherein the T cell composition is an input composition that does not comprise T cells genetically engineered with the recombinant receptor.

25. The method of claim 24, wherein the input composition is obtained by isolating a population of cells comprising the T cells from a biological sample.

26. The method of any of claims 1-25, wherein the T cell composition is an output composition comprising T cells genetically engineered with the recombinant receptor.

27. The method of claim 26, wherein the output composition is the cell therapy that had been administered to the subject in (a).

28. The method of claim 26 or claim 27, wherein the output composition is produced by a process comprising:
(i) incubating an input composition comprising T cells with an agent comprising a nucleic acid molecule encoding the recombinant receptor under conditions to introduce the nucleic acid encoding the recombinant receptor into cells in the composition; and
(ii) stimulating the T cells, prior to, during and/or subsequent to said incubation, wherein stimulating comprises incubating the cells in the presence of a stimulating condition that induces a primary signal, signaling, stimulation, activation and/or expansion of the T cells.

29. The method of claim 28, wherein the process further comprises, prior to (i), isolating a population comprising the T cells from a biological sample.

30. The method of claim 25 or claim 29, wherein the isolating comprises, selecting the T cells from the biological sample based on surface expression of CD3 or based on surface expression of one or both of CD4 and CD8, optionally by positive or negative selection.

31. The method of claim 25, 29 or 30, wherein the isolating comprises carrying out immunoaffinity-based selection.

32. The method of any of claims 25 and 29-31, wherein the biological sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product.

33. The method of any of claims 28-32, wherein the stimulating condition comprises incubation with a stimulatory reagent capable of activating one or more intracellular signaling domains of one or more components of a TCR complex and/or one or more intracellular signaling domains of one or more costimulatory molecules.

34. The method of claim 33, wherein the stimulatory reagent comprises a primary agent that specifically binds to a member of a TCR complex and a secondary agent that specifically binds to a T cell costimulatory molecule.

35. The method of claim 34, wherein the primary agent specifically binds to CD3 and/or the costimulatory molecule is selected from the group consisting of CD28, CD137 (4-1-BB), OX40, or ICOS.

36. The method of claim 34 or claim 35, wherein the primary and secondary agents comprise antibodies, optionally an anti-CD3 antibody and an anti-CD28 antibody.

37. The method of any of claims 34-36, wherein the primary and secondary agent are present on the surface of a solid support, optionally a bead.

38. The method of any of claims 28-37, wherein the stimulating of the T cells is carried out or is initiated prior to the incubating, optionally for 18-24 hours at or about 37 degrees Celsius, wherein the T cells have not been introduced with the nucleic acid encoding the recombinant receptor.

39. The method of any of claims 28-38, wherein the stimulating condition comprises a cytokine selected from among IL-2, IL-15 and IL-7.

40. The method of any of claims 28-39, wherein the stimulating of the T cells is carried out subsequent to the incubating, said T cells comprising cells introduced with the nucleic acid encoding the recombinant receptor.

41. The method of claim 40, wherein the stimulating the cells is carried out under conditions to cultivate or expand T cells introduced optionally for a period of time to achieve a threshold concentration.

42. The method of any of claims 28-40, wherein the agent comprising a nucleic acid molecule encoding the recombinant receptor is a viral vector, optionally a lentiviral vector or a gamma retroviral vector.

43. The method of any of claims 28-42, wherein:
the incubating and/or stimulating is carried out in the presence of one or more test agents or conditions; or
the process further comprises culturing the input composition and/or stimulated cells in the presence of one or more test agents or conditions.

44. The method of claim 43, wherein the one or more test agents or conditions comprises presence or concentration of serum; time in culture; presence or amount of a stimulating agent; the type or extent of a stimulating agent; presence or amount of amino acids;
temperature; the source or cell types of the input composition; the ratio or percentage of cell types in the input composition, optionally the CD4+/CD8+ cell ratio; the presence or amount of beads; cell density; static culture; rocking culture; perfusion; the type of viral vector; the vector copy number; the presence of a transduction adjuvant; cell density of the input composition in cryopreservation; the extent of expression of the recombinant receptor; or the presence of a compound to modulate cell phenotype.

45. The method of claim 43 or claim 44, wherein the one or more test agents or conditions comprises one or more compounds from a library of test compounds.

46. The method of any of claims 1-45, wherein the test biological sample is a serum, blood or plasma sample.

47. The method of any of claims 1-46, wherein the test biological sample is or comprises a tumor sample.

48. The method of any of claims 1-47, wherein the test biological sample is obtained from the subject greater than or greater than about 7 days, 10 days, 14 days, 21 days, 28 days, 2 months, 3 months, 4 months, 5 months, 6 months, 1 year or 2 years or more after initiation of administration of the cell therapy.

49. The method of any of claims 1-48, wherein the test biological sample is obtained from the subject greater than or greater than about 28 days after initiation of administration of the cell therapy, optionally at or about at day 29 or greater after initiation of administration of the cell therapy.

50. The method of any of claims 1-49, wherein the at least one test biological sample comprises a plurality of test biological samples, optionally at least 2, 3, 4, 5, 6, 7, 8, 9, 10 or more test biological samples.

51. The method of claim 50, wherein each of the plurality of test biological samples is obtained from the subject on different days after initiation of administration of the cell therapy, optionally on consecutive days, every other day, every third day, or once a week for a predetermined time after initiation of administration of the cell therapy.

52. The method of claim 51, wherein the identified clonotype is one whose frequency or relative frequency is retained or increased among the plurality of assessed tested biological samples over the predetermined period of time.

53. The method of any of claims 1-52, wherein the clonotype in a) and/or b) is determined by high-throughput single cell immune sequencing of nucleic acid encoding natively paired TCR chains.

54. The method of claim 53, wherein the natively paired TCR chains comprise TCR α-β or TCR γ-δ pairs.

55. The method of any of claims 1-54, wherein the test biological sample comprises a plurality of T cells and the one or more clonotype is identified simultaneously or from a single reaction.

56. The method of any of claims 1-55, wherein the T cell composition comprises a plurality of T cells and the one or more clonotype is identified from a single reaction.

57. The method of any of claims 1-56, wherein the at least one property or parameter is determined by single cell gene expression profiling and/or single cell surface phenotyping.

58. The method of claim 57, wherein the at least one property or parameter is determined by single cell gene expression profiling, wherein the single cell gene expression profiling is of at least one gene product or is of the whole-transcriptome or a portion thereof.

59. The method of claim 58, wherein the at least one gene product is selected from CD4, ICOS, FOXP3, FOXP3V1, PMCH, CD80, FOXP3Y, CD86, CD70, CD40, IL-6, CD2, CD3D, GPR171, CXCL13, PD-1 (CD279), IL-2, IL-4, IL-10, CD8B, KLRK1, CCL4, RUNX3V1, RUNX3, NKG7, CD45RA, CD45RO, CD62L, CD69, CD25, CCR7, CD27, CD28, CD56, CD122, CD127, CD95, CXCR3, LFA-1, KLRG1, T-bet, CD8, IL-7Rα, IL-2Rβ, CD3, CD14, ROR1, granzyme B, granzyme H, CD20, CD11b, CD16, HLA-DR, PD-L1, IFNγ, KIRK1, caspase 2, caspase 3, caspase 6, caspase 7, caspase 8, caspase 9, caspase 10, Bcl-2, Bax, Bad, Bid, CD196 (CCR6), CTLA-4 (CD152), TIGIT (VSIG9, VSTM3), LAG-3 (CD223), 2B4 (CD244), BTLA (CD272), TIM3 (HAVCR2), VISTA (PD1-H) and CD96.

60. The method of claim 59, wherein the at least one property or parameter is determined by single cell surface phenotyping of at least one T cell surface marker.

61. The method of claim 60, wherein the at least one T cell surface marker is selected from CD4, CD8, CD45RA, CD45RO, CD62L, CD69, CD25, CCR7, CD27, CD28, CD56, CD122, CD127, T-bet, IL-7Rα, CD95, IL-2Rβ, CXCR3, LFA-1 or KLRG1.

62. The method of any of claims 53-61, wherein the single cell gene expression profiling or single cell surface phenotyping is coupled to or carried out in the same reaction as the single cell immune sequencing.

63. The method of any of claims 1-62, wherein the recombinant receptor is or comprises a chimeric receptor.

64. The method of claim 63, wherein the chimeric receptor is capable of binding to a target antigen that is associated with, specific to, and/or expressed on a cell or tissue of a disease, disorder or condition.

65. The method of claim 64, wherein the disease, disorder or condition is an infectious disease or disorder, an autoimmune disease, an inflammatory disease, or a tumor or a cancer.

66. The method of claim 64 or claim 65, wherein the target antigen is a tumor antigen.

67. The method of any of claims 64-66, wherein the target antigen is selected from among ROR1, B cell maturation antigen (BCMA), carbonic anhydrase 9 (CAIX), Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRLS, FCRHS, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-Al, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2 NY-ESO-1, PSCA, folate receptor-a, CD44v6, CD44v7/8, avb6 integrin, 8H9, NCAM, VEGF receptors, 5T4, Fetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag.

68. The method of any of claims 64-66, wherein the target antigen is selected from among αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD133, CD138, CD171, chondroitin sulfate proteoglycan 4 (CSPG4), epidermal growth factor protein (EGFR), type III epidermal growth factor receptor mutation (EGFR vIII), epithelial glycoprotein 2 (EPG-2), epithelial glycoprotein 40 (EPG-40), ephrinB2, ephrin receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), glypican-3 (GPC3), G Protein Coupled Receptor 5D (GPRC5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Rα), IL-13 receptor alpha 2 (IL-13Rα2), kinase insert domain receptor (kdr), kappa light chain, L1 cell adhesion molecule (L1-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, MAGE-A10, mesothelin (MSLN), c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), Tyrosinase related protein 1 (TRP1, also known as TYRP1 or gp75), Tyrosinase related protein 2 (TRP2, also known as dopachrome tautomerase, dopachrome delta-isomerase or DCT), vascular endothelial growth factor receptor (VEGFR), vascular endothelial growth factor receptor 2 (VEGFR2), Wilms Tumor 1 (WT-1), a pathogen-specific or pathogen-expressed antigen, or an antigen associated with a universal tag, and/or biotinylated molecules, and/or molecules expressed by HIV, HCV, HBV or other pathogens.

69. The method of any of claims 63-68, wherein the chimeric receptor is a chimeric antigen receptor (CAR).

70. The method of any of claims 63-69, wherein the chimeric receptor comprises an extracellular domain comprising an antigen-binding domain that specifically binds a target antigen and an intracellular signaling domain comprising an ITAM.

71. The method of claim 70, wherein the antigen-binding domain is or comprises an antibody or an antibody fragment thereof, which optionally is a single chain fragment.

72. The method of claim 71, wherein the fragment comprises antibody variable regions joined by a flexible linker.

73. The method of claim 71 or claim 72, wherein the fragment comprises an scFv.

74. The method of any of claims 63-73, wherein the chimeric receptor further comprises a spacer and/or a hinge region.

75. The method of any of claims 63-74, wherein chimeric receptor comprises an intracellular signaling region.

76. The method of claim 75, wherein the intracellular signaling region comprises an intracellular signaling domain.

77. The method of claim 76, wherein the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

78. The method of claim 77, wherein the intracellular signaling domain is or comprises an intracellular signaling domain of a CD3 chain, optionally a CD3-zeta (CD3ζ) chain, or a signaling portion thereof.

79. The method of any of claims 75-78, wherein chimeric receptor further comprises a transmembrane domain disposed between the extracellular domain and the intracellular signaling region.

80. The method of any of claims 75-79, wherein the intracellular signaling region further comprises a costimulatory signaling region.

81. The method of claim 80, wherein the costimulatory signaling region comprises an intracellular signaling domain of a T cell costimulatory molecule or a signaling portion thereof.

82. The method of claim 80 or claim 81, wherein the costimulatory signaling region comprises an intracellular signaling domain of a CD28, a 4-1BB or an ICOS or a signaling portion thereof.

83. The method of any of claims 69-82, wherein the CAR comprises an extracellular antigen-recognition domain that specifically binds to the antigen and an intracellular signaling domain comprising an ITAM, wherein optionally, the intracellular signaling domain comprises an intracellular domain of a CD3-zeta (CD3ζ) chain; and/or wherein the CAR further comprises a costimulatory signaling region, which optionally comprises a signaling domain of CD28 or 4-1BB.

84. The method of any of claims 1-83, wherein the T cell composition and/or cell therapy comprises CD4 and/or CD8 T cells.

85. The method of any of claims 1-84, wherein the clonotype comprises the TCR sequences of all or a portion of a native TCR alpha and/or beta variable region or pair thereof.

86. The method of any of claims 1-85, wherein the clonotype and/or TCR sequence is of a T cell genetically engineered with or expressing the recombinant receptor.

87. The method of any of claims 1-86, wherein the clonotype and/or TCR sequence is of a CD8+ T cell.
